SEQUENCE LISTING (6 sequences)

SEQ ID NO: 1 - DNA, 24 bp, Artificial Sequence
  Forward primer for hBD-2
gatgcctcttccaggtgtttttgg

SEQ ID NO: 2 - DNA, 20 bp, Artificial Sequence
  Reverse primer for hBD-2
ttgttccaggaccacaggtg

SEQ ID NO: 3 - DNA, 28 bp, Artificial Sequence
  Forward primer for IL-8
gcagctctgtgtgaaggtgcagttttgc

SEQ ID NO: 4 - DNA, 25 bp, Artificial Sequence
  Reverse primer for IL-8
tttctgtgttggcgcagtgtggtcc

SEQ ID NO: 5 - DNA, 19 bp, Artificial Sequence
  Forward primer for b-2-microgloublin(control)
ctccgtggccttagctgtg

SEQ ID NO: 6 - DNA, 21 bp, Artificial Sequence
  Reverse primer for b-2-microgloublin(control)
ttggagtacgctggatagcct

(19) United States
(12) Patent Application Publication
(10) Pub. No.: US 2012/0039945 A1
(43) Pub. Date: Feb. 16, 2012

(54) Methods Of Immune Modulation

(75) Inventors: Richard W. Scott, Radnor, PA (US); Gill Diamond, Newark, NJ (US)

(73) Assignees: UNIVERSITY OF MEDICINE AND DENTISTRY OF NEW JERSEY, Somerset, NJ (US); POLYMEDIX, INC., Radnor, PA (US)

(21) Appl. No.: 13/177,056

(22) Filed: Jul. 6, 2011

Related U.S. Application Data
(60) Provisional application No. 61/362,088, filed on Jul. 7, 2010.

(51) Int. Cl. A61K 31/167; A61P 31/04; A61K 31/197; A61P 37/02; A61K 31/166; A61K 31/505

(52) U.S. Cl. 424/278.1

(57) ABSTRACT

The present invention provides compounds and compositions thereof that modulate the immune system.

### CROSS-REFERENCE TO RELATED APPLICATIONS

This application claims priority to U.S. provisional application Ser. No. 61/362,088 filed Jul. 7, 2010, which is incorporated herein by reference in its entirety.

### REFERENCE TO GOVERNMENT GRANTS

The present invention was supported by funds from the U.S. Government (U.S. Public Health Service grant R43 DE18371) and the U.S. Government may therefore have certain rights in the invention.

### FIELD OF THE INVENTION

The present invention is directed, in part, to methods of modulating an immune response in an animal.

### BACKGROUND OF THE INVENTION

Periodontitis is the most common cause of tooth loss in adults in the United States (Borrell et al., J. Dent. Res., 2005, 84, 924-930), occurring in 15-25% of the US population. Its etiology can be considered due to bacterial colonization by a variety of pathogenic microorganisms, including Porphyromonas gingivalis, which is associated with chronic periodontitis, and Aggregatibacter actinomycetemcomitans, which is associated with aggressive periodontitis. This colonization and subsequent invasion into the gingival epithelium leads to an innate immune response, including the production of such mediators as IL-1 and tumor necrosis factor (TNF)-α (Graves et al., J. Periodontol., 2003, 74, 391-401). This leads to inflammation, which ultimately results in the bone loss seen in this disease (reviewed in Cochran, J. Periodontol., 2008, 79, 1569-1576). While standard treatment involves mechanical removal of the biofilm, the use of systemic antibiotics has also been examined (reviewed in Herrera et al., J. Clin. Periodontol., 2008, 35, 45-66), as has the identification of therapeutic targets in the inflammatory response (reviewed in Kirkwood et al., Periodontol. 2000, 2007, 43, 294-315). While periodontal disease is ultimately of bacterial etiology, from multispecies biofilms of Gram-negative anaerobic microorganisms, much of the deleterious effects are due to the resultant epithelial inflammatory response. Thus, development of a treatment that combines both anti-biofilm antibiotic activity with anti-inflammatory activity would be of great utility. While development of new antibiotics can temporarily address the bacterial colonization, the increase in antibiotic-resistant organisms makes this approach less effective.

Antimicrobial peptides (AMPs) such as defensins are naturally occurring peptides that exhibit broad-spectrum activity as well as a variety of immunomodulatory activities. Naturally occurring antimicrobial peptides have been proposed as a novel alternative to standard antibiotics, as they exhibit broad-spectrum activity, with little development of antibiotic resistance. However, their development as exogenous antibiotics has been hampered by a variety of factors, including their difficulty in large-scale production, poor tissue distribution and systemic toxicity. Small-molecule mimetics of these AMPs exhibit similar activities as the parent peptides, in addition to low toxicity, high stability and low cost. The development of small molecule antimicrobial peptide mimetics has provided a novel direction for the development of new antibiotics (reviewed in Som et al., Biopolymers, 2008, 90, 83-93). We recently demonstrated the potent activity of one such compound, mPE, a mimetic whose design was based on the amphiphilic structure of the peptide magainin (Beckloff et al., Antimicrob. Agents Chemother., 2007, 51, 4125-4132). This compound was active against numerous oral pathogens, both Gram-positive and -negative, including biofilm cultures of Streptococcus mutans. It also inhibited LPS-mediated induction of TNF-α from a macrophage cell line, presumably due to its predicted binding of LPS.

To determine whether AMP mimetics have potential as immune modulators and diseases and conditions related thereto, such as treatment of periodontal disease, the activity of one mimetic, mPE, against biofilm cultures of Aggregatibacter actinomycetemcomitans and Porphyromonas gingivalis was determined. Metabolic assays as well as culture and biomass measurement assays demonstrated that mPE exhibits potent activity against biofilm cultures of both species. Furthermore, as little as 2 μg/ml mPE was sufficient to inhibit IL-1β-induced secretion of IL-8 in both gingival epithelial cells and THP-1 cells. This anti-inflammatory activity is associated with a reduction in activation of NF-κB, suggesting that mPE can act both as an anti-biofilm agent in an anaerobic environment as well as an anti-inflammatory agent in infected tissues. The ability to suppress the inflammatory response of epithelial and myeloid-derived cells was also studied.

### SUMMARY OF THE INVENTION

The present invention provides methods of modulating an immune response in a mammal comprising administering to the mammal in need thereof a therapeutically effective amount of a compound of Formula I:

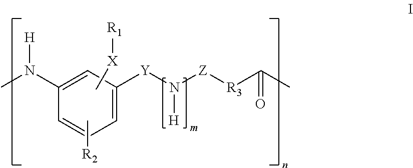

or a pharmaceutically acceptable salt thereof,
wherein:

X is O or S;

R₁ is C₁-C₉ straight or branched chain alkyl, optionally substituted with one or more —NH₂ or —NH—C(═NH)NH₂;

Y is a bond or a carbonyl;

Z is a bond or a carbonyl;

R₂ is hydrogen or C₁-C₉ straight or branched chain alkyl optionally substituted with one or more —NH, or —NH—C(═NH)NH₂;

or R₂ is —X—R₁;

R₃ is methylene or

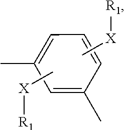

wherein the methylene is substituted with C₁-C₉ straight or branched chain alkyl, wherein the C₁-C₉ straight or branched chain alkyl is optionally substituted with one or more —NH₂ or —NH—C(═NH)NH₂;

n is 2-10; and

m is 1 or 2.

The present invention also provides methods of modulating an immune response in a mammal comprising administering to the mammal in need thereof a therapeutically effective amount of a compound of Formula II:

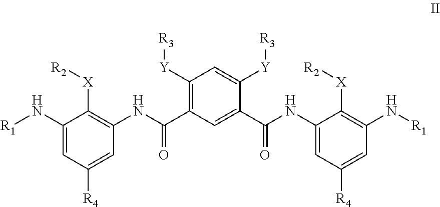

or a pharmaceutically acceptable salt thereof,
wherein:

X is O or S;

Y is O or S;

R₁ is H or —C(═O)-A, where A is C₁-C₉ straight or branched alkyl optionally substituted with one or more —NH₂, —N(CH₃)₂ or —NH—C(═NH)NH₂;

R₂ is C₁-C₉ straight or branched alkyl optionally substituted with one or more —NH₂, —N(CH₃)₂ or —NH—C(═NH)NH₂;

R₃ is C₁-C₉ straight or branched alkyl optionally substituted with one or more —NH₂, —N(CH₃)₂ or —NH—C(═NH)NH₂; and

R₄ is H, —B, or —C(═O)—O—B, where B is C₁-C₉ straight or branched alkyl.

The present invention also provides methods of modulating an immune response in a mammal comprising administering to the mammal in need thereof a therapeutically effective amount of a compound chosen from:

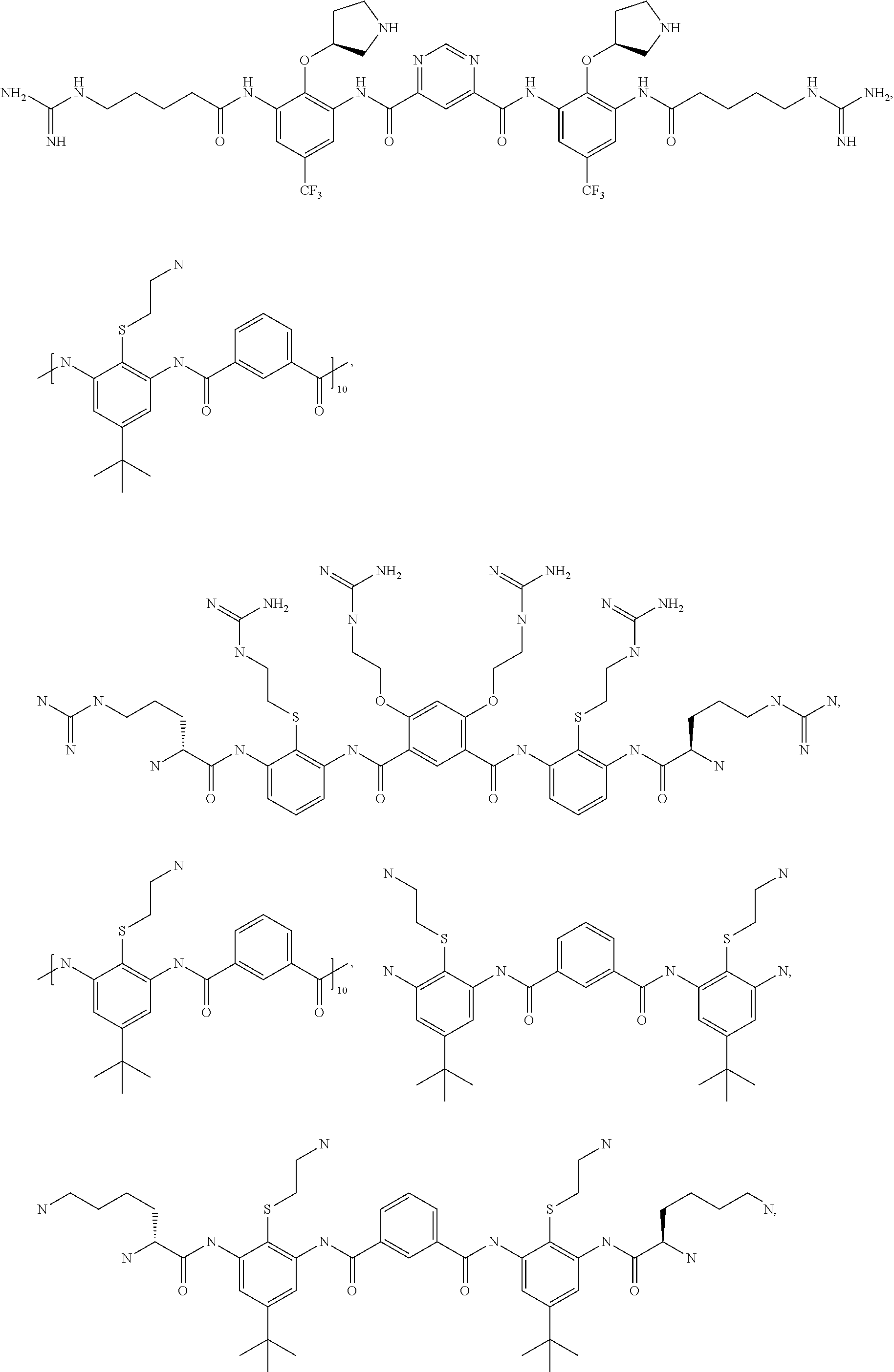

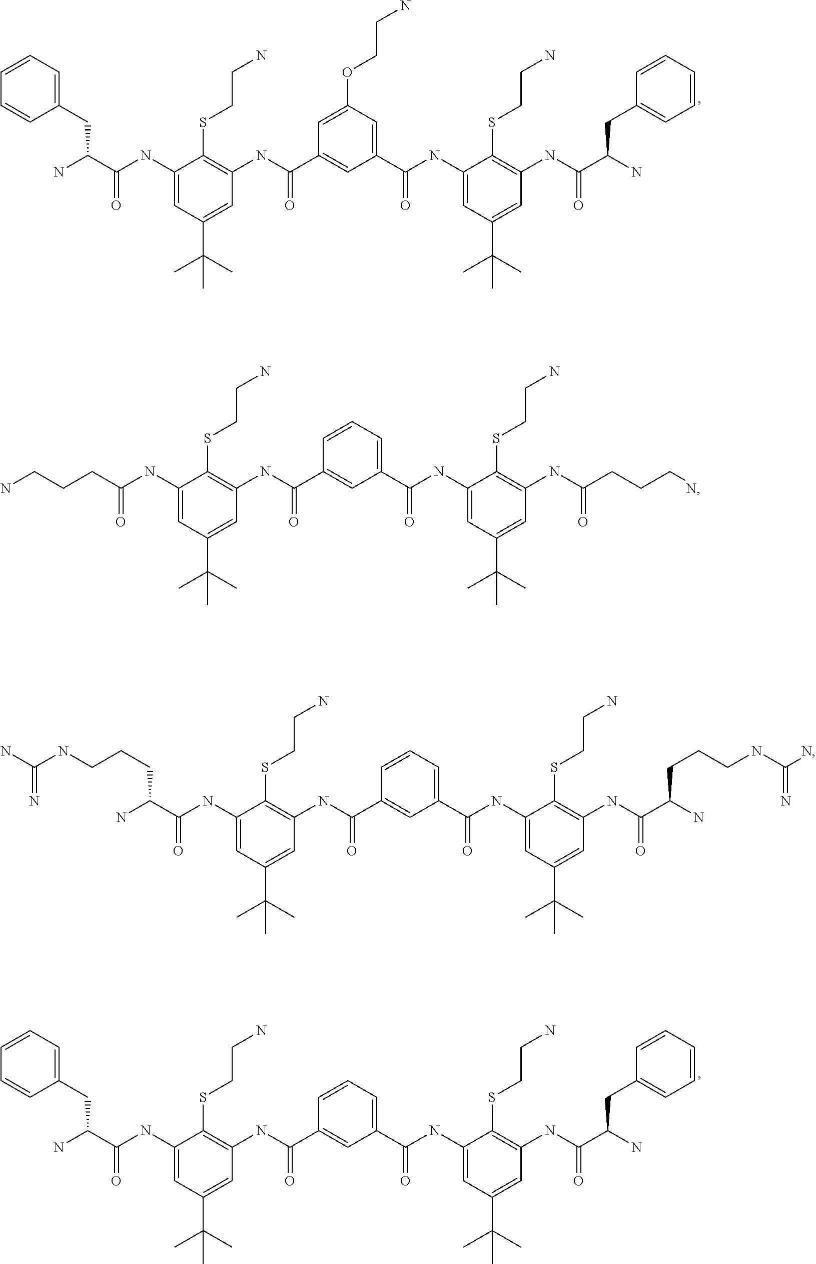

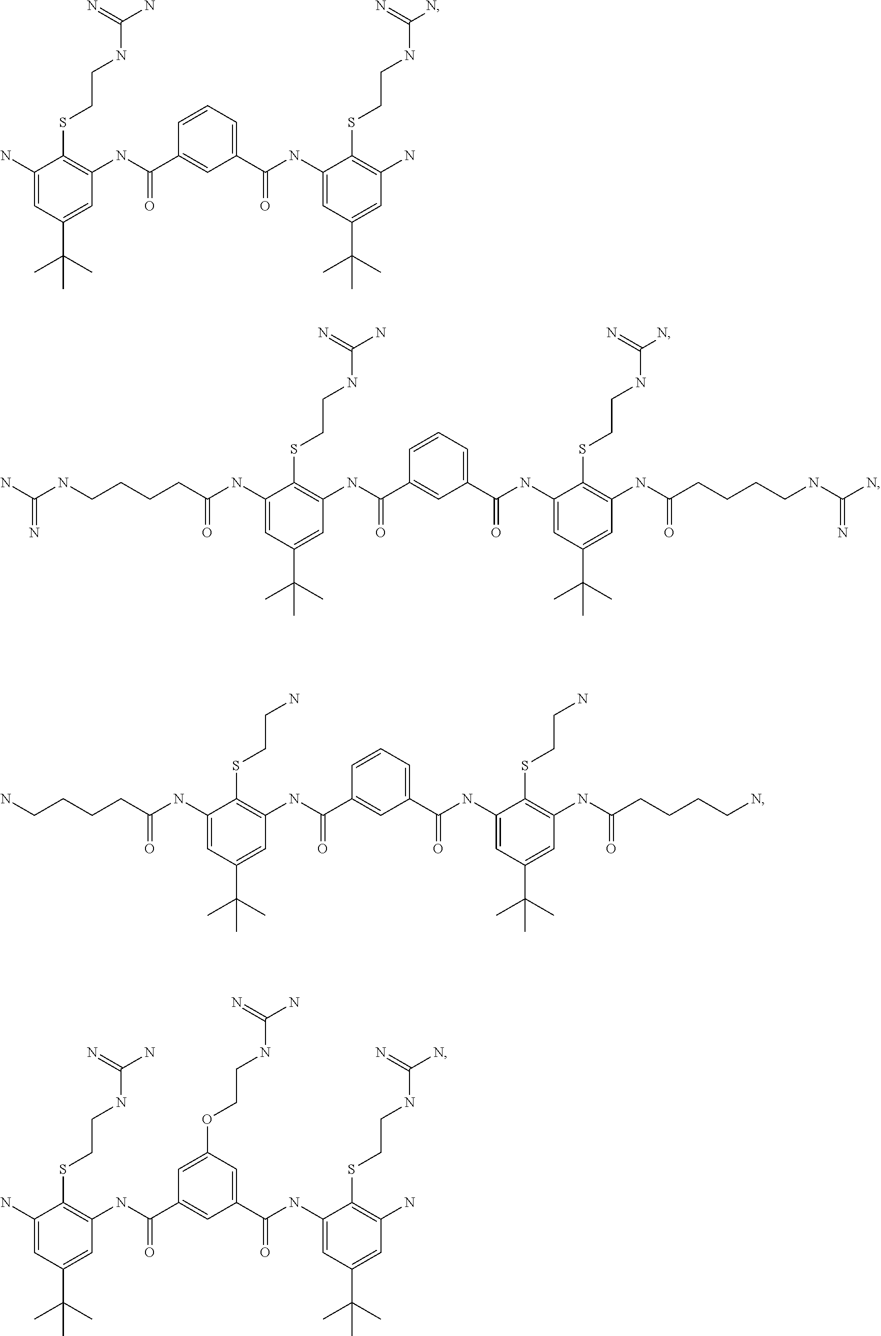

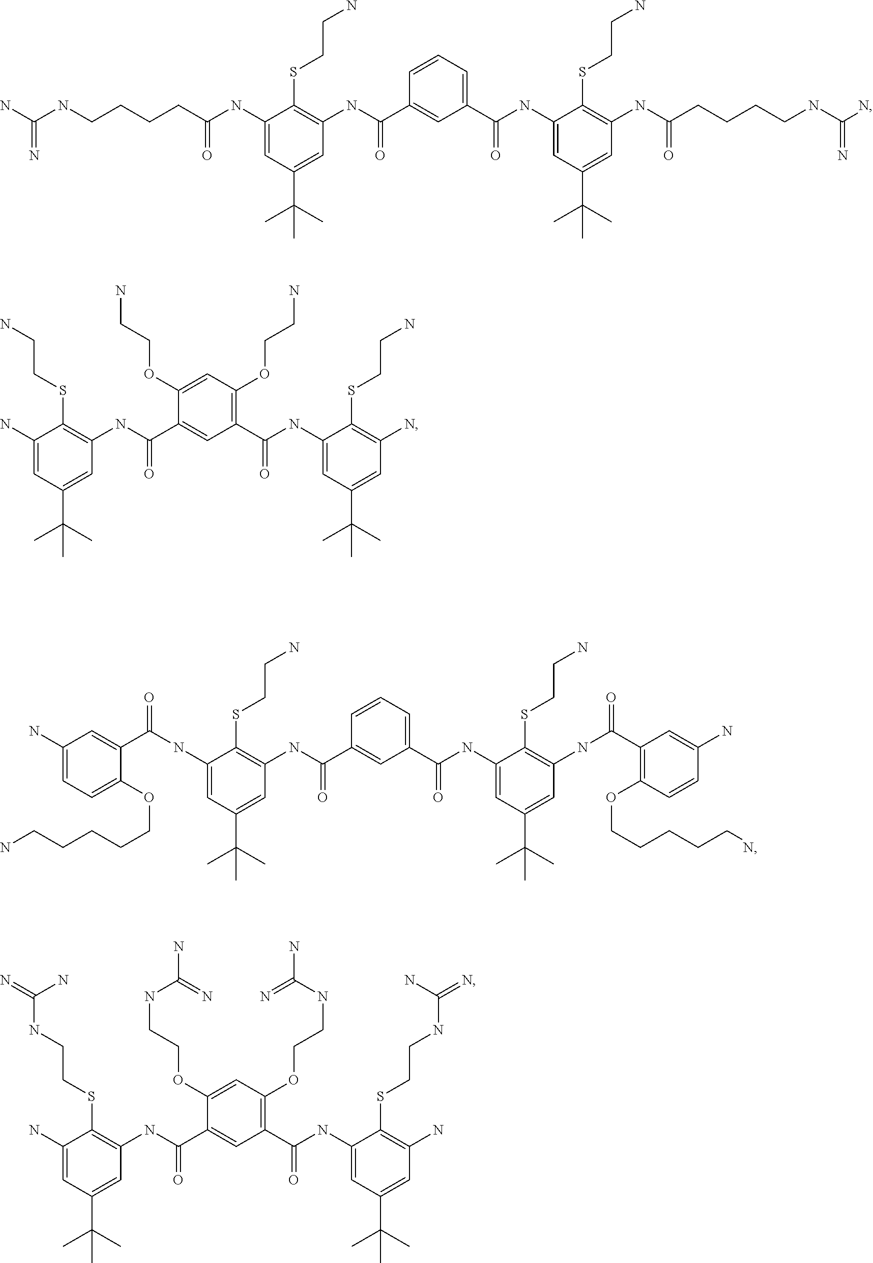

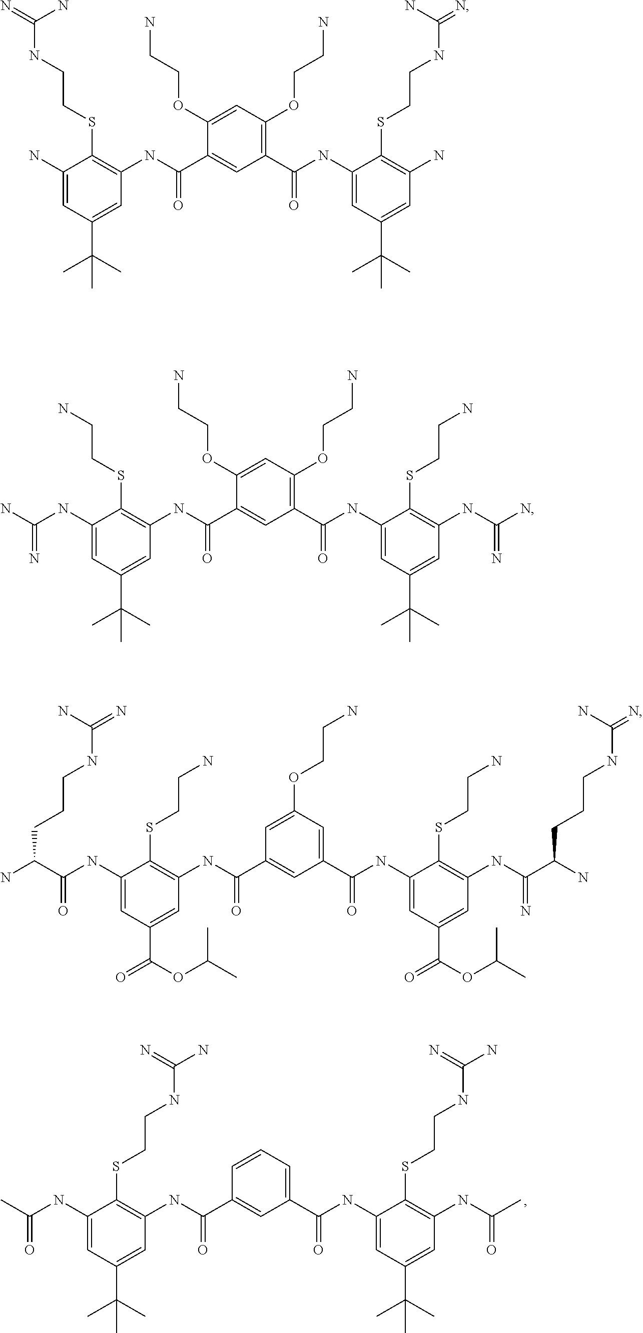

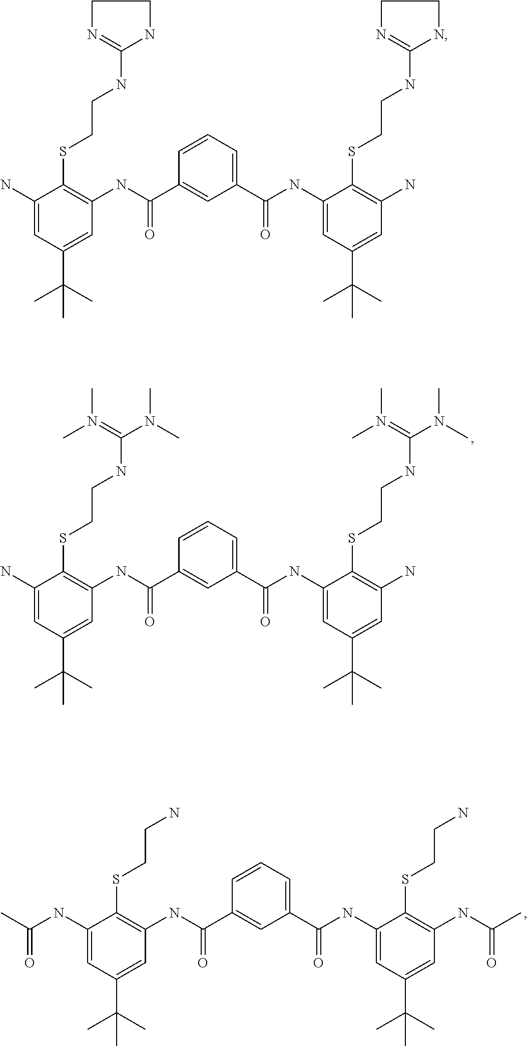

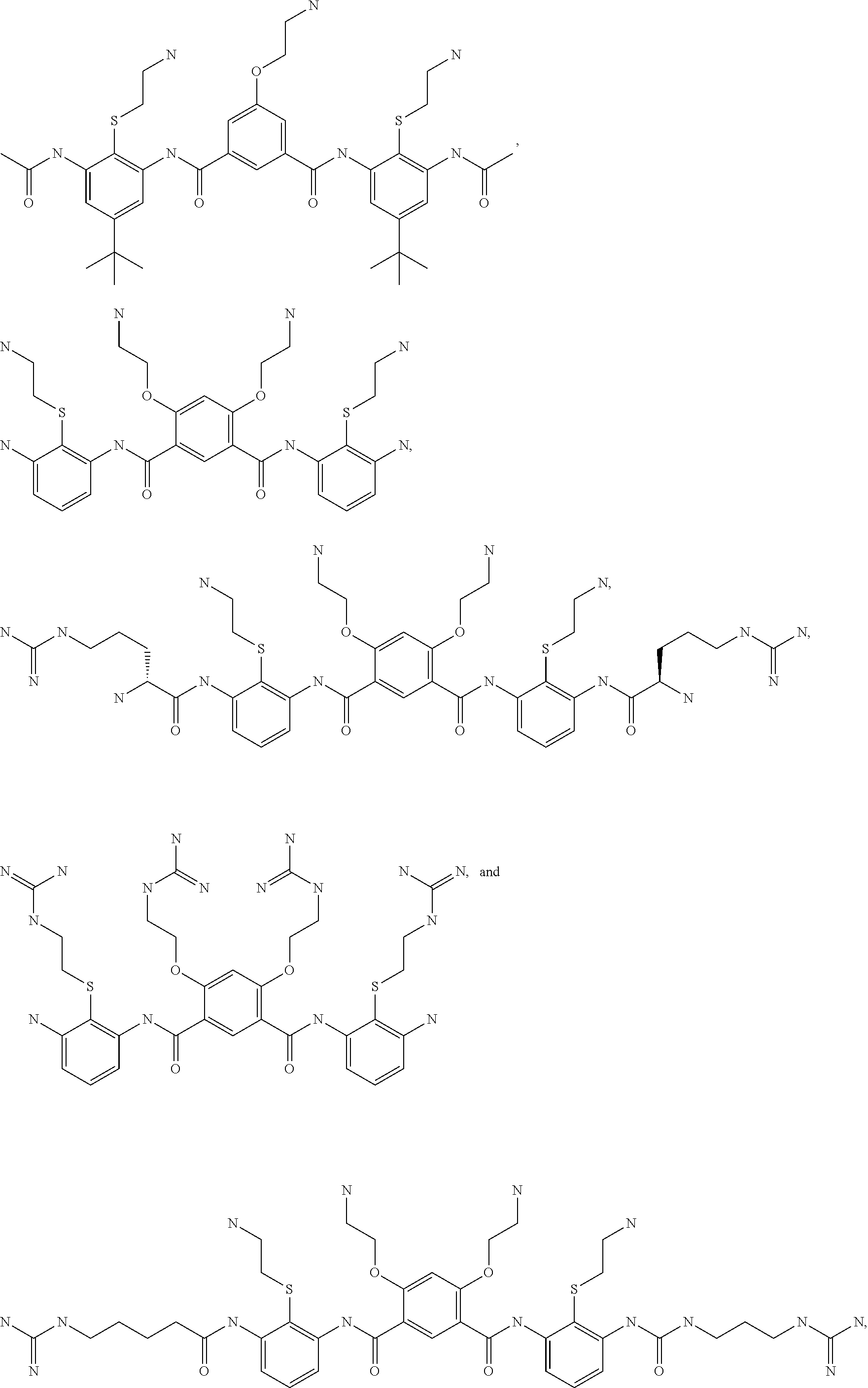

or a pharmaceutically acceptable salt thereof.

The present invention also provides methods of modulating an immune response in a mammal comprising administering to the mammal in need thereof a therapeutically effective amount of a compound of Formula III:

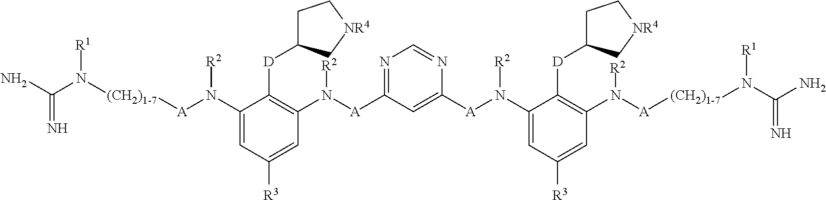

or a pharmaceutically acceptable salt thereof,
wherein:

each A is, independently, —C═O, —C═S, or CH₂;

each D is, independently, O or S;

each R¹ is, independently, hydrogen, C₁₋₃alkyl, C₁₋₃alkoxy, halo, or haloC₁₋₃alkyl;

each R² is, independently, hydrogen, C₁₋₃ alkyl, C₁₋₃alkoxy, halo, or haloC₁₋₃alkyl;

each R³ is, independently, hydrogen, C₁₋₄alkyl, C₁₋₄alkoxy, halo, or haloC₁₋₄alkyl; and

each R⁴ is, independently, hydrogen, C₁₋₃ alkyl, C₁₋₃alkoxy, halo, or haloC₁₋₃alkyl.

The present invention also provides methods of modulating an immune response in a mammal comprising administering to the mammal in need thereof a therapeutically effective amount of a compound of Formula IV:

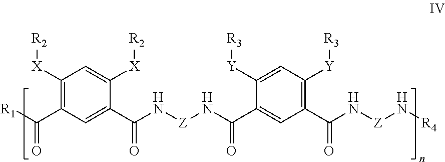

or a pharmaceutically acceptable salt thereof,
wherein:

n=1 to 10;

X is O or S;

Y is O or S;

Z is a bond, C₁-C₉ straight or branched alkyl, or a 1,4-cyclohexyl;

R₁ is NH₂ or NH-A, where A is C₁-C₉ straight or branched alkyl, where A is optionally substituted with —NH₂, —N(CH₃)₂ or —NH—C(═NH)NH₂;

R₂ is C₁-C₉ straight or branched alkyl, where R₂ is optionally substituted with one or more —NH₂, —N(CH₃)₂ or —NH—C(═NH)NH₂;

R₃ is C₁-C₉ straight or branched alkyl, where R₃ is optionally substituted with one or more —NH₂, —N(CH₃)₂ or —NH—C(═NH)NH₂;

R₄ is H or

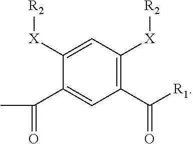

The present invention also provides methods of modulating an immune response in a mammal comprising administering to the mammal in need thereof a therapeutically effective amount of a compound chosen from:

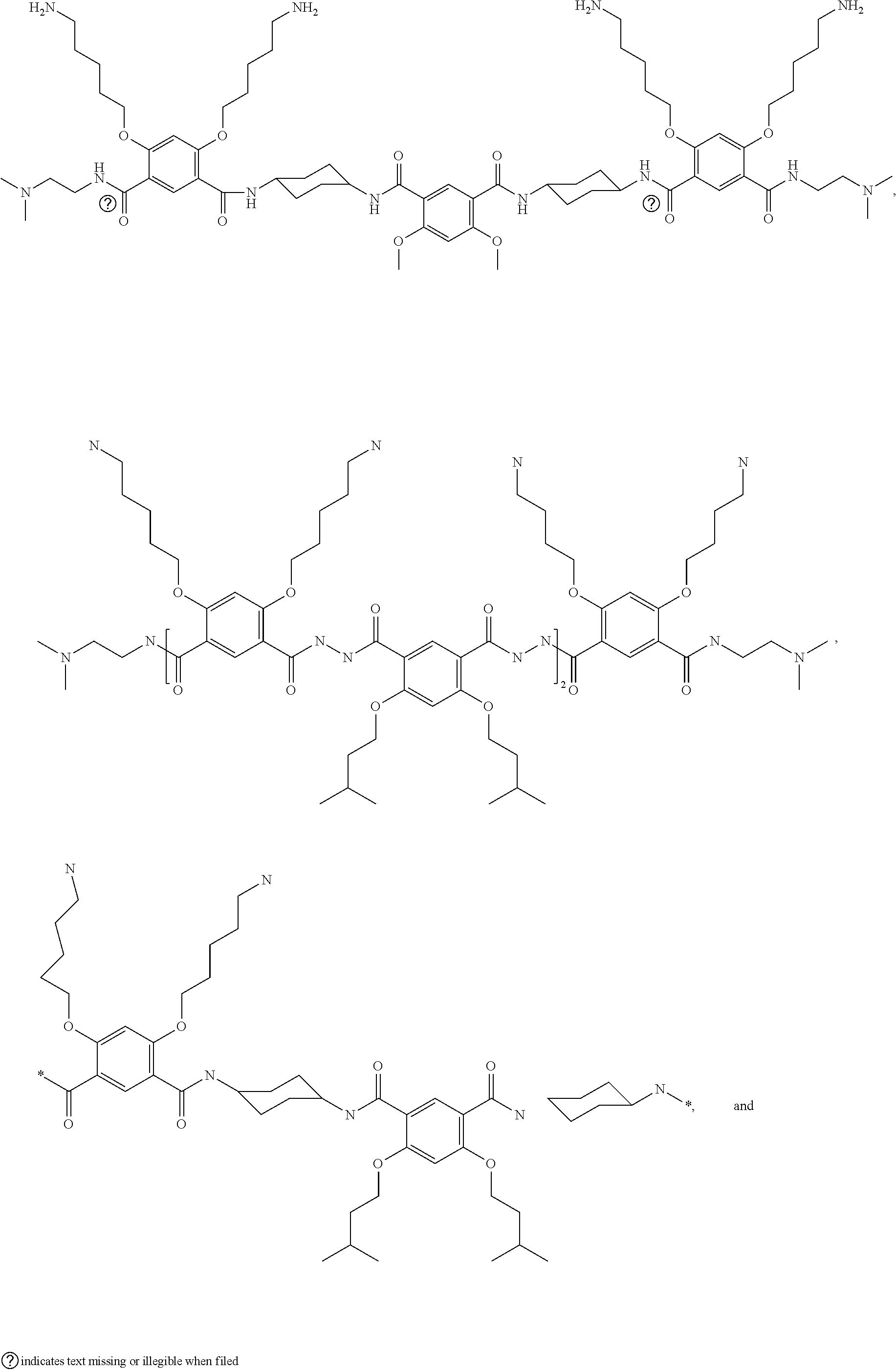

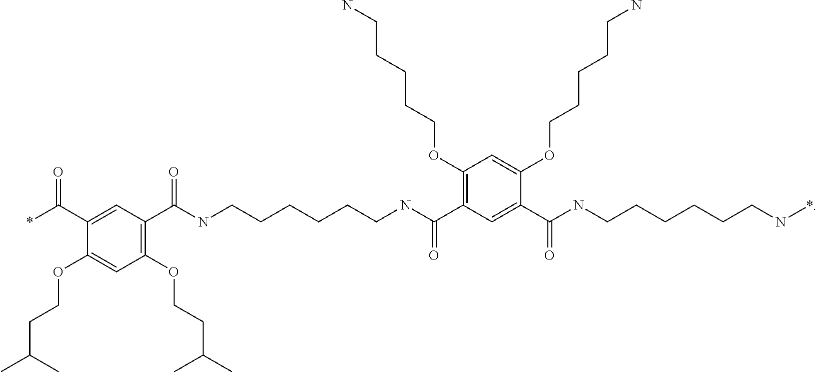

The present invention also provides methods of modulating an immune response in a mammal comprising administering to the mammal in need thereof a therapeutically effective amount of a compound of Formula V:

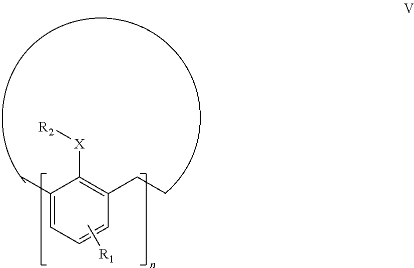

or a pharmaceutically acceptable salt thereof,
wherein:

n is 2-8;

X is a bond, O or —O—CH₂—C(═O)—O—,

R₁ is -A or —O-A, where A is C₁-C₉ straight or branched alkyl; and

R₂ is C₁-C₉ straight or branched alkyl, where R₂ is optionally substituted with one or more —NH₂, —N(CH₃)₂, or —NH—C(═NH)NH₂.

The present invention also provides methods of modulating an immune response in a mammal comprising administering to the mammal in need thereof a therapeutically effective amount of a compound chosen from:

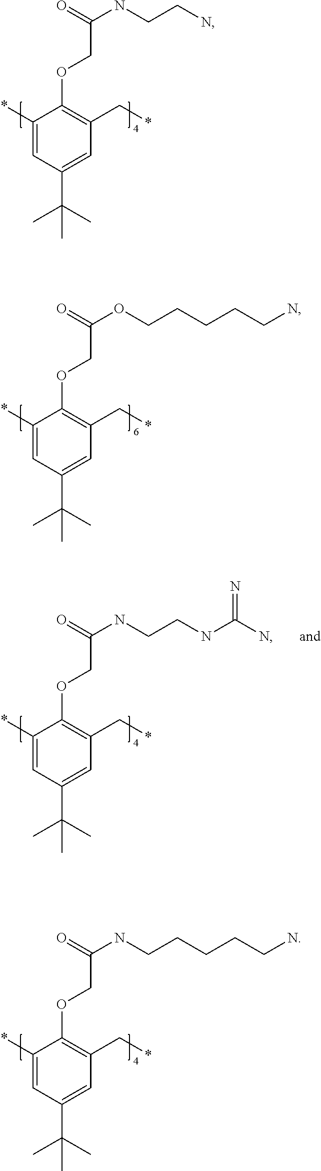

The present invention also provides methods of modulating an immune response in a mammal comprising administering to the mammal in need thereof a therapeutically effective amount of a compound of Formula VI:

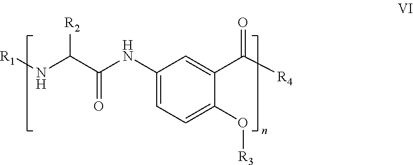

or a pharmaceutically acceptable salt thereof,
wherein:

n is 2 to 10;

R₁ is H or

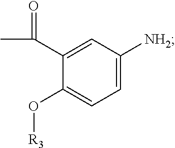

R₂ is C₁-C₉ straight or branched alkyl, where R₂ is optionally substituted with one or more —NH₂, —N(CH₃)₂ or —NH—C(═NH)NH₂;

R₃ is C₁-C₉ straight or branched alkyl, where R₂ is optionally substituted with one or more —N(CH₃)₂ or —NH—C(═NH)NH₂;

R₄ is OH, NH₂ or

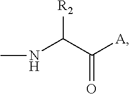

where A is OH or NH₂.

The present invention also provides methods of modulating an immune response in a mammal comprising administering to the mammal in need thereof a therapeutically effective amount of a compound chosen from:

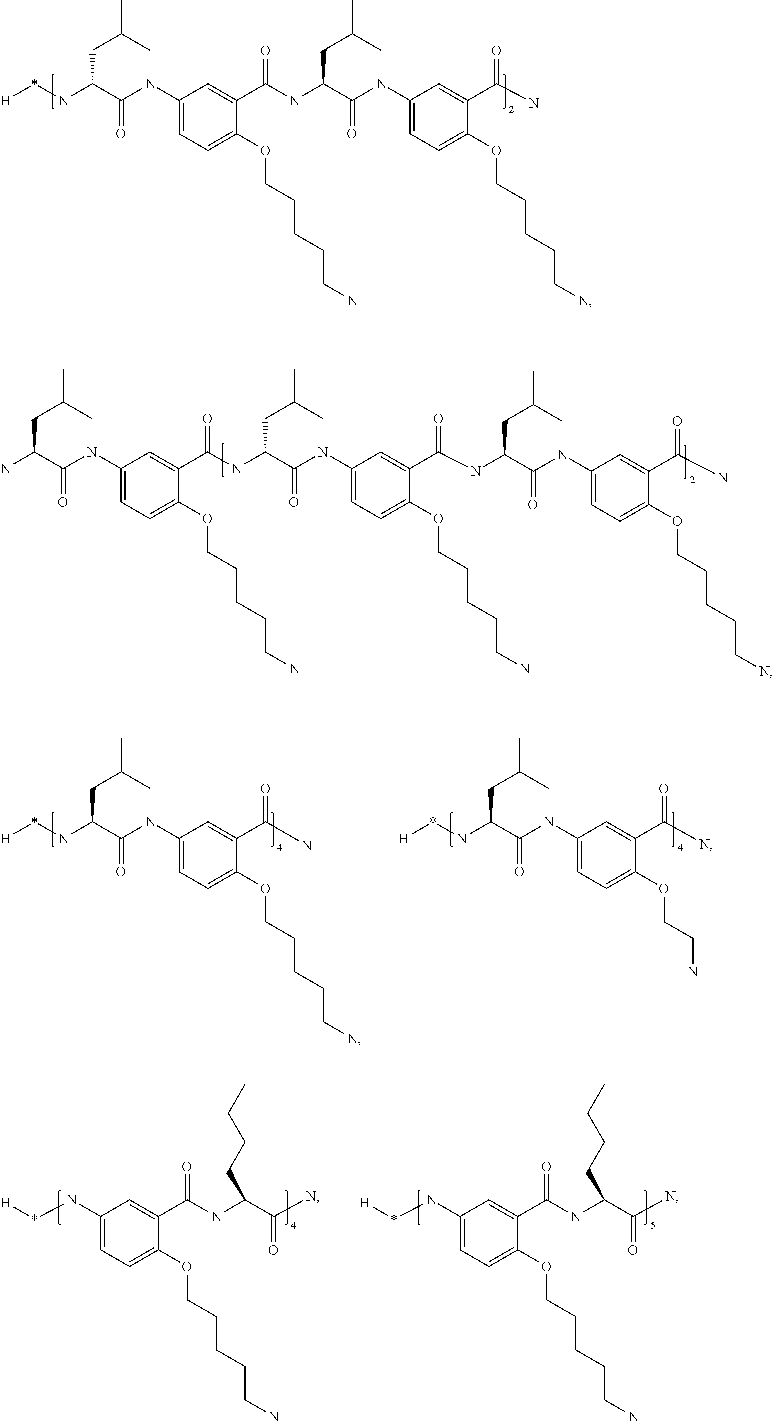

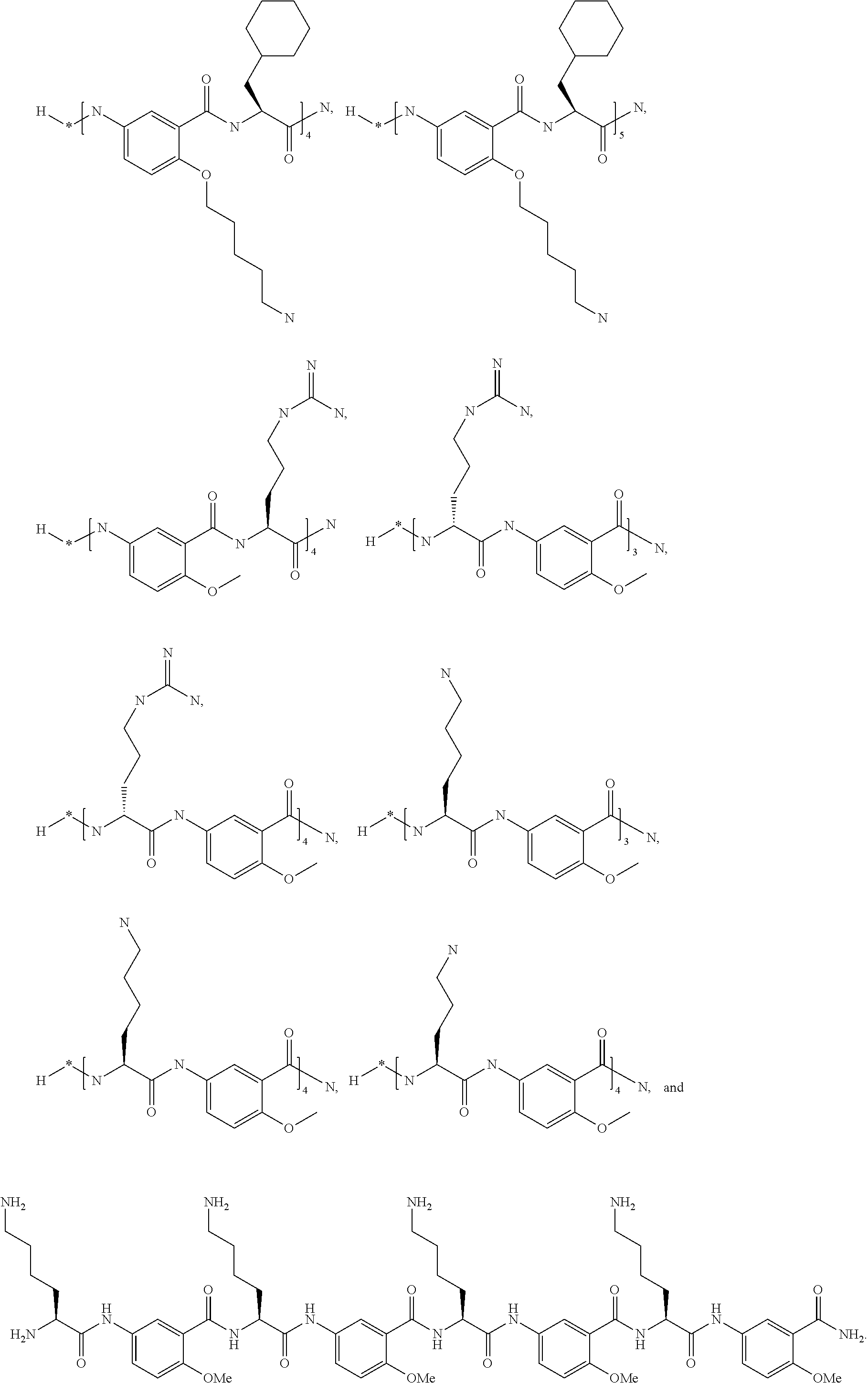

The present invention also provides methods of modulating an immune response in a mammal comprising administering to the mammal in need thereof a therapeutically effective amount of a compound of Formula VII:

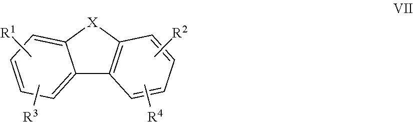

or a pharmaceutically acceptable salt thereof,
wherein:

X is C(R⁷)C(R⁸), C(═O), N(R⁹), O, S, S(═O), or S(═O)₂;

R⁷, R⁸, and R⁹ are, independently, H, C₁-C₈alkyl, C₁-C₈alkoxy, halo, OH, CF₃, or aromatic group;

R¹ and R² are, independently, H, C₁-C₈alkyl, C₁-C₈alkoxy, halo, OH, haloC₁-C₈alkyl, or CN;

R³ and R⁴ are, independently, carbocycle(R⁵)(R⁶);

each R⁵ and each R⁶ are, independently, H, C₁-C₈alkyl, C₁-C₈alkoxy, halo, OH, CF₃, aromatic group, heterocycle, or the free base or salt form of —(CH₂)ₙ—NH₂, or —(CH₂)—NH—(CH₂)ₙ—NH₂, or —(CH₂)—NH—C(═NH)NH₂, where each n is, independently, 1 to 8;

or a pharmaceutically acceptable salt thereof.

The present invention also provides methods of modulating an immune response in a mammal comprising administering to the mammal in need thereof a therapeutically effective amount of a compound chosen from:

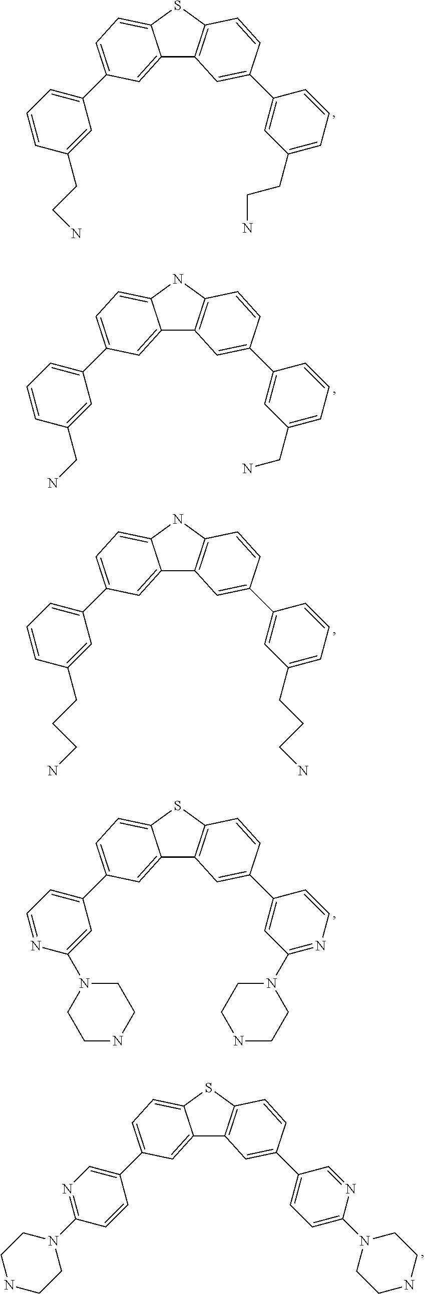

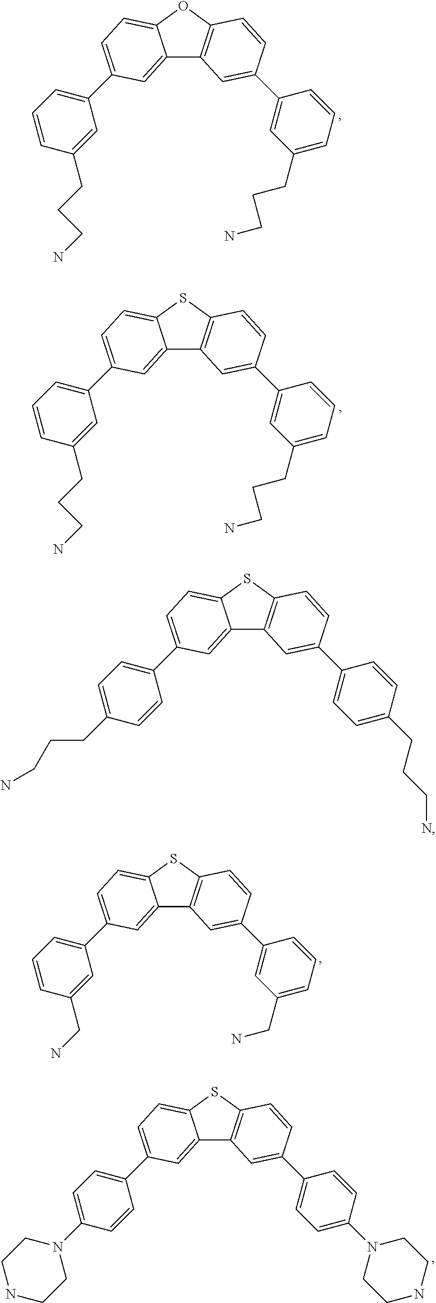

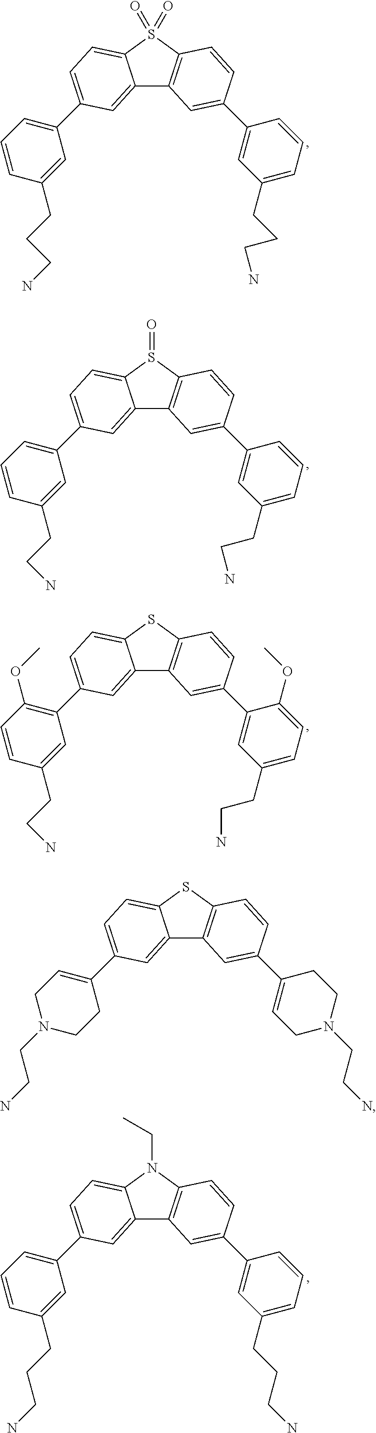

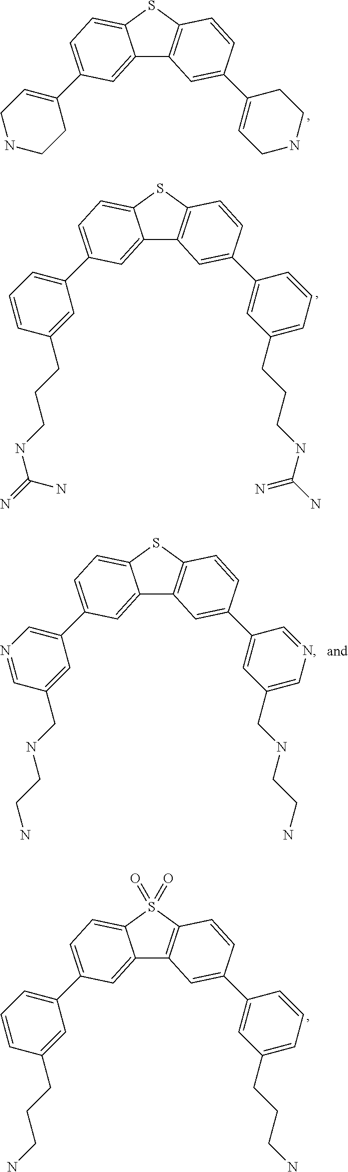

or pharmaceutically acceptable salt thereof.

The present invention also provides methods of modulating an immune response in a mammal comprising administering to the mammal in need thereof a therapeutically effective amount of a compound of Formula VIII:

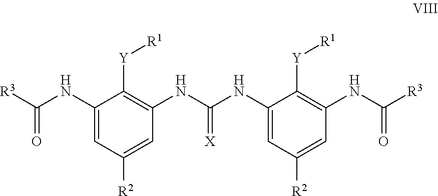

or a pharmaceutically acceptable salt thereof,
wherein:

X is O or S;

each Y is, independently, O, S, or N;

each R¹ is, independently, H, 5- or 6-membered heterocycle, or the free base or salt form of —(CH₂)ₙ—NH, or —(CH₂)ₙ—NH—C(═NH)NH₂, where each n is, independently, 1 to 4; or each R¹ is, independently, together with Y a 5- or 6-membered heterocycle;

each R² is, independently, H, CF₃, C(CH₃)₃, halo, or OH; and

each R³ is, independently, —(CH₂)ₙ—NH₂ or —(CH₂)—NH—C(═NH)NH₂, where each n is, independently, 1 to 4;

or a pharmaceutically acceptable salt thereof.

The present invention also provides methods of modulating an immune response in a mammal comprising administering to the mammal in need thereof a therapeutically effective amount of a compound chosen from:

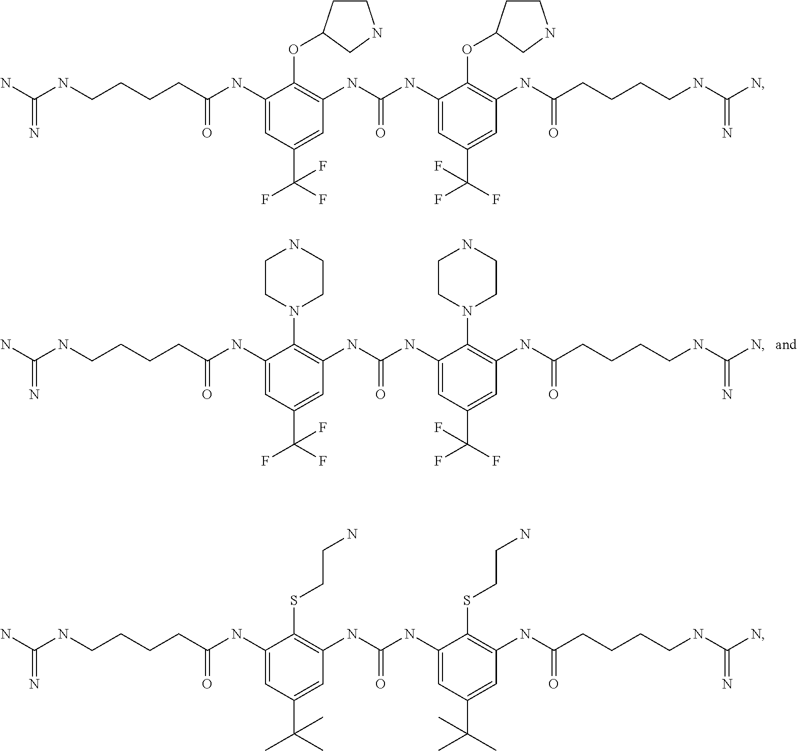

or pharmaceutically acceptable salt thereof.

The present invention also provides methods of modulating an immune response in a mammal comprising administering to the mammal in need thereof a therapeutically effective amount of a compound of Formula IX:

Q-X—Z—X-Q IX

or a pharmaceutically acceptable salt thereof,
wherein:

Z is

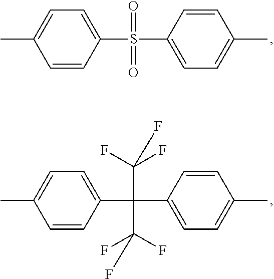

or phenyl;

each Q is, independently,

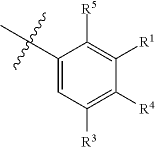

or —C(═O)—(CH₂)_{b}—NH—C(═NH)—NH₂, where each b is, independently, 1 to 4;

each X is, independently, O, S, or N;

each R¹ is, independently, H, CF₃, C(CH₃)₃, halo, or OH;

each R³ is, independently, H, —NH—R², —(CH₂)₁—NH₂, —NH₂, —NH—(CH₂)ᵣ—NH₂, or

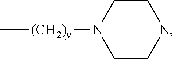

where each r is, independently, 1 or 2, each w is, independently, 1 to 3, and each y is, independently, 1 or 2;

each R² is, independently, H, or the free base or salt form of —(CH₂)ₙ—NH₂ or —(CH₂)ₙ—NH—C(═NH)NH₂, where each n is, independently, 1 to 4;

each R⁴ is, independently, H, —NH—C(═O)—(CH₂)ₚ—NH—C(═NH)—NH₂ or

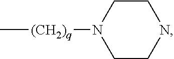

where each p is, independently, 1 to 6, and each q is, independently, 1 or 2; and

each R⁵ is, independently, H or CF₃;

or a pharmaceutically acceptable salt thereof.

The present invention also provides methods of modulating an immune response in a mammal comprising administering to the mammal in need thereof a therapeutically effective amount of a compound chosen from:

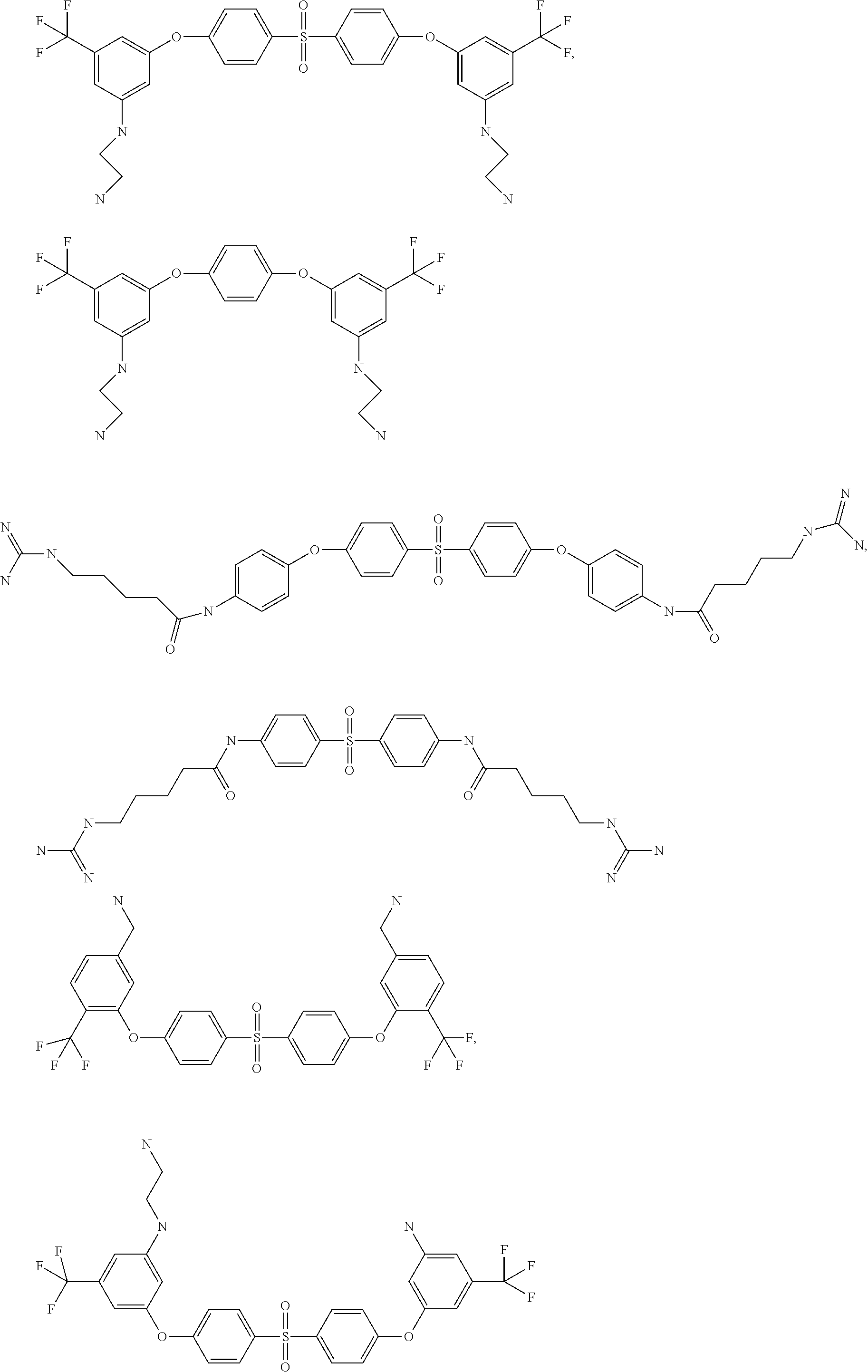

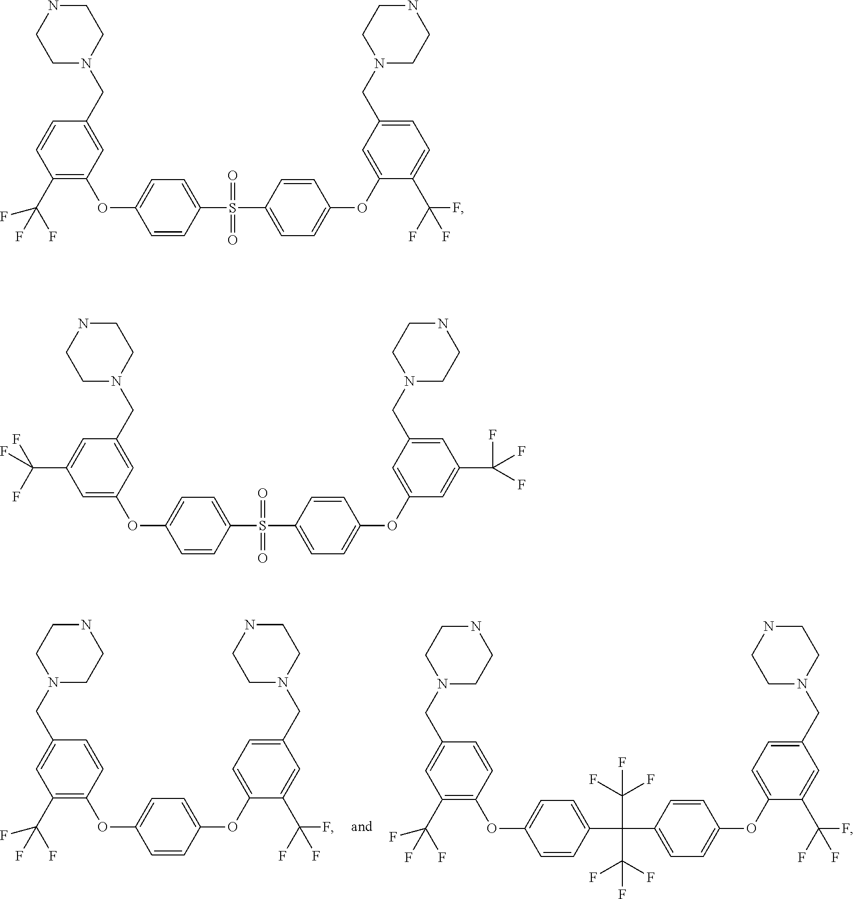

or a pharmaceutically acceptable salt thereof.

The present invention also provides methods of modulating an immune response in a mammal comprising administering to the mammal in need thereof a therapeutically effective amount of a compound of Formula X:

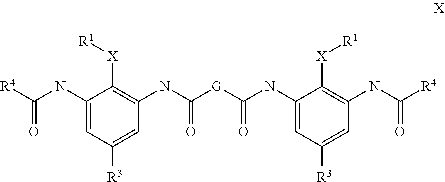

or a pharmaceutically acceptable salt thereof,
wherein:

G is

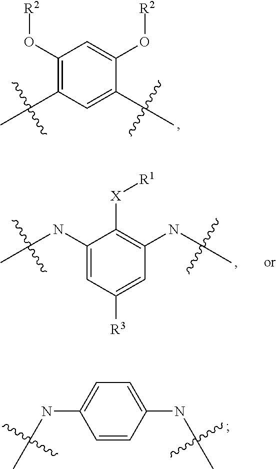

each X is, independently, O or S;

each R¹ is, independently,

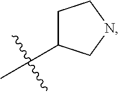

or the free base or salt form of —(CH₂)ₙ—NH₂ or —(CH₂)—NH—C(═NH)NH₂, where each n is, independently, 1 to 4;

each R² is, independently, H, C₁-C₈alkyl, or the free base or salt form of —(CH₂)ₙ—NH₂ or —(CH₂)ₙ—NH—C(═NH)NH₂, where each n is, independently, 1 to 4;

each R³ is, independently, H, CF₃, C(CH₃)₃, halo, or OH; and

each R⁴ is, independently, —(CH₂)ₙ—NH₂ or —(CH₂)ₙ—NH—C(═NH)NH₂, where each n is, independently, 1 to 4.

The present invention also provides methods of modulating an immune response in a mammal comprising administering to the mammal in need thereof a therapeutically effective amount of a compound chosen from:

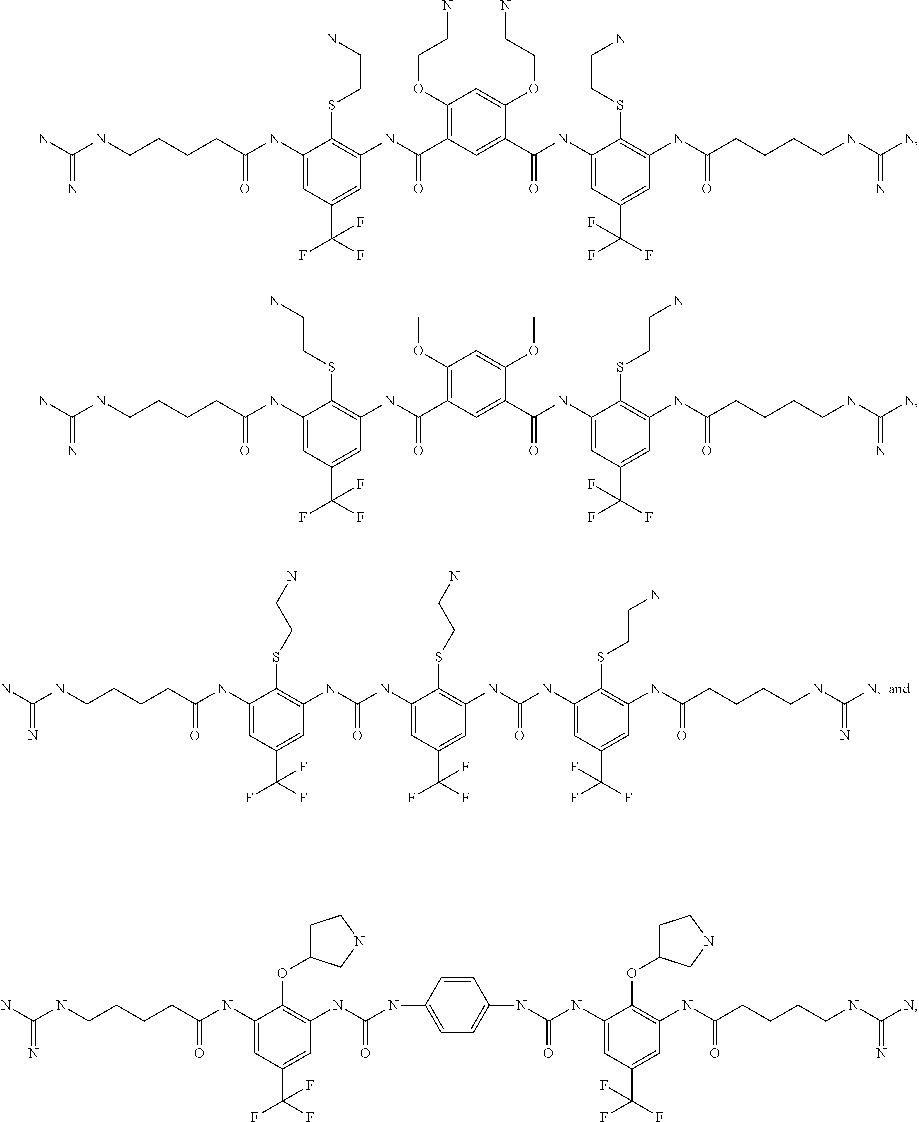

or a pharmaceutically acceptable salt thereof.

The present invention also provides methods of modulating an immune response in a mammal comprising administering to the mammal in need thereof a therapeutically effective amount of a compound of Formula XI:

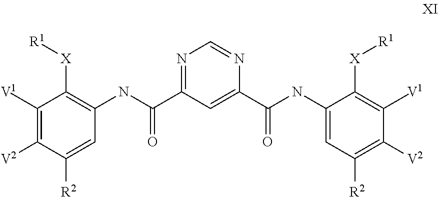

or a pharmaceutically acceptable salt thereof,
wherein:

each X is, independently, O, S, or S(═O)₂;

each R¹ is, independently, —(CH₂)ₙ—NH₂, —(CH₂)ₙ—NH—C(═NH)NH₂, or —(CH₂)ₙ—NH—C(═O)—R⁴, where each n is, independently, 1 to 4, and each R⁴ is, independently, H, C₁-C₃alkyl, or —(CH₂)ₚ—NH₂, where each p is, independently, 1 or 2;

each R² is, independently, H, halo, CF₃, or C(CH₃)₃; and

each V² is H, and each V¹ is, independently, —N—C(═O)—R³, where each R³ is, independently, —(CH₂)ₙ—NH₂ or —(CH₂)—NH—C(═NH)NH₂, where each n is, independently, 1 to 4; or each V¹ is H and each V² is, independently, —S—R⁵, where each R⁵ is, independently, —(CH₂)ₙ—NH₂ or —(CH₂)ₙ—NH—C(═NH)NH₂, where each n is, independently, 1 to 4;

or a pharmaceutically acceptable salt thereof.

The present invention also provides methods of modulating an immune response in a mammal comprising administering to the mammal in need thereof a therapeutically effective amount of a compound chosen from:

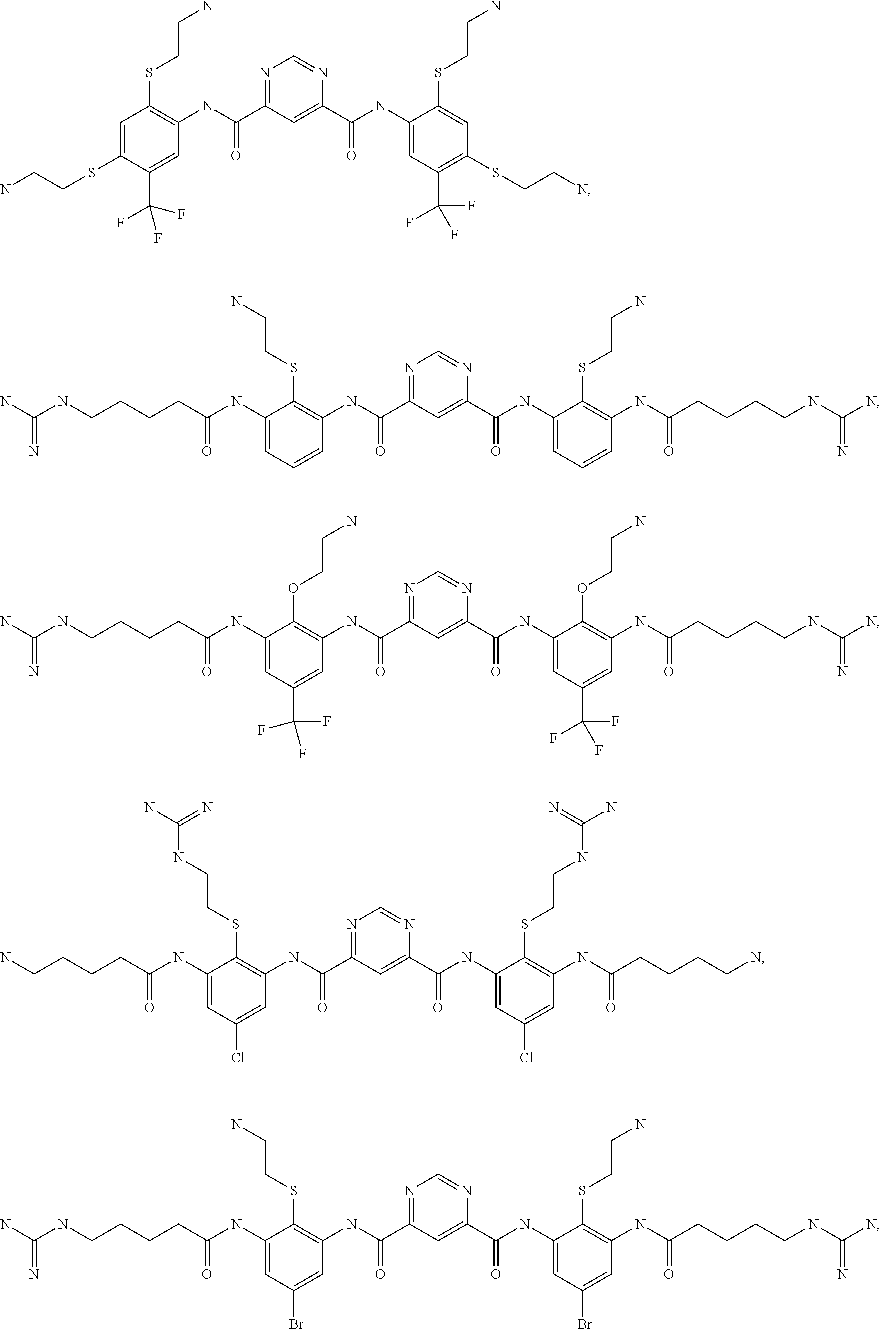

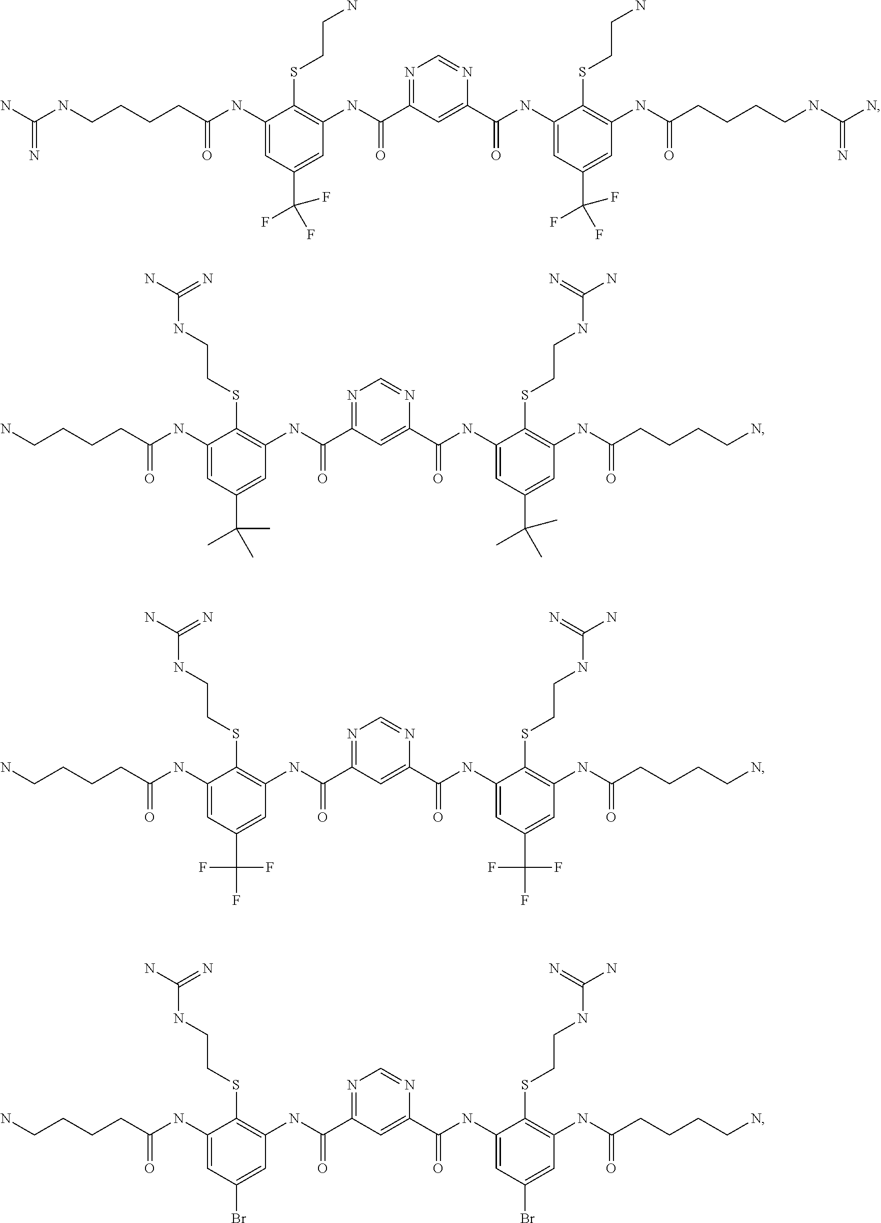

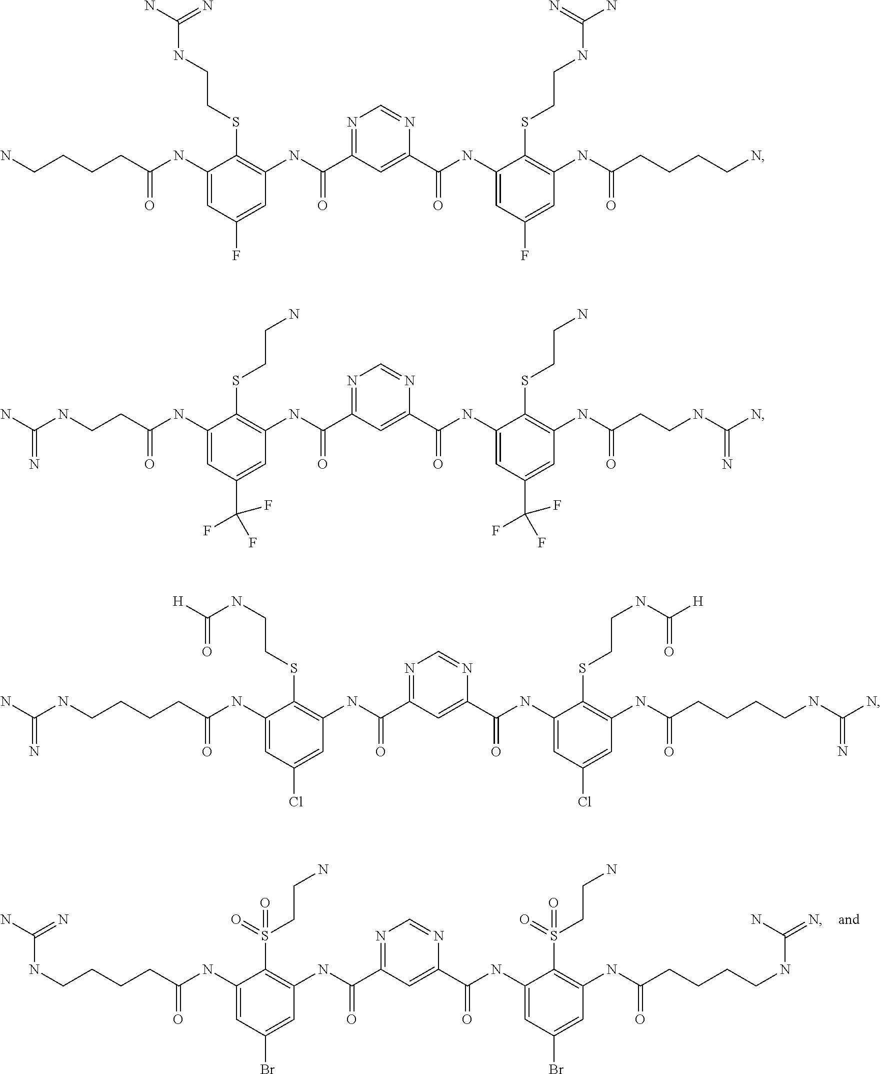

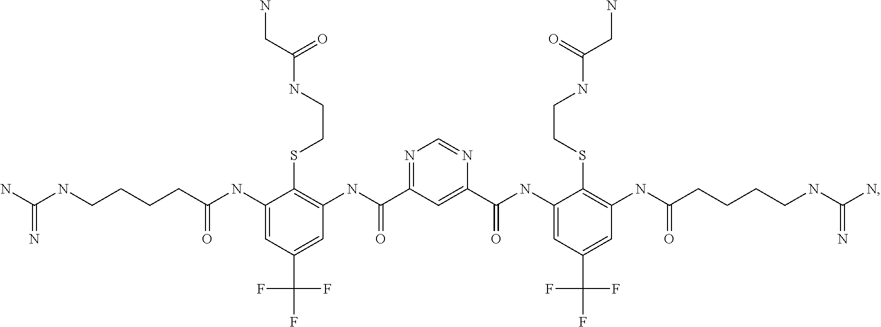

or a pharmaceutically acceptable salt thereof.

The present invention also provides methods of modulating an immune response in a mammal comprising administering to the mammal in need thereof a therapeutically effective amount of a compound of Formula XII:

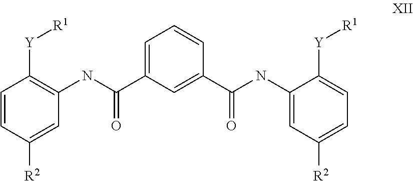

or a pharmaceutically acceptable salt thereof,
wherein:

each Y is, independently, O, S, or NH;

each R¹ is, independently, —(CH₂)ₙ—NH₂ or —(CH₂)ₙ—NH—C(═NH)NH₂, where each n is, independently, 1 to 4; and

each R² is, independently, H, halo, CF₃, or C(CH₃)₃;

or a pharmaceutically acceptable salt thereof.

The present invention also provides methods of modulating an immune response in a mammal comprising administering to the mammal in need thereof a therapeutically effective amount of a compound which is:

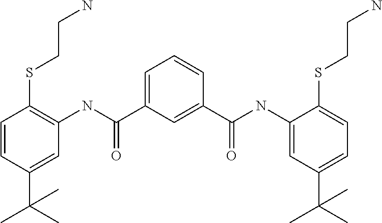

or a pharmaceutically acceptable salt thereof.

The present invention also provides methods of modulating an immune response in a mammal comprising administering to the mammal in need thereof a therapeutically effective amount of a compound of Formula XIII:

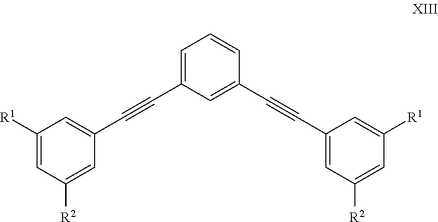

or a pharmaceutically acceptable salt thereof,
wherein:

each R¹ is, independently, H, C₁-C₈alkyl, C₁-C₈alkoxy, halo, OH, CF₃, or CN;

each R² is, independently, —(CH₂)ₙ—NH₂ or —(CH₂)—NH—C(═NH)NH₂, where each n is, independently, 1 to 4;

or a pharmaceutically acceptable salt thereof.

The present invention also provides methods of modulating an immune response in a mammal comprising administering to the mammal in need thereof a therapeutically effective amount of a compound which is:

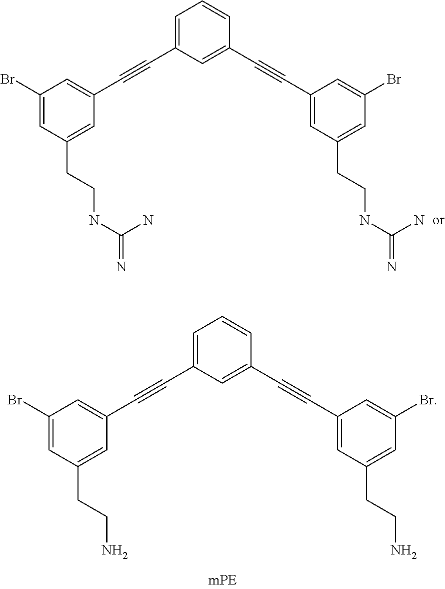

or a pharmaceutically acceptable salt thereof.

The present invention also provides methods of modulating an immune response in a mammal comprising administering to the mammal in need thereof a therapeutically effective amount of a compound of Formula XIV:

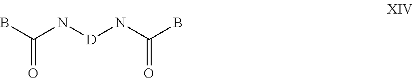

or a pharmaceutically acceptable salt thereof,
wherein:

D is

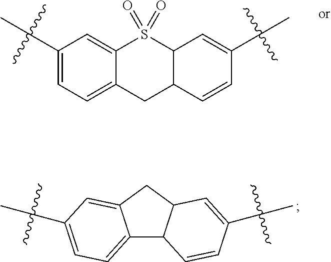

each B is, independently, —(CH₂)ₙ—NH—C(═NH)NH₂, where each n is, independently, 1 to 4,

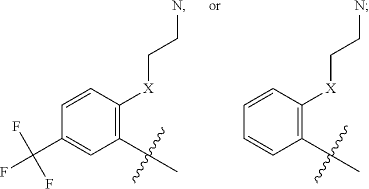

and

each X is, independently, O or S;

or a pharmaceutically acceptable salt thereof.

The present invention also provides methods of modulating an immune response in a mammal comprising administering to the mammal in need thereof a therapeutically effective amount of a compound which is:

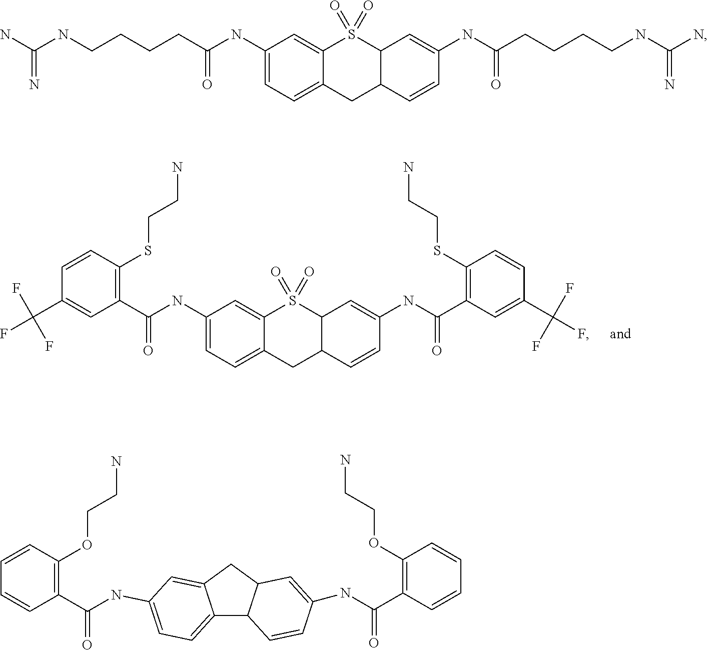

or a pharmaceutically acceptable salt thereof.

The present invention also provides methods of modulating an immune response in a mammal comprising administering to the mammal in need thereof a therapeutically effective amount of a compound of Formula XV:

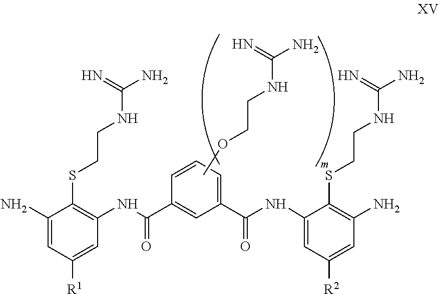

or a pharmaceutically acceptable salt thereof,
wherein:

R¹ is H or C₁₋₁₀ alkyl;

R² is H or C₁₋₁₀ alkyl; and

m is 1 or 2.

The present invention also provides methods of modulating an immune response in a mammal comprising administering to the mammal in need thereof a therapeutically effective amount of a compound of Formula XVI:

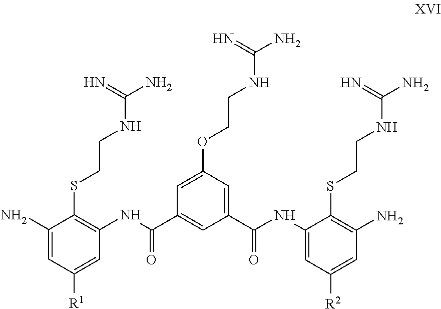

or a pharmaceutically acceptable salt thereof,
wherein:

R¹ is H or C₁₋₈ alkyl; and

R² is H or C₁₋₈ alkyl.

The present invention also provides methods of modulating an immune response in a mammal comprising administering to the mammal in need thereof a therapeutically effective amount of a compound of Formula XVII:

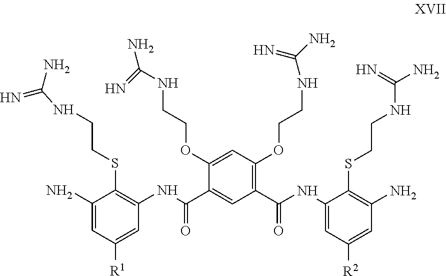

or a pharmaceutically acceptable salt thereof,
wherein:

R¹ is H or C₁₋₈ alkyl; and

R² is H or C₁₋₈ alkyl.

The present invention also provides methods of modulating an immune response in a mammal comprising administering to the mammal in need thereof a therapeutically effective amount of a compound which is:

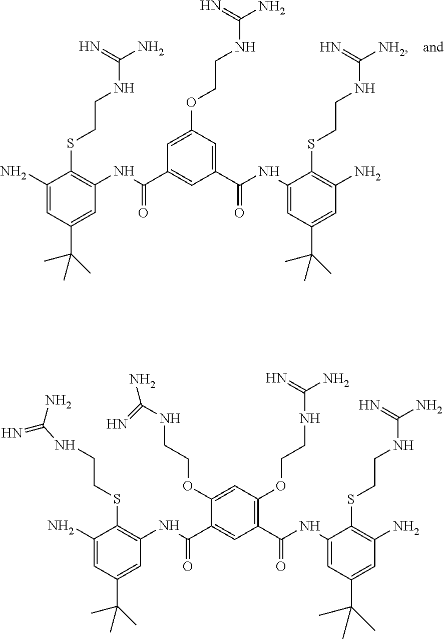

or a pharmaceutically acceptable salt thereof.

The present invention also provides methods of modulating an immune response in a mammal comprising administering to the mammal in need thereof a therapeutically effective amount of a compound of Formula XVIII:

R¹—[—X-A₁-Y—X-A₂-Y—]ₘ—R² XVIII

or a pharmaceutically acceptable salt thereof,
wherein:

each X is, independently, NR⁸, —N(R⁸)N(R⁸)—, O, or S;

each Y is, independently, C═O, C═S, O═S═O, —C(═O)C(═O)—, or —CR^{a}R^{b}—;

R^{a} and R^{b} are each, independently, hydrogen, a PL group, or an NPL group;

each R⁸ is, independently, hydrogen or alkyl;

A₁ and A₂ are each, independently, optionally substituted arylene or optionally substituted heteroarylene, wherein A₁ and A₂ are, independently, optionally substituted with one or more PL group(s), one or more NPL group(s), or a combination of one or more PL group(s) and one or more NPL group(s); or

each A₁ is, independently, optionally substituted arylene or optionally substituted heteroarylene, and each A₂ is a C₃ to C₈ cycloalkyl or —(CH₂)_{q}—, wherein q is 1 to 7, wherein A₁ and A₂ are, independently, optionally substituted with one or more PL group(s), one or more NPL group(s), or a combination of one or more PL group(s) and one or more NPL group(s); or

each A₂ is optionally substituted arylene or optionally substituted heteroarylene, and each A₁ is a C₃ to C₈ cycloalkyl or —(CH₂)_{q}—, wherein q is 1 to 7, wherein A₁ and A₂ are each, independently, optionally substituted with one or more PL group(s), one or more NPL group(s), or a combination of one or more PL group(s) and one or more NPL group(s);

R¹ is hydrogen, a PL group, or an NPL group, and R² is —X-A₁-Y—R¹¹, wherein R¹¹ is hydrogen, a PL group, or an NPL group; or

R¹ and R² are each, independently, hydrogen, a PL group, or an NPL group; or

R¹ and R² together are a single bond; or

R¹ is —Y-A₂-X—R¹², wherein R¹² is hydrogen, a PL group, or an NPL group, and R² is hydrogen, a PL group, or an NPL group;

each NPL group is, independently, —B(OR⁴)₂ or

—(NR³′)_{q1NPL}—U^{NPL}-LK^{NPL}—(NR³″)_{q2NPL}—R⁴′, wherein:

R³, R³′, and R³″ are each, independently, hydrogen, alkyl, or alkoxy;

R⁴ and R⁴′ are each, independently, hydrogen, alkyl, alkenyl, alkynyl, cycloalkyl, aryl, or heteroaryl, wherein each of the alkyl, alkenyl, alkynyl, cycloalkyl, aryl, and heteroaryl is optionally substituted with one or more substitutents, wherein each substituent is, independently, alkyl, halo, or haloalkyl;

each U^{NPL} is, independently, absent or O, S, S(═O), S(═O)₂, NR³, —C(═O)—, —C(═O)—NR³—, —C(═O)—N═N—NR³—, —C(═O)—NR³—N═N—, —N═N—NR³—, —C(═N—N(R³)₂)—, —C(═NR³)—, —C(═O)O—, —C(═O)S—, —C(═S)—, —O—P(═O)₂O—, —S—C═N—, or —C(═O)—NR³—O—, wherein groups with two chemically nonequivalent termini can adopt both possible orientations;

each LK^{NPL} is, independently, —(CH₂)_{pNPL}— or C₂₋₈ alkenylenyl, wherein each of the —(CH₂)_{pNPL} and C₂₋₈ alkenylenyl is optionally substituted with one or more substituents, wherein each substituent is, independently, amino, hydroxyl, aminoalkyl, hydroxylalkyl, or alkyl;

each pNPL is, independently, an integer from 0 to 8;

q1NPL and q2NPL are each, independently, 0, 1, or 2;

each PL group is, independently, halo, hydroxyethoxymethyl, methoxyethoxymethyl, polyoxyethylene, or —(NR⁵′)_{q1PL}—U^{PL}-LK^{PL}—(NR⁵″)_{q2PL}—V, wherein:

R⁵, R⁵′, and R⁵″ are each, independently, hydrogen, alkyl, or alkoxy;

each U^{PL} is, independently, absent or O, S, S(═O), S(═O)₂, NR⁵, —C(═O)—, —C(═O)—NR⁵—, —C(═O)—N═N—NR⁵—, —C(═O)—NR⁵—N═N—, —N═N—NR⁵—, —C(═N—N(R⁵)₂)—, —C(═NR⁵)—, —C(═O)O—, —C(═O)S—, —C(═S)—, —O—P(═O)₂O—, —S—C═N—, or —C(═O)—NR⁵—O—, wherein groups with two chemically nonequivalent termini can adopt either of the two possible orientations;

each V is, independently, nitro, cyano, amino, halo, hydroxy, alkoxy, alkylthio, alkylamino, dialkylamino, —NH(CH₂)ₚNH₂ wherein p is 1 to 5, —C(═O)NH(CH₂)ₚNH₂ wherein p is 1 to 5, —C(═O)NH(CH₂)ₚNHC(═NH)NH₂ wherein p is 1 to 5, —C(═O)NH(CH₂)ₚNHC(═O)NH₂ wherein p is 1 to 5, —NHC(═O)-alkyl, —N(CH₂CH₂NH₂)₂, diazamino, amidino, guanidino, ureido, carbamoyl, —C(═O)OH, —C(═O)R^{c}, —C(═O)NH—OH, —O—NH—C(═NH)NH₂, —NH—S(═O)₂OH, S(═O)₂OH, NR^{d}R^{e}, semicarbazone, aryl, cycloalkyl, heterocycloalkyl, or heteroaryl, wherein each of the aryl and cycloalkyl is substituted with one or more substitutents, wherein each of the heterocycloalkyl and heteroaryl is optionally substituted with one or more substituents, and wherein each of the substituents for the aryl, cycloalkyl, heterocycloalkyl, and heteroaryl is, independently, nitro, cyano, amino, halo, hydroxy, alkoxy, alkylthio, alkylamino, dialkylamino, —NH(CH₂)ₚNH₂ wherein p is 1 to 5, —N(CH₂CH₂NH₂)₂, diazamino, amidino, guanidino, ureido, carbamoyl, —C(═O)OH, —C(═O)OR^{c}, —C(═O)NH—OH, —O—NH—C(═NH)NH₂, —NH—S(═O)₂OH, S(═O)₂OH, NR^{d}R^{e}, semicarbazone, aminosulfonyl, aminoalkoxy, aminoalkylthio, lower acylamino, or benzyloxycarbonyl;

each R^{c} is, independently, C₁₋₆ alkyl, C₁₋₆ haloalkyl, C₂₋₆ alkenyl, C₂₋₆ alkynyl, aryl, cycloalkyl, heteroaryl, heterocycloalkyl, arylalkyl, heteroarylalkyl, cycloalkylalkyl, or heterocycloalkylalkyl, each optionally substituted by one or more substitutents, wherein each substituent is, independently, OH, amino, halo, C₁₋₆ alkyl, C₁₋₆ haloalkyl, aryl, arylalkyl, heteroaryl, heteroarylalkyl, cycloalkyl, or heterocycloalkyl;

R^{d} and R^{e} are, independently, H, C₁₋₆ alkyl, C₁₋₆ haloalkyl, C₂₋₆ alkenyl, C₂₋₆ alkynyl, aryl, heteroaryl, cycloalkyl, heterocycloalkyl, arylalkyl, heteroarylalkyl, cycloalkylalkyl, or heterocycloalkylalkyl, wherein each of the C₁₋₆ alkyl, C₁₋₆ haloalkyl, C₂₋₆ alkenyl, C₂₋₆ alkynyl, aryl, heteroaryl, cycloalkyl, heterocycloalkyl, arylalkyl, heteroarylalkyl, cycloalkylalkyl and heterocycloalkylalkyl is optionally substituted by OH, amino, halo, C₁₋₆ alkyl, C₁₋₆ haloalkyl, C₁₋₆ haloalkyl, aryl, arylalkyl, heteroaryl, heteroarylalkyl, cycloalkyl, or heterocycloalkyl;

or R^{d} and R^{e} together with the N atom to which they are attached form a 4-, 5-, 6-, 7-, or 8-membered heterocycloalkyl;

each LK^{PL} is, independently, —(CH₂)_{pPL}— or C₂₋₈ alkenylenyl, wherein each of the —(CH₂)_{pNPL}— and C₂₋₈ alkenylenyl is optionally substituted with one or more substituents, wherein each substituent is, independently, amino, hydroxyl, aminoalkyl, hydroxylalkyl, or alkyl;

each pPL is, independently, an integer from 0-8;

q1PL and q2PL are each, independently, 0, 1, or 2; and

m is an integer from 1 to about 20.

The present invention also provides methods of modulating an immune response in a mammal comprising administering to the mammal in need thereof a therapeutically effective amount of a compound of Formula XIX:

R¹—[—X-A₁-X—Y-A₂-Y—]ₘ—R² XIX

or a pharmaceutically acceptable salt thereof,
wherein:

each X is, independently, NR⁸, O, S, —N(R⁸)N(R⁸)—, —N(R⁸)—(N═N)—, —(N═N)—N(R⁸)—, —C(R⁷R⁷′)NR⁸—, —C(R⁷R⁷)O—, or —C(R⁷R⁷)S—;

each Y is, independently, C═O, C═S, O═S═O, —C(═O)C(═O)—, C(R⁶R⁶′)C═O, or C(R⁶R⁶′)C═S;

each R⁸ is, independently, hydrogen or alkyl;

each R⁷ and each R⁷′ are, independently, hydrogen or alkyl; or R⁷ and R⁷′ together form —(CH₂)ₚ—, wherein p is 4 to 8;

each R⁶ and each R⁶′ are, independently, hydrogen or alkyl; or R⁶ and R⁶′ together form —(CH₂)₂NR′²(CH₂)₂—, wherein R¹² is hydrogen, —C(═N)CH₃, or —C(═NH)—NH₂;

A₁ and A₂ are each, independently, optionally substituted arylene or optionally substituted heteroarylene, wherein A₁ and A₂ are each, independently, optionally substituted with one or more PL group(s), one or more NPL group(s), or a combination of one or more PL group(s) and one or more NPL group(s);

or each A₂ is, independently, optionally substituted arylene or optionally substituted heteroarylene, and each A₁ is, independently, optionally substituted C₃ to C₈ cycloalkyl,

wherein A₁ and A₂ are each, independently, optionally substituted with one or more PL group(s), one or more NPL group(s), or a combination of one or more PL group(s) and one or more NPL group(s);

R¹ is hydrogen, a PL group, or an NPL group, and R² is —X-A₁-X—R¹, wherein A₁ is as defined above and is optionally substituted with one or more PL group(s), one or more NPL group(s), or a combination of one or more PL group(s) and one or more NPL group(s); or

R¹ is hydrogen, a PL group, or an NPL group, and R² is —X-A′-X—R¹, wherein A′ is C₃ to C₈ cycloalkyl, aryl, or heteroaryl and is optionally substituted with one or more PL group(s), one or more NPL group(s), or a combination of one or more PL group(s) and one or more NPL group(s); or

R¹ is —Y-A₂-Y—R², and each R² is, independently, hydrogen, a PL group, or an NPL group; or

R¹ is —Y-A¹ and R² is —X-A′, wherein each A′ is, independently, C₃ to C₈ cycloalkyl, aryl, or heteroaryl and is optionally substituted with one or more PL group(s), one or more NPL group(s), or a combination of one or more PL group(s) and one or more NPL group(s); or

R¹ and R² are, independently, a PL group or an NPL group; or

R¹ and R² together form a single bond; each NPL is, independently, —B(OR⁴)₂ or —(NR³)_{q1NPL}—U^{NPL}-LK^{NPL}—(NR³″)_{q2NPL}—R⁴′, wherein:

R³, R³′, and R³″ are each, independently, hydrogen, alkyl, or alkoxy;

R⁴ and R⁴′ are each, independently, hydrogen, alkyl, alkenyl, alkynyl, cycloalkyl, aryl, and heteroaryl, wherein each of the alkyl, alkenyl, alkynyl, cycloalkyl, aryl, and heteroaryl is optionally substituted with one or more alkyl or halo groups;

each U^{NPL} is, independently, absent or O, S, S(═O), S(═O)₂, NR³, —C(═O)—, —C(═O)—NR³—, —C(═O)—N═N—NR³—, —C(═O)—NR³—N═N—, —N═N—NR³—, —C(═N—N(R³)₂)—, —C(═NR³)—, —C(═O)O—, —C(═O)S—, —C(═S)—, —O—P(═O)₂O—, —S—C═N—, or —C(═O)—NR³—O—, wherein groups with two chemically nonequivalent termini can adopt both possible orientations;

each LK^{NPL} is, independently, —(CH₂)_{pNPL}— or C₂₋₈ alkenylenyl, wherein each of the (CH₂)_{pNPL}— and C₂₋₈ alkenylenyl is optionally substituted with one or more substituents, wherein each substituent is, independently, amino, hydroxyl, aminoalkyl, hydroxylalkyl, or alkyl;

each pNPL is, independently, an integer from 0 to 8;

q1NPL and q2NPL are each, independently, 0, 1, or 2;

each PL is, independently, halo, hydroxyethoxymethyl, methoxyethoxymethyl, polyoxyethylene, or —(NR⁵)_{q1PL}—U^{PL}-LK^{PL}—(NR⁵)_{q2PL}—V, wherein:

R⁵, R⁵′, and R⁵″ are each, independently, hydrogen, alkyl, and alkoxy;

each U^{PL} is, independently, absent or O, S, S(═O), S(═O)₂, NR⁵, —C(═O)—, —C(═O)—NR⁵—, —C(═O)—N═N—NR⁵—, —C(═O)—NR⁵—N═N—, —N═N—NR⁵—, —C(═N—N(R⁵)₂)—, —C(═NR⁵)—, —C(═O)O—, —C(═O)S—, —C(═S)—, —O—P(═O)₂O—, —S—C═N—, or —C(═O)—NR⁵—O—, wherein groups with two chemically nonequivalent termini can adopt either of the two possible orientations;

each V is, independently, nitro, cyano, amino, halo, hydroxy, alkoxy, alkylthio, alkylamino, dialkylamino, —NH(CH₂)ₚNH₂ wherein p is 1 to 5, —C(═O)NH(CH₂)ₚNH₂ wherein p is 1 to 5, —C(═O)NH(CH₂)ₚNHC(═NH)NH₂ wherein p is 1 to 5, —C(═O)NH(CH₂)ₚNHC(═O)NH₂ wherein p is 1 to 5, —NHC(═O)-alkyl, —N(CH₂CH₂NH₂)₂, diazamino, amidino, guanidino, ureido, carbamoyl, —C(═O)OH, —C(═O)OR¹, —C(═O)NH—OH, —O—NH—C(═NH)NH₂, —NH—S(═O)₂OH, S(═O)₂OH, NR^{d}R^{e}, semicarbazone, aryl, cycloalkyl, heterocycloalkyl, or heteroaryl, wherein each of the aryl and cycloalkyl is substituted with one or more substitutents, wherein each of the heterocycloalkyl, and heteroaryl is optionally substituted with one or more substituents, and wherein each of the substituents for the aryl, cycloalkyl, heterocycloalkyl, and heteroaryl is, independently, nitro, cyano, amino, halo, hydroxy, alkoxy, alkylthio, alkylamino, dialkylamino, —NH(CH₂)ₚNH₂ wherein p is 1 to 5, —N(CH₂CH₂NH₂)₂, diazamino, amidino, guanidino, ureido, carbamoyl, —C(═O)OH, —C(═O)OR^{c}, —C(═O)NH—OH, —O—NH—C(═NH)NH₂, —NH—S(═O)₂OH, S(═O)₂OH, NR^{d}R^{e}, semicarbazone, aminosulfonyl, aminoalkoxy, aminoalkylthio, lower acylamino, or benzyloxycarbonyl;

each LK^{PL} is, independently, —(CH₂)_{pPL}— or C₂₋₈ alkenylenyl, wherein each of the —(CH₂)_{pNPL}— and C₂₋₈alkenylenyl is optionally substituted with one or more substituents, wherein each substituent is, independently, amino, hydroxyl, aminoalkyl, hydroxylalkyl, or alkyl;

each pPL is, independently, an integer from 0 to 8;

q1PL and q2PL are each, independently, 0, 1, or 2; and

m is an integer from 1 to about 20.

The present invention also provides methods of modulating an immune response in a mammal comprising administering to the mammal in need thereof a therapeutically effective amount of a compound chosen from:

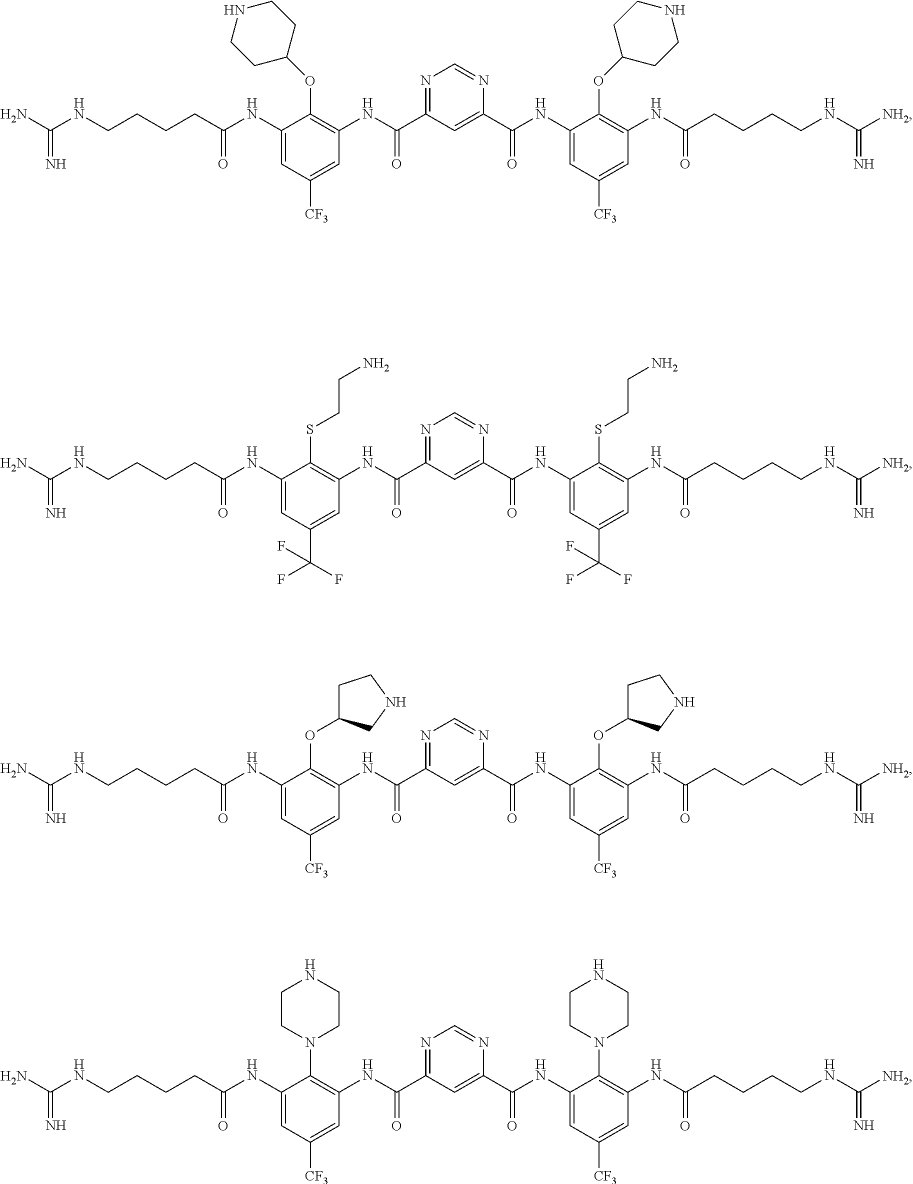

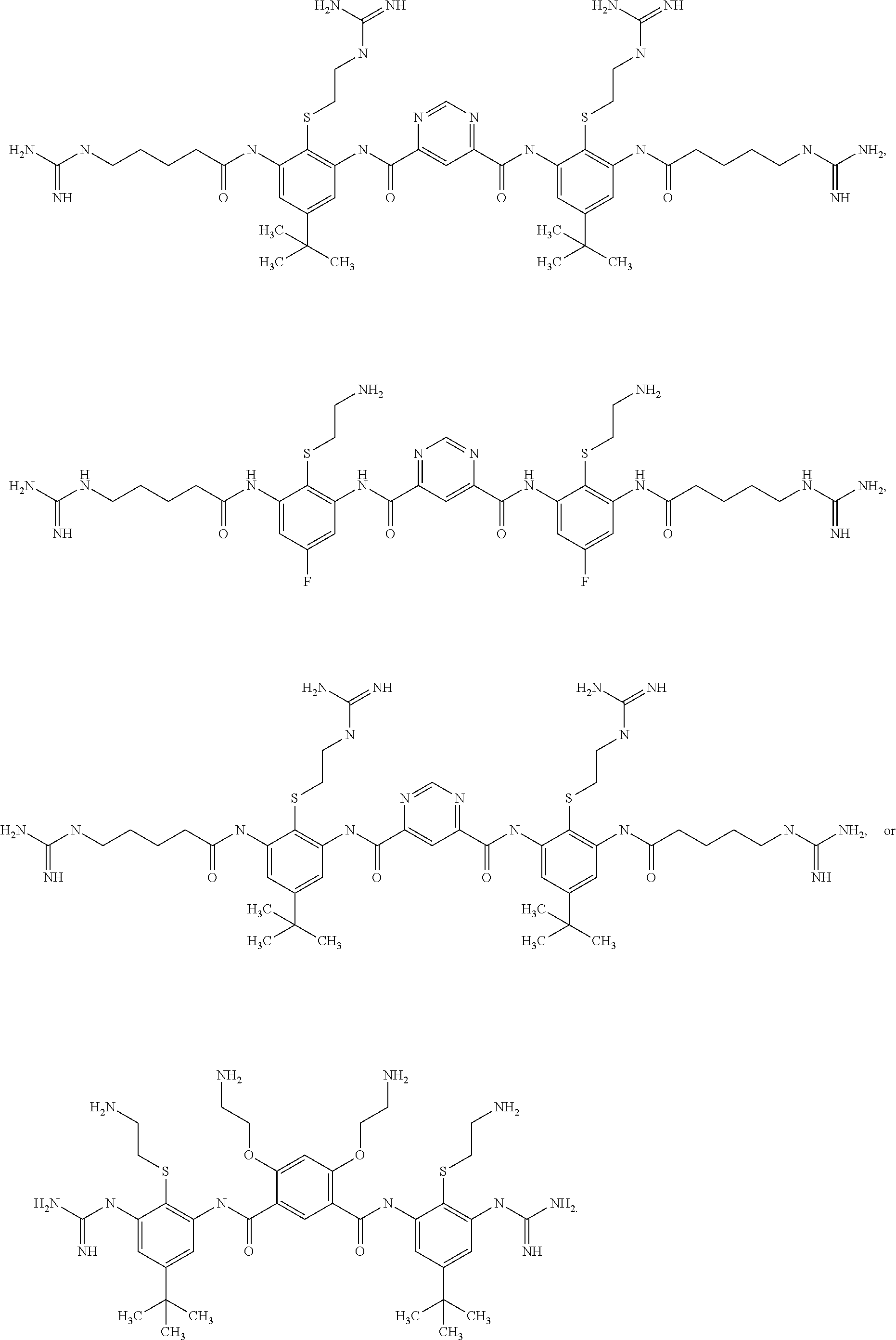

or a pharmaceutically acceptable salt thereof.

The present invention also provides methods of modulating an immune response in a mammal comprising administering to the mammal in need thereof a therapeutically effective amount of a compound of Formula XX:

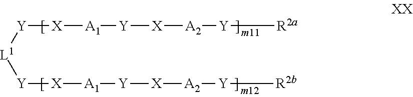

or a pharmaceutically acceptable salt thereof,
wherein:

each X is, independently, NR⁸;

each Y is C═O;

each R⁸ is, independently, hydrogen or alkyl;

each A₂ is optionally substituted arylene or optionally substituted heteroarylene, and

each A₁ is —(CH₂)_{q}—, wherein q is 1 to 7, wherein A₁ and A₂ are each, independently, optionally substituted with one or more PL group(s), one or more NPL group(s), or a combination of one or more PL group(s) and one or more NPL group(s);

R² and R^{2a} are each, independently, hydrogen, a PL group, an NPL group or —X-A₁-Y—R¹¹, wherein R¹¹ is hydrogen, a PL group, or an NPL group;

L¹ is C₁₋₁₀alkylene optionally substituted with one or more substitutents, wherein each substituent is, independently, alkyl, halo, haloalkyl, aminoalkyl, hydroxylalkyl, V, or —(CH₂)_{pPL}—V, wherein pPL is an integer from 1 to 5;

each NPL group is, independently, —B(OR⁴)₂ or

—(NR³′)_{q1NPL}—U^{NPL}-LK^{NPL}—(NR³″)_{q2NPL}—R⁴′, wherein:

R³, R³′, and R³″ are each, independently, hydrogen, alkyl, or alkoxy;

R⁴ and R⁴′ are each, independently, hydrogen, alkyl, alkenyl, alkynyl, cycloalkyl, aryl, or heteroaryl, wherein each of the alkyl, alkenyl, alkynyl, cycloalkyl, aryl, and heteroaryl is optionally substituted with one or more substitutents, wherein each substituent is, independently, alkyl, halo, or haloalkyl;

each U^{NPL} is, independently, absent or O, S, S(═O), S(═O)₂, NR³, —C(═O)—, —C(═O)—NR³—, —C(═O)—N═N—NR³—, —C(═O)—NR³—N═N—, —N═N—NR³—, —C(═N—N(R³)₂)—, —C(═NR³)—, —C(═O)O—, —C(═O)S—, —C(═S)—, —O—P(═O)₂O—, —S—C═N—, or —C(═O)—NR³—O—, wherein groups with two chemically nonequivalent termini can adopt both possible orientations;

each LK^{NPL} is, independently, —(CH₂)_{pNPL}— and C₂₋₈ alkenylenyl, wherein each of the —(CH₂)_{pNPL} and C₂₋₈ alkenylenyl is optionally substituted with one or more substituents, wherein each substituent is, independently, amino, hydroxyl, aminoalkyl, hydroxylalkyl, or alkyl;

each pNPL is, independently, an integer from 0 to 8;

q1NPL and q2NPL are each, independently, 0, 1, or 2;

each PL group is, independently, halo, hydroxyethoxymethyl, methoxyethoxymethyl, polyoxyethylene, or —(NR⁵)_{q1PL}—U^{PL}-LK^{PL}—(NR⁵″)_{q2PL}—V, wherein:

R⁵, R⁵′, and R⁵″ are each, independently, hydrogen, alkyl, or alkoxy;

each U^{PL} is, independently, absent or O, S, S(═O), S(═O)₂, NR⁵, —C(═O)—, —C(═O)—NR⁵—, —C(═O)—N═N—NR⁵—, —C(═O)—NR⁵—N═N—, —N═N—NR⁵—, —C(═N—N(R⁵)₂)—, —C(═NR⁵)—, —C(═O)O—, —C(═O)S—, —C(═S)—, —O—P(═O)₂O—, —S—C═N—, or —C(═O)—NR⁵—O—, wherein groups with two chemically nonequivalent termini can adopt either of the two possible orientations;

each V is, independently, nitro, cyano, amino, halo, hydroxy, alkoxy, alkylthio, alkylamino, dialkylamino, —NH(CH₂)ₚNH₂ wherein p is 1 to 5, —C(═O)NH(CH₂)ₚNH₂ wherein p is 1 to 5, —C(═O)NH(CH₂)ₚNHC(═NH)NH₂ wherein p is 1 to 5, —C(═O)NH(CH₂)ₚNHC(═O)NH₂ wherein p is 1 to 5, —NHC(═O)-alkyl, —N(CH₂CH₂NH₂)₂, diazamino, amidino, guanidino, ureido, carbamoyl, —C(═O)OH, —C(═O)OR^{c}, —C(═O)NH—OH, —O—NH—C(═NH)NH₂, —NH—S(═O)₂OH, S(═O)₂OH, NR^{d}R^{e}, semicarbazone, aryl, cycloalkyl, heterocycloalkyl, or heteroaryl, wherein each of the aryl and cycloalkyl is substituted with one or more substitutents, wherein each of the heterocycloalkyl and heteroaryl is optionally substituted with one or more substituents, and wherein each of the substituents for the aryl, cycloalkyl, heterocycloalkyl, and heteroaryl is, independently, nitro, cyano, amino, halo, hydroxy, alkoxy, alkylthio, alkylamino, dialkylamino, —NH(CH₂)ₚNH₂ wherein p is 1 to 5, —N(CH₂CH₂NH₂)₂, diazamino, amidino, guanidino, ureido, carbamoyl, —C(═O)OH, —C(═O)OR^{c}, —C(═O)NH—OH, —O—NH—C(═NH)NH₂, —NH—S(═O)₂OH, S(═O)₂OH, NR^{d}R^{e}, semicarbazone, aminosulfonyl, aminoalkoxy, aminoalkylthio, lower acylamino, or benzyloxycarbonyl;

each R^{c} is, independently, C₁₋₆ alkyl, C₁₋₆ haloalkyl, C₂₋₆ alkenyl, C₂₋₆ alkynyl, aryl, cycloalkyl, heteroaryl, heterocycloalkyl, arylalkyl, heteroarylalkyl, cycloalkylalkyl, or heterocycloalkylalkyl, each optionally substituted by one or more substitutents, wherein each substituent is, independently, OH, amino, halo, C₁₋₆ alkyl, C₁₋₆haloalkyl, aryl, arylalkyl, heteroaryl, heteroarylalkyl, cycloalkyl, or heterocycloalkyl;

R^{d} and R^{e} are, independently, H, C₁₋₆ alkyl, C₁₋₆ haloalkyl, C₂₋₆ alkenyl, C₂₋₆ alkynyl, aryl, heteroaryl, cycloalkyl, heterocycloalkyl, arylalkyl, heteroarylalkyl, cycloalkylalkyl, or heterocycloalkylalkyl, wherein each of the C₁₋₆ alkyl, C₁₋₆ haloalkyl, C₂₋₆ alkenyl, C₂₋₆ alkynyl, aryl, heteroaryl, cycloalkyl, heterocycloalkyl, arylalkyl, heteroarylalkyl, cycloalkylalkyl, or heterocycloalkylalkyl is optionally substituted by OH, amino, halo, C₁₋₆ alkyl, C₁₋₆ haloalkyl, C₁₋₆ haloalkyl, aryl, arylalkyl, heteroaryl, heteroarylalkyl, cycloalkyl, or heterocycloalkyl;

or R^{d} and R^{e} together with the N atom to which they are attached form a 4-, 5-, 6-, 7-, or 8-membered heterocycloalkyl;

each LK^{PL} is, independently, —(CH₂)_{pPL}— or C₂₋₈ alkenylenyl, wherein each of the —(CH₂)_{pNPL}— and C₂₋₈ alkenylenyl is optionally substituted with one or more substituents, wherein each substituent is, independently, amino, hydroxyl, aminoalkyl, hydroxylalkyl, or alkyl;

each pPL is, independently, an integer from 0 to 8;

q1 PL and q2PL are each, independently, 0, 1, or 2;

m11 is an integer from 1 to about 20; and

m12 is an integer from 1 to about 20.

The present invention also provides methods of modulating an immune response in a mammal comprising administering to the mammal in need thereof a therapeutically effective amount of a compound of Formula XXI:

R¹—[—X-A₁-Y—X-A₂-Y—]ₘ₁₃—X-L¹-Y—[X-A₁-Y—X-A₂-Y—]ₘ₁₄—R² XXI

or a pharmaceutically acceptable salt thereof,
wherein:

each X is, independently, NR⁸;

each Y is C═O;

each R⁸ is, independently, hydrogen or alkyl;

each A₂ is optionally substituted arylene or optionally substituted heteroarylene, and each A₁ is —(CH₂)_{q}—, wherein q is 1 to 7, wherein A₁ and A₂ are each, independently, optionally substituted with one or more PL group(s), one or more NPL group(s), or a combination of one or more PL group(s) and one or more NPL group(s);

R¹ is hydrogen, a PL group, or an NPL group, and R² is —X-A₁-Y—R¹¹, wherein R¹¹ is hydrogen, a PL group, or an NPL group; or

R¹ and R² are each, independently, hydrogen, a PL group, or an NPL group; or

R¹ and R² together are a single bond; or

R¹ is —Y-A₂-X—R¹², wherein R¹² is hydrogen, a PL group, or an NPL group, and R² is hydrogen, a PL group, or an NPL group;

L¹ is C₁₋₁₀alkylene optionally substituted with one or more substitutents, wherein each substituent is, independently, alkyl, halo, haloalkyl, aminoalkyl, hydroxylalkyl, V, or —(CH₂)_{pPL}—V wherein pPL is an integer from 1 to 5;

each V is, independently, hydroxy, amino, alkylamino, dialkylamino, —NH(CH₂)ₚNH₂ wherein p is 1 to 5, —C(═O)NH(CH₂)ₚNH₂ wherein p is 1 to 5, —C(═O)NH(CH₂)ₚNHC(═NH)NH₂ wherein p is 1 to 5, —C(═O)NH(CH₂)ₚNHC(═O)NH₂ wherein p is 1 to 5, —NHC(═O)-alkyl, —N(CH₂CH₂NH₂)₂, guanidino, amidino, ureido, carbamoyl, —C(═O)OH, —C(═O)OR^{c}, —C(═O)NH—OH, —O—NH—C(═NH)NH₂, —NH—S(═O)₂OH, S(═O)₂OH, NR^{d}R^{e}, a substituted aryl group, heterocycloalkyl, or heteroaryl, wherein each of the heterocycloalkyl and heteroaryl is optionally substituted with one more substituents, wherein each substituent is, independently, amino, halo, cyano, nitro, hydroxy, —NH(CH₂)ₚNH₂ wherein p is 1 to 5, —N(CH₂CH₂NH₂)₂, amidino, guanidino, aminosulfonyl, aminoalkoxy, aminoalkylthio, lower acylamino, or benzyloxycarbonyl; and wherein the substituted aryl group is substituted with one more substituents, wherein each substituent is, independently, amino, halo, cyano, nitro, hydroxy, —NH(CH₂)ₚNH₂ wherein p is 1 to 5, —N(CH₂CH₂NH₂)₂, amidino, guanidino, aminosulfonyl, aminoalkoxy, aminoalkylthio, lower acylamino, or benzyloxycarbonyl;

each NPL group is, independently, —B(OR⁴)₂ or

—(NR³)_{q1NPL}—U^{NPL}-LK^{NPL}—(NR³″)_{q}2NPL —R⁴′, wherein:

R³, R³′, and R³″ are each, independently, hydrogen, alkyl, or alkoxy;

R⁴ and R⁴′ are each, independently, hydrogen, alkyl, alkenyl, alkynyl, cycloalkyl, aryl, or heteroaryl, wherein each of the alkyl, alkenyl, alkynyl, cycloalkyl, aryl, and heteroaryl is optionally substituted with one or more substitutents, wherein each substituent is, independently, alkyl, halo, or haloalkyl;

each U^{NPL} is, independently, absent or O, S, S(═O), S(═O)₂, NR³, —C(═O)—, —C(═O)—NR³—, —C(═O)—N═N—NR³—, —C(═O)—NR³—N═N—, —N═N—NR³—, —C(═N—N(R³)₂)—, C(═NR³)—, —C(═O)O—, —C(═O)S—, —C(═S)—, —O—P(═O)₂O—, —S—C═N—, or —C(═O)—NR³—O—, wherein groups with two chemically nonequivalent termini can adopt both possible orientations;

each LK^{NPL} is, independently, —(CH₂)_{pNPL}— or C₂₋₈ alkenylenyl, wherein each of the —(CH₂)_{pNPL} and C₂₋₈ alkenylenyl is optionally substituted with one or more substituents, wherein each substituent is, independently, amino, hydroxyl, aminoalkyl, hydroxylalkyl, or alkyl;

each pNPL is, independently, an integer from 0 to 8;

q1NPL and q2NPL are each, independently, 0, 1, or 2;

each PL group is, independently, halo, hydroxyethoxymethyl, methoxyethoxymethyl, polyoxyethylene, or —(NR⁵′)_{q1PL}—U^{PL}-LK^{PL}—(NR⁵″)_{q1PL}—V, wherein:

R⁵, R⁵′, and R⁵″ are each, independently, hydrogen, alkyl, or alkoxy;

each U^{PL} is, independently, absent or O, S, S(═O), S(═O)₂, NR⁵, —C(═O)—NR⁵—, —C(═O)—N═N—NR⁵—, —C(═O)—NR⁵—N═N—, —N═N—NR⁵—, —C(═N—N(R⁵)₂)—, —C(═NR⁵)—, —C(═O)O—, —C(═O)S—, —C(═S)—, —O—P(═O)₂O—, —S—C═N—, or —C(═O)—NR⁵—O—, wherein groups with two chemically nonequivalent termini can adopt either of the two possible orientations;

each R^{c} is, independently, C₁₋₆ alkyl, C₁₋₆ haloalkyl, C₂₋₆ alkenyl, C₂₋₆ alkynyl, aryl, cycloalkyl, heteroaryl, heterocycloalkyl, arylalkyl, heteroarylalkyl, cycloalkylalkyl, or heterocycloalkylalkyl, each optionally substituted by one or more substitutents, wherein each substituent is, independently, OH, amino, halo, C₁₋₆ alkyl, C₁₋₆ haloalkyl, aryl, arylalkyl, heteroaryl, heteroarylalkyl, cycloalkyl, or heterocycloalkyl;

R^{d} and R^{e} are, independently, H, C₁₋₆ alkyl, C₁₋₆ haloalkyl, C₂₋₆ alkenyl, C₂₋₆ alkynyl, aryl, heteroaryl, cycloalkyl, heterocycloalkyl, arylalkyl, heteroarylalkyl, cycloalkylalkyl, or heterocycloalkylalkyl, wherein each of the C₁₋₆ alkyl, C₁₋₆ haloalkyl, C₂₋₆ alkenyl, C₂₋₆ alkynyl, aryl, heteroaryl, cycloalkyl, heterocycloalkyl, arylalkyl, heteroarylalkyl, cycloalkylalkyl, and heterocycloalkylalkyl is optionally substituted by OH, amino, halo, C₁₋₆ alkyl, C₁₋₆ haloalkyl, C₁₋₆ haloalkyl, aryl, arylalkyl, heteroaryl, heteroarylalkyl, cycloalkyl or heterocycloalkyl;

or R^{d} and R^{e} together with the N atom to which they are attached form a 4-, 5-, 6-, 7-, or 8-membered heterocycloalkyl;

each LK^{PL} is, independently, —(CH₂)_{pPL}— or C₂₋₈ alkenylenyl, wherein each of the —(CH₂)_{pNPL}— and C₂₋₈ alkenylenyl is optionally substituted with one or more substituents, wherein each substituent is, independently, amino, hydroxyl, aminoalkyl, hydroxylalkyl, or alkyl;

each pPL is, independently, an integer from 0 to 8;

q1PL and q2PL are each, independently, 0, 1, or 2;

m13 is an integer from 1 to about 10; and

m14 is an integer from 1 to about 10.

The present invention also provides methods of modulating an immune response in a mammal comprising administering to the mammal in need thereof a therapeutically effective amount of a compound of Formula XXII:

R¹—[—X-A₁-X—Z—Y-A₂-Y—Z]ₘ—R² XXII

or a pharmaceutically acceptable salt thereof,
wherein:

X is NR⁸, —NR⁸NR⁸—, C═O, or O;

Y is NR⁸, —NR⁸NR⁸—, C═O, S, or O;

R⁸ is hydrogen or alkyl;

Z is C═O, C═S, O═S═O, —NR⁸NR⁸—, or —C(═O)C(═O)—;

A₁ and A₂ are, independently, optionally substituted arylene or optionally substituted heteroarylene, wherein A₁ and A₂ are, independently, optionally substituted with one or more polar (PL) group(s), one or more non-polar (NPL) group(s), or a combination of one or more polar (PL) group(s) and one or more non-polar (NPL) group(s);

R¹ is
(i) hydrogen, a polar group (PL), or a non-polar group (NPL), and R² is —X-A₁-X—R¹, wherein A₁ is as defined above and is optionally substituted with one or more polar (PL) group(s), one or more non-polar (NPL) group(s), or a combination of one or more polar (PL) group(s) and one or more non-polar (NPL) group(s); or

(ii) hydrogen, a polar group (PL), or a non-polar group (NPL), and R² is —X-A₁-X—Z—Y-A₂-Y—R¹, wherein A₁ and A₂ are as defined above, and each of which is optionally substituted with one or more polar (PL) group(s), one or more non-polar (NPL) group(s), or a combination of one or more polar (PL) group(s) and one or more non-polar (NPL) group(s); or
(iii) hydrogen, a polar group (PL), or a non-polar group (NPL), and R² is —X-A′-X—R¹, wherein A′ is aryl or heteroaryl and is optionally substituted with one or more polar (PL) group(s), one or more non-polar (NPL) group(s), or a combination of one or more polar (PL) group(s) and one or more non-polar (NPL) group(s); or
(iv) hydrogen, a polar group (PL), or a non-polar group (NPL), and R² is —X-A₁-X—Z—Y-A′-Y—R¹, wherein A₁ is as defined above, A′ is aryl or heteroaryl, and each of A₁ and A′ is optionally substituted with one or more polar (PL) group(s), one or more non-polar (NPL) group(s), or a combination of one or more polar (PL) group(s) and one or more non-polar (NPL) group(s); or
(v) —Z—Y-A¹ and R² is hydrogen, a polar group (PL), or a non-polar group (NPL), wherein A′ is aryl or heteroaryl and is optionally substituted with one or more polar (PL) group(s), one or more non-polar (NPL) group(s), or a combination of one or more polar (PL) group(s) and one or more non-polar (NPL) group(s); or
(vi) —Z—Y-A′, and R² is —X-A″, wherein A′ and A″ are, independently, aryl or heteroaryl, and each of A and A″ is optionally substituted with one or more polar (PL) group(s), one or more non-polar (NPL) group(s), or a combination of one or more polar (PL) group(s) and one or more non-polar (NPL) group(s); or
(vii) R¹ and R² are, independently, a polar group (PL) or a non-polar group (NPL); or
(viii) R¹ and R² together form a single bond;

NPL is a nonpolar group independently selected from —B(OR⁴)₂ and —(NR³)_{q1NPL}—U^{NPL}—(CH₂)_{pNPL}—(NR³′)—_{q2NPL}—R⁴′, wherein:

R³, R³′, and R³″ are, independently, selected from hydrogen, alkyl, and alkoxy;

R⁴ and R⁴′ are, independently, selected from hydrogen, alkyl, alkenyl, alkynyl, cycloalkyl, aryl, and heteroaryl, any of which is optionally substituted with one or more alkyl or halo groups;

U^{NPL} is absent or selected from O, S, S(═O), S(═O)₂, NR³, —C(═O)—, —C(═O)—N═N—NR³—, —C(═O)—NR³—N═N—, —N═N—NR³—, —C(═N—N(R³)₂)—, —C(═NR³)—, —C(═O)O—, —C(═O)S—, —C(═S)—, —O—P(═O)₂O—, —R³⁰—, —R³S—, —S—C═N—, and —C(═O)—NR³—O—, wherein groups with two chemically nonequivalent termini can adopt both possible orientations;

the —(CH₂)_{pNPL}— alkylene chain is optionally substituted with one or more amino or hydroxy groups, or is unsaturated;

pNPL is 0 to 8;

q1NPL and q2NPL are, independently, 0, 1, or 2;

PL is a polar group selected from halo, hydroxyethoxymethyl, methoxyethoxymethyl, polyoxyethylene, and —(NR⁵)_{q1PL}—U^{PL}—(CH₂)_{pPL}(NR⁵)_{q2PL}—V, wherein:

R⁵, R⁵′, and R⁵″ are, independently, selected from hydrogen, alkyl, and alkoxy;

U^{PL} is absent or selected from O, S, S(═O), S(═O)₂, NR⁵, —C(═O)—, —C(═O)—N═N—NR⁵—, —C(═O)—NR⁵—N═N—, —N═N—NR⁵—, —C(═N—N(R⁵)₂)—, —C(═NR⁵)—, —C(═O)O—, —C(═O)S—, —C(═S)—, —O—P(═O)₂O—, —R⁵O—, —R⁵S—, —S—C═N—, and —C(═O)—NR⁵—O—, wherein groups with two chemically nonequivalent termini can adopt both possible orientations;

V is selected from nitro, cyano, amino, hydroxy, alkoxy, alkylthio, alkylamino, dialkylamino, —NH(CH₂)ₚNH₂ wherein p is 1 to 4, —N(CH₂CH₂NH₂)₂, diazamino, amidino, guanidino, guanyl, semicarbazone, aryl, heterocycle, and heteroaryl, any of which is optionally substituted with one or more of amino, halo, cyano, nitro, hydroxy, —NH(CH₂)ₚNH₂ wherein p is 1 to 4, —N(CH₂CH₂NH₂)₂, amidino, guanidino, guanyl, aminosulfonyl, aminoalkoxy, aminoalkylthio, lower acylamino, or benzyloxycarbonyl;

the —(CH₂)_{pPL}— alkylene chain is optionally substituted with one or more amino or hydroxy groups, or is unsaturated;

pPL is 0 to 8;

q1 PL and q2PL are, independently, 0, 1, or 2; and

m is 1 to about 20.

The present invention also provides methods of modulating an immune response in a mammal comprising administering to the mammal in need thereof a therapeutically effective amount of a compound of Formula XXIII:

R¹-[-A₁-W-A₂-W—]ₘ—R² XXIII

or a pharmaceutically acceptable salt thereof,
wherein:

A₁ and A₂ are, independently, optionally substituted arylene or optionally substituted heteroarylene, wherein:

(i) A₁ and A₂ are, independently, optionally substituted with one or more polar (PL) group(s), one or more non-polar (NPL) group(s), or a combination of one or more polar (PL) group(s) and one or more non-polar (NPL) group(s); or

(ii) one of A₁ or A₂ is as defined above and is optionally substituted with one or more polar (PL) group(s), one or more non-polar (NPL) group(s), or a combination of one or more polar (PL) group(s) and one or more non-polar (NPL) group(s); and the other of A₁ or A₂ is the group —C≡C(CH₂)ₚC≡C—, wherein p is 0 to 8, and the —(CH₂)ₚ— alkylene chain is optionally substituted with one or more amino or hydroxyl groups;

W is absent, or represents —CH₂—, —CH₂—CH₂—, —CH═CH—, or —C≡C—;

R¹ is
(i) hydrogen, a polar group (PL), or a non-polar group (NPL), and R² is -A₁-R¹, wherein A₁ is as defined above and is optionally substituted with one or more polar (PL) group(s), one or more non-polar (NPL) group(s), or a combination of one or more polar (PL) group(s) and one or more non-polar (NPL) group(s); or
(ii) hydrogen, a polar group (PL), or a non-polar group (NPL), and R² is -A₁-W-A₂-R¹, wherein each of A₁ and A₂ is as defined above and is optionally substituted with one or more polar (PL) group(s), one or more non-polar (NPL) group(s), or a combination of one or more polar (PL) group(s) and one or more non-polar (NPL) group(s); or
(iii) A′-W— and R² is -A₁-W-A′, wherein A′ is aryl or heteroaryl, either of which is optionally substituted with one or more polar (PL) group(s), one or more non-polar (NPL) group(s), or a combination of one or more polar (PL) group(s) and one or more non-polar (NPL) group(s); or
(iv) A′-W— and R² is -A′, wherein A′ is aryl or heteroaryl, either of which is optionally substituted with one or more polar (PL) group(s), one or more non-polar (NPL) groups(s), or a combination of one or more polar (PL) group(s) and one or more non-polar (NPL) group(s); or
(iv) R¹ and R² together form a single bond; NPL is a nonpolar group independently selected from —B(OR⁴)₂ or —(NR³′)_{q1NPL}—U^{NPL}—(CH₂)_{pNPL}—(NR³)_{q2NPL}—R⁴, wherein:

R³, R³′, and R³″ are, independently, selected from hydrogen, alkyl, and alkoxy;

R⁴ is selected from hydrogen, alkyl, alkenyl, alkynyl, cycloalkyl, aryl, and heteroaryl, any of which is optionally substituted with one or more alkyl or halo groups;

U^{NPL} is absent or selected from O, S, S(═O), S(═O)₂, NR³, —(C═O)—, —(C═O)—N═N—NR³—, —(C═O)—NR³—N═N—, —N═N—NR³—, —C(═N—N(R³)₂)—, —C(═NR³)—, —C(═O)O—, —C(═O)S—, —C(═S)—, —O—P(═O)₂O—, —R³⁰—, —R³S—, —S—C═N— and —(C═O)—NR³—O—, wherein groups with two chemically nonequivalent termini can adopt both possible orientations;

the —(CH₂)_{pNPL}— alkylene chain is optionally substituted with one or more alkyl, amino or hydroxyl groups, or the alkylene chain is unsaturated;

pNPL is 0 to 8;

q1NPL and q2NPL are, independently, 0 to 2;

PL is a polar group selected from halo, hydroxyethoxymethyl, methoxyethoxymethyl, polyoxyethylene, and —(NR⁵′)_{q1PL}—U^{PL}—(CH₂)_{pPL}—(NR⁵)_{q2PL}—V, wherein:

R⁵, R⁵′, and R⁵″ are, independently, selected from hydrogen, alkyl, and alkoxy;

U^{PL} is absent or selected from O, S, S(═O), S(═O)₂, NR⁵, —(C═O)—, —(C═O)—N═N—NR⁵—, —(C═O)—NR⁵—N═N—, —N═N—NR⁵—, —C(═N—N(R⁵)₂)—, —C(═NR⁵)—, —C(═O)O—, —C(═O)S—, —C(═S)—, —O—P(═O)₂O—, —R⁵—O—, —R⁵S—, —S—C═N—, and —(C═O)—NR⁵—O—, wherein groups with two chemically nonequivalent termini can adopt both possible orientations;

V is selected from nitro, cyano, amino, hydroxyl, alkoxy, alkylthio, alkylamino, dialkylamino, —NH(CH₂)ₚNH₂, —N(CH₂CH₂NH₂)₂, diazamino, amidino, guanidino, guanyl, semicarbazone, aryl, heterocycle, and heteroaryl, any of which is optionally substituted with one or more of amino, halo, cyano, nitro, hydroxyl, —NH(CH₂)ₚNH₂, —N(CH₂CH₂NH₂)₂, amidino, guanidino, guanyl, aminosulfonyl, aminoalkoxy, aminoalkylthio, lower acylamino, or benzyloxycarbonyl;

the —(CH₂)_{pPL}— alkylene chain is optionally substituted with one or more amino or hydroxyl groups, or the alkylene chain is unsaturated;

pPL is 0 to 8;

q1PL and q2PL are, independently, 0 to 2; and

m is 1 to about 25.

The present invention also provides methods of modulating an immune response in a mammal comprising administering to the mammal in need thereof a therapeutically effective amount of a compound chosen from:

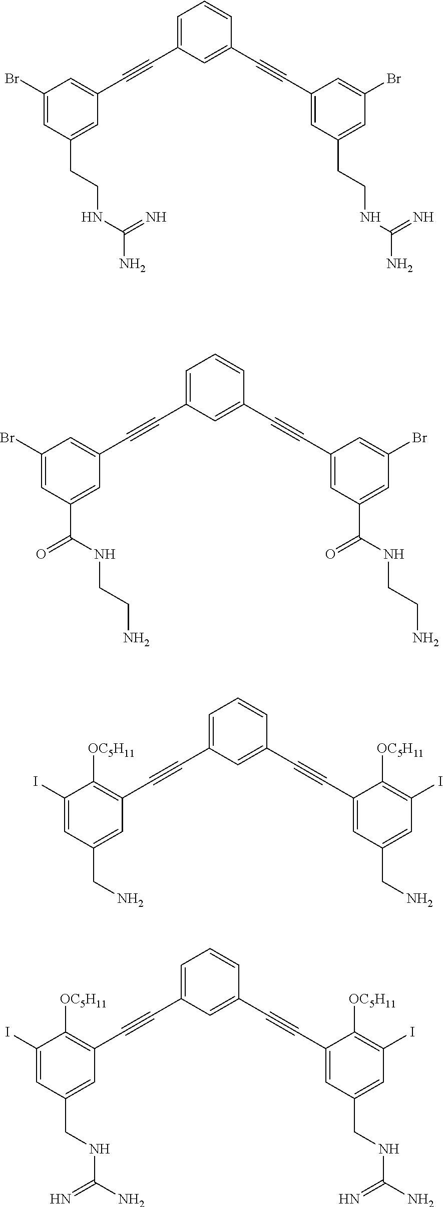

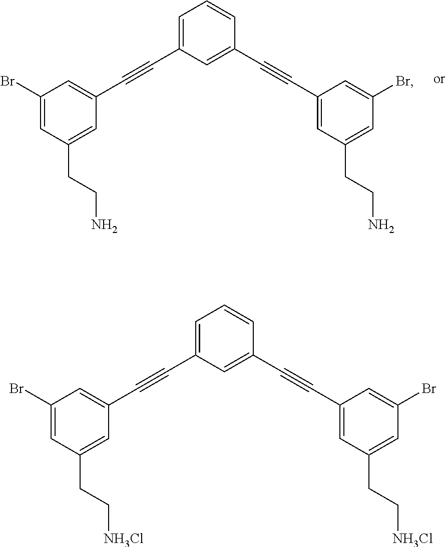

or a pharmaceutically acceptable salt thereof.

The present invention also provides methods of modulating an immune response in a mammal comprising administering to the mammal in need thereof a therapeutically effective amount of a compound of Formula XXIV:

R¹—X-A₁-X—Y-A₂-Y—X-A₁-X—R² XXIV

or a pharmaceutically acceptable salt thereof,
wherein:

X is NR⁸, O, S, or —N(R⁸)N(R⁸)—;

Y is C═O, C═S, or O═S═O;

R⁸ is hydrogen or alkyl;

A₁ and A₂ are, independently, optionally substituted arylene or optionally substituted heteroarylene, wherein A₁ and A₂ are, independently, optionally substituted with one or more polar (PL) group(s), one or more non-polar (NPL) group(s), or a combination of one or more polar (PL) group(s) and one or more non-polar (NPL) group(s);

R¹ is a polar group (PL) or a non-polar group (NPL);

R² is R¹;

NPL is a nonpolar group independently selected from —B(OR⁴)₂ and —(NR³′)_{q1NPL}—U^{NPL}—(CH₂)_{pNPL}(NR³″)_{q2NPL}—R⁴′, wherein:

R³, R³′, and R³″ are, independently, selected from hydrogen, alkyl, and alkoxy;

R⁴ and R⁴′ are, independently, selected from the group consisting of hydrogen, alkyl, alkenyl, alkynyl, cycloalkyl, aryl, and heteroaryl, any of which is optionally substituted with one or more alkyl or halo groups;

U^{NPL} is absent or selected from O, S, S(═O), S(═O)₂, NR³, —C(═O)—, —C(═O)—N═N—NR³—, —C(═O)—NR³—N═N—, —N═N—NR³—, —C(═N—N(R³)₂)—, —C(═NR³)—, —C(═O)O—, —C(═O)S—, —C(═S)—, —O—P(═O)₂O—, —R³⁰—, —R³S—, —S—C═N—, and —C(═O)—NR³—O—, wherein groups with two chemically nonequivalent termini can adopt both possible orientations; the —(CH₂)_{pNPL}— alkylene chain is optionally substituted with one or more amino or hydroxy groups, or is unsaturated;

pNPL is 0 to 8;

q1NPL and q2NPL are, independently, 0, 1, or 2;

PL is a polar group selected from halo, hydroxyethoxymethyl, methoxyethoxymethyl, polyoxyethylene, and —(NR⁵′)_{q1PL}—U^{PL}—(CH₂)_{pPL}—(NR⁵)_{q2PL}—V, wherein:

R⁵, R⁵′, and R⁵″ are, independently, selected from hydrogen, alkyl, and alkoxy;

U^{PL} is absent or selected from O, S, S(═O), S(═O)₂, NR⁵, —C(═O)—, —C(═O)—N═N—NR⁵—, —C(═O)—NR⁵—N═N—, —N═N—NR⁵—, —C(═N—N(R⁵)₂)—, —C(═NR⁵)—, —C(═O)O—, —C(═O)S—, —C(═S)—, —O—P(═O)₂O—, —R⁵O—, —R⁵S—, —S—C═N—, and —C(═O)—NR⁵—O—, wherein groups with two chemically nonequivalent termini can adopt both possible orientations;

V is selected from nitro, cyano, amino, hydroxy, alkoxy, alkylthio, alkylamino, dialkylamino, —NH(CH₂)ₚNH₂ wherein p is 1 to 4, —N(CH₂CH₂NH₂)₂, diazamino, amidino, guanidino, guanyl, semicarbazone, aryl, heterocycle and heteroaryl, any of which is optionally substituted with one or more of amino, halo, cyano, nitro, hydroxy, —NH(CH₂)ₚNH₂ wherein p is 1 to 4, —N(CH₂CH₂NH₂)₂, amidino, guanidino, guanyl, aminosulfonyl, aminoalkoxy, aminoalkylthio, lower acylamino, or benzyloxycarbonyl; the —(CH₂)_{pPL}— alkylene chain is optionally substituted with one or more amino or hydroxy groups, or is unsaturated;

pPL is 0 to 8; and

q1PL and q2PL are, independently, 0, 1, or 2.

The present invention also provides methods of modulating an immune response in a mammal comprising administering to the mammal in need thereof a therapeutically effective amount of a compound chosen from:

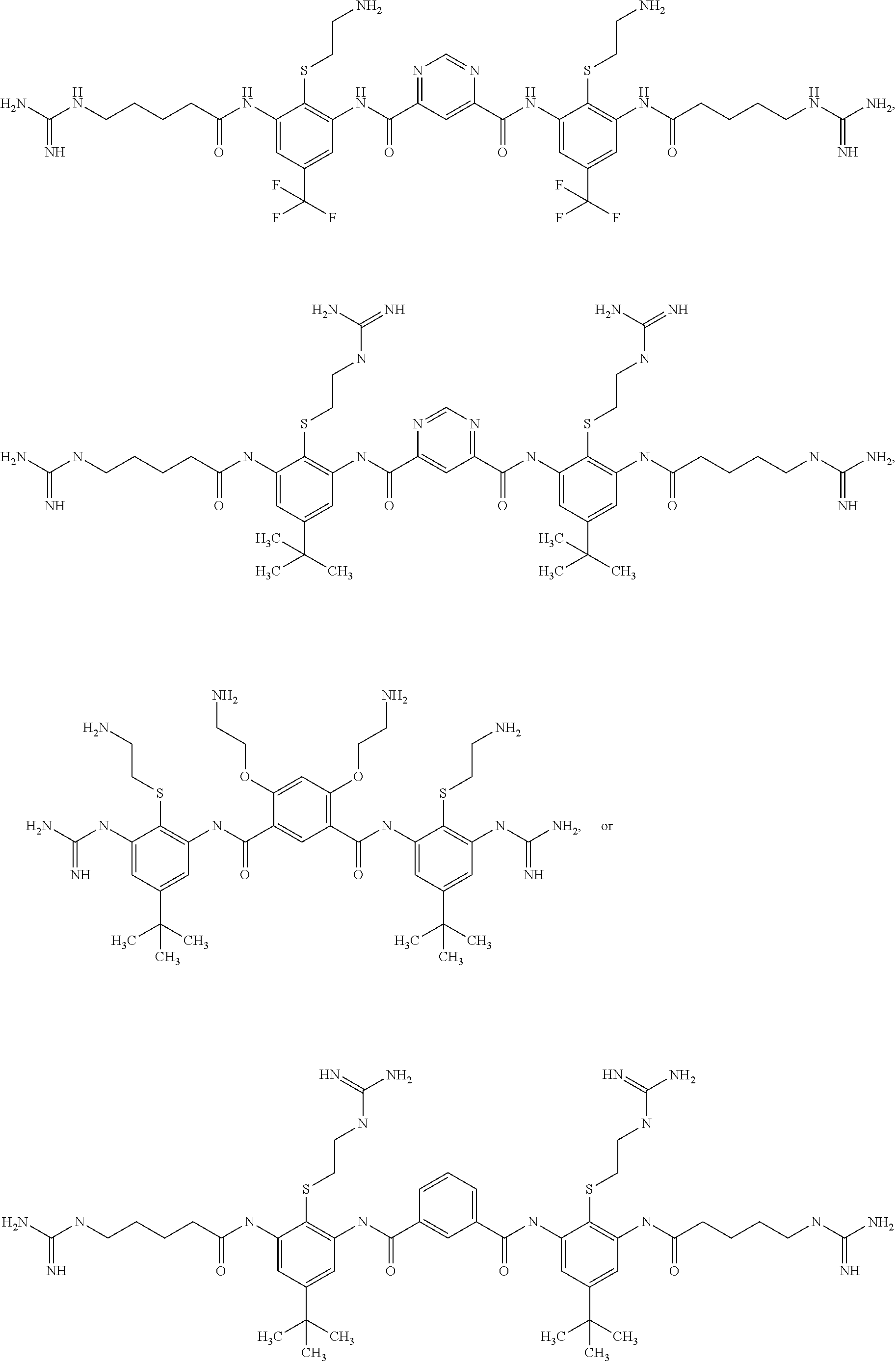

or a pharmaceutically acceptable salt thereof.

The present invention also provides methods of modulating an immune response in a mammal comprising administering to the mammal in need thereof a therapeutically effective amount of a compound of Formula XXV:

A-(B)ₙ₁-(D)ₘ₁-H XXV

or a pharmaceutically acceptable salt thereof,
wherein:

A is the residue of a chain transfer agent;

B is —[CH₂—C(R¹¹)(B₁₁)]—, wherein B₁₁ is —X₁₁—Y₁₁—Z₁₁, wherein

X₁₁ is carbonyl (—C(═O)—) or optionally substituted C₁₋₆ alkylene; or X₁₁ is absent;

Y₁₁ is O, NH, or optionally substituted C₁₋₆ alkylene; or Y₁₁ is absent;

Z₁ₗ is —Z_{11A}—Z_{11B}, wherein Z_{11A} is alkylene, arylene, or heteroarylene, any of which is optionally substituted; or Z_{11A} is absent; and Z_{11B} is -guanidine, -amidino, —N(R³)(R⁴), or —N⁺(R³)(R⁴)(R⁵), wherein R³, R⁴, and R⁵ are, independently, hydrogen, alkyl, aminoalkyl, aryl, heteroaryl, heterocyclic, or aralkyl; or

Z₁₁ is pyridinium

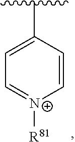

or phosphonium

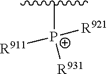

wherein R⁸¹, R⁹¹¹, R⁹²¹, and R⁹³¹ are, independently, hydrogen or alkyl;

R¹¹ is hydrogen or C₁₋₄ alkyl;

D is —[CH₂—C(R²¹)(D₂₁)]—, wherein D₂₁ is —X₂₁—Y₂₁—Z₂₁, wherein

X₂₁ is carbonyl (—C(═O)—) or optionally substituted C₁₋₆ alkylene; or X₂₁ is absent;

Y₂₁ is O, NH, or optionally substituted C₁₋₆ alkylene, or Y₂₁ is absent;

Z₂₁ is alkyl, cycloalkyl, alkoxy, aryl, or aralkyl, any of which is optionally substituted;

R²¹ is hydrogen or C₁₋₄ alkyl;

m₁, the mole fraction of D, is about 0.1 to about 0.9; and

n₁, the mole fraction of B, is 1−m₁;

wherein the compound is a random copolymer of B and D, and

wherein the copolymer has a degree of polymerization of about 5 to about 50.

The present invention also provides methods of modulating an immune response in a mammal comprising administering to the mammal in need thereof a therapeutically effective amount of a compound chosen from:

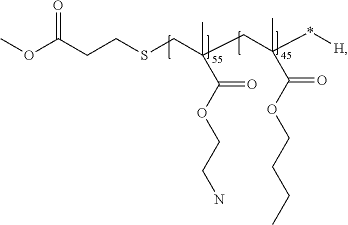

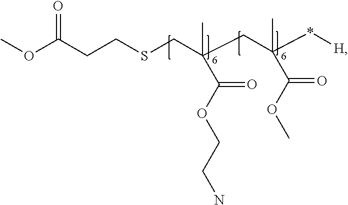

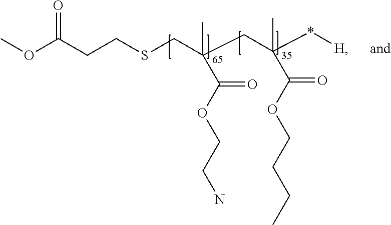

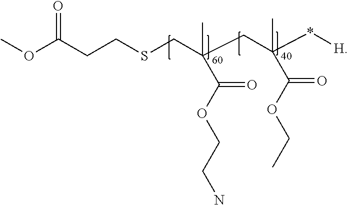

The present invention also provides methods of modulating an immune response in a mammal comprising administering to the mammal in need thereof a therapeutically effective amount of a compound chosen from Compound 1-Compound 146.

For each of the above-mentioned methods, the method of modulating an immune response comprises decreasing the production of a cytokine. In some embodiments, the cytokine is chosen from TNFalpha, IL-1Beta, IL-1alpha, IL-8, IL-6, IL-10, IL-11, IL-12, TGF-Beta, and IFNgamma. In some embodiments, the immune response is against an oral pathogen. In some embodiments, the oral pathogen is chosen from: Aggregatibacter spp. such as, for example, Aggregatibacter actinomycetemcomitans; Porphyromonas spp. such as, for example, Porphyromonas gingivalis; Streptococcus spp. such as, for example, Streptococcus sanguis and Streptococcus mutans, Candida spp. such as, for example, Candida albicans, Candida glabrata, Candida krusei, Candida dubliniensis, Candida parapsilosis, and Candida tropicalis; Actinomyces spp. such as, for example, Actinomyces viscosus; and Lactobacillus spp. such as, for example, Lactobacillus casei. In some embodiments, the immune response is against a bacterial pathogen. In some embodiments, the bacterial pathogen is chosen from: Staphylococcus spp., such as, for example, Staphylococcus aureus, methicillin-resistant Staphylococcus aureus, and Staphylococcus epidermidis; Streptococcus spp. such as, for example, Streptococcus pneumoniae, Streptococcus pyogenes, and Streptococcus viridans; Escherichia spp. such as, for example, E. coli; Enterococcus spp. such as, for example, Enterococcus faecalis and Enterococcus faecium; Psuedomonas spp. such as, for example, Pseudomonas aeruginosa; Acinetobacter spp. such as, for example, A. baumannii; Haemophilus spp. such as, for example, Haemophilus influenzae; Serratia spp. such as, for example, Serratia marcescens; Moraxella spp. such as, for example, Moraxella catarrhalis; Klebsiella spp. such as, for example, Klebsiella pneumoniae; Proteus spp. such as, for example, Proteus vulgaris and Proteus mirabilis; Bacteroides spp. such as, for example, Bacteroides fragalis; Clostridium spp. such as, for example, Clostridium difficile and Clostridium perfringens; and Propionibacterium spp. such as, for example, Propionibacterium acnes.

### BRIEF DESCRIPTION OF THE DRAWINGS

FIGS. 1A, 1B, and 1C show activity of mPE against Aa biofilms.

FIG. 2 shows activity of mPE against biofilms of P. gingivalis.

FIGS. 3A and 3B shows anti-inflammatory activity of mPE in gingival epithelial cell line OKF6/TERT or THP-1 cells, respectively.

FIGS. 4A and 4B shows affect on NF-κB-regulated genes.

### DESCRIPTION OF EMBODIMENTS

Unless defined otherwise, all technical and scientific terms have the same meaning as is commonly understood by one of ordinary skill in the art to which the embodiments disclosed belongs.

As used herein, the terms “comprising” (and any form of comprising, such as “comprise”, “comprises”, and “comprised”), “having” (and any form of having, such as “have” and “has”), “including” (and any form of including, such as “includes” and “include”), or “containing” (and any form of containing, such as “contains” and “contain”), are inclusive or open-ended and do not exclude additional, un-recited elements or method steps.

As used herein, the terms “a” or “an” means “at least one” or “one or more” unless the context clearly indicates otherwise.

As used herein, the term “about” means that the numerical value is approximate and small variations would not significantly affect the practice of the disclosed embodiments. Where a numerical limitation is used, unless indicated otherwise by the context, “about” means the numerical value can vary by ±10% and remain within the scope of the disclosed embodiments.

As used herein, the term “n-membered”, where n is an integer, typically describes the number of ring-forming atoms in a moiety, where the number of ring-forming atoms is n. For example, pyridine is an example of a 6-membered heteroaryl ring and thiophene is an example of a 5-membered heteroaryl ring.

As used herein, the term “alkyl” refers to a saturated hydrocarbon group which is straight-chained or branched. An alkyl group can contain from 1 to 20, from 2 to 20, from 1 to 10, from 1 to 8, from 1 to 6, from 1 to 4, or from 1 to 3 carbon atoms. Examples of alkyl groups include, but are not limited to, methyl (Me), ethyl (Et), propyl (e.g., n-propyl and isopropyl), butyl (e.g., n-butyl, isobutyl, t-butyl), pentyl (e.g., n-pentyl, isopentyl, neopentyl), and the like.

As used herein, the term “alkylene” or “alkylenyl” refers to a divalent alkyl linking group. An example of an alkylene (or alkylenyl) is methylene or methylenyl

As used herein, the term “alkenyl” refers to an alkyl group having one or more double carbon-carbon bonds. Examples of alkenyl groups include, but are not limited to, ethenyl, propenyl, cyclohexenyl, and the like.

As used herein, the term “alkenylenyl” refers to a divalent linking alkenyl group.

As used herein, the term “alkynyl” refers to an alkyl group having one or more triple carbon-carbon bonds. Examples of alkynyl groups include, but are not limited to, ethynyl, propynyl, and the like.

As used herein, the term “alkynylenyl” refers to a divalent linking alkynyl group.

As used herein, the term “haloalkyl” refers to an alkyl group having one or more halogen substituents. Examples of haloalkyl groups include, but are not limited to, CF₃, C₂F₅, CH₃, CHCl₂, C₂Cl₅, CH₂CF₃, and the like.

As used herein, the term “aryl” refers to monocyclic or polycyclic (e.g., having 2, 3 or 4 fused rings) aromatic hydrocarbons. In some embodiments, aryl groups have from 6 to about 20 carbon atoms. In some embodiments, aryl groups have from 6 to 10 carbon atoms. Examples of aryl groups include, but are not limited to, phenyl, naphthyl, anthracenyl, phenanthrenyl, indanyl, indenyl, and the like.

As used herein, the term “cycloalkyl” refers to non-aromatic cyclic hydrocarbons including cyclized alkyl, alkenyl, and alkynyl groups that contain up to 20 ring-forming carbon atoms. Cycloalkyl groups can include mono- or polycyclic ring systems such as fused ring systems, bridged ring systems, and spiro ring systems. In some embodiments, polycyclic ring systems include 2, 3, or 4 fused rings. A cycloalkyl group can contain from 3 to about 15, from 3 to 10, from 3 to 8, from 3 to 6, from 4 to 6, from 3 to 5, or from 5 to 6 ring-forming carbon atoms. Ring-forming carbon atoms of a cycloalkyl group can be optionally substituted by oxo or sulfido. Examples of cycloalkyl groups include, but are not limited to, cyclopropyl, cyclobutyl, cyclopentyl, cyclohexyl, cycloheptyl, cyclopentenyl, cyclohexenyl, cyclohexadienyl, cycloheptatrienyl, norbornyl, norpinyl, norcarnyl, adamantyl, and the like. Also included in the definition of cycloalkyl are moieties that have one or more aromatic rings fused (having a bond in common with) to the cycloalkyl ring, for example, benzo or thienyl derivatives of pentane, pentene, hexane, and the like (e.g., 2,3-dihydro-1H-indene-1-yl, or 1H-inden-2(3H)-one-1-yl).

As used herein, the term “heteroaryl” refers to an aromatic heterocycle having up to 20 ring-forming atoms and having at least one heteroatom ring member (ring-forming atom) such as sulfur, oxygen, or nitrogen. In some embodiments, the heteroaryl group has at least one or more heteroatom ring-forming atoms, each of which are, independently, sulfur, oxygen, or nitrogen. In some embodiments, the heteroaryl group has from 1 to about 20 carbon atoms, from 1 to 5, from 1 to 4, from 1 to 3, or from 1 to 2, carbon atoms as ring-forming atoms. In some embodiments, the heteroaryl group contains 3 to 14, 3 to 7, or 5 to 6 ring-forming atoms. In some embodiments, the heteroaryl group has 1 to 4, 1 to 3, or 1 to 2 heteroatoms. Heteroaryl groups include monocyclic and polycyclic (e.g., having 2, 3 or 4 fused rings) systems. Examples of heteroaryl groups include, but are not limited to, pyridyl, pyrimidinyl, pyrazinyl, pyridazinyl, triazinyl, furyl, quinolyl, isoquinolyl, thienyl, imidazolyl, thiazolyl, indolyl (such as indol-3-yl), pyrryl, oxazolyl, benzofuryl, benzothienyl, benzthiazolyl, isoxazolyl, pyrazolyl, triazolyl, tetrazolyl, indazolyl, 1,2,4-thiadiazolyl, isothiazolyl, benzothienyl, purinyl, carbazolyl, benzimidazolyl, indolinyl, and the like.

As used herein, the term “heterocycloalkyl” refers to non-aromatic heterocycles having up to 20 ring-forming atoms including cyclized alkyl, alkenyl, and alkynyl groups, where one or more of the ring-forming carbon atoms is replaced by a heteroatom such as an O, N, or S atom. Heterocycloalkyl groups can be mono or polycyclic (e.g., fused, bridged, or spiro systems). In some embodiments, the heterocycloalkyl group has from 1 to about 20 carbon atoms, or 3 to about 20 carbon atoms. In some embodiments, the heterocycloalkyl group contains 3 to 14, 3 to 7, or 5 to 6 ring-forming atoms. In some embodiments, the heterocycloalkyl group has 1 to 4, 1 to 3, or 1 to 2 heteroatoms. In some embodiments, the heterocycloalkyl group contains 0 to 3 double bonds. In some embodiments, the heterocycloalkyl group contains 0 to 2 triple bonds. Examples of heterocycloalkyl groups include, but are not limited to, morpholino, thiomorpholino, piperazinyl, tetrahydrofuranyl, tetrahydrothienyl, 2,3-dihydrobenzofuryl, 1,3-benzodioxole, benzo-1,4-dioxane, piperidinyl, pyrrolidinyl, isoxazolidinyl, isothiazolidinyl, pyrazolidinyl, oxazolidinyl, thiazolidinyl, imidazolidinyl, pyrrolidin-2-one-3-yl, and the like. In addition, ring-forming carbon atoms and heteroatoms of a heterocycloalkyl group can be optionally substituted by oxo or sulfido. For example, a ring-forming S atom can be substituted by 1 or 2 oxo (form a S(O) or S(O)₂). For another example, a ring-forming C atom can be substituted by oxo (form carbonyl). Also included in the definition of heterocycloalkyl are moieties that have one or more aromatic rings fused (having a bond in common with) to the nonaromatic heterocyclic ring including, but not limited to, pyridinyl, thiophenyl, phthalimidyl, naphthalimidyl, and benzo derivatives of heterocycles such as indolene, isoindolene, 4,5,6,7-tetrahydrothieno[2,3-c]pyridine-5-yl, 5,6-dihydrothieno[2,3-c]pyridin-7(4H)-one-5-yl, isoindolin-1-one-3-yl, and 3,4-dihydroisoquinolin-1(2H)-one-3yl groups. Ring-forming carbon atoms and heteroatoms of the heterocycloalkyl group can be optionally substituted by oxo or sulfido.

As used herein, the term “halo” refers to halogen groups including, but not limited to fluoro, chloro, bromo, and iodo.

As used herein, the term “alkoxy” refers to an —O-alkyl group. Examples of alkoxy groups include, but are not limited to, methoxy, ethoxy, propoxy (e.g., n-propoxy and isopropoxy), t-butoxy, and the like.

As used herein, the term “haloalkoxy” refers to an —O-haloalkyl group. An example of an haloalkoxy group is OCF₃.

As used herein, the term “alkylthio” refers to an —S-alkyl group. An example of an alkylthio group is —SCH₂CH₃.

As used herein, the term “arylalkyl” refers to a C₁₋₆ alkyl substituted by aryl and “cycloalkylalkyl” refers to C₁₋₆ alkyl substituted by cycloalkyl.

As used herein, the term “heteroarylalkyl” refers to a C₁₋₆ alkyl group substituted by a heteroaryl group, and “heterocycloalkylalkyl” refers to a C₁₋₆ alkyl substituted by heterocycloalkyl.

As used herein, the term “amino” refers to NH₂.

As used herein, the term “alkylamino” refers to an amino group substituted by an alkyl group. An example of an alkylamino is —NHCH₂CH₃.

As used herein, the term “arylamino” refers to an amino group substituted by an aryl group. An example of an alkylamino is —NH(phenyl).

As used herein, the term “aminoalkyl” refers to an alkyl group substituted by an amino group. An example of an aminoalkyl is —CH₂CH₂NH₂.

As used herein, the term “aminosulfonyl” refers to —S(═O)₂NH₂.

As used herein, the term “aminoalkoxy” refers to an alkoxy group substituted by an amino group. An example of an aminoalkoxy is —OCH₂CH₂NH₂.

As used herein, the term “aminoalkylthio” refers to an alkylthio group substituted by an amino group. An example of an aminoalkylthio is —SCH₂CH₂NH₂.

As used herein, the term “amidino” refers to —C(═NH)NH₂.

As used herein, the term “acylamino” refers to an amino group substituted by an acyl group (e.g., —O—C(═O)—H or —O—C(═O)-alkyl). An example of an acylamino is —NHC(═O)H or —NHC(═O)CH₃. The term “lower acylamino” refers to an amino group substituted by a loweracyl group (e.g., —O—C(═O)—H or —O—C(═O)—C₁₋₆alkyl). An example of a lower acylamino is —NHC(═O)H or —NHC(═O)CH₃.

As used herein, the term “carbamoyl” refers to —C(═O)—NH₂.

As used herein, the term “cyano” refers to —CN.

As used herein, the term “dialkylamino” refers to an amino group substituted by two alkyl groups.

As used herein, the term “diazamino” refers to —N(NH₂)₂.

As used herein, the term “guanidino” refers to —NH(═NH)NH₂.

As used herein, the term “heteroarylamino” refers to an amino group substituted by a heteroaryl group. An example of an alkylamino is —NH-(2-pyridyl).

As used herein, the term “hydroxyalkyl” or “hydroxylalkyl” refers to an alkyl group substituted by a hydroxyl group. Examples of a hydroxylalkyl include, but are not limited to, —CH₂OH and —CH₂CH₂OH.

As used herein, the term “nitro” refers to —NO₂.

As used herein, the term “semicarbazone” refers to ═NNHC(═O)NH₂.

As used herein, the term “ureido” refers to —NHC(═O)—NH₂.

As used herein, the phrase “optionally substituted” means that substitution is optional and therefore includes both unsubstituted and substituted atoms and moieties. A “substituted” atom or moiety indicates that any hydrogen on the designated atom or moiety can be replaced with a selection from the indicated substituent group, provided that the normal valency of the designated atom or moiety is not exceeded, and that the substitution results in a stable compound. For example, if a methyl group is optionally substituted, then 3 hydrogen atoms on the carbon atom can be replaced with substituent groups.

As used herein, the term, “compound” refers to all stereoisomers, tautomers, and isotopes of the compounds described in the present invention.

As used herein, the phrase “substantially isolated” refers to a compound that is at least partially or substantially separated from the environment in which it is formed or detected.

As used herein, the phrase “pharmaceutically acceptable” refers to those compounds, materials, compositions, and/or dosage forms which are, within the scope of sound medical judgment, suitable for use in contact with tissues of humans and animals.

As used herein, the term “animal” includes, but is not limited to, humans and non-human vertebrates such as wild, domestic and farm animals.

As used herein, the term “contacting” refers to the bringing together of an indicated moiety in an in vitro system or an in vivo system.

As used herein, the term “individual” or “patient,” used interchangeably, refers to any animal, including mammals, such as mice, rats, other rodents, rabbits, dogs, cats, swine, cattle, sheep, horses, or primates, such as humans.

As used herein, the phrase “therapeutically effective amount” refers to the amount of active compound or pharmaceutical agent that elicits the biological or medicinal response that is being sought in a tissue, system, animal, individual or human by a researcher, veterinarian, medical doctor or other clinician.

As used herein, the phrase “in need thereof” means that the subject or animal or human has been previously diagnosed with having a need of modulation of an immune response or otherwise identified as having a need of modulation of an immune response, or that the subject or animal or human has been previously diagnosed with having a need of reducing production of a cytokine or otherwise identified as having a need of reducing production of a cytokine.

At various places in the present specification, substituents of compounds of the invention are disclosed in groups or in ranges. It is specifically intended that the invention include each and every individual subcombination of the members of such groups and ranges. For example, the term “C₁₋₆ alkyl” is specifically intended to individually disclose methyl, ethyl, C₃ alkyl, C₄ alkyl, C₈ alkyl, and C₆ alkyl.

For compounds of the invention in which a variable appears more than once, each variable can be a different moiety selected from the Markush group defining the variable. For example, where a structure is described having two R groups that are simultaneously present on the same compound, the two R groups can represent different moieties selected from the Markush groups defined for R. In another example, when an optionally multiple substituent is designated in the form:

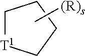

then it is understood that substituent R can occur s number of times on the ring, and R can be a different moiety at each occurrence. Further, in the above example, where the variable T¹ is defined to include hydrogens, such as when T¹ is CH₂, NH, etc., any floating substituent such as R in the above example, can replace a hydrogen of the T¹ variable as well as a hydrogen in any other non-variable component of the ring.

It is further appreciated that certain features of the invention, which are, for clarity, described in the context of separate embodiments, can also be provided in combination in a single embodiment. Conversely, various features of the invention which are, for brevity, described in the context of a single embodiment, can also be provided separately or in any suitable subcombination.

The compounds described herein can be asymmetric (e.g., having one or more stereocenters). All stereoisomers, such as enantiomers and diastereomers, are intended to be included within the scope of the invention unless otherwise indicated. Compounds of the present invention that contain asymmetrically substituted carbon atoms can be isolated in optically active or racemic forms. Methods of preparation of optically active forms from optically active starting materials are known in the art, such as by resolution of racemic mixtures or by stereoselective synthesis. Many geometric isomers of olefins, C═N double bonds, and the like can also be present in the compounds described herein, and all such stable isomers are contemplated in the present invention. Cis and trans geometric isomers of the compounds of the present invention are also included within the scope of the invention and can be isolated as a mixture of isomers or as separated isomeric forms. Where a compound capable of stereoisomerism or geometric isomerism is designated in its structure or name without reference to specific R/S or cis/trans configurations, it is intended that all such isomers are contemplated.

Resolution of racemic mixtures of compounds can be carried out by any of numerous methods known in the art, including, for example, fractional recrystallization using a chiral resolving acid which is an optically active, salt-forming organic acid. Suitable resolving agents for fractional recrystallization methods include, but are not limited to, optically active acids, such as the D and L forms of tartaric acid, diacetyltartaric acid, dibenzoyltartaric acid, mandelic acid, malic acid, lactic acid, and the various optically active camphorsulfonic acids such as β-camphorsulfonic acid. Other resolving agents suitable for fractional crystallization methods include, but are not limited to, stereoisomerically pure forms of α-methylbenzylamine (e.g., S and R forms, or diastereomerically pure forms), 2-phenylglycinol, norephedrine, ephedrine, N-methylephedrine, cyclohexylethylamine, 1,2-diaminocyclohexane, and the like. Resolution of racemic mixtures can also be carried out by elution on a column packed with an optically active resolving agent (e.g., dinitrobenzoylphenylglycine). Suitable elution solvent compositions can be determined by one skilled in the art.

Compounds of the invention may also include tautomeric forms. Tautomeric forms result from the swapping of a single bond with an adjacent double bond together with the concomitant migration of a proton. Tautomeric forms include prototropic tautomers which are isomeric protonation states having the same empirical formula and total charge. Examples of prototropic tautomers include, but are not limited to, ketone-enol pairs, amide-imidic acid pairs, lactam-lactim pairs, amide-imidic acid pairs, enamine-imine pairs, and annular forms where a proton can occupy two or more positions of a heterocyclic system including, but not limited to, 1H- and 3H-imidazole, 1H-, 2H- and 4H-1,2,4-triazole, 1H- and 2-isoindole, and 1H- and 2H-pyrazole. Tautomeric forms can be in equilibrium or sterically locked into one form by appropriate substitution.

Compounds of the invention also include hydrates and solvates, as well as anhydrous and non-solvated forms.

All compounds and pharmaceutically acceptable salts thereof can be prepared or be present together with other substances such as water and solvents (e.g., hydrates and solvates) or can be isolated.

Compounds of the invention can also include all isotopes of atoms occurring in the intermediates or final compounds. Isotopes include those atoms having the same atomic number but different mass numbers. For example, isotopes of hydrogen include tritium and deuterium.

In some embodiments, the compounds of the invention, or salts thereof, are substantially isolated. Partial separation can include, for example, a composition enriched in the compound of the invention. Substantial separation can include compositions containing at least about 50%, at least about 60%, at least about 70%, at least about 80%, at least about 90%, at least about 95%, at least about 97%, or at least about 99% by weight of the compound of the invention, or salt thereof. Methods for isolating compounds and their salts are routine in the art.

Compounds of the invention are intended to include compounds with stable structures. As used herein, the phrases “stable compound” and “stable structure” refer to a compound that is sufficiently robust to survive isolation to a useful degree of purity from a reaction mixture, and formulation into an efficacious therapeutic agent.

The present invention also includes quaternary ammonium salts of the compounds described herein, where the compounds have one or more tertiary amine moiety. As used herein, the phrase “quaternary ammonium salts” refers to derivatives of the disclosed compounds with one or more tertiary amine moieties wherein at least one of the tertiary amine moieties in the parent compound is modified by converting the tertiary amine moiety to a quaternary ammonium cation via alkylation (and the cations are balanced by anions such as Cl⁻, CH₃COO⁻, and CF₃COO⁻), for example methylation or ethylation.

The present invention provides methods of modulating an immune response in a mammal comprising administering to the mammal in need thereof a therapeutically effective amount of a compound of Formula I:

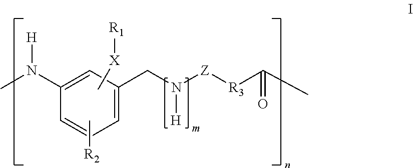

or a pharmaceutically acceptable salt thereof,
wherein:

X is O or S;

R₁ is C₁-C₉ straight or branched chain alkyl, optionally substituted with one or more —NH₂ or —NH—C(═NH)NH₂;

Y is a bond or a carbonyl;

Z is a bond or a carbonyl;

R₂ is hydrogen or C₁-C₉ straight or branched chain alkyl optionally substituted with one or more —NH₂ or —NH—C(═NH)NH₂;

or R₂ is —X—R₁;

R₃ is methylene or

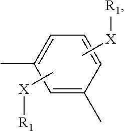

wherein the methylene is substituted with C₁-C₉ straight or branched chain alkyl, wherein the C₁-C₉ straight or branched chain alkyl is optionally substituted with one or more —NH₂ or —NH—C(═NH)NH₂;

n is 2-10; and

m is 1 or 2.

The present invention also provides methods of modulating an immune response in a mammal comprising administering to the mammal in need thereof a therapeutically effective amount of a compound of Formula II:

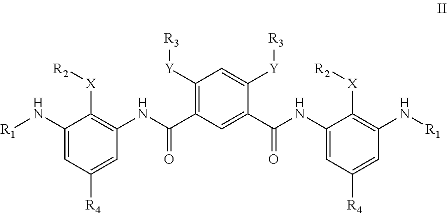

or a pharmaceutically acceptable salt thereof,
wherein:

X is O or S;

Y is O or S;

R₁ is H or —C(═O)-A, where A is C₁-C₉ straight or branched alkyl optionally substituted with one or more —NH₂, —N(CH₃)₂ or —NH—C(═NH)NH₂;

R₂ is C₁-C₉ straight or branched alkyl optionally substituted with one or more —NH₂, —N(CH₃)₂ or —NH—C(═NH)NH₂;

R₃ is C₁-C₉ straight or branched alkyl optionally substituted with one or more —NH₂, —N(CH₃)₂ or —NH—C(═NH)NH₂; and

R₄ is H, —B, or —C(═O)—O—B, where B is C₁-C₉ straight or branched alkyl.

The present invention also provides methods of modulating an immune response in a mammal comprising administering to the mammal in need thereof a therapeutically effective amount of a compound chosen from:

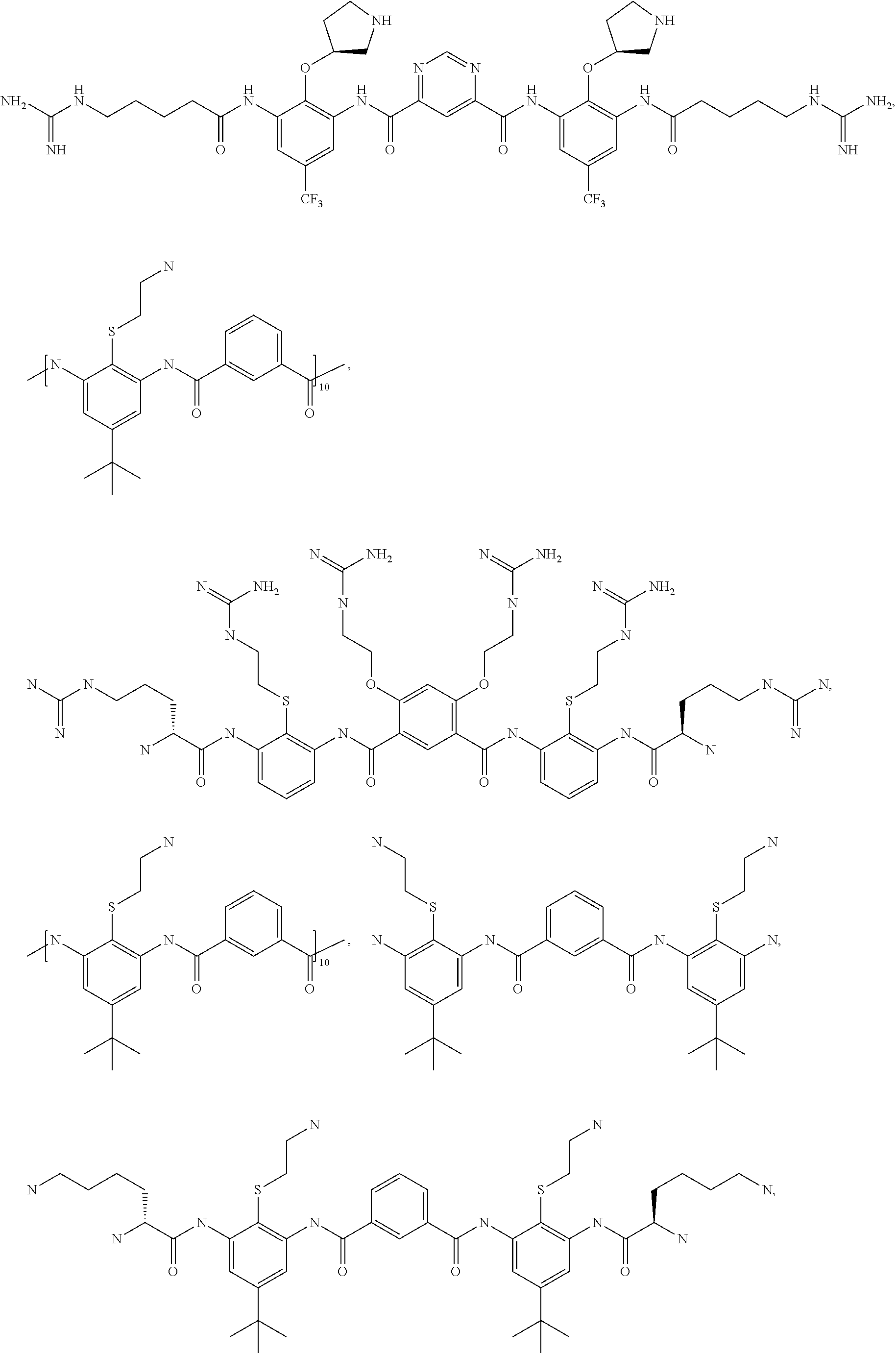

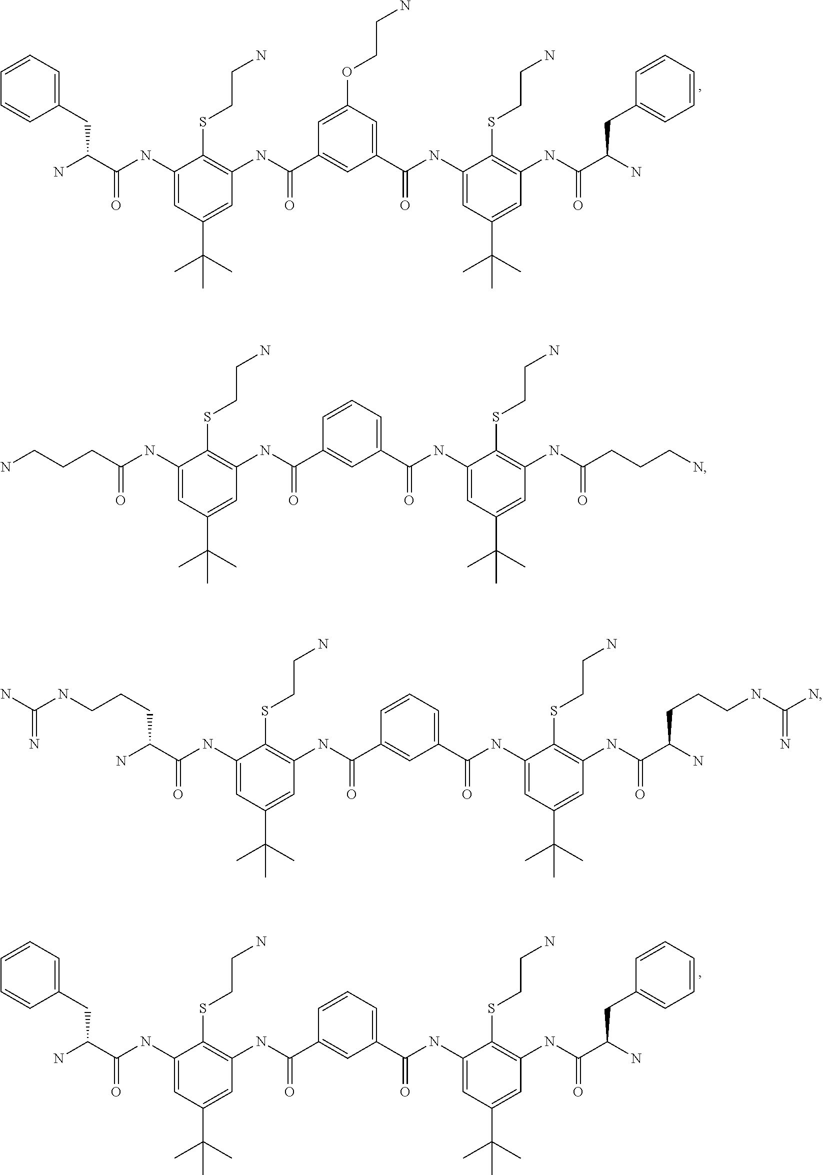

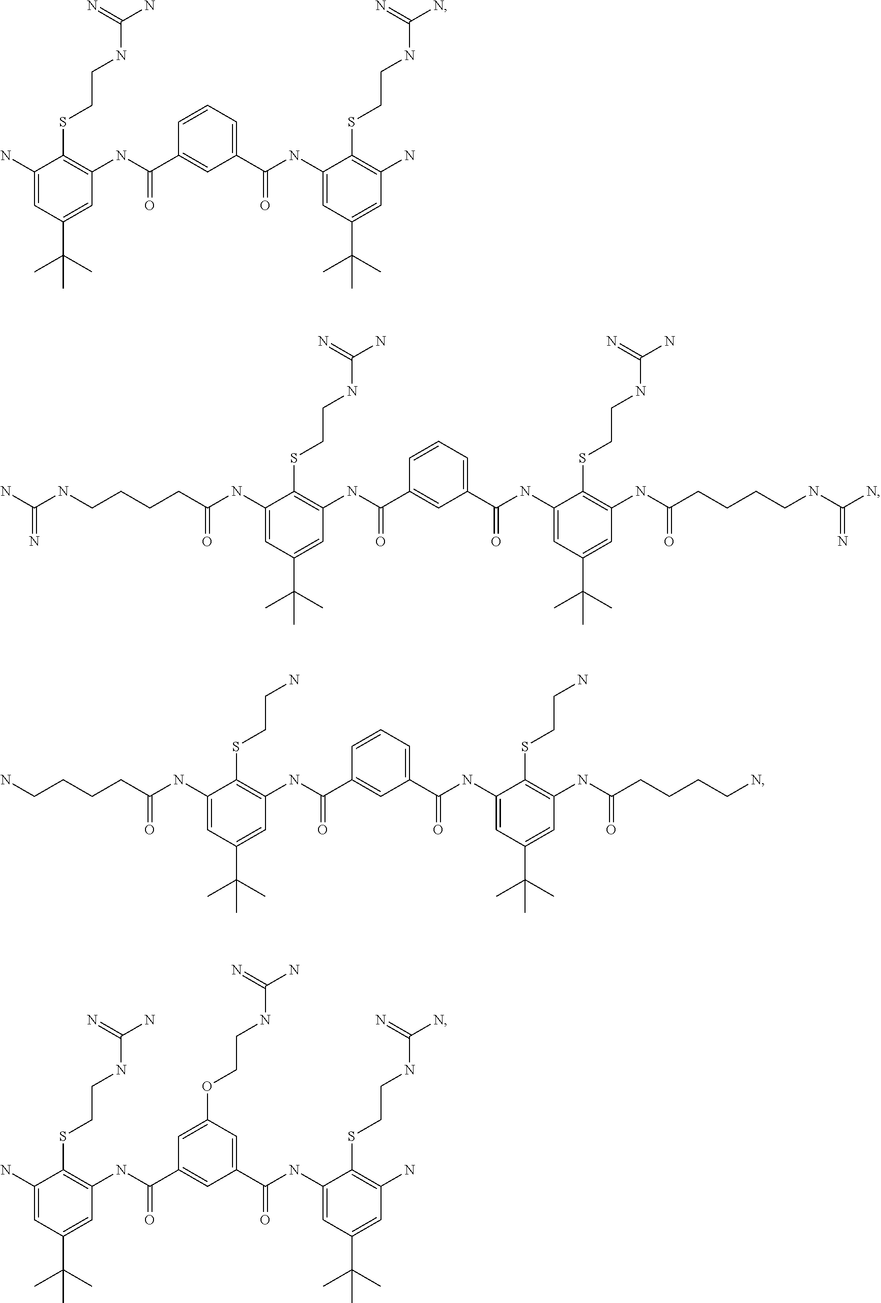

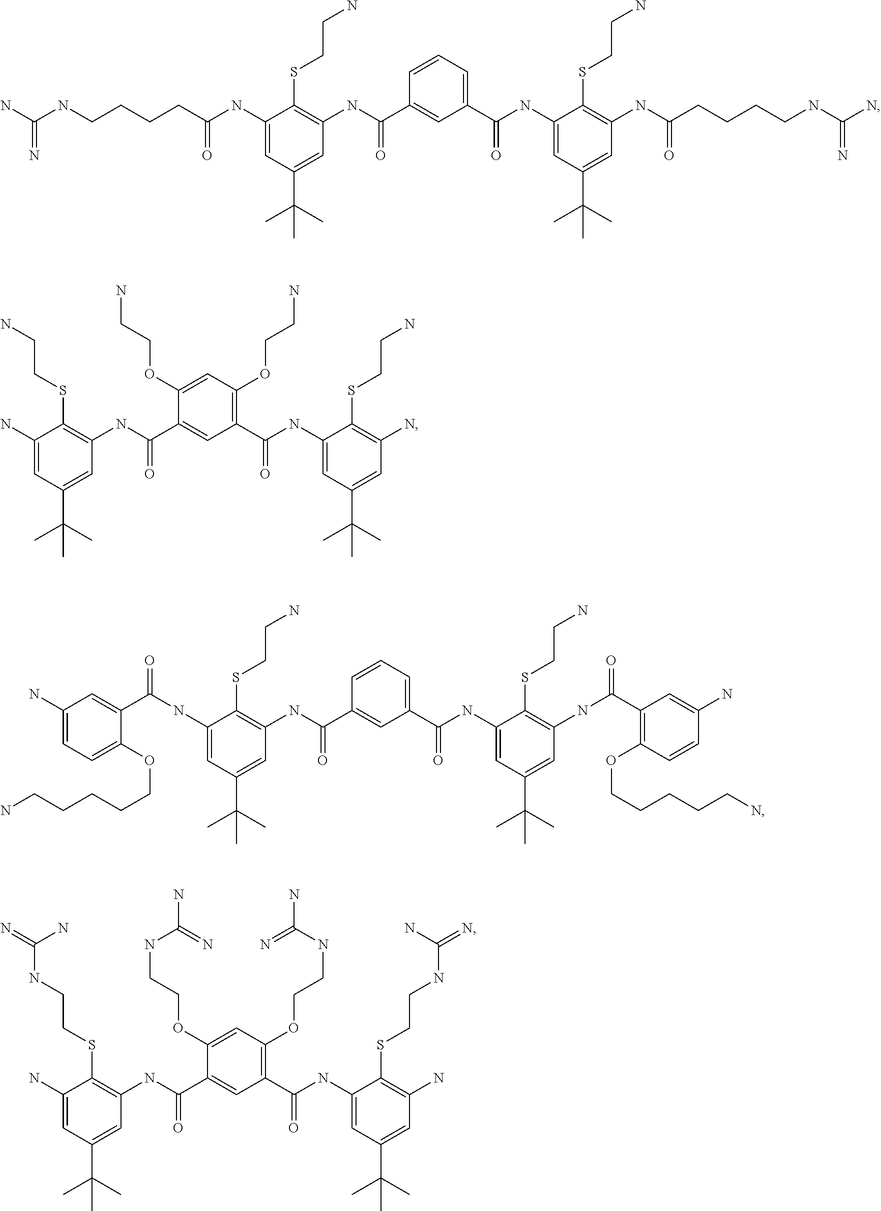

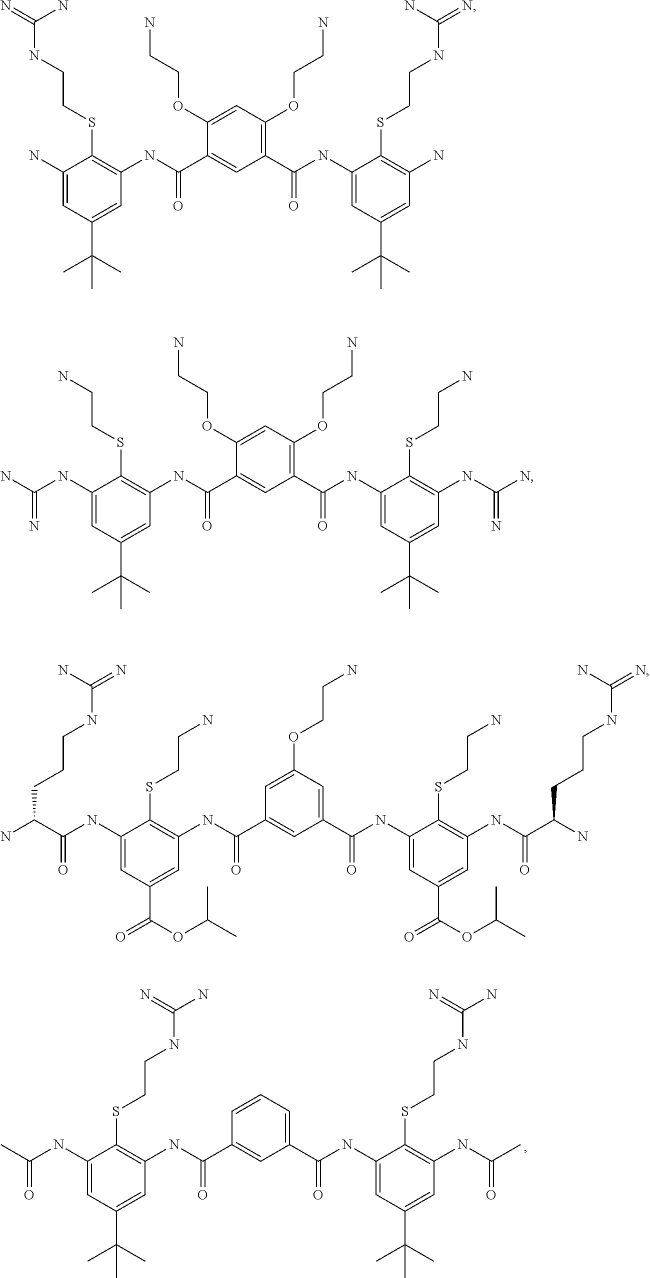

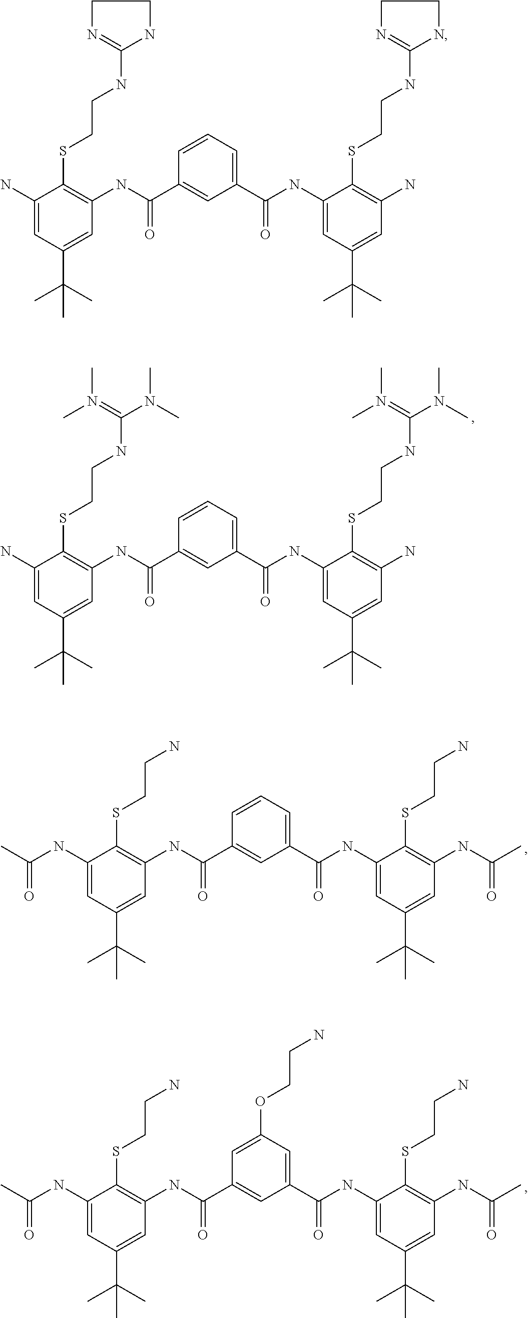

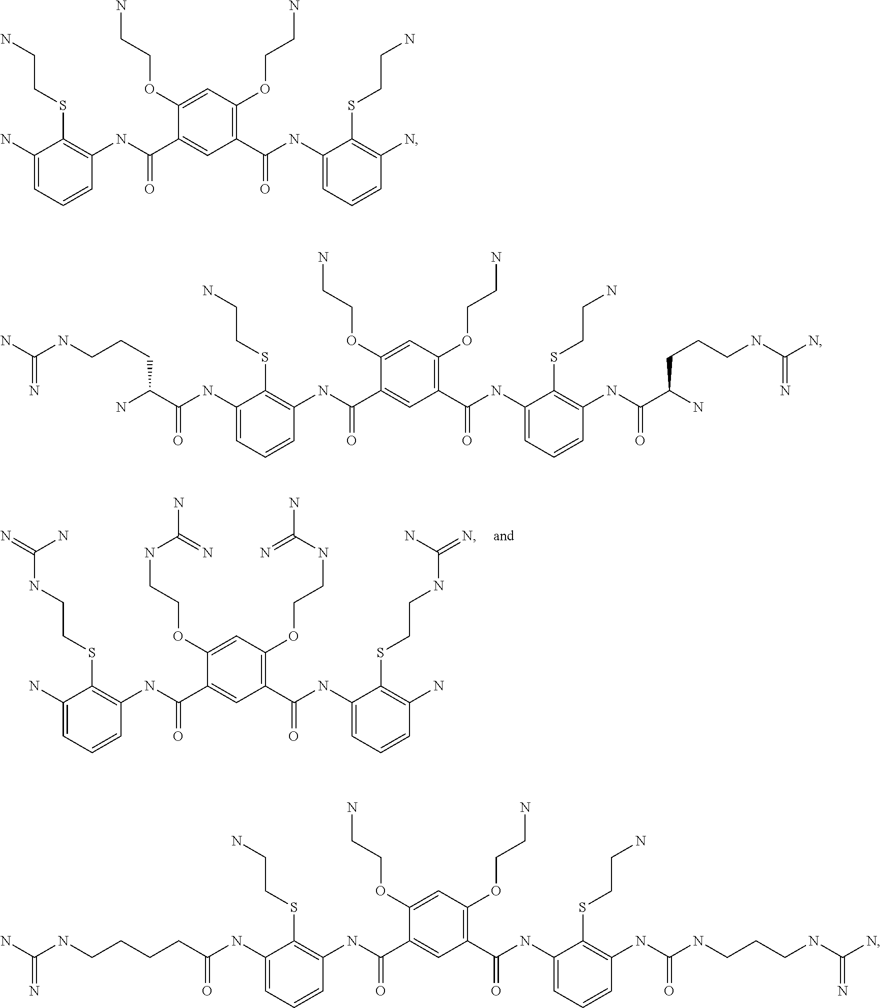

or a pharmaceutically acceptable salt thereof.

The present invention also provides methods of modulating an immune response in a mammal comprising administering to the mammal in need thereof a therapeutically effective amount of a compound of Formula III:

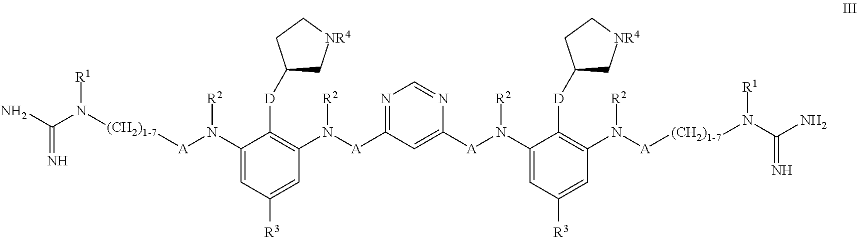

or a pharmaceutically acceptable salt thereof,
wherein:

each A is, independently, —C═O, —C═S, or CH₂;

each D is, independently, O or S;

each R¹ is, independently, hydrogen, C₁₋₃alkyl, C₁₋₃alkoxy, halo, or haloC₁₋₃alkyl;

each R² is, independently, hydrogen, C₁₋₃alkyl, C₁₋₃alkoxy, halo, or haloC₁₋₃alkyl;

each R³ is, independently, hydrogen, C₁₋₄alkyl, C₁₋₄alkoxy, halo, or haloC₁₋₄alkyl; and

each R⁴ is, independently, hydrogen, C₁₋₃ alkyl, C₁₋₃alkoxy, halo, or haloC₁₋₃alkyl.

In some embodiments, at least one A is —C═O. In some embodiments, each A is —C═O.

In some embodiments, at least one D is O. In some embodiments, each D is O.

In some embodiments, each R¹ is, independently, hydrogen, methyl, ethyl, methoxy, ethoxy, halo, or haloC₁₋₃alkyl. In some embodiments, each R¹ is, independently, hydrogen, methyl, methoxy, halo, or haloC₁₋₃alkyl. In some embodiments, each R¹ is, independently, hydrogen, methyl, or methoxy. In some embodiments, at least one R¹ is hydrogen. In some embodiments, each R¹ is hydrogen.

In some embodiments, each R² is, independently, hydrogen, methyl, ethyl, methoxy, ethoxy, halo, or haloC₁₋₃alkyl. In some embodiments, each R² is, independently, hydrogen, methyl, methoxy, or halo. In some embodiments, at least one R² is hydrogen. In some embodiments, each R² is hydrogen.

In some embodiments, each R³ is, independently, hydrogen, methyl, ethyl, methoxy, ethoxy, halo, or haloC₁₋₃alkyl. In some embodiments, each R³ is, independently, methyl, methoxy, halo, or haloC₁₋₃alkyl. In some embodiments, each R³ is, independently, halo or haloC₁₋₃alkyl. In some embodiments, each R³ is, independently, haloC₁₋₃alkyl. In some embodiments, at least one R³ is trifluoromethyl. In some embodiments, each R³ is trifluoromethyl.

In some embodiments, each R⁴ is, independently, hydrogen, methyl, ethyl, methoxy, ethoxy, or haloC₁₋₃alkyl. In some embodiments, each R⁴ is, independently, hydrogen, methyl, methoxy, halo, or haloC₁₋₃alkyl. In some embodiments, each R⁴ is, independently, hydrogen, methyl, methoxy, or halo. In some embodiments, at least one R⁴ is hydrogen. In some embodiments, each R⁴ is hydrogen.

In some embodiments, each A is, independently, —C═O or —C═S; each D is, independently, O or S; each R¹ is, independently, hydrogen, methyl, ethyl, methoxy, ethoxy, halo, halomethyl, or haloethyl; each R² is, independently, hydrogen, methyl, methoxy, halo, or halomethyl; each R³ is, independently, C₁₋₃alkyl, C₁₋₃alkoxy, halo, or haloalkyl; and each R⁴ is, independently, hydrogen, methyl, ethyl, methoxy, ethoxy, halo, halomethyl, or haloethyl.

In some embodiments, each A is, independently, —C═O or —C═S; each D is, independently, O or S; each R¹ is, independently, hydrogen, methyl, methoxy, halo, or halomethyl; each R² is, independently, hydrogen, halo, or halomethyl; each R³ is, independently, methyl, ethyl, methoxy, ethoxy, halo, halomethyl, or haloethyl; and each R⁴ is, independently, hydrogen, methyl, ethyl, methoxy, ethoxy, halo, halomethyl, or haloethyl.

In some embodiments, each A is —C═O; each D is O; each R¹ is, independently, hydrogen, halo, or halomethyl; each R² is, independently, hydrogen or halo; each R³ is, independently, methyl, methoxy, halo, or halomethyl; and each R⁴ is, independently, hydrogen, methyl, methoxy, halo, or halomethyl.

In some embodiments, each A is —C═O; each D is O; each R¹ is, independently, hydrogen or halo; each R² is, independently, hydrogen or halo; each R³ is, independently, methyl, halo, or halomethyl; and each R⁴ is, independently, hydrogen, methyl, halo, or halomethyl.

In some embodiments, each A is —C═O; each D is O; each R¹ is, independently, hydrogen or halo; each R² is, independently, hydrogen or halo; each R³ is, independently, halo or halomethyl; and each R⁴ is, independently, hydrogen or halo.

In some embodiments, each A is —C═O; each D is O; each R¹ is, independently, hydrogen or halo; each R² is, independently, hydrogen or halo; each R³ is, independently, methyl, halo, or halomethyl; and each R⁴ is, independently, hydrogen, methyl, halo, or halomethyl.

In some embodiments, each A is —C═O; each D is O; each R¹ is, independently, hydrogen or halo; each R² is, independently, hydrogen or halo; each R³ is, independently, halo or halomethyl; and each R⁴ is, independently, hydrogen, halo, or halomethyl.

In some embodiments, the method of modulating an immune response comprises decreasing the production of a cytokine. In some embodiments, the cytokine is chosen from TNFalpha, IL-1Beta, IL-1alpha, IL-8, IL-6, IL-10, IL-11, IL-12, TGF-Beta, and IFNgamma.

In some embodiments, the immune response is against an oral pathogen. In some embodiments, the oral pathogen is chosen from Aggregatibacter spp. such as, for example, Aggregatibacter actinomycetemcomitans; Porphyromonas spp. such as, for example, Porphyromonas gingivalis; Streptococcus spp. such as, for example, Streptococcus sanguis and Streptococcus mutans, Candida spp. such as, for example, Candida albicans, Candida glabrata, Candida krusei, Candida dubliniensis, Candida parapsilosis, and Candida tropicalis; Actinomyces spp. such as, for example, Actinomyces viscosus; and Lactobacillus spp. such as, for example, Lactobacillus casei. In some embodiments, the immune response is against a bacterial pathogen. In some embodiments, the bacterial pathogen is chosen from Staphylococcus spp., such as, for example, Staphylococcus aureus, methicillin-resistant Staphylococcus aureus, and Staphylococcus epidermidis; Streptococcus spp. such as, for example, Streptococcus pneumoniae, Streptococcus pyogenes, and Streptococcus viridans; Escherichia spp. such as, for example, E. coli; Enterococcus spp. such as, for example, Enterococcus faecalis and Enterococcus faecium; Psuedomonas spp. such as, for example, Pseudomonas aeruginosa; Acinetobacter spp. such as, for example, A. baumannii; Haemophilus spp. such as, for example, Haemophilus influenzae; Serratia spp. such as, for example, Serratia marcescens; Moraxella spp. such as, for example, Moraxella catarrhalis; Klebsiella spp. such as, for example, Klebsiella pneumoniae; Proteus spp. such as, for example, Proteus vulgaris and Proteus mirabilis; Bacteroides spp. such as, for example, Bacteroides fragalis; Clostridium spp. such as, for example, Clostridium difficile and Clostridium perfringens; and Propionibacterium spp. such as, for example, Propionibacterium acnes.

The present invention also provides methods of modulating an immune response in a mammal comprising administering to the mammal in need thereof a therapeutically effective amount of a compound of Formula IV:

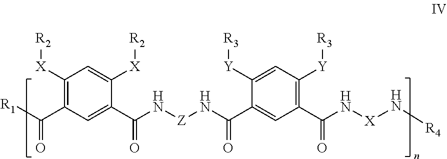

or a pharmaceutically acceptable salt thereof,
wherein:

n=1 to 10;

X is O or S;

Y is O or S;

Z is a bond, C₁-C₉ straight or branched alkyl, or a 1,4-cyclohexyl;

R₁ is NH₂ or NH-A, where A is C₁-C₉ straight or branched alkyl, where A is optionally substituted with —NH₂, —N(CH₃)₂ or —NH—C(═NH)NH₂;

R₂ is C₁-C₉ straight or branched alkyl, where R₂ is optionally substituted with one or more —NH₂, —N(CH₃)₂ or —NH—C(═NH)NH₂;

R₃ is C₁-C₉ straight or branched alkyl, where R₃ is optionally substituted with one or more —NH₂, —N(CH₃)₂ or —NH—C(═NH)NH₂;

R₄ is H or

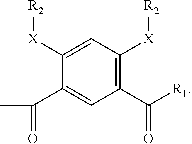

The present invention also provides methods of modulating an immune response in a mammal comprising administering to the mammal in need thereof a therapeutically effective amount of a compound chosen from:

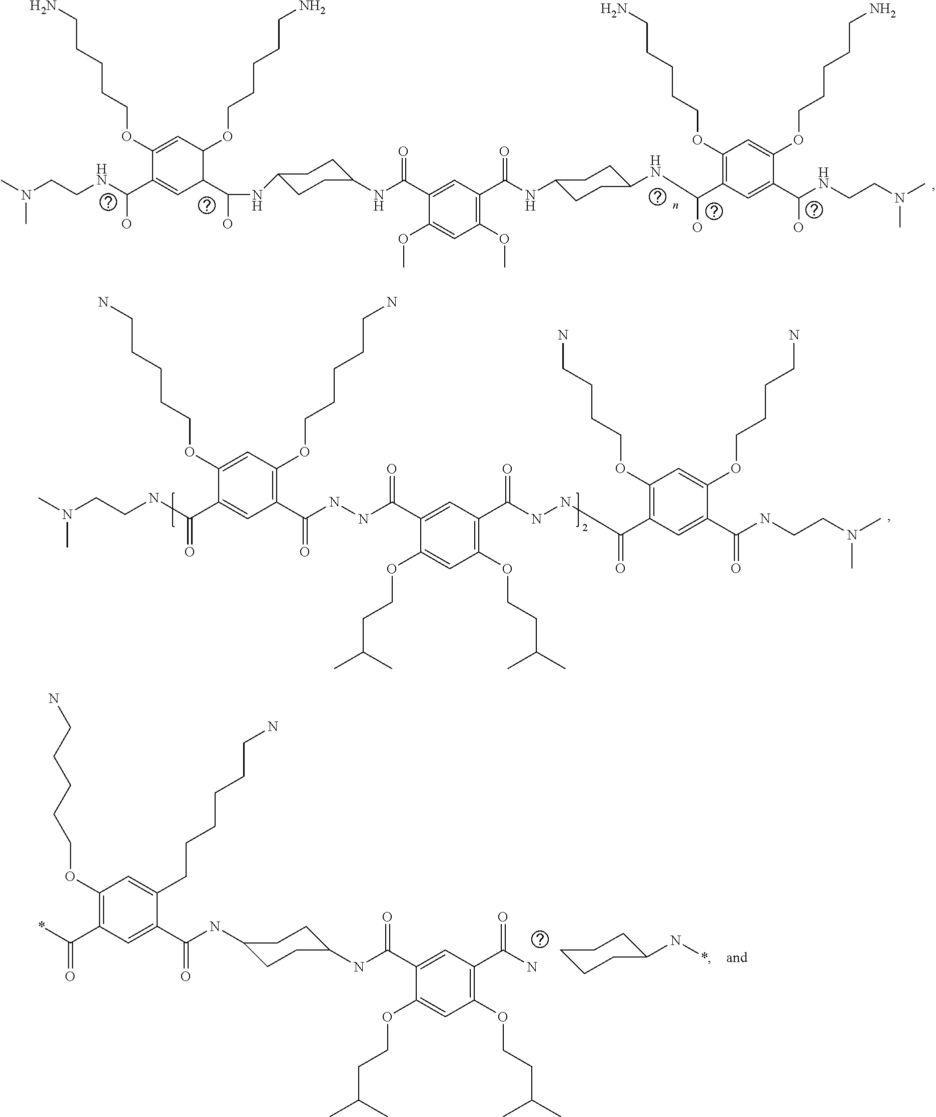

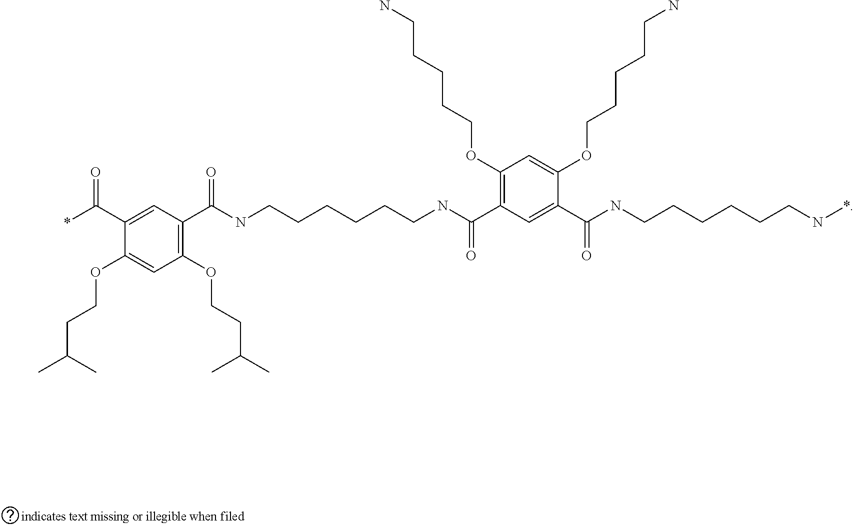

In some embodiments, the method of modulating an immune response comprises decreasing the production of a cytokine. In some embodiments, the cytokine is chosen from TNFalpha, IL-1Beta, IL-1alpha, IL-8, IL-6, IL-10, IL-11, IL-12, TGF-Beta, and IFNgamma.

In some embodiments, the immune response is against an oral pathogen. In some embodiments, the oral pathogen is chosen from Aggregatibacter spp. such as, for example, Aggregatibacter actinomycetemcomitans; Porphyromonas spp. such as, for example, Porphyromonas gingivalis; Streptococcus spp. such as, for example, Streptococcus sanguis and Streptococcus mutans, Candida spp. such as, for example, Candida albicans, Candida glabrata, Candida krusei, Candida dubliniensis, Candida parapsilosis, and Candida tropicalis; Actinomyces spp. such as, for example, Actinomyces viscosus; and Lactobacillus spp. such as, for example, Lactobacillus casei. In some embodiments, the immune response is against a bacterial pathogen. In some embodiments, the bacterial pathogen is chosen from Staphylococcus spp., such as, for example, Staphylococcus aureus, methicillin-resistant Staphylococcus aureus, and Staphylococcus epidermidis; Streptococcus spp. such as, for example, Streptococcus pneumoniae, Streptococcus pyogenes, and Streptococcus viridans; Escherichia spp. such as, for example, E. coli; Enterococcus spp. such as, for example, Enterococcus faecalis and Enterococcus faecium; Psuedomonas spp. such as, for example, Pseudomonas aeruginosa; Acinetobacter spp. such as, for example, A. baumannii; Haemophilus spp. such as, for example, Haemophilus influenzae; Serratia spp. such as, for example, Serratia marcescens; Moraxella spp. such as, for example, Moraxella catarrhalis; Klebsiella spp. such as, for example, Klebsiella pneumoniae; Proteus spp. such as, for example, Proteus vulgaris and Proteus mirabilis; Bacteroides spp. such as, for example, Bacteroides fragalis; Clostridium spp. such as, for example, Clostridium difficile and Clostridium perfringens; and Propionibacterium spp. such as, for example, Propionibacterium acnes.

The present invention also provides methods of modulating an immune response in a mammal comprising administering to the mammal in need thereof a therapeutically effective amount of a compound of Formula V:

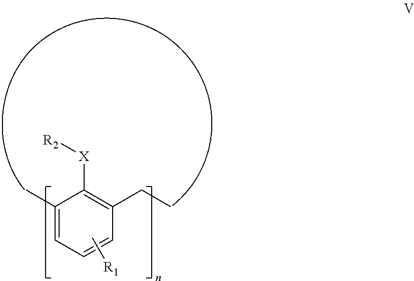

or a pharmaceutically acceptable salt thereof,
wherein:

n is 2-8;

X is a bond, O or —O—CH₂—C(═O)—O—,

R₁ is -A or —O-A, where A is C₁-C₉ straight or branched alkyl; and

R₂ is C₁-C₉ straight or branched alkyl, where R₂ is optionally substituted with one or more —NH₂, —N(CH₃)₂, or —NH—C(═NH)NH₂.

In some embodiments, n is 4-8.

The present invention also provides methods of modulating an immune response in a mammal comprising administering to the mammal in need thereof a therapeutically effective amount of a compound chosen from:

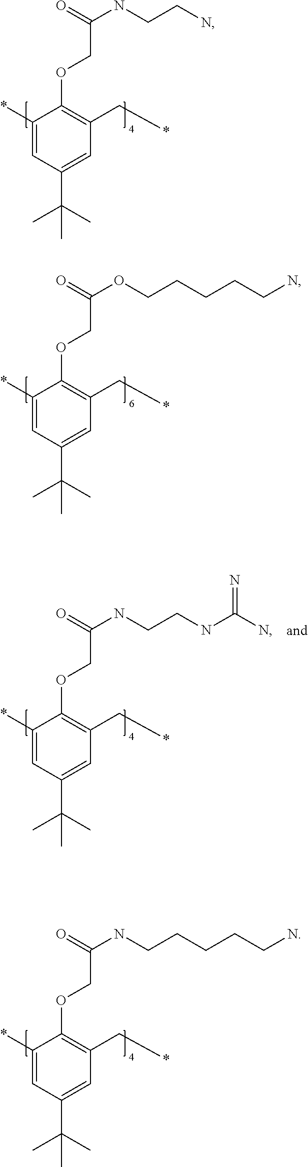

In some embodiments, the method of modulating an immune response comprises decreasing the production of a cytokine. In some embodiments, the cytokine is chosen from TNFalpha, IL-1Beta, IL-1alpha, IL-8, IL-6, IL-10, IL-11, IL-12, TGF-Beta, and IFNgamma.

In some embodiments, the immune response is against an oral pathogen. In some embodiments, the oral pathogen is chosen from Aggregatibacter spp. such as, for example, Aggregatibacter actinomycetemcomitans; Porphyromonas spp. such as, for example, Porphyromonas gingivalis; Streptococcus spp. such as, for example, Streptococcus sanguis and Streptococcus mutans, Candida spp. such as, for example, Candida albicans, Candida glabrata, Candida krusei, Candida dubliniensis, Candida parapsilosis, and Candida tropicalis; Actinomyces spp. such as, for example, Actinomyces viscosus; and Lactobacillus spp. such as, for example, Lactobacillus casei. In some embodiments, the immune response is against a bacterial pathogen. In some embodiments, the bacterial pathogen is chosen from Staphylococcus spp., such as, for example, Staphylococcus aureus, methicillin-resistant Staphylococcus aureus, and Staphylococcus epidermidis; Streptococcus spp. such as, for example, Streptococcus pneumoniae, Streptococcus pyogenes, and Streptococcus viridans; Escherichia spp. such as, for example, E. coli; Enterococcus spp. such as, for example, Enterococcus faecalis and Enterococcus faecium; Psuedomonas spp. such as, for example, Pseudomonas aeruginosa; Acinetobacter spp. such as, for example, A. baumannii; Haemophilus spp. such as, for example, Haemophilus influenzae; Serratia spp. such as, for example, Serratia marcescens; Moraxella spp. such as, for example, Moraxella catarrhalis; Klebsiella spp. such as, for example, Klebsiella pneumoniae; Proteus spp. such as, for example, Proteus vulgaris and Proteus mirabilis; Bacteroides spp. such as, for example, Bacteroides fragalis; Clostridium spp. such as, for example, Clostridium difficile and Clostridium perfringens; and Propionibacterium spp. such as, for example, Propionibacterium acnes.

The present invention also provides methods of modulating an immune response in a mammal comprising administering to the mammal in need thereof a therapeutically effective amount of a compound of Formula VI:

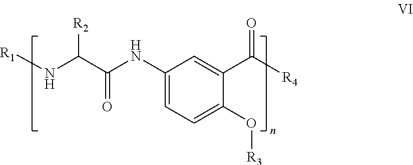

or a pharmaceutically acceptable salt thereof,
wherein:

n is 2 to 10;

R₁ is H or

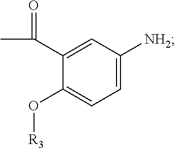

R₂ is C₁-C₉ straight or branched alkyl, where R₂ is optionally substituted with one or more —NH₂, —N(CH₃)₂ or —NH—C(═NH)NH₂;

R₃ is C₁-C₉ straight or branched alkyl, where R₂ is optionally substituted with one or more —NH₂, —N(CH₃)₂ or —NH—C(═NH)NH₂;

R₄ is OH, NH₂ or

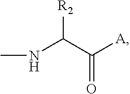

where A is OH or NH₂.

The present invention also provides methods of modulating an immune response in a mammal comprising administering to the mammal in need thereof a therapeutically effective amount of a compound chosen from:

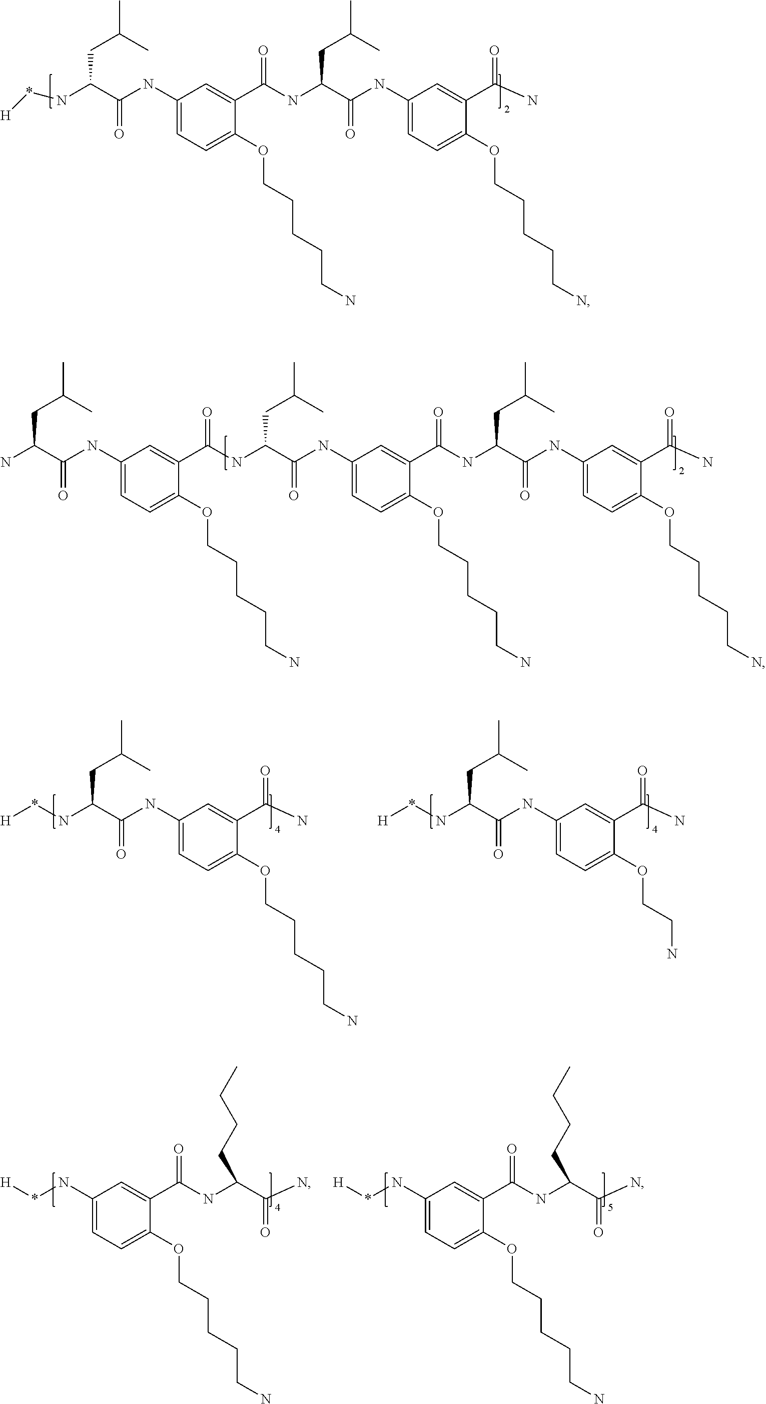

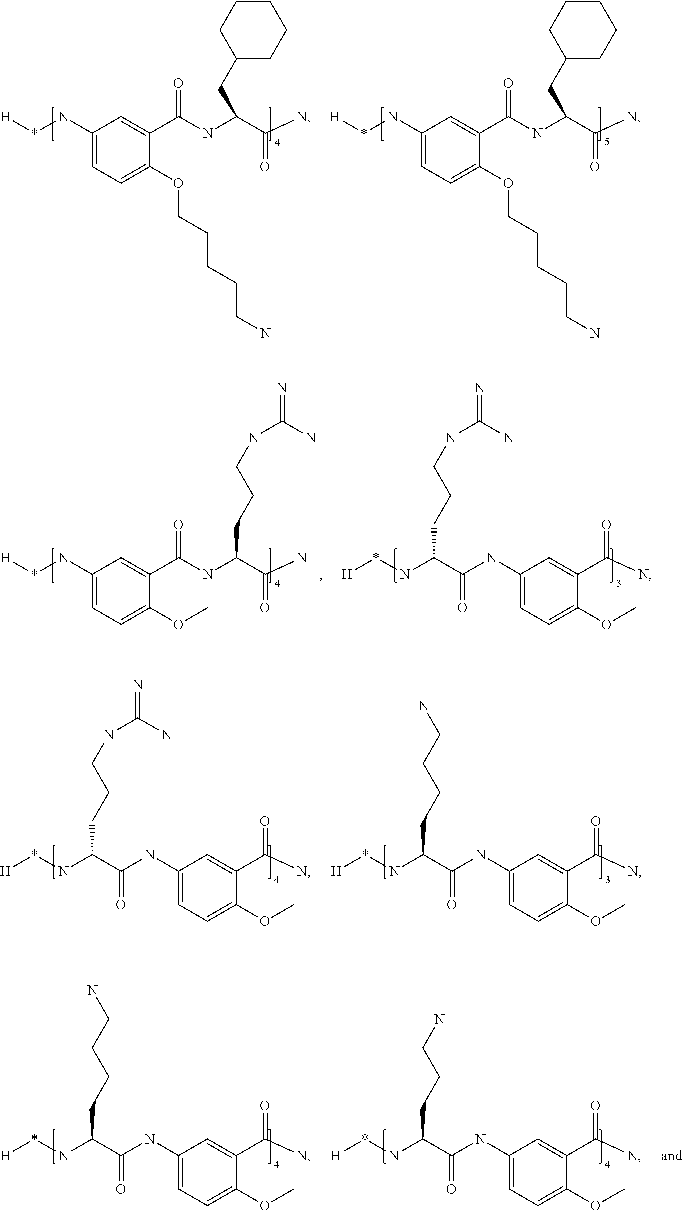

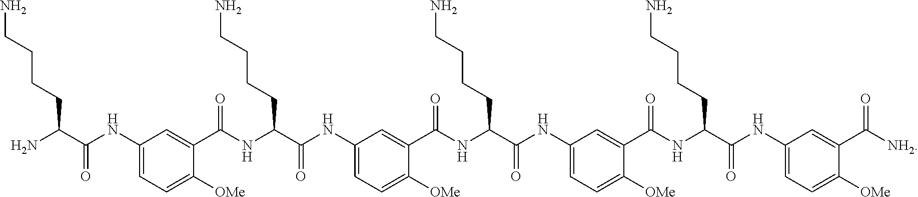

In some embodiments, the method of modulating an immune response comprises decreasing the production of a cytokine. In some embodiments, the cytokine is chosen from TNFalpha, IL-1Beta, IL-1alpha, IL-8, IL-6, IL-10, IL-11, IL-12, TGF-Beta, and IFNgamma.

In some embodiments, the immune response is against an oral pathogen. In some embodiments, the oral pathogen is chosen from Aggregatibacter spp. such as, for example, Aggregatibacter actinomycetemcomitans; Porphyromonas spp. such as, for example, Porphyromonas gingivalis; Streptococcus spp. such as, for example, Streptococcus sanguis and Streptococcus mutans, Candida spp. such as, for example, Candida albicans, Candida glabrata, Candida krusei, Candida dubliniensis, Candida parapsilosis, and Candida tropicalis; Actinomyces spp. such as, for example, Actinomyces viscosus; and Lactobacillus spp. such as, for example, Lactobacillus casei. In some embodiments, the immune response is against a bacterial pathogen. In some embodiments, the bacterial pathogen is chosen from Staphylococcus spp., such as, for example, Staphylococcus aureus, methicillin-resistant Staphylococcus aureus, and Staphylococcus epidermidis; Streptococcus spp. such as, for example, Streptococcus pneumoniae, Streptococcus pyogenes, and Streptococcus viridans; Escherichia spp. such as, for example, E. coli; Enterococcus spp. such as, for example, Enterococcus faecalis and Enterococcus faecium; Psuedomonas spp. such as, for example, Pseudomonas aeruginosa; Acinetobacter spp. such as, for example, A. baumannii; Haemophilus spp. such as, for example, Haemophilus influenzae; Serratia spp. such as, for example, Serratia marcescens; Moraxella spp. such as, for example, Moraxella catarrhalis; Klebsiella spp. such as, for example, Klebsiella pneumoniae; Proteus spp. such as, for example, Proteus vulgaris and Proteus mirabilis; Bacteroides spp. such as, for example, Bacteroides fragalis; Clostridium spp. such as, for example, Clostridium difficile and Clostridium perfringens; and Propionibacterium spp. such as, for example, Propionibacterium acnes.

The present invention also provides methods of modulating an immune response in a mammal comprising administering to the mammal in need thereof a therapeutically effective amount of a compound of Formula VII:

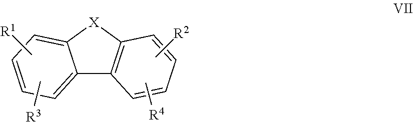

or a pharmaceutically acceptable salt thereof,
wherein:

X is C(R⁷)C(R⁸), C(═O), N(R⁹), O, S, S(═O), or S(═O)₂;

R⁷, R⁸, and R⁹ are, independently, H, C₁-C₈alkyl, C₁-C₈alkoxy, halo, OH, CF₃, or aromatic group;

R¹ and R² are, independently, H, C₁-C₈alkyl, C₁-C₈alkoxy, halo, OH, haloC₁-C₈alkyl, or CN;

R³ and R⁴ are, independently, carbocycle(R⁵)(R⁶);

each R⁵ and each R⁶ are, independently, H, C₁-C₈alkyl, C₁-C₈alkoxy, halo, OH, CF₃, aromatic group, heterocycle, or the free base or salt form of —(CH₂)ₙ—NH₂, or —(CH₂)—NH—(CH₂)ₙ—NH₂, or —(CH₂)ₙ—NH—C(═NH)NH₂, where each n is, independently, 1 to 8;

or a pharmaceutically acceptable salt thereof.

In some embodiments, X is N(R⁹), O, S, or S(═O)₂. In some embodiments, X is NH, O, or S. In some embodiments, X is NH or S.

In some embodiments, R¹ and R² are, independently, H, C₁-C₃alkyl, C₁-C₃alkoxy, halo, OH, haloC₁-C₃alkyl, or CN. In some embodiments, R¹ and R² are, independently, H, C₁-C₃alkyl, C₁-C₃alkoxy, halo, or OH. In some embodiments, R¹ and R² are, independently, H, C₁-C₃alkyl, or halo. In some embodiments, R¹ and R² are H.

In some embodiments, R³ and R⁴ are, independently,

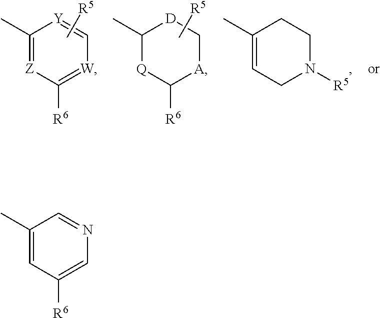

wherein:

each W, Y, and Z are, independently, C or N;

each A, D, and Q are, independently, C(R¹⁰)C(R¹¹), C(═O), N(R¹²), O, or S; and

each R¹⁰, R¹¹, and R¹² are, independently, H, C₁-C₈alkyl, C₁-C₈alkoxy, halo, OH, CF₃, or aromatic group. In some embodiments, R³ and R⁴ are, independently,

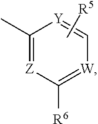

wherein each W, Y, and Z are, independently, C or N. In some embodiments, R³ and R⁴ are, independently,

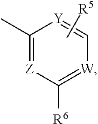

wherein each W, Y, and Z are C; or each Y and Z are C and each W is N.

In some embodiments, each R⁵ is, independently, H, C₁-C₈alkyl, C₁-C₈alkoxy, halo, OH, CF₃, or the free base or salt form of —(CH₂)ₙ—NH₂, —(CH₂)ₙ—NH₄—CH₂)ₙ—NH₂, or —(CH₂)—NH—C(═NH)NH₂, where each n is, independently, 1 to 8; and each R⁶ is, independently, heterocycle or the free base or salt form of —(CH₂)ₙ—NH₂, —(CH₂)—NH—(CH₂)ₙ—NH₂, or —(CH₂)ₙ—NH—C(═NH)NH₂, where each n is, independently, 1 to 8.

In some embodiments, each R⁵ is, independently, H, C₁-C₃alkyl, C₁-C₃alkoxy, halo, OH, or CF₃; and each R⁶ is, independently, heterocycle or the free base or salt form of —(CH₂)ₙ—NH₂, where each n is, independently, 1 to 8.

In some embodiments, each R⁵ is, independently, H, C₁-C₃alkyl, halo, or OH; and each R⁶ is, independently, heterocycle or the free base or salt form of —(CH₂)ₙ—NH₂, where each n is, independently, 1 to 4.

In some embodiments, each R⁵ is, independently, H, C₁-C₃alkyl, halo, or OH; and each R⁶ is, independently, 6-membered heterocycle or the free base or salt form of —(CH₂)ₙ—NH₂, where each n is, independently, 1 to 3.

In some embodiments, each R⁵ is, independently, H or halo; and each R⁶ is piperazinyl or the free base or salt form of —(CH₂)ₙ—NH₂ where each n is, independently, 1 to 3.

In some embodiments, each R⁵ is piperazinyl; and each R⁶ is, independently, H, C₁-C₃alkyl, C₁-C₃alkoxy, halo, OH, or CF₃.

In some embodiments, each R⁵ is piperazinyl; and each R⁶ is H, C₁-C₃alkyl, halo, OH, or CF₃.

In some embodiments, X is NH, O, S, or S(═O)₂; R¹ and R² are H; R³ and R⁴ are, independently,

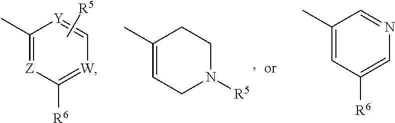

wherein: each W, Y, and Z are, independently, C or N; and each R⁵ and each R⁶ are, independently, H, heterocycle, or the free base or salt form of —(CH₂)ₙ—NH₂, where each n is, independently, 1 to 3.

In some embodiments, X is NH, O, or S; R¹ and R² are H; R³ and R⁴ are

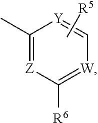

where each Z and Y are C, and each W is N; or each W, Y, and Z are C; and each R⁵ is, independently, H or halo, and each R⁶ is piperazinyl or the free base or salt form of —(CH₂)ₙ—NH₂, where each n is, independently, 1 to 3; or each R⁵ is piperazinyl, and each R⁶ is, independently, H, C₁-C₃alkyl, C₁-C₃alkoxy, halo, OH, or CF₃.

In some embodiments, X is NH, O, or S; R¹ and R² are H; R³ and R⁴ are

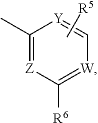

where each Z and Y are C, and each W is N; or each W, Y, and Z are C; and each R⁵ is H, and each R⁶ is piperazinyl or the free base or salt form of —(CH₂)ₙ—NH₂, where each n is, independently, 1 to 3; or each R⁵ is piperazinyl; and each R⁶ is H.

The present invention also provides methods of modulating an immune response in a mammal comprising administering to the mammal in need thereof a therapeutically effective amount of a compound chosen from:

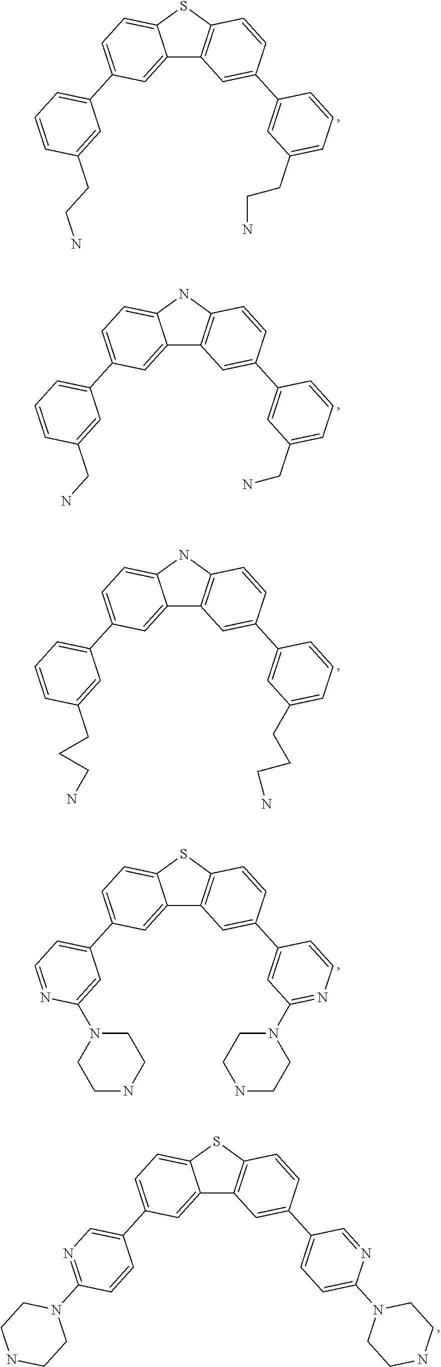

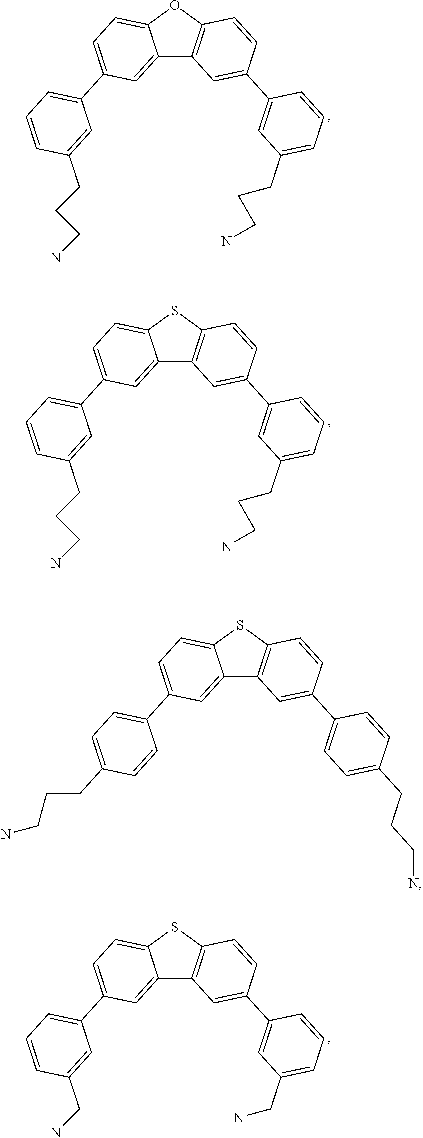

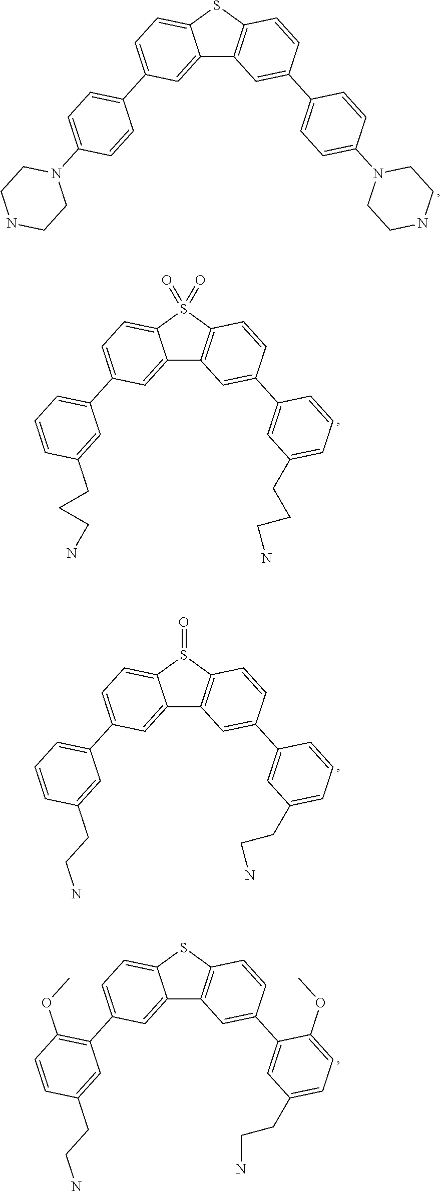

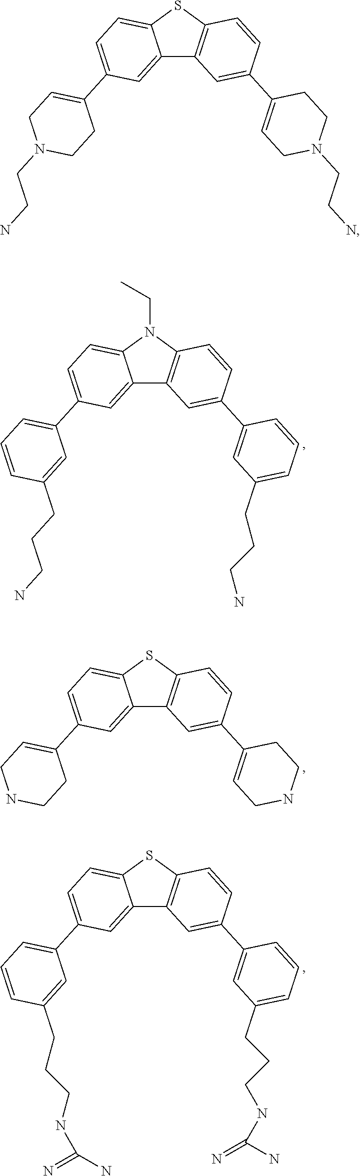

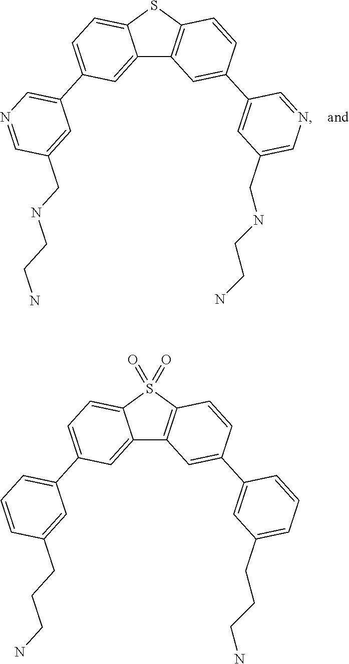

or pharmaceutically acceptable salt thereof.

In some embodiments, the method of modulating an immune response comprises decreasing the production of a cytokine. In some embodiments, the cytokine is chosen from TNFalpha, IL-1Beta, IL-1alpha, IL-8, IL-6, IL-10, IL-11, IL-12, TGF-Beta, and IFNgamma.

In some embodiments, the immune response is against an oral pathogen. In some embodiments, the oral pathogen is chosen from Aggregatibacter spp. such as, for example, Aggregatibacter actinomycetemcomitans; Porphyromonas spp. such as, for example, Porphyromonas gingivalis; Streptococcus spp. such as, for example, Streptococcus sanguis and Streptococcus mutans, Candida spp. such as, for example, Candida albicans, Candida glabrata, Candida krusei, Candida dubliniensis, Candida parapsilosis, and Candida tropicalis; Actinomyces spp. such as, for example, Actinomyces viscosus; and Lactobacillus spp. such as, for example, Lactobacillus casei. In some embodiments, the immune response is against a bacterial pathogen. In some embodiments, the bacterial pathogen is chosen from Staphylococcus spp., such as, for example, Staphylococcus aureus, methicillin-resistant Staphylococcus aureus, and Staphylococcus epidermidis; Streptococcus spp. such as, for example, Streptococcus pneumoniae, Streptococcus pyogenes, and Streptococcus viridans; Escherichia spp. such as, for example, E. coli; Enterococcus spp. such as, for example, Enterococcus faecalis and Enterococcus faecium; Psuedomonas spp. such as, for example, Pseudomonas aeruginosa; Acinetobacter spp. such as, for example, A. baumannii; Haemophilus spp. such as, for example, Haemophilus influenzae; Serratia spp. such as, for example, Serratia marcescens; Moraxella spp. such as, for example, Moraxella catarrhalis; Klebsiella spp. such as, for example, Klebsiella pneumoniae; Proteus spp. such as, for example, Proteus vulgaris and Proteus mirabilis; Bacteroides spp. such as, for example, Bacteroides fragalis; Clostridium spp. such as, for example, Clostridium difficile and Clostridium perfringens; and Propionibacterium spp. such as, for example, Propionibacterium acnes.

The present invention also provides methods of modulating an immune response in a mammal comprising administering to the mammal in need thereof a therapeutically effective amount of a compound of Formula VIII:

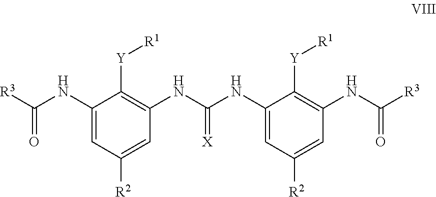

or a pharmaceutically acceptable salt thereof,
wherein:

X is O or S;

each Y is, independently, O, S, or N;

each R¹ is, independently, H, 5- or 6-membered heterocycle, or the free base or salt form of —(CH₂)ₙ—NH₂ or —(CH₂)—NH—C(═NH)NH₂, where each n is, independently, 1 to 4; or

each R¹ is, independently, together with Y a 5- or 6-membered heterocycle;

each R² is, independently, H, CF₃, C(CH₃)₃, halo, or OH; and

each R³ is, independently, —(CH₂)ₙ—NH₂ or —(CH₂)—NH—C(═NH)NH₂, where each n is, independently, 1 to 4;

or a pharmaceutically acceptable salt thereof.

In some embodiments, X is O.

In some embodiments, Y is O or S.

In some embodiments, each R¹ is, independently, 5-membered heterocycle or the free base or salt form of —(CH₂)ₙ—NH₂, where each n is, independently, 1 to 4. In some embodiments, each R¹ is, independently, 3-pyrrolyl or the free base or salt form of —(CH₂)ₙ—NH₂, where each n is, independently, 1 or 2.

In some embodiments, each R² is, independently, CF₃, C(CH₃)₃, or halo.

In some embodiments, each R³ is, independently, —(CH₂)ₙ—NH—C(═NH)NH₂, where each n is, independently, 1 to 4. In some embodiments, each R³ is —(CH₂)ₙ—NH—C(═NH)NH₂, where each n is 4.

In some embodiments, X is O or S; each Y is, independently, O or S; each R¹ is, independently, 5-membered heterocycle, or the free base or salt form of —(CH₂)ₙ—NH₂, where each n is, independently, 1 to 4; each R² is, independently, CF₃ or C(CH₃)₃; and each R³ is, independently, —(CH₂)—NH—C(═NH)NH₂, where each n is, independently, 1 to 4.

In some embodiments, X is O or S; each Y is O or S; each R¹ is 5-membered heterocycle, or the free base or salt form of —(CH₂)ₙ—NH₂, where each n is 1 to 4; each R² is CF₃ or C(CH₃)₃; and each R³ is —(CH₂)—NH—C(═NH)NH₂, where each n is 1 to 4.

In some embodiments, X is O or S; each Y is O or S; each R¹ is 3-pyrrolyl, or the free base or salt form of —(CH₂)ₙ—NH₂, where each n is 2; each R² is CF₃ or C(CH₃)₃; and each R³ is —(CH₂)ₙ—NH—C(═NH)NH₂, where each n is 4.

The present invention also provides methods of modulating an immune response in a mammal comprising administering to the mammal in need thereof a therapeutically effective amount of a compound chosen from:

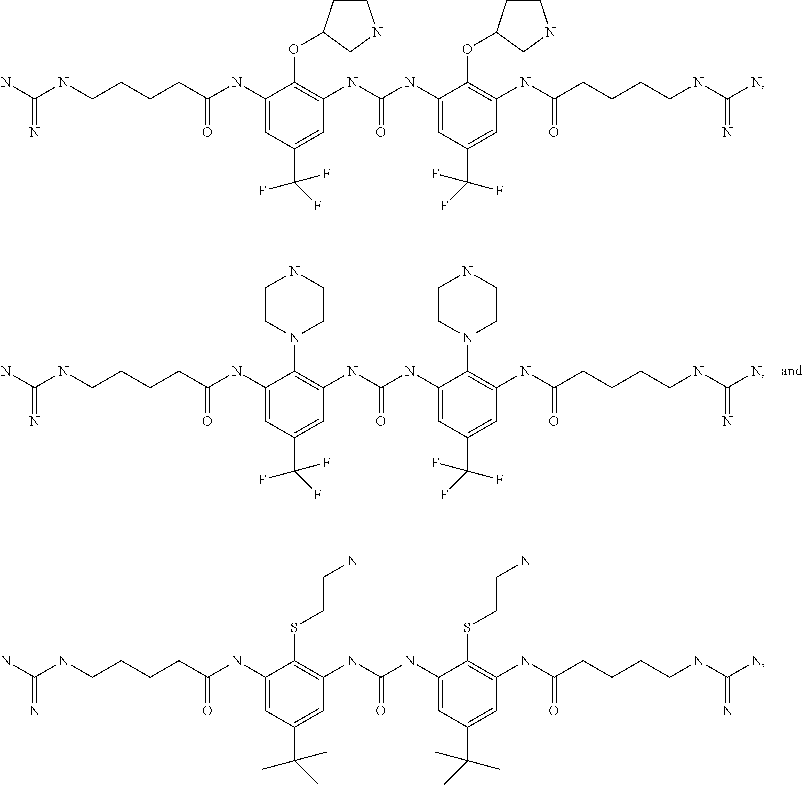

or pharmaceutically acceptable salt thereof.

In some embodiments, the method of modulating an immune response comprises decreasing the production of a cytokine. In some embodiments, the cytokine is chosen from TNFalpha, IL-1Beta, IL-1alpha, IL-8, IL-6, IL-10, IL-11, IL-12, TGF-Beta, and IFNgamma.

In some embodiments, the immune response is against an oral pathogen. In some embodiments, the oral pathogen is chosen from Aggregatibacter spp. such as, for example, Aggregatibacter actinomycetemcomitans; Porphyromonas spp. such as, for example, Porphyromonas gingivalis; Streptococcus spp. such as, for example, Streptococcus sanguis and Streptococcus mutans, Candida spp. such as, for example, Candida albicans, Candida glabrata, Candida krusei, Candida dubliniensis, Candida parapsilosis, and Candida tropicalis; Actinomyces spp. such as, for example, Actinomyces viscosus; and Lactobacillus spp. such as, for example, Lactobacillus casei. In some embodiments, the immune response is against a bacterial pathogen. In some embodiments, the bacterial pathogen is chosen from Staphylococcus spp., such as, for example, Staphylococcus aureus, methicillin-resistant Staphylococcus aureus, and Staphylococcus epidermidis; Streptococcus spp. such as, for example, Streptococcus pneumoniae, Streptococcus pyogenes, and Streptococcus viridans; Escherichia spp. such as, for example, E. coli; Enterococcus spp. such as, for example, Enterococcus faecalis and Enterococcus faecium; Psuedomonas spp. such as, for example, Pseudomonas aeruginosa; Acinetobacter spp. such as, for example, A. baumannii; Haemophilus spp. such as, for example, Haemophilus influenzae; Serratia spp. such as, for example, Serratia marcescens; Moraxella spp. such as, for example, Moraxella catarrhalis; Klebsiella spp. such as, for example, Klebsiella pneumoniae; Proteus spp. such as, for example, Proteus vulgaris and Proteus mirabilis; Bacteroides spp. such as, for example, Bacteroides fragalis; Clostridium spp. such as, for example, Clostridium difficile and Clostridium perfringens; and Propionibacterium spp. such as, for example, Propionibacterium acnes.

The present invention also provides methods of modulating an immune response in a mammal comprising administering to the mammal in need thereof a therapeutically effective amount of a compound of Formula IX:

Q—X—Z—X-Q IX

or a pharmaceutically acceptable salt thereof,
wherein:

Z is

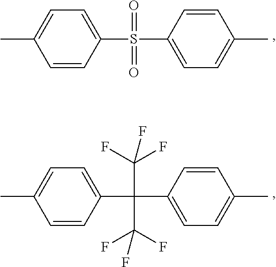

or phenyl;

each Q is, independently,

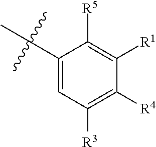

or —C(═O)—(CH₂)_{b}—NH—C(═NH)—NH₂, where each b is, independently, 1 to 4;

each X is, independently, O, S, or N;

each R¹ is, independently, H, CF₃, C(CH₃)₃, halo, or OH;

each R³ is, independently, H, —NH—R², —(CH₂)ᵣ—NH₂, —NH₂, —NH—(CH₂)_{w}—NH₂, or

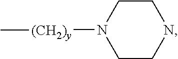

where each r is, independently, 1 or 2, each w is, independently, 1 to 3, and each y is, independently, 1 or 2;

each R² is, independently, H, or the free base or salt form of —(CH₂)ₙ—NH₂ or —(CH₂)ₙ—NH—C(═NH)NH₂, where each n is, independently, 1 to 4;

each R⁴ is, independently, H, —NH—C(═O)—(CH₂)ₚ—NH—C(═NH)—NH₂ or

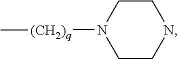

where each p is, independently, 1 to 6, and each q is, independently, 1 or 2; and

each R⁵ is, independently, H or CF₃;

or a pharmaceutically acceptable salt thereof.

In some embodiments, Z is

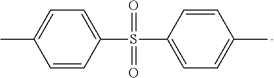

In some embodiments, each Q is, independently,

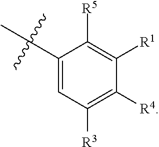

In some embodiments, each X is O.

In some embodiments, each R¹ is, independently, H, CF₃, or halo. In some embodiments, each R¹ is CF₃.

In some embodiments, each R³ is, independently, —NH—R².

In some embodiments, each R² is, independently, H, or the free base or salt form of —(CH₂)ₙ—NH₂, where each n is, independently, 1 to 4. In some embodiments, each R² is, independently, the free base or salt form of —(CH₂)—NH₂, where each n is, independently, 1 or 2. In some embodiments, each R² is the free base or salt form of —(CH₂)ₙ—NH₂, where each n is 2.

In some embodiments, each R⁴ and each R⁵ is H.

In some embodiments, Z is

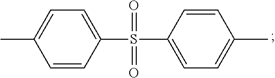

each Q is, independently,

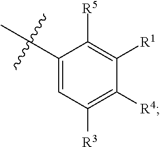

each X is O or S; each R¹ is, independently, CF₃, C(CH₃)₃, or halo; each R³ is, independently, —NH—R²; each R² is, independently, H, or the free base or salt form of —(CH₂)ₙ—NH₂, where each n is, independently, 1 to 4; and each R⁴ and each R⁵ is H.

In some embodiments, Z is

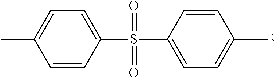

each Q is, independently,

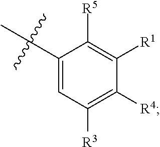

each X is O; each R¹ is CF₃, C(CH₃)₃, or halo; each R³ is, independently, —NH—R²; each R² is, independently, the free base or salt form of —(CH₂)ₙ—NH₂, where each n is 1 or 2; and each R⁴ and each R⁵ is H.

In some embodiments, Z is

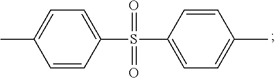

each Q is, independently,

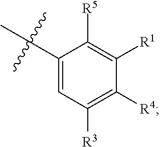

each X is O; each R¹ is CF₃ or halo; each R³ is, independently, —NH—R²; each R² is the free base or salt form of —(CH₂)ₙ—NH₂, where each n is 2; and each R⁴ and each R⁵ is H.

In some embodiments, Z is

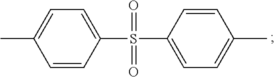

each Q is, independently,

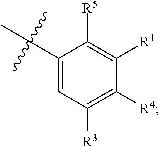

each X is, independently, O, or S; each R¹ is, independently, H, or CF₃; each R³ is H; each R⁴ is, independently, H or —NH—C(═O)—(CH₂)ₚ—NH—C(═NH)—NH₂, where each p is, independently, 3 or 4; and each R⁵ is, independently, H or CF₃.

In some embodiments, Z is

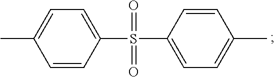

each Q is, independently, —C(═O)—(CH₂)_{b}—NH—C(═NH)—NH₂, where each b is, independently, 3 or 4; and each X is N.

In some embodiments, Z is

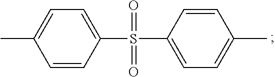

each Q is, independently,

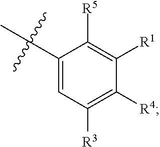

each X is O or S; each R¹ is, independently, H or CF₃; each R³ is, independently, —(CH₂), NH₂, —NH₂, —NH—(CH₂)_{w}—NH₂,

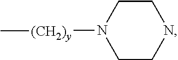

or where each r is, independently, 1 or 2, each w is, independently, 1 to 3, and each y is, independently, 1 or 2; each R⁴ is H; and each R⁵ is, independently, H or CF₃.

In some embodiments, Z is

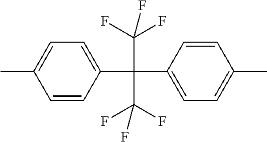

or phenyl; each Q is, independently,

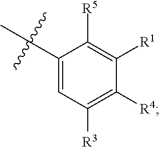

each X is, independently, O or S; each R¹ is, independently, H or CF₃; each R³ is H; each R⁴ is, independently,

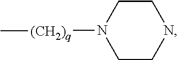

where each q is, independently, 1 or 2; and each R⁵ is, independently, H or CF₃.

The present invention also provides methods of modulating an immune response in a mammal comprising administering to the mammal in need thereof a therapeutically effective amount of a compound chosen from:

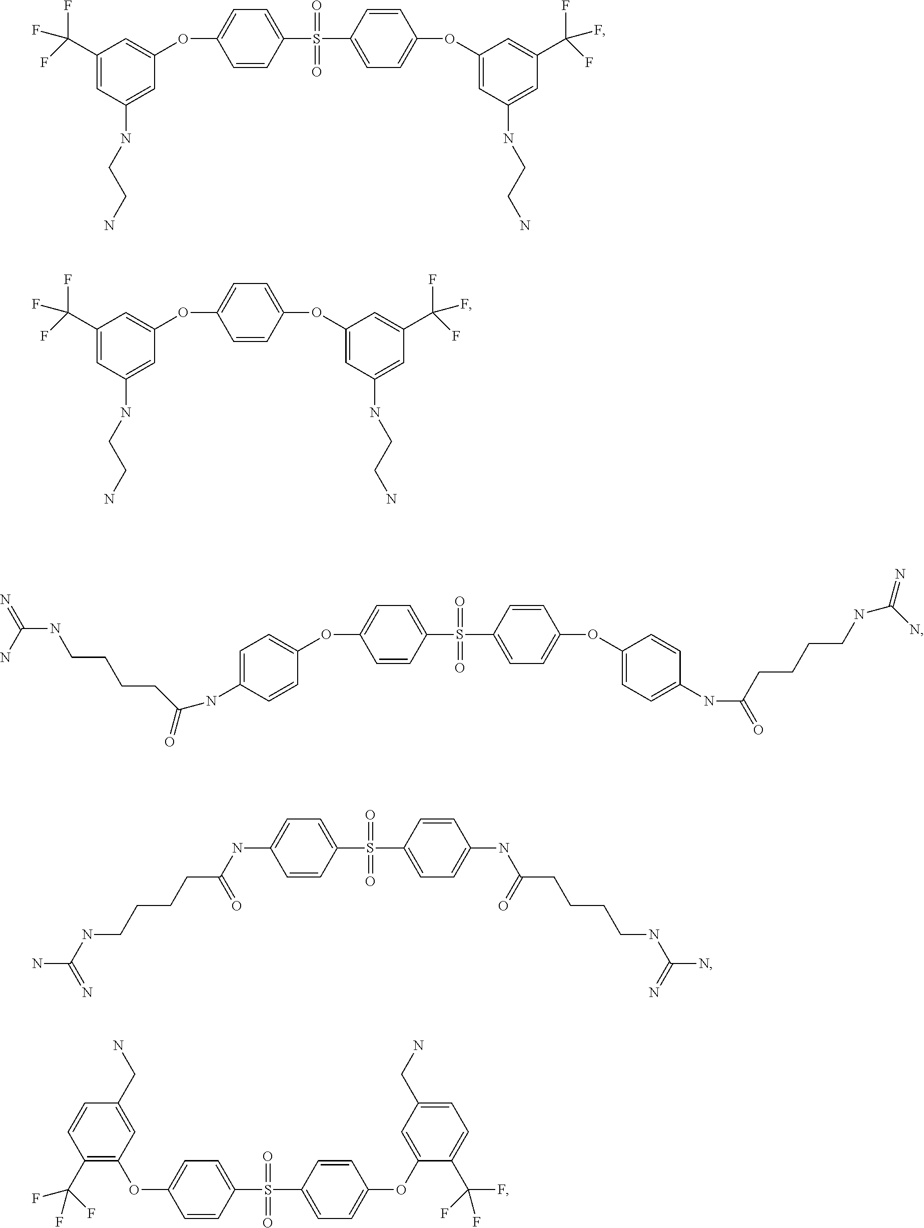

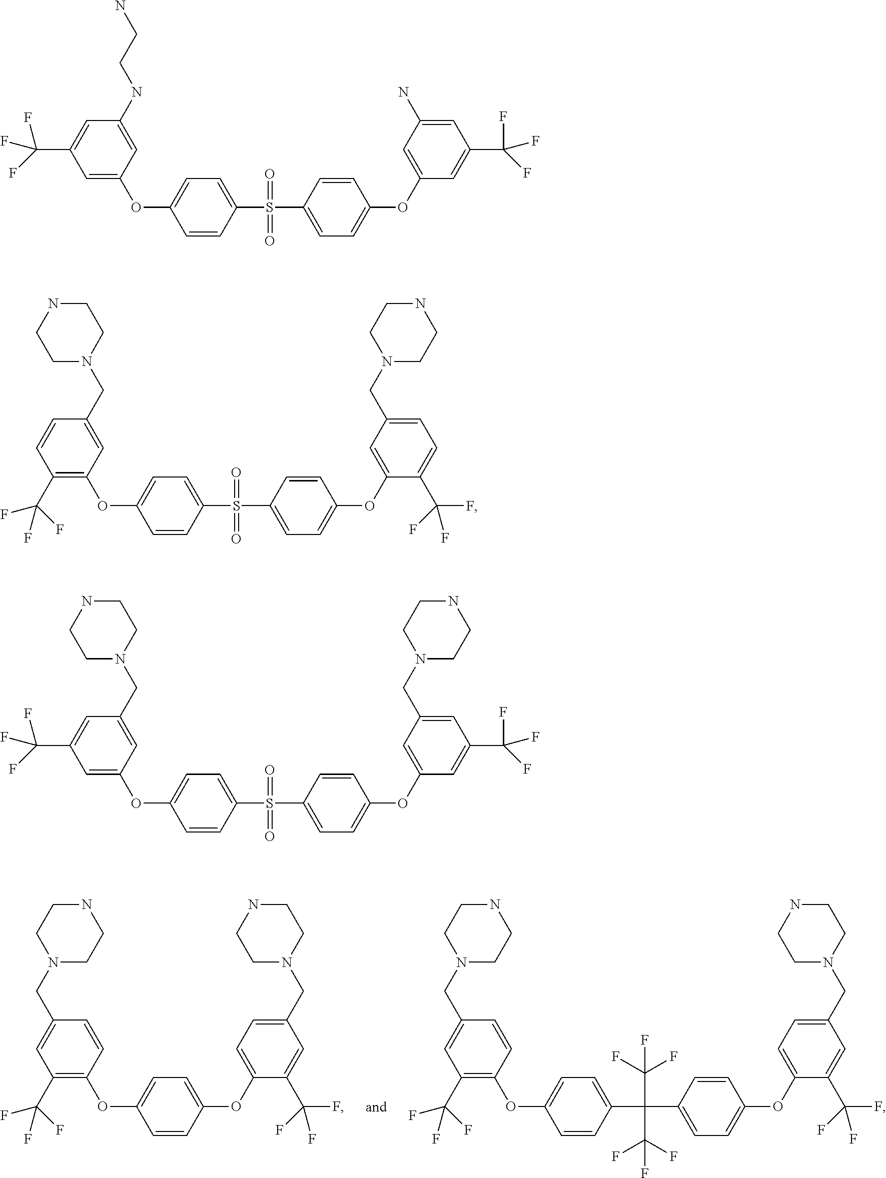

or a pharmaceutically acceptable salt thereof.

In some embodiments, the immune response is against an oral pathogen. In some embodiments, the oral pathogen is chosen from Aggregatibacter spp. such as, for example, Aggregatibacter actinomycetemcomitans; Porphyromonas spp. such as, for example, Porphyromonas gingivalis; Streptococcus spp. such as, for example, Streptococcus sanguis and Streptococcus mutans, Candida spp. such as, for example, Candida albicans, Candida glabrata, Candida krusei, Candida dubliniensis, Candida parapsilosis, and Candida tropicalis; Actinomyces spp. such as, for example, Actinomyces viscosus; and Lactobacillus spp. such as, for example, Lactobacillus casei. In some embodiments, the immune response is against a bacterial pathogen. In some embodiments, the bacterial pathogen is chosen from Staphylococcus spp., such as, for example, Staphylococcus aureus, methicillin-resistant Staphylococcus aureus, and Staphylococcus epidermidis; Streptococcus spp. such as, for example, Streptococcus pneumoniae, Streptococcus pyogenes, and Streptococcus viridans; Escherichia spp. such as, for example, E. coli; Enterococcus spp. such as, for example, Enterococcus faecalis and Enterococcus faecium; Psuedomonas spp. such as, for example, Pseudomonas aeruginosa; Acinetobacter spp. such as, for example, A. baumannii; Haemophilus spp. such as, for example, Haemophilus influenzae; Serratia spp. such as, for example, Serratia marcescens; Moraxella spp. such as, for example, Moraxella catarrhalis; Klebsiella spp. such as, for example, Klebsiella pneumoniae; Proteus spp. such as, for example, Proteus vulgaris and Proteus mirabilis; Bacteroides spp. such as, for example, Bacteroides fragalis; Clostridium spp. such as, for example, Clostridium difficile and Clostridium perfringens; and Propionibacterium spp. such as, for example, Propionibacterium acnes.

The present invention also provides methods of modulating an immune response in a mammal comprising administering to the mammal in need thereof a therapeutically effective amount of a compound of Formula X:

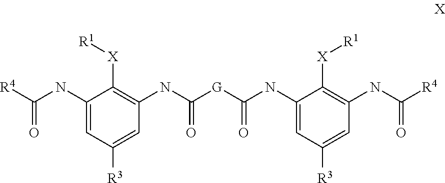

or a pharmaceutically acceptable salt thereof,
wherein:

G is

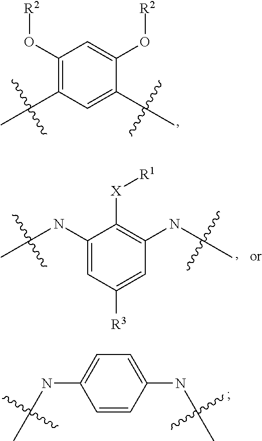

each X is, independently, O or S;

each R¹ is, independently,

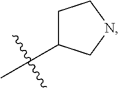

or the free base or salt form of —(CH₂)ₙ—NH₂ or —(CH₂)ₙ—NH—C(═NH)NH₂, where each n is, independently, 1 to 4;

each R² is, independently, H, C₁-C₈alkyl, or the free base or salt form of —(CH₂)ₙ—NH₂ or —(CH₂)ₙ—NH—C(═NH)NH₂, where each n is, independently, 1 to 4;

each R³ is, independently, H, CF₃, C(CH₃)₃, halo, or OH; and

each R⁴ is, independently, —(CH₂)ₙ—NH₂ or —(CH₂)ₙ—NH—C(═NH)NH₂, where each n is, independently, 1 to 4.

In some embodiments, G is

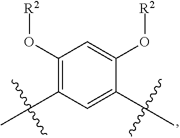

and each X is S.

In some embodiments, each R¹ is, independently, the free base or salt form of —(CH₂)ₙ—NH₂, where each n is, independently, 1 to 4. In some embodiments, each R¹ is, independently, the free base or salt form of —(CH₂)ₙ—NH₂, where each n is, independently, 1 or 2. In some embodiments, each R¹ is the free base or salt form of —(CH₂)ₙ—NH₂, where each n is 2.

In some embodiments, each R² is, independently, C₁-C₃alkyl or the free base or salt form of —(CH₂)ₙ—NH₂ where n is 1 to 4. In some embodiments, each R² is, independently, C₁-C₃alkyl or the free base or salt form of —(CH₂)ₙ—NR₂, where each n is, independently, 1 or 2. In some embodiments, each R² is, independently, methyl or the free base or salt form of —(CH₂)ₙ—NH₂, where each n is, independently, 2. In some embodiments, each R² is methyl or the free base or salt form of —(CH₂)ₙ—NH₂, where each n is 2.

In some embodiments, each R³ is, independently, CF₃, C(CH₃)₃, or halo. In some embodiments, each R³ is CF₃.

In some embodiments, each R⁴ is, independently, —(CH₂)—NH—C(═NH)NH₂, where each n is, independently, 1 to 4. In some embodiments, each R⁴ is —(CH₂)ₙ—NH—C(═NH)NH₂, where each n is 4.

In some embodiments, G is

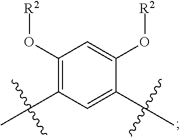

each X is S; each R¹ is, independently, the free base or salt form of —(CH₂)ₙ—NH₂, where each n is, independently, 1 or 2; each R² is, independently, C₁-C₈alkyl or the free base or salt form of —(CH₂)ₙ—NH₂, where each n is, independently, 1 or 2; each R³ is, independently, CF₃, C(CH₃)₃, or halo; and each R⁴ is, independently, —(CH₂)ₙ—NH—C(═NH)NH₂, where each n is, independently, 3 or 4.

In some embodiments, G is

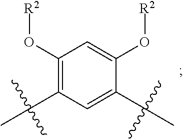

each X is S; each R¹ is the free base or salt form of —(CH₂)ₙ—NH₂, where each n is 1 or 2; each R² is, independently, C₁-C₃alkyl or the free base or salt form of —(CH₂)ₙ—NH₂, where each n is 2; each R³ is, independently, CF₃ or C(CH₃)₃; and each R⁴ is —(CH₂)ₙ—NH—C(═NH)NH₂, where each n is 3 or 4.

In some embodiments, G is

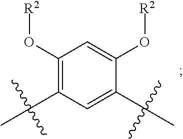

each X is S; each R¹ is the free base or salt form of —(CH₂)ₙ—NH₂, where each n is 2; each R² is, independently, methyl or the free base or salt form of —(CH₂)ₙ—NH₂, where each n is 2; each R³ is, independently, CF₃ or C(CH₃)₃; and each R⁴ is —(CH₂)ₙ—NH—C(═NH)NH₂, where each n is 4.

In some embodiments, G is

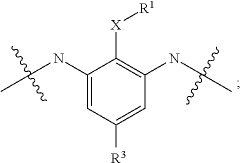

each X is, independently, O or S; each R¹ is, independently, the free base or salt form of —(CH₂)ₙ—NH₂ or —(CH₂)ₙ—NH—C(═NH)NH₂, where each n is, independently, 1 to 4; each R³ is, independently, H or CF₃; and each R⁴ is, independently, —(CH₂)ₙ—NH₂ or —(CH₂)ₙ—NH—C(═NH)NH₂, where each n is, independently, 1 to 4.

In some embodiments, G is

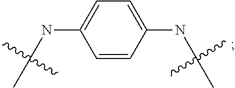

each X is, independently, O or S; each R¹ is

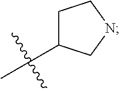

each R³ is, independently, H or CF₃; and each R⁴ is, independently, —(CH₂)ₙ—NH₂ or —(CH₂)ₙ—NH—C(═NH)NH₂, where each n is, independently, 1 to 4.

The present invention also provides methods of modulating an immune response in a mammal comprising administering to the mammal in need thereof a therapeutically effective amount of a compound chosen from:

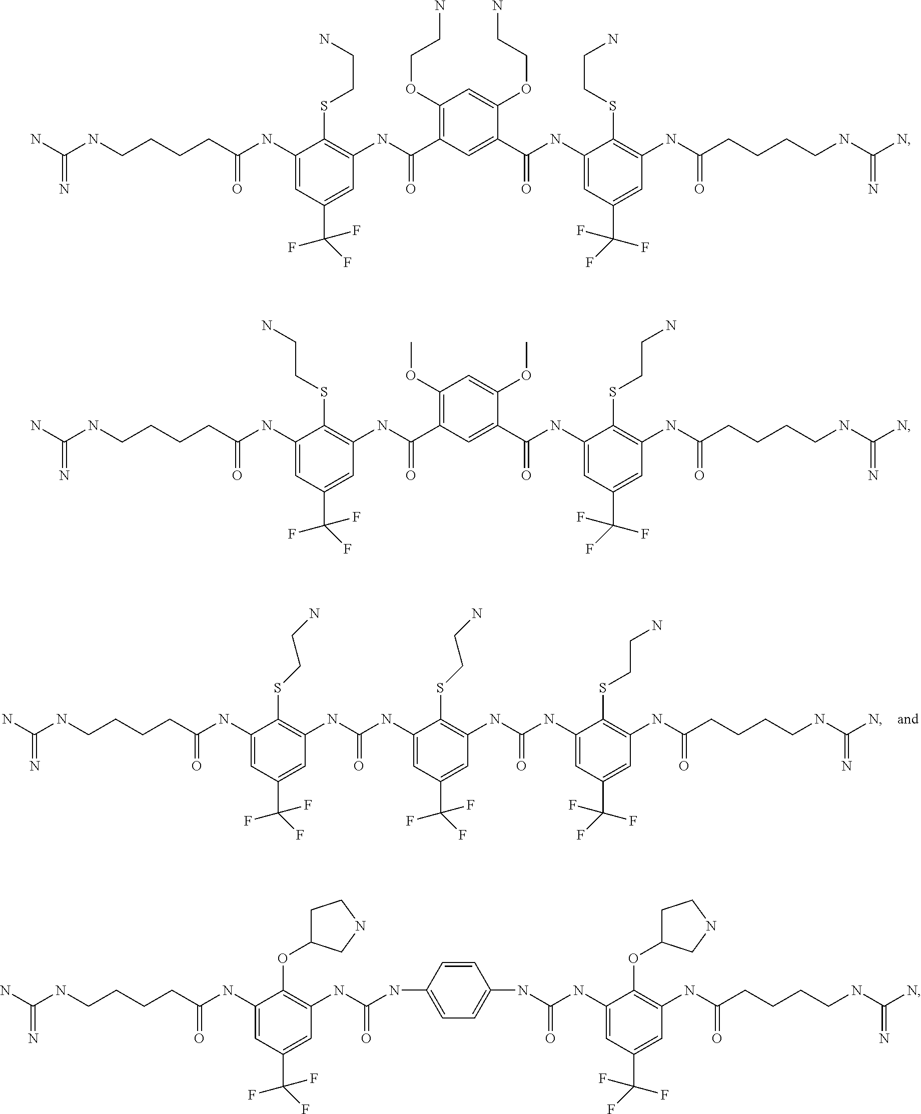

or a pharmaceutically acceptable salt thereof.

In some embodiments, the immune response is against an oral pathogen. In some embodiments, the oral pathogen is chosen from Aggregatibacter spp. such as, for example, Aggregatibacter actinomycetemcomitans; Porphyromonas spp. such as, for example, Porphyromonas gingivalis; Streptococcus spp. such as, for example, Streptococcus sanguis and Streptococcus mutans, Candida spp. such as, for example, Candida albicans, Candida glabrata, Candida krusei, Candida dubliniensis, Candida parapsilosis, and Candida tropicalis; Actinomyces spp. such as, for example, Actinomyces viscosus; and Lactobacillus spp. such as, for example, Lactobacillus casei. In some embodiments, the immune response is against a bacterial pathogen. In some embodiments, the bacterial pathogen is chosen from Staphylococcus spp., such as, for example, Staphylococcus aureus, methicillin-resistant Staphylococcus aureus, and Staphylococcus epidermidis; Streptococcus spp. such as, for example, Streptococcus pneumoniae, Streptococcus pyogenes, and Streptococcus viridans; Escherichia spp. such as, for example, E. coli; Enterococcus spp. such as, for example, Enterococcus faecalis and Enterococcus faecium; Psuedomonas spp. such as, for example, Pseudomonas aeruginosa; Acinetobacter spp. such as, for example, A. baumannii; Haemophilus spp. such as, for example, Haemophilus influenzae; Serratia spp. such as, for example, Serratia marcescens; Moraxella spp. such as, for example, Moraxella catarrhalis; Klebsiella spp. such as, for example, Klebsiella pneumoniae; Proteus spp. such as, for example, Proteus vulgaris and Proteus mirabilis; Bacteroides spp. such as, for example, Bacteroides fragalis; Clostridium spp. such as, for example, Clostridium difficile and Clostridium perfringens; and Propionibacterium spp. such as, for example, Propionibacterium acnes.

The present invention also provides methods of modulating an immune response in a mammal comprising administering to the mammal in need thereof a therapeutically effective amount of a compound of Formula XI:

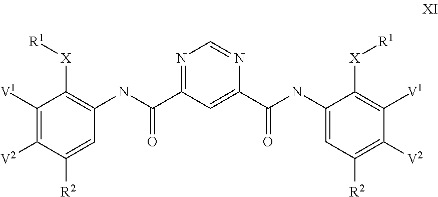

or a pharmaceutically acceptable salt thereof,
wherein:

each X is, independently, O, S, or S(═O)₂;

each R¹ is, independently, —(CH₂)ₙ—NH₂, —(CH₂)ₙ—NH—C(═NH)NH₂, or —(CH₂)ₙ—NH—C(═O)—R⁴, where each n is, independently, 1 to 4, and each R⁴ is, independently, H, C₁-C₃alkyl, or —(CH₂)ₚ—NH₂, where each p is, independently, 1 or 2;

each R² is, independently, H, halo, CF₃, or C(CH₃)₃; and

each V² is H, and each V¹ is, independently, —N—C(═O)—R³, where each R³ is, independently, —(CH₂)ₙ—NH₂ or —(CH₂)—NH—C(═NH)NH₂, where each n is, independently, 1 to 4; or each V¹ is H and each V² is, independently, —S—R⁵, where each R⁵ is, independently, —(CH₂)ₙ—NH₂ or —(CH₂)ₙ—NH—C(═NH)NH₂, where each n is, independently, 1 to 4;

or a pharmaceutically acceptable salt thereof.

In some embodiments, each X is S.

In some embodiments, each R¹ is, independently, —(CH₂)ₙ—NH₂, —(CH₂)ₙ—NH—C(═NH)NH₂, or —(CH₂)ₙ—NH—C(═O)—R⁴, where each n is, independently, 1 or 2, and each R⁴ is, independently, H or methyl. In some embodiments, each R¹ is, independently, —(CH₂)ₙ—NH₂, —(CH₂)ₙ—NH—C(═NH)NH₂, or —(CH₂)ₙ—NH—C(═O)—R⁴, where each n is 2 and each R⁴ is H. In some embodiments, each R¹ is, independently, —(CH₂)ₙ—NH₂ or —(CH₂)ₙ—NH—C(═NH)NH₂, where each n is 2. In some embodiments, each R¹ is —(CH₂)ₙ—NH₂ or —(CH₂)ₙ—NH—C(═NH)NH₂, where each n is 2.

In some embodiments, each R² is, independently, H, Br, F, Cl, CF₃, or C(CH₃)₃. In some embodiments, each R² is Br, F, Cl, CF₃, or C(CH₃)₃.

In some embodiments, each V² is H and each V¹ is, independently, —N—C(═O)—R³, where each R³ is, independently, —(CH₂)ₙ—NH₂ or —(CH₂)—NH—C(═NH)NH₂, where each n is, independently, 1 to 4. In some embodiments, each V² is H and each V¹ is, independently, —N—C(═O)—R³, where each R³ is, independently, —(CH₂)ₙ—NH₂ or —(CH₂)ₙ—NH—C(═NH)NH₂, where each n is, independently, 1 or 2. In some embodiments, each V² is H and each V¹ is, independently, —N—C(═O)—R³, where each R³ is, independently, —(CH₂)ₙ—NH₂ or —(CH₂)ₙ—NH—C(═NH)NH₂, where each n is 2. In some embodiments, each V² is H and each V¹ is —N—C(═O)—R³, where each R³ is —(CH₂)ₙ—NH₂ or —(CH₂)ₙ—NH—C(═NH)NH₂, where n is 2.

In some embodiments, each V¹ is H and each V² is, independently, —S—R⁵, where each R⁵ is, independently, —(CH₂)ₙ—NH₂ or —(CH₂)ₙ—NH—C(═NH)NH₂, where each n is, independently, 1 to 4. In some embodiments, each V¹ is H and each V² is, independently, —S—R⁵, where each R⁵ is, independently, —(CH₂)ₙ—NH₂ or —(CH₂)ₙ—NH—C(═NH)NH₂, where each n is 1 or 2. In some embodiments, each V¹ is H and each V² is, independently, —S—R⁵, where each R⁵ is, independently, —(CH₂)ₙ—NH₂ or —(CH₂)ₙ—NH—C(═NH)NH₂, where each n is 2. In some embodiments, each V¹ is H and each V² is —S—R⁵, where each R⁵ is —(CH₂)ₙ—NH₂ or —(CH₂)ₙ—NH—C(═NH)NH₂, where each n is 2.

In some embodiments, each X is S; each R¹ is, independently, —(CH₂)ₙ—NH₂ or —(CH₂)ₙ—NH—C(═NH)NH₂, where each n is, independently, 1 to 4; each R² is, independently, halo, CF₃, or C(CH₃)₃; and each V¹ is H and each V² is, independently, —S—R⁵, where each R⁵ is, independently, —(CH₂)ₙ—NH₂, where each n is, independently, 1 to 4.

In some embodiments, each X is S; each R¹ is, independently, —(CH₂)ₙ—NH₂, where each n is, independently, 1 or 2; each R² is, independently, CF₃ or C(CH₃)₃; and each V¹ is H and each V² is, independently, —S—R⁵, where each R⁵ is, independently, —(CH₂)ₙ—NH₂, where each n is, independently, 1 or 2.

In some embodiments, each X is S; each R¹ is —(CH₂)ₙ—NH₂, where each n is 1 or 2; each R² is, independently, CF₃ or C(CH₃)₃; and each V¹ is H and each V² is —S—R⁵, where each R⁵ is —(CH₂)ₙ—NH₂, where each n is 1 or 2.

In some embodiments, each X is O or S; each R¹ is, independently, —(CH₂)ₙ—NH₂, —(CH₂)ₙ—NH—C(═NH)NH₂, or —(CH₂)ₙ—NH—C(═O)—R⁴, where each n is, independently, 1 to 4, and each R⁴ is, independently, H or methyl; each R² is, independently, halo, CF₃, or C(CH₃)₃; and each V² is H, and each V¹ is, independently, —N—C(═O)—R³, where each R³ is, independently, —(CH₂)ₙ—NH₂ or —(CH₂)ₙ—NH—C(═NH)NH₂, where each n is, independently, 1 to 4.

In some embodiments, each X is S; each R¹ is, independently, —(CH₂)—NH—C(═O)—R⁴, where each n is, independently, 1 or 2, and each R⁴ is, independently, H or methyl; each R² is, independently, halo; and each V² is H, and each V¹ is —N—C(═O)—R³, where each R³ is —(CH₂)ₙ—NH₂ or —(CH₂)ₙ—NH—C(═NH)NH₂, where each n is 4.

In some embodiments, each X is O or S; each R¹ is, independently, —(CH₂)ₙ—NH₂ or —(CH₂)ₙ—NH—C(═NH)NH₂, where each n is, independently, 1 to 4; each R² is, independently, halo, CF₃, or C(CH₃)₃; and each V² is H, and each V¹ is, independently, —N—C(═O)—R³, where each R³ is, independently, —(CH₂)ₙ—NH₂ or —(CH₂)—NH—C(═NH)NH₂, where each n is, independently, 1 to 4.

In some embodiments, each X is O or S; each R¹ is —(CH₂)ₙ—NH₂ or —(CH₂)ₙ—NH—C(═NH)NH₂, where each n is 1 or 2; each R² is halo, CF₃, or C(CH₃)₃; and each V² is H, and each V¹ is —N—C(═O)—R³, where each R³ is —(CH₂)ₙ—NH₂ or —(CH₂)ₙ—NH—C(═NH)NH₂, where each n is 3 or 4.

In some embodiments, each X is, independently, S or S(═O)₂; each R¹ is, independently, —(CH₂)ₙ—NH₂ or —(CH₂)—NH—C(═O)—R⁴, where each n is, independently, 1 or 2, and each R⁴ is, independently, —(CH₂)ₚ—NH₂, where each p is, independently, 1 or 2; each R² is, independently, halo or CF₃; and each V² is H, and each V¹ is, independently, —N—C(═O)—R³, where each R³ is, independently, —(CH₂)ₙ—NH₂ or —(CH₂)ₙ—NH—C(═NH)NH₂, where each n is, independently, 3 or 4.

The present invention also provides methods of modulating an immune response in a mammal comprising administering to the mammal in need thereof a therapeutically effective amount of a compound chosen from:

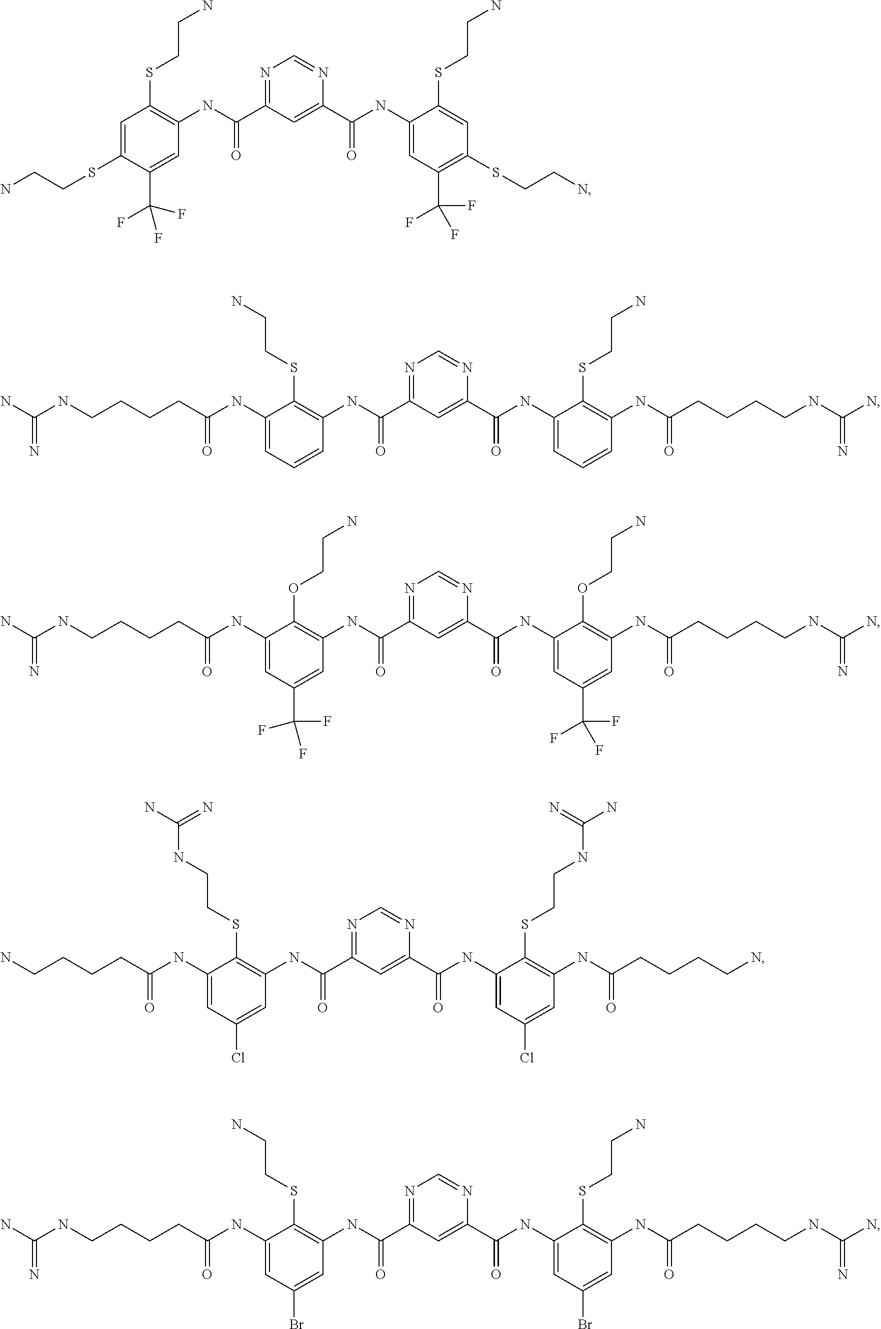

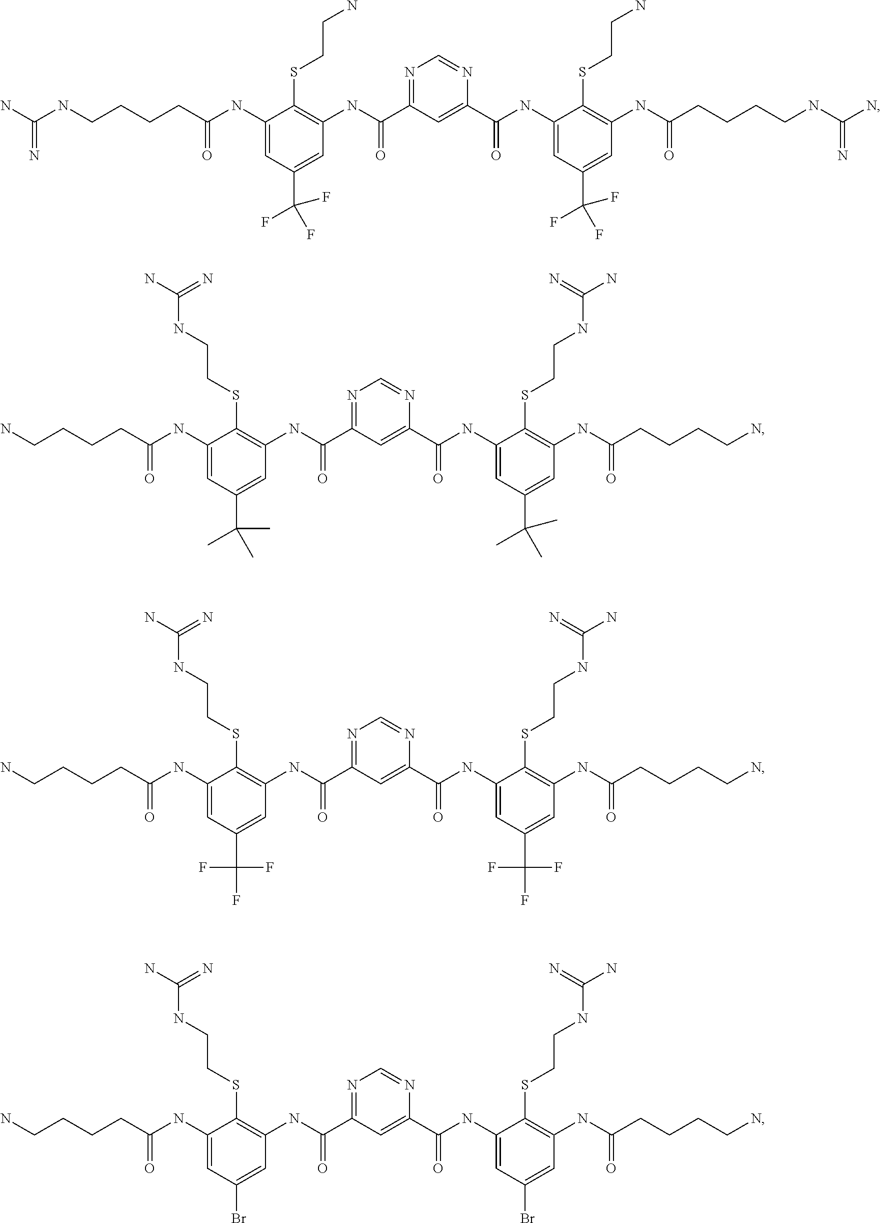

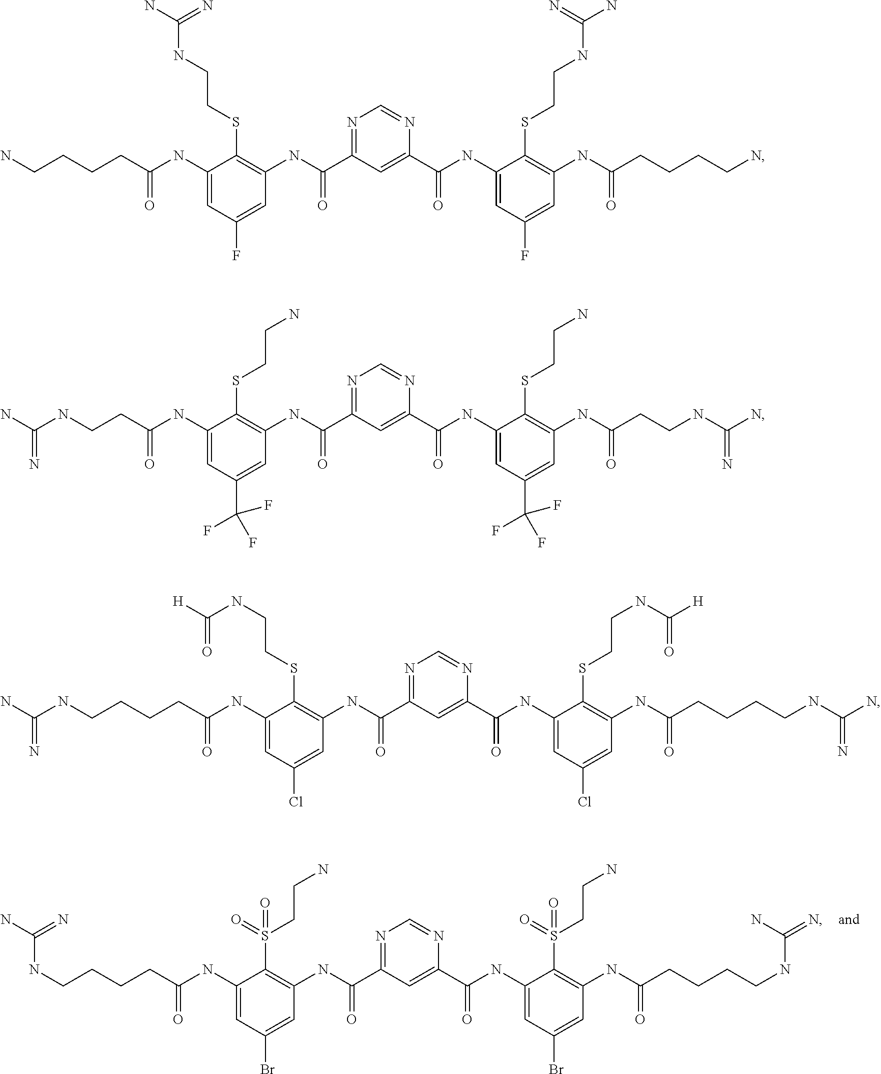

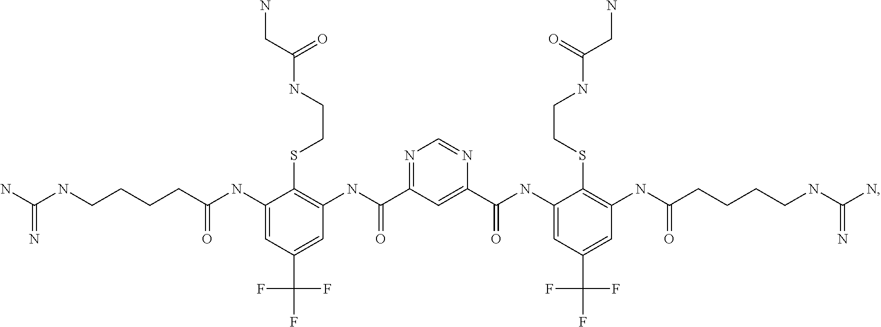

or a pharmaceutically acceptable salt thereof.

In some embodiments, the immune response is against an oral pathogen. In some embodiments, the oral pathogen is chosen from Aggregatibacter spp. such as, for example, Aggregatibacter actinomycetemcomitans; Porphyromonas spp. such as, for example, Porphyromonas gingivalis; Streptococcus spp. such as, for example, Streptococcus sanguis and Streptococcus mutans, Candida spp. such as, for example, Candida albicans, Candida glabrata, Candida krusei, Candida dubliniensis, Candida parapsilosis, and Candida tropicalis; Actinomyces spp. such as, for example, Actinomyces viscosus; and Lactobacillus spp. such as, for example, Lactobacillus casei. In some embodiments, the immune response is against a bacterial pathogen. In some embodiments, the bacterial pathogen is chosen from Staphylococcus spp., such as, for example, Staphylococcus aureus, methicillin-resistant Staphylococcus aureus, and Staphylococcus epidermidis; Streptococcus spp. such as, for example, Streptococcus pneumoniae, Streptococcus pyogenes, and Streptococcus viridans; Escherichia spp. such as, for example, E. coli; Enterococcus spp. such as, for example, Enterococcus faecalis and Enterococcus faecium; Psuedomonas spp. such as, for example, Pseudomonas aeruginosa; Acinetobacter spp. such as, for example, A. baumannii; Haemophilus spp. such as, for example, Haemophilus influenzae; Serratia spp. such as, for example, Serratia marcescens; Moraxella spp. such as, for example, Moraxella catarrhalis; Klebsiella spp. such as, for example, Klebsiella pneumoniae; Proteus spp. such as, for example, Proteus vulgaris and Proteus mirabilis; Bacteroides spp. such as, for example, Bacteroides fragalis; Clostridium spp. such as, for example, Clostridium difficile and Clostridium perfringens; and Propionibacterium spp. such as, for example, Propionibacterium acnes.

The present invention also provides methods of modulating an immune response in a mammal comprising administering to the mammal in need thereof a therapeutically effective amount of a compound of Formula XII:

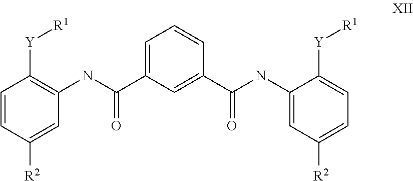

or a pharmaceutically acceptable salt thereof,
wherein:

each Y is, independently, O, S, or NH;

each R¹ is, independently, —(CH₂)ₙ—NH₂ or —(CH₂)ₙ—NH—C(═NH)NH₂, where each n is, independently, 1 to 4; and

each R² is, independently, H, halo, CF₃, or C(CH₃)₃;

or a pharmaceutically acceptable salt thereof.

In some embodiments, each Y is, independently, O, or S. In some embodiments, each Y is O or S.

In some embodiments, each R¹ is, independently, —(CH₂)ₙ—NH₂, where each n is, independently, 2 to 4. In some embodiments, each R¹ is —(CH₂)ₙ—NH₂, where each n is 2 to 4.

In some embodiments, each R² is, independently, halo, CF₃, or C(CH₃)₃. In some embodiments, each R² is halo, CF₃, or C(CH₃)₃.

The present invention also provides methods of modulating an immune response in a mammal comprising administering to the mammal in need thereof a therapeutically effective amount of a compound which is:

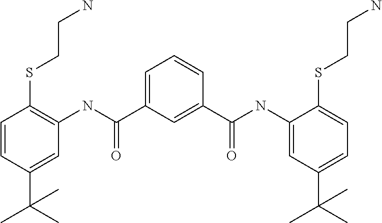

or a pharmaceutically acceptable salt thereof.

In some embodiments, the immune response is against an oral pathogen. In some embodiments, the oral pathogen is chosen from Aggregatibacter spp. such as, for example, Aggregatibacter actinomycetemcomitans; Porphyromonas spp. such as, for example, Porphyromonas gingivalis; Streptococcus spp. such as, for example, Streptococcus sanguis and Streptococcus mutans, Candida spp. such as, for example, Candida albicans, Candida glabrata, Candida krusei, Candida dubliniensis, Candida parapsilosis, and Candida tropicalis; Actinomyces spp. such as, for example, Actinomyces viscosus; and Lactobacillus spp. such as, for example, Lactobacillus casei. In some embodiments, the immune response is against a bacterial pathogen. In some embodiments, the bacterial pathogen is chosen from Staphylococcus spp., such as, for example, Staphylococcus aureus, methicillin-resistant Staphylococcus aureus, and Staphylococcus epidermidis; Streptococcus spp. such as, for example, Streptococcus pneumoniae, Streptococcus pyogenes, and Streptococcus viridans; Escherichia spp. such as, for example, E. coli; Enterococcus spp. such as, for example, Enterococcus faecalis and Enterococcus faecium; Psuedomonas spp. such as, for example, Pseudomonas aeruginosa; Acinetobacter spp. such as, for example, A. baumannii; Haemophilus spp. such as, for example, Haemophilus influenzae; Serratia spp. such as, for example, Serratia marcescens; Moraxella spp. such as, for example, Moraxella catarrhalis; Klebsiella spp. such as, for example, Klebsiella pneumoniae; Proteus spp. such as, for example, Proteus vulgaris and Proteus mirabilis; Bacteroides spp. such as, for example, Bacteroides fragalis; Clostridium spp. such as, for example, Clostridium difficile and Clostridium perfringens; and Propionibacterium spp. such as, for example, Propionibacterium acnes.

The present invention also provides methods of modulating an immune response in a mammal comprising administering to the mammal in need thereof a therapeutically effective amount of a compound of Formula XIII:

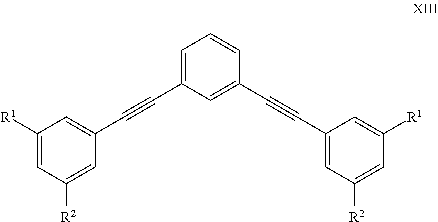

or a pharmaceutically acceptable salt thereof,
wherein:

each R¹ is, independently, H, C₁-C₈alkyl, C₁-C₈alkoxy, halo, OH, CF₃, or CN;

each R² is, independently, —(C₁₋₁₂)ₙ—NH₂ or —(CH₂)—NH—C(═NH)NH₂, where each n is, independently, 1 to 4;

or a pharmaceutically acceptable salt thereof.

In some embodiments, each R¹ is, independently, C₁-C₈alkyl, halo, OH, CF₃, or CN.

In some embodiments, each R¹ is, independently, C₁-C₃alkyl, halo, CF₃, or CN. In some embodiments, each R¹ is methyl or halo. In some embodiments, each R¹ is Br, F, or Cl.

In some embodiments, each R² is, independently, —(CH₂)ₙ—NH—C(═NH)NH₂, where each n is, independently, 1 to 4. In some embodiments, each R² is —(CH₂)—NH—C(═NH)NH₂, where each n is 1 to 4. In some embodiments, each R² is —(CH₂)ₙ—NH—C(═NH)NH₂, where each n is 1 or 2.

In some embodiments, each R¹ is, independently, C₁-C₈alkyl, halo, OH, CF₃, or CN; and each R² is, independently, —(CH₂)—NH—C(═NH)NH₂, where each n is, independently, 1 to 4.

In some embodiments, each R¹ is, independently, C₁-C₃alkyl, halo, CF₃, or CN; and each R² is —(CH₂)—NH—C(═NH)NH₂, where each n is 1 to 4.

In some embodiments, each R¹ is methyl or halo; and each R² is —(CH₂)ₙ—NH—C(═NH)NH₂, where each n is 1 or 2.

The present invention also provides methods of modulating an immune response in a mammal comprising administering to the mammal in need thereof a therapeutically effective amount of a compound which is:

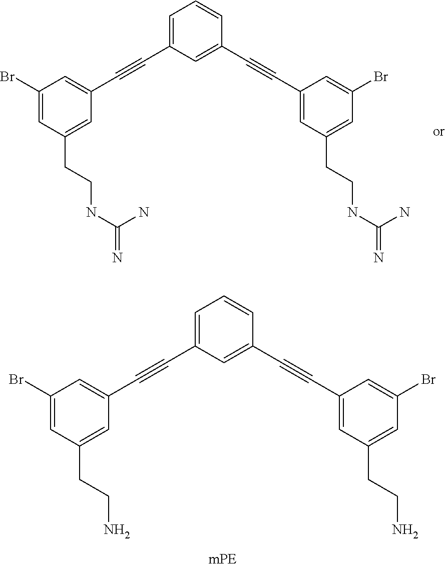

or a pharmaceutically acceptable salt thereof.

In some embodiments, the immune response is against an oral pathogen. In some embodiments, the oral pathogen is chosen from Aggregatibacter spp. such as, for example, Aggregatibacter actinomycetemcomitans; Porphyromonas spp. such as, for example, Porphyromonas gingivalis; Streptococcus spp. such as, for example, Streptococcus sanguis and Streptococcus mutans, Candida spp. such as, for example, Candida albicans, Candida glabrata, Candida krusei, Candida dubliniensis, Candida parapsilosis, and Candida tropicalis; Actinomyces spp. such as, for example, Actinomyces viscosus; and Lactobacillus spp. such as, for example, Lactobacillus casei. In some embodiments, the immune response is against a bacterial pathogen. In some embodiments, the bacterial pathogen is chosen from Staphylococcus spp., such as, for example, Staphylococcus aureus, methicillin-resistant Staphylococcus aureus, and Staphylococcus epidermidis; Streptococcus spp. such as, for example, Streptococcus pneumoniae, Streptococcus pyogenes, and Streptococcus viridans; Escherichia spp. such as, for example, E. coli; Enterococcus spp. such as, for example, Enterococcus faecalis and Enterococcus faecium; Psuedomonas spp. such as, for example, Pseudomonas aeruginosa; Acinetobacter spp. such as, for example, A. baumannii; Haemophilus spp. such as, for example, Haemophilus influenzae; Serratia spp. such as, for example, Serratia marcescens; Moraxella spp. such as, for example, Moraxella catarrhalis; Klebsiella spp. such as, for example, Klebsiella pneumoniae; Proteus spp. such as, for example, Proteus vulgaris and Proteus mirabilis; Bacteroides spp. such as, for example, Bacteroides fragalis; Clostridium spp. such as, for example, Clostridium difficile and Clostridium perfringens; and Propionibacterium spp. such as, for example, Propionibacterium acnes.

The present invention also provides methods of modulating an immune response in a mammal comprising administering to the mammal in need thereof a therapeutically effective amount of a compound of Formula XIV:

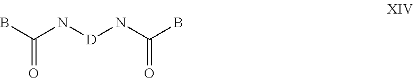

or a pharmaceutically acceptable salt thereof,
wherein:

D is

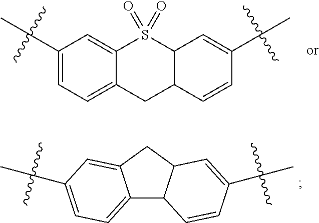

each B is, independently, —(CH₂)ₙ—NH—C(═NH)NH₂, where each n is, independently, 1 to 4,

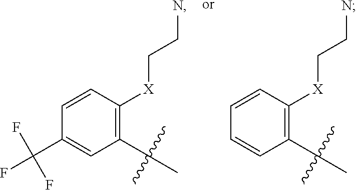

and

each X is, independently, O or S;

or a pharmaceutically acceptable salt thereof.

In some embodiments, D is

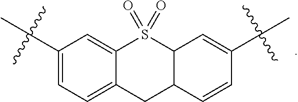

In some embodiments, each B is, independently, —(CH₂)ₙ—NH—C(═NH)NH₂, where each n is, independently, 1 to 4.

In some embodiments, each X is S.

In some embodiments, D is

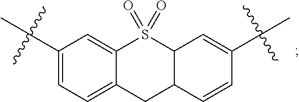

each B is, independently, —(CH₂)ₙ—NH—C(═NH)NH₂, where each n is, independently, 3 or 4, or

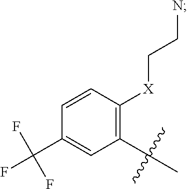

and each X is S.

In some embodiments, D is

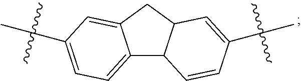

each B is, independently,

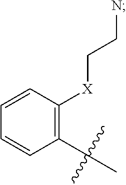

and each X is, independently, O or S.

The present invention also provides methods of modulating an immune response in a mammal comprising administering to the mammal in need thereof a therapeutically effective amount of a compound which is:

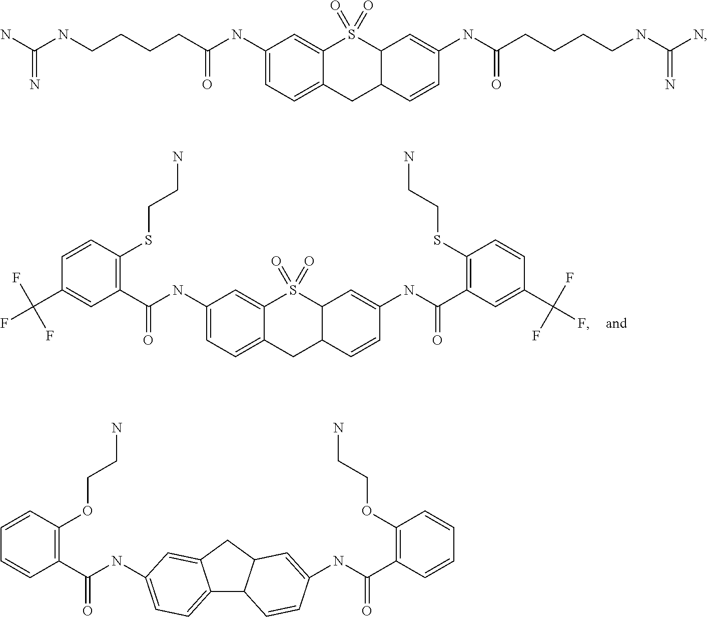

or a pharmaceutically acceptable salt thereof.

In some embodiments, the immune response is against an oral pathogen. In some embodiments, the oral pathogen is chosen from Aggregatibacter spp. such as, for example,

Aggregatibacter actinomycetemcomitans; Porphyromonas spp. such as, for example, Porphyromonas gingivalis; Streptococcus spp. such as, for example, Streptococcus sanguis and Streptococcus mutans, Candida spp. such as, for example, Candida albicans, Candida glabrata, Candida krusei, Candida dubliniensis, Candida parapsilosis, and Candida tropicalis; Actinomyces spp. such as, for example, Actinomyces viscosus; and Lactobacillus spp. such as, for example, Lactobacillus casei. In some embodiments, the immune response is against a bacterial pathogen. In some embodiments, the bacterial pathogen is chosen from Staphylococcus spp., such as, for example, Staphylococcus aureus, methicillin-resistant Staphylococcus aureus, and Staphylococcus epidermidis; Streptococcus spp. such as, for example, Streptococcus pneumoniae, Streptococcus pyogenes, and Streptococcus viridans; Escherichia spp. such as, for example, E. coli; Enterococcus spp. such as, for example, Enterococcus faecalis and Enterococcus faecium; Psuedomonas spp. such as, for example, Pseudomonas aeruginosa; Acinetobacter spp. such as, for example, A. baumannii; Haemophilus spp. such as, for example, Haemophilus influenzae; Serratia spp. such as, for example, Serratia marcescens; Moraxella spp. such as, for example, Moraxella catarrhalis; Klebsiella spp. such as, for example, Klebsiella pneumoniae; Proteus spp. such as, for example, Proteus vulgaris and Proteus mirabilis; Bacteroides spp. such as, for example, Bacteroides fragalis; Clostridium spp. such as, for example, Clostridium difficile and Clostridium perfringens; and Propionibacterium spp. such as, for example, Propionibacterium acnes.

The present invention also provides methods of modulating an immune response in a mammal comprising administering to the mammal in need thereof a therapeutically effective amount of a compound of Formula XV:

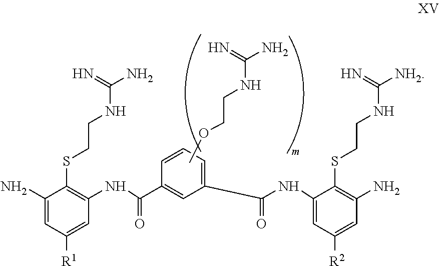

or a pharmaceutically acceptable salt thereof,
wherein:

R¹ is H or C₁₋₁₀ alkyl;

R² is H or C₁₋₁₀ alkyl; and

m is 1 or 2.

The present invention also provides methods of modulating an immune response in a mammal comprising administering to the mammal in need thereof a therapeutically effective amount of a compound of Formula XVI:

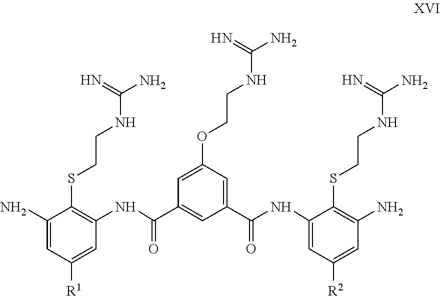

or a pharmaceutically acceptable salt thereof,
wherein:

R¹ is H or C₁₋₈ alkyl; and

R² is H or C₁₋₈ alkyl.

In some embodiments, R¹ and R² are each, independently, H or C₁₋₈ alkyl. In some embodiments, R¹ and R² are each, independently, C₁₋₈ alkyl, C₂₋₇ alkyl, C₃₋₇ alkyl, or C₃₋₆ alkyl. In some embodiments, R¹ and R² are each, independently, 2-methylpropan-2-yl, propan-2-yl, 2-methylbutan-2-yl, 2,3-dimethylbutan-2-yl, or 2,3,3-trimethylbutan-2-yl. In some embodiments, R¹ and R² are each, independently, branched C₃₋₇ alkyl or branched C₃₋₆ alkyl. In some embodiments, R¹ and R² are each, independently, H or C₁₋₄ alkyl. In some embodiments, R¹ and R² are each independently, H, methyl, ethyl, propan-1yl, propan-2-yl, butan-1-yl, butan-2-yl, or 2-methylpropan-2-yl. In some embodiments, R¹ and R² are each independently, H, methyl, or ethyl. In some embodiments, R¹ and R² are the same. In some embodiments, R¹ and R² are different. In some embodiments, R¹ and R² are each 2-methylpropan-2-yl.

The present invention also provides methods of modulating an immune response in a mammal comprising administering to the mammal in need thereof a therapeutically effective amount of a compound of Formula XVII:

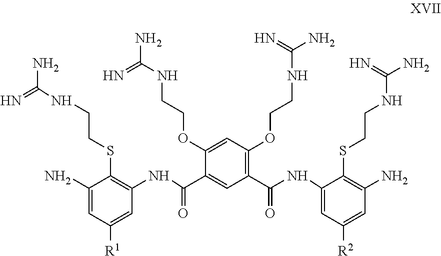

or a pharmaceutically acceptable salt thereof,
wherein:

R¹ is H or C₁₋₈ alkyl; and

R² is H or C₁₋₈ alkyl.

In some embodiments, R¹ and R² are each, independently, H or C₁₋₈ alkyl. In some embodiments, R¹ and R² are each, independently, C₁₋₈ alkyl, C₂₋₇ alkyl, C₃₋₇ alkyl, or C₃₋₆ alkyl. In some embodiments, R¹ and R² are each, independently, propan-2-yl, 2-methylpropan-2-yl, 2-methylbutan-2-yl, 2,3-dimethylbutan-2-yl, or 2,3,3-trimethylbutan-2-yl. In some embodiments, R¹ and R² are each, independently, branched C₃₋₇ alkyl or branched C₃₋₆ alkyl. In some embodiments, R¹ and R² are each, independently, H or C₁₋₄ alkyl. In some embodiments, R¹ and R² are each independently, H, methyl, ethyl, propan-1yl, propan-2-yl, butan-1-yl, butan-2-yl, or 2-methylpropan-2-yl. In some embodiments, R¹ and R² are each independently, H, methyl, or ethyl. In some embodiments, R¹ and R² are the same. In some embodiments, R¹ and R² are different. In some embodiments, R¹ and R² are each 2-methylpropan-2-yl.

The present invention also provides methods of modulating an immune response in a mammal comprising administering to the mammal in need thereof a therapeutically effective amount of a compound which is:

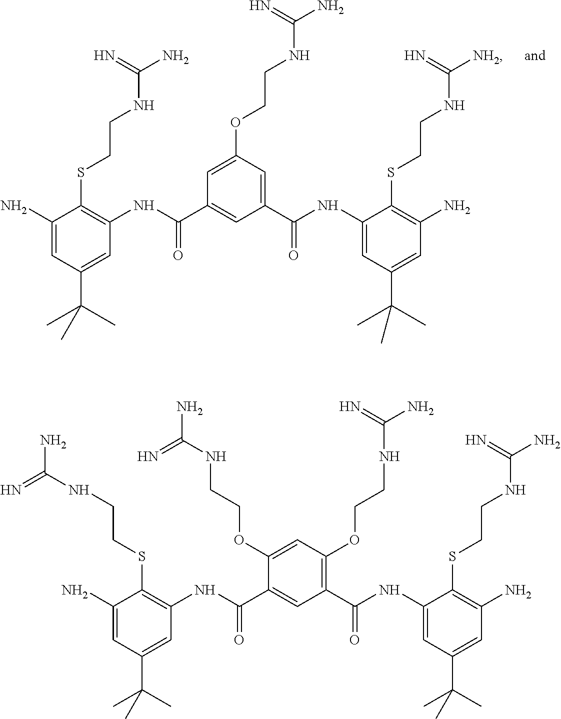

or a pharmaceutically acceptable salt thereof.

In some embodiments, the immune response is against an oral pathogen. In some embodiments, the oral pathogen is chosen from Aggregatibacter spp. such as, for example, Aggregatibacter actinomycetemcomitans; Porphyromonas spp. such as, for example, Porphyromonas gingivalis; Streptococcus spp. such as, for example, Streptococcus sanguis and Streptococcus mutans, Candida spp. such as, for example, Candida albicans, Candida glabrata, Candida krusei, Candida dubliniensis, Candida parapsilosis, and Candida tropicalis; Actinomyces spp. such as, for example, Actinomyces viscosus; and Lactobacillus spp. such as, for example, Lactobacillus casei. In some embodiments, the immune response is against a bacterial pathogen. In some embodiments, the bacterial pathogen is chosen from Staphylococcus spp., such as, for example, Staphylococcus aureus, methicillin-resistant Staphylococcus aureus, and Staphylococcus epidermidis; Streptococcus spp. such as, for example, Streptococcus pneumoniae, Streptococcus pyogenes, and Streptococcus viridans; Escherichia spp. such as, for example, E. coli; Enterococcus spp. such as, for example, Enterococcus faecalis and Enterococcus faecium; Psuedomonas spp. such as, for example, Pseudomonas aeruginosa; Acinetobacter spp. such as, for example, A. baumannii; Haemophilus spp. such as, for example, Haemophilus influenzae; Serratia spp. such as, for example, Serratia marcescens; Moraxella spp. such as, for example, Moraxella catarrhalis; Klebsiella spp. such as, for example, Klebsiella pneumoniae; Proteus spp. such as, for example, Proteus vulgaris and Proteus mirabilis; Bacteroides spp. such as, for example, Bacteroides fragalis; Clostridium spp. such as, for example, Clostridium difficile and Clostridium perfringens; and Propionibacterium spp. such as, for example, Propionibacterium acnes.

The present invention also provides methods of modulating an immune response in a mammal comprising administering to the mammal in need thereof a therapeutically effective amount of a compound of Formula XVIII:

R¹—[—X-A₁-Y—X-A₂-Y—]ₘ—R²XVIII

or a pharmaceutically acceptable salt thereof,
wherein:

each X is, independently, NR⁸, —N(R⁸)N(R⁸)—, O, or S;

each Y is, independently, C═O, C═S, O═S═O, —C(═O)C(═O)—, or —CR^{a}R^{b}—;

R^{a} and R^{b} are each, independently, hydrogen, a PL group, or an NPL group;

each R⁸ is, independently, hydrogen or alkyl;

A₁ and A₂ are each, independently, optionally substituted arylene or optionally substituted heteroarylene, wherein A₁ and A₂ are, independently, optionally substituted with one or more PL group(s), one or more NPL group(s), or a combination of one or more PL group(s) and one or more NPL group(s); or

each A₁ is, independently, optionally substituted arylene or optionally substituted heteroarylene, and each A₂ is a C₃ to C₈ cycloalkyl or —(CH₂)_{q}—, wherein q is 1 to 7, wherein

A₁ and A₂ are, independently, optionally substituted with one or more PL group(s), one or more NPL group(s), or a combination of one or more PL group(s) and one or more NPL group(s); or

each A₂ is optionally substituted arylene or optionally substituted heteroarylene, and each A₁ is a C₃ to C₈ cycloalkyl or —(CH₂)_{q}—, wherein q is 1 to 7, wherein A₁ and A₂ are each, independently, optionally substituted with one or more PL group(s), one or more NPL group(s), or a combination of one or more PL group(s) and one or more NPL group(s);

R¹ is hydrogen, a PL group, or an NPL group, and R² is —X-A₁-Y—R¹¹, wherein R¹¹ is hydrogen, a PL group, or an NPL group; or

R¹ and R² are each, independently, hydrogen, a PL group, or an NPL group; or

R¹ and R² together are a single bond; or

R¹ is —Y-A₂-X—R¹², wherein R¹² is hydrogen, a PL group, or an NPL group, and R² is hydrogen, a PL group, or an NPL group;

each NPL group is, independently, —B(OR⁴)₂ or

—(NR³′)_{q1NPL}—U^{NPL}-LK^{NPL}—(NR³″)_{q2NPL}—R⁴′, wherein:

R³, R³′, and R³″ are each, independently, hydrogen, alkyl, or alkoxy;

R⁴ and R⁴′ are each, independently, hydrogen, alkyl, alkenyl, alkynyl, cycloalkyl, aryl, or heteroaryl, wherein each of the alkyl, alkenyl, alkynyl, cycloalkyl, aryl, and heteroaryl is optionally substituted with one or more substitutents, wherein each substituent is, independently, alkyl, halo, or haloalkyl;

each U^{NPL} is, independently, absent or O, S, S(═O), S(═O)₂, NR³, —C(═O)—, —C(═O)—NR³—, —C(═O)—N═N—NR³—, —C(═O)—NR³—N═N—, —N═N—NR³—, —C(═N—N(R³)₂)—, —C(═NR³)—, —C(═O)O—, —C(═O)S—, —C(═S)—, —O—P(═O)₂O—, —S—C═N—, or —C(═O)—NR³—O—, wherein groups with two chemically nonequivalent termini can adopt both possible orientations;

each LK^{NPL} is, independently, —(CH₂)_{pNPL}— or C₂₋₈ alkenylenyl, wherein each of the —(CH₂)_{pNPL} and C₂₋₈ alkenylenyl is optionally substituted with one or more substituents, wherein each substituent is, independently, amino, hydroxyl, aminoalkyl, hydroxylalkyl, or alkyl;

each pNPL is, independently, an integer from 0 to 8;

q1NPL and q2NPL are each, independently, 0, 1, or 2;

each PL group is, independently, halo, hydroxyethoxymethyl, methoxyethoxymethyl, polyoxyethylene, or —(NR⁵′)_{q1PL}—U^{PL}-LK^{PL}—(NR⁵″)_{q2PL}—V, wherein:

R⁵, R⁵′, and R⁵″ are each, independently, hydrogen, alkyl, or alkoxy;

each U^{PL} is, independently, absent or O, S, S(═O), S(═O)₂, NR⁵, —C(═O)—, —C(═O)—NR⁵—, —C(═O)—N═N—NR⁵—, —C(═O)—NR⁵—N═N—, —N═N—NR⁵—, —C(═N—N(R⁵)₂)—, —C(═NR⁵)—, —C(═O)O—, —C(═O)S—, —C(═S)—, —O—P(═O)₂O—, —S—C═N—, or —C(═O)—NR⁵—O—, wherein groups with two chemically nonequivalent termini can adopt either of the two possible orientations;

each V is, independently, nitro, cyano, amino, halo, hydroxy, alkoxy, alkylthio, alkylamino, dialkylamino, —NH(CH₂)ₚNH₂ wherein p is 1 to 5, —C(═O)NH(CH₂)ₚNH₂ wherein p is 1 to 5, —C(═O)NH(CH₂)ₚNHC(═NH)NH₂ wherein p is 1 to 5, —C(═O)NH(CH₂)ₚNHC(═O)NH₂ wherein p is 1 to 5, —NHC(═O)-alkyl, —N(CH₂CH₂NH₂)₂, diazamino, amidino, guanidino, ureido, carbamoyl, —C(═O)OH, —C(═O)OR^{e}, —C(═O)NH—OH, —O—NH—C(═NH)NH₂, —NH—S(═O)₂OH, S(═O)₂OH, NR^{d}R^{e}, semicarbazone, aryl, cycloalkyl, heterocycloalkyl, or heteroaryl, wherein each of the aryl and cycloalkyl is substituted with one or more substitutents, wherein each of the heterocycloalkyl and heteroaryl is optionally substituted with one or more substituents, and wherein each of the substituents for the aryl, cycloalkyl, heterocycloalkyl, and heteroaryl is, independently, nitro, cyano, amino, halo, hydroxy, alkoxy, alkylthio, alkylamino, dialkylamino, —NH(CH₂)ₚNH₂ wherein p is 1 to 5, —N(CH₂CH₂NH₂)₂, diazamino, amidino, guanidino, ureido, carbamoyl, —C(═O)OH, —C(═O)OR^{c}, —C(═O)NH—OH, —O—NH—C(═NH)NH₂, —NH—S(═O)₂OH, S(═O)₂OH, NR^{d}R^{e}, semicarbazone, aminosulfonyl, aminoalkoxy, aminoalkylthio, lower acylamino, or benzyloxycarbonyl;

each R^{c} is, independently, C₁₋₆ alkyl, C₁₋₆ haloalkyl, C₂₋₆ alkenyl, C₂₋₆ alkynyl, aryl, cycloalkyl, heteroaryl, heterocycloalkyl, arylalkyl, heteroarylalkyl, cycloalkylalkyl, or heterocycloalkylalkyl, each optionally substituted by one or more substitutents, wherein each substituent is, independently, OH, amino, halo, C₁₋₆ alkyl, C₁₋₆ haloalkyl, aryl, arylalkyl, heteroaryl, heteroarylalkyl, cycloalkyl, or heterocycloalkyl;

R^{d} and R^{e} are, independently, H, C₁₋₆ alkyl, C₁₋₆ haloalkyl, C₂₋₆ alkenyl, C₂₋₆ alkynyl, aryl, heteroaryl, cycloalkyl, heterocycloalkyl, arylalkyl, heteroarylalkyl, cycloalkylalkyl, or heterocycloalkylalkyl, wherein each of the C₁₋₆ alkyl, C₁₋₆ haloalkyl, C₂₋₆ alkenyl, C₂₋₆ alkynyl, aryl, heteroaryl, cycloalkyl, heterocycloalkyl, arylalkyl, heteroarylalkyl, cycloalkylalkyl and heterocycloalkylalkyl is optionally substituted by OH, amino, halo, C₁₋₆ alkyl, C₁₋₆ haloalkyl, C₁₋₆ haloalkyl, aryl, arylalkyl, heteroaryl, heteroarylalkyl, cycloalkyl, or heterocycloalkyl;

or R^{d} and R^{e} together with the N atom to which they are attached form a 4-, 5-, 6-, 7-, or 8-membered heterocycloalkyl;

each LK^{PL} is, independently, —(CH₂)_{pPL}— or C₂₋₈ alkenylenyl, wherein each of the —(CH₂)_{pNPL}— and C₂₋₈ alkenylenyl is optionally substituted with one or more substituents, wherein each substituent is, independently, amino, hydroxyl, aminoalkyl, hydroxylalkyl, or alkyl;

each pPL is, independently, an integer from 0-8;

q1 PL and q2PL are each, independently, 0, 1, or 2; and

m is an integer from 1 to about 20.

In some embodiments, each X is, independently, NR⁸; each Y is C═O; and each A₂ is optionally substituted arylene or optionally substituted heteroarylene, and each A₁ is a C₃ to C₈ cycloalkyl or —(CH₂)_{q}—, wherein q is 1 to 7, wherein A₁ and A₂ are each, independently, optionally substituted with one or more PL group(s), one or more NPL group(s), or a combination of one or more PL group(s) and one or more NPL group(s).

In some embodiments, each A₂ is optionally substituted phenyl, and each A₁ is a —(CH₂)—, wherein A₁ and A₂ are each, independently, optionally substituted with one or more PL group(s), one or more NPL group(s), or a combination of one or more PL group(s) and one or more NPL group(s).

In some embodiments, each NPL group is, independently, —(NR³)_{q1NPL}—U^{NPL}-LK^{NPL}—(NR³′)_{q2NPL}—R⁴′, wherein: R³, R³′, and R³″ are each, independently, hydrogen, alkyl, or alkoxy; and R⁴ and R⁴′ are each, independently, hydrogen, alkyl, alkenyl, alkynyl, cycloalkyl, aryl, or heteroaryl, wherein each of the alkyl, alkenyl, alkynyl, cycloalkyl, aryl, and heteroaryl is optionally substituted with one or more substitutents, wherein each substituent is, independently, alkyl, halo, or haloalkyl.

In some embodiments, each NPL group is, independently, —B(OR⁴)₂, R⁴′, or OR⁴′, and R⁴ and R⁴′ are each, independently, alkyl, alkenyl, alkynyl, cycloalkyl, or aryl, each is optionally substituted with one or more substitutents, wherein each substituent is, independently, alkyl, halo, or haloalkyl.

In some embodiments, each NPL group is, independently, R⁴′ or OR⁴′, and each R⁴′ is, independently, alkyl, alkenyl, alkynyl, cycloalkyl, or aryl, each is optionally substituted with one or more substitutents, wherein each substituent is, independently, alkyl, halo, or haloalkyl.

In some embodiments, each NPL group is, independently, alkyl, alkenyl, alkynyl, cycloalkyl, aryl, or alkoxy, each is optionally substituted with one or more substitutents, wherein each substituent is, independently, alkyl, halo, or haloalkyl. In some embodiments, each NPL group is, independently, alkyl, haloalkyl, alkoxy, or haloalkoxy.

In some embodiments, each V is, independently, nitro, cyano, amino, hydroxy, alkoxy, alkylthio, alkylamino, dialkylamino, —NH(CH₂)ₚNH₂ wherein p is 1 to 5, —N(CH₂CH₂NH₂)₂, diazamino, amidino, guanidino, ureido, carbamoyl, —C(═O)OH, —C(═O)OR^{c}, —C(═O)NH—OH, —O—NH—C(═NH)NH₂, —NH—S(═O)₂OH, S(═O)₂OH, NR^{d}R^{e}, semicarbazone, aryl, heterocycloalkyl, or heteroaryl, wherein the aryl is substituted with one or more substitutents, wherein each of the heterocycloalkyl and heteroaryl is optionally substituted with one or more substituents, and wherein each of the substituents for the aryl, heterocycloalkyl, and heteroaryl is, independently, nitro, cyano, amino, hydroxy, alkoxy, alkylthio, alkylamino, dialkylamino, —NH(CH₂)ₚNH₂ wherein p is 1 to 5, —N(CH₂CH₂NH₂)₂, diazamino, amidino, guanidino, ureido, carbamoyl, —C(═O)OH, —C(═O)OR^{c}, —C(═O)NH—OH, —O—NH—C(═NH)NH₂, —NH—S(═O)₂OH, S(═O)₂OH, NR^{d}R^{e}, semicarbazone, aminosulfonyl, aminoalkoxy, aminoalkylthio, lower acylamino, or benzyloxycarbonyl.

In some embodiments, each V is, independently, hydroxy, amino, alkylamino, dialkylamino, —NH(CH₂)ₚNH₂ wherein p is 1 to 5, —N(CH₂CH₂NH₂)₂, guanidino, amidino, ureido, carbamoyl, —C(═O)OH, —C(═O)OR^{c}, —C(═O)NH—OH, —O—NH—C(═NH)NH₂, —NH—S(═O)₂OH, S(═O)₂OH, NR^{d}R^{e}, a substituted aryl group, heterocycloalkyl, or heteroaryl, wherein each of the heterocycloalkyl and heteroaryl is optionally substituted with one more substituents, wherein each substituent is, independently, alkyl, haloalkyl, alkoxy, haloalkoxy, amino, cyano, nitro, hydroxy, —NH(CH₂)ₚNH₂ wherein p is 1 to 5, —N(CH₂CH₂NH₂)₂, amidino, guanidino, aminosulfonyl, aminoalkoxy, aminoalkylthio, lower acylamino, or benzyloxycarbonyl; and wherein the substituted aryl group is substituted with one more substituents, wherein each substituent is, independently, amino, cyano, nitro, hydroxy, —NH(CH₂)ₚNH₂ wherein p is 1 to 5, —N(CH₂CH₂NH₂)₂, amidino, guanidino, aminosulfonyl, aminoalkoxy, aminoalkylthio, lower acylamino, or benzyloxycarbonyl.

In some embodiments, each V is, independently, hydroxy, amino, alkylamino, dialkylamino, —NH(CH₂)ₚNH₂ wherein p is 1 to 5, —N(CH₂CH₂NH₂)₂, guanidino, amidino, ureido, carbamoyl, —C(═O)OH, —C(═O)OR^{c}, —C(═O)NH—OH, —O—NH—C(═NH)NH₂, —NH—S(═O)₂OH, S(═O)₂OH, NR^{d}R^{e}, a substituted aryl group, heterocycloalkyl, or heteroaryl, wherein each of the heterocycloalkyl and heteroaryl is optionally substituted with one more substituents, wherein each substituent is, independently, amino, cyano, nitro, hydroxy, —NH(CH₂)ₚNH₂ wherein p is 1 to 5, —N(CH₂CH₂NH₂)₂, amidino, guanidino, aminosulfonyl, aminoalkoxy, aminoalkylthio, lower acylamino, or benzyloxycarbonyl; and wherein the substituted aryl group is substituted with one more substituents, wherein each substituent is, independently, amino, cyano, nitro, hydroxy, —NH(CH₂)ₚNH₂ wherein p is 1 to 5, —N(CH₂CH₂NH₂)₂, amidino, guanidino, aminosulfonyl, aminoalkoxy, aminoalkylthio, lower acylamino, or benzyloxycarbonyl.

In some embodiments, each V is, independently, hydroxy, amino, alkylamino, dialkylamino, —NH(CH₂)ₚNH₂ wherein p is 1 to 5, —N(CH₂CH₂NH₂)₂, guanidino, amidino, ureido, carbamoyl, —C(═O)NH—OH, —O—NH—C(═NH)NH₂, —NH—S(═O)₂OH, NR^{d}R^{e}, heterocycloalkyl, or heteroaryl, wherein each of the heterocycloalkyl and heteroaryl is optionally substituted with one more substituents, wherein each substituent is, independently, alkyl, haloalkyl, alkoxy, haloalkoxy, amino, cyano, nitro, hydroxy, —NH(CH₂)ₚNH₂ wherein p is 1 to 5, —N(CH₂CH₂NH₂)₂, amidino, guanidino, aminosulfonyl, aminoalkoxy, aminoalkylthio, lower acylamino, or benzyloxycarbonyl.

In some embodiments, each V is, independently, hydroxy, amino, alkylamino, dialkylamino, —NH(CH₂)ₚNH₂ wherein p is 1 to 5, —N(CH₂CH₂NH₂)₂, guanidino, amidino, ureido, carbamoyl, —C(═O)NH—OH, —O—NH—C(═NH)NH₂, —NH—S(═O)₂OH, NR^{d}R^{e}, heterocycloalkyl, or heteroaryl, wherein each of the heterocycloalkyl and heteroaryl is optionally substituted with one more substituents, wherein each substituent is, independently, amino, cyano, nitro, hydroxy, —NH(CH₂)ₚNH₂ wherein p is 1 to 5, —N(CH₂CH₂NH₂)₂, amidino, guanidino, aminosulfonyl, aminoalkoxy, aminoalkylthio, lower acylamino, or benzyloxycarbonyl.

In some embodiments, each V is, independently, hydroxy, amino, alkylamino, arylamino, heteroarylamino, ureido, guanidino, carbamoyl, —C(═O)OH, —C(═O)OR^{c}, —C(═O)NH—OH, —O—NH—C(═NH)NH₂, —NH—S(═O)₂OH, S(═O)₂OH, a 3-8 membered heterocycloalkyl, a 5- to 10-membered heteroaryl, or a 6- to 10-membered substituted aryl, wherein the substituted aryl is substituted with one or more substituents, wherein each substituent is, independently, OH, amino, hydroxylalkyl, or aminoalkyl, and wherein each of the 3-8 membered heterocycloalkyl and the 5- to 10-membered heteroaryl is optionally substituted with one or more substituents, wherein each substituent is, independently, alkyl, haloalkyl, alkoxy, haloalkoxy, amino, cyano, nitro, hydroxy, —NH(CH₂)ₚNH₂ wherein p is 1 to 5, —N(CH₂CH₂NH₂)₂, amidino, guanidino, aminosulfonyl, aminoalkoxy, aminoalkylthio, lower acylamino, or benzyloxycarbonyl.

In some embodiments, each V is, independently, hydroxy, amino, alkylamino, arylamino, heteroarylamino, ureido, guanidino, carbamoyl, —C(═O)OH, —C(═O)OR^{c}, —C(═O)NH—OH, —O—NH—C(═NH)NH₂, —NH—S(═O)₂OH, S(═O)₂OH, a 3-8 membered heterocycloalkyl, a 5- to 10-membered heteroaryl, or a 6- to 10-membered substituted aryl, wherein the substituted aryl is substituted with one or more substituents, wherein each substituent is, independently, OH, amino, hydroxylalkyl, or aminoalkyl.

In some embodiments, each V is, independently, amino, heteroarylamino, ureido, guanidino, carbamoyl, C(═O)OH, —C(═O)OR^{c}, —C(═O)NH—OH, —O—NH—C(═NH)NH₂, —NH—S(═O)₂OH, S(═O)₂OH, aziridinyl, azetidinyl, pyrrolidinyl, piperidinyl, piperazinyl, morpholino, azepanyl, azocanyl, tetrazolyl, 1,2,4-oxadiazolyl, 1,3,4-oxadiazolyl, imidazolyl, pyridinyl, indolyl, or a substituted phenyl, wherein the substituted phenyl is substituted with one or more substituents, wherein each substituent is, independently, OH or amino.

In some embodiments, each V is, independently, amino, alkylamino, dialkylamino, —NH(CH₂)ₚNH₂ wherein p is 1 to 5, —N(CH₂CH₂NH₂)₂, guanidino, amidino, ureido, heterocycloalkyl, or heteroaryl, wherein each of the heterocycloalkyl and heteroaryl is optionally substituted with one more substituents, wherein each substituent is, independently, amino, cyano, nitro, hydroxy, —NH(CH₂)ₚNH₂ wherein p is 1 to 5, —N(CH₂CH₂NH₂)₂, amidino, guanidino, aminosulfonyl, aminoalkoxy, aminoalkylthio, lower acylamino, or benzyloxycarbonyl.

In some embodiments, each V is, independently, amino, alkylamino, dialkylamino, —NH(CH₂)ₚNH₂ wherein p is 1 to 5, —N(CH₂CH₂NH₂)₂, guanidino, amidino, ureido, pyrrodinyl, piperidinyl, piperazinyl, 4-methylpiperazinyl, pyridinyl, pyrimidinyl, pyrazinyl, or indolyl. In some embodiments, each V is, independently, amino, alkylamino, dialkylamino, —NH(CH₂)ₚNH₂ wherein p is 1 to 5, —N(CH₂CH₂NH₂)₂, guanidino, amidino, ureido, or indolyl.

In some embodiments, each PL group is, independently, halo, ydroxyethoxymethyl, methoxyethoxymethyl, polyoxyethylene, or —(NR⁵′)_{q1PL}—U^{PL}—(CH₂)_{pPL}—(NR⁵″)_{q2PL}—V.

In some embodiments, each PL group is, independently, halo, —(CH₂)_{pPL}—V, O—(CH₂)_{pPL}—V, and S—(CH₂)_{pPL}—V; each pPL is an integer from 0 to 5; and each V is, independently, hydroxy, amino, halo, alkylamino, dialkylamino, —NH(CH₂)ₚNH₂ wherein p is 1 to 5, —N(CH₂CH₂NH₂)₂, guanidino, amidino, ureido, carbamoyl, —C(═O)OH, —C(═O)OR^{c}, —C(═O)NH—OH, O—NH—C(═NH)NH₂, —NH—S(═O)₂OH, S(═O)₂OH, NR^{d}R^{e}, a substituted aryl group, heterocycloalkyl, or heteroaryl, wherein each of the heterocycloalkyl and heteroaryl is optionally substituted with one more substituents, wherein each substituent is, independently, amino, halo, cyano, nitro, hydroxy, —NH(CH₂)ₚNH₂ wherein p is 1 to 5, —N(CH₂CH₂NH₂)₂, amidino, guanidino, aminosulfonyl, aminoalkoxy, aminoalkylthio, lower acylamino, or benzyloxycarbonyl; and wherein the substituted aryl group is substituted with one more substituents, wherein each substituent is, independently, amino, halo, cyano, nitro, hydroxy, —NH(CH₂)ₚNH₂ wherein p is 1 to 5, —N(CH₂CH₂NH₂)₂, amidino, guanidino, aminosulfonyl, aminoalkoxy, aminoalkylthio, lower acylamino, or benzyloxycarbonyl.

In some embodiments, each PL group is, independently, halo, —(CH₂)_{pPL}—V, O—(CH₂)_{pPL}—V, and S—(CH₂)_{pPL}—V; each pPL is an integer from 0 to 5; and each V is, independently, hydroxy, amino, alkylamino, dialkylamino, —NH(CH₂)ₚNH₂ wherein p is 1 to 5, —N(CH₂CH₂NH₂)₂, guanidino, amidino, ureido, carbamoyl, —C(═O)NH—OH, —O—NH—C(═NH)NH₂, —NH—S(═O)₂OH, NR^{d}R^{e}, heterocycloalkyl, or heteroaryl, wherein each of the heterocycloalkyl and heteroaryl is optionally substituted with one more substituents, wherein each substituent is, independently, amino, halo, cyano, nitro, hydroxy, —NH(CH₂)ₚNH₂ wherein p is 1 to 5, —N(CH₂CH₂NH₂)₂, amidino, guanidino, aminosulfonyl, aminoalkoxy, aminoalkylthio, lower acylamino, or benzyloxycarbonyl.

In some embodiments, each NPL group is, independently, —B(OR⁴)₂, R⁴′, or OR⁴′, R⁴ and R⁴′ are each, independently, alkyl, alkenyl, alkynyl, cycloalkyl, or aryl, each is optionally substituted with one or more substitutents, wherein each substituent is, independently, alkyl, halo, or haloalkyl; each PL group is, independently, halo, —(CH₂)_{pPL}—V, O—(CH₂)_{pPL}—V, or S—(CH₂)_{pPL}—V; each pPL is an integer from 0 to 5; and each V is, independently, hydroxy, amino, halo, alkylamino, dialkylamino, —NH(CH₂)ₚNH₂ wherein p is 1 to 5, —N(CH₂CH₂NH₂)₂, guanidino, amidino, ureido, carbamoyl, —C(═O)OH, —C(═O)OR^{c}, C(═O)NH—OH, —O—NH—C(═NH)NH₂, —NH—S(═O)₂OH, S(═O)₂OH, NR^{d}R^{e}, a substituted aryl group, heterocycloalkyl, or heteroaryl, wherein each of the heterocycloalkyl and heteroaryl is optionally substituted with one more substituents, wherein each substituent is, independently, amino, halo, cyano, nitro, hydroxy, —NH(CH₂)ₚNH₂ wherein p is 1 to 5, —N(CH₂CH₂NH₂)₂, amidino, guanidino, aminosulfonyl, aminoalkoxy, aminoalkylthio, lower acylamino, or benzyloxycarbonyl; and wherein the substituted aryl group is substituted with one more substituents, wherein each substituent is, independently, amino, halo, cyano, nitro, hydroxy, —NH(CH₂)ₚNH₂ wherein p is 1 to 5, —N(CH₂CH₂NH₂)₂, amidino, guanidino, aminosulfonyl, aminoalkoxy, aminoalkylthio, lower acylamino, or benzyloxycarbonyl.

In some embodiments, each NPL group is, independently, R⁴′ or OR⁴′, R⁴ and R⁴′ are each, independently, alkyl, alkenyl, alkynyl, cycloalkyl, or aryl, each is optionally substituted with one or more substitutents, wherein each substituent is, independently, alkyl, halo, or haloalkyl; each PL group is, independently, halo, —(CH₂)_{pPL}—V, O—(CH₂)_{pPL}—V, or S—(CH₂)_{pPL}—V; each pPL is an integer from 0 to 5; and each V is, independently, hydroxy, amino, alkylamino, dialkylamino, —NH(CH₂)ₚNH₂ wherein p is 1 to 5, —N(CH₂CH₂NH₂)₂, guanidino, amidino, ureido, carbamoyl, —C(═O)NH—OH, —O—NH—C(═NH)NH₂, —NH—S(═O)₂OH, NR^{d}R^{e}, heterocycloalkyl, or heteroaryl, wherein each of the heterocycloalkyl and heteroaryl is optionally substituted with one more substituents, wherein each substituent is, independently, amino, halo, cyano, nitro, hydroxy, —NH(CH₂)ₚNH₂ wherein p is 1 to 5, —N(CH₂CH₂NH₂)₂, amidino, guanidino, aminosulfonyl, aminoalkoxy, aminoalkylthio, lower acylamino, or benzyloxycarbonyl.

In some embodiments, each A₂ is phenyl optionally substituted with one or more substituents, wherein each substituent is, independently, OR⁴′, halo, O—(CH₂)_{pPL}—V, or S—(CH₂)_{pPL}—V; and each A₁ is a —(CH₂)— group optionally substituted with one or more substituents, wherein each substituent is, independently, alkyl or —(CH₂)_{pPL}—V.

In some embodiments, each A₂ is phenyl optionally substituted with one or more substituents, wherein each substituent is, independently, O-alkyl, halo, or O—(CH₂)_{pPL}—V, wherein pPL is an integer from 1 to 5; each A₁ is a —(CH₂)— group optionally substituted with one or more substituents, wherein each substituent is, independently, CH₃ or —(CH₂)_{pPL}—V, wherein pPL is an integer from 1 to 5; and each V is, independently, hydroxy, amino, alkylamino, dialkylamino, —NH(CH₂)ₚNH₂ wherein p is 1 to 5, —N(CH₂CH₂NH₂)₂, guanidino, amidino, ureido, carbamoyl, —C(═O)OH, —C(═O)OR^{c}, —C(═O)NH—OH, —O—NH—C(═NH)NH₂, —NH—S(═O)₂OH, S(═O)₂OH, NR^{d}R^{e}, a substituted aryl group, a substituted cycloalkyl group, heterocycloalkyl, or heteroaryl, wherein each of the heterocycloalkyl and heteroaryl is optionally substituted with one more substituents, wherein each substituent is, independently, amino, halo, cyano, nitro, hydroxy, —NH(CH₂)ₚNH₂ wherein p is 1 to 5, —N(CH₂CH₂NH₂)₂, amidino, guanidino, aminosulfonyl, aminoalkoxy, aminoalkylthio, lower acylamino, or benzyloxycarbonyl; and wherein each of the substituted aryl group and the substituted cycloalkyl group is substituted with one more substituents, wherein each substituent is, independently, amino, halo, cyano, nitro, hydroxy, —NH(CH₂)ₚNH₂ wherein p is 1 to 5, —N(CH₂CH₂NH₂)₂, amidino, guanidino, aminosulfonyl, aminoalkoxy, aminoalkylthio, lower acylamino, or benzyloxycarbonyl.

In some embodiments, each A₂ is phenyl optionally substituted with one or more substituents, wherein each substituent is, independently, O-alkyl, halo, or O—(CH₂)_{pPL}—V, wherein pPL is an integer from 1 to 5; each A₁ is a —(CH₂)— group optionally substituted with one or more substituents, wherein each substituent is, independently, CH₃ or —(CH₂)_{pPL}—V, wherein pPL is an integer from 1 to 5; and each V is, independently, hydroxy, amino, alkylamino, dialkylamino, —NH(CH₂)ₚNH₂ wherein p is 1 to 5, —N(CH₂CH₂NH₂)₂, guanidino, amidino, ureido, carbamoyl, —C(═O)OH, —C(═O)OR^{c}, —C(═O)NH—OH, —O—NH—C(═NH)NH₂, —NH—S(═O)₂OH, S(═O)₂OH, NR^{d}R^{e}, a substituted aryl group, a substituted cycloalkyl group, heterocycloalkyl, or heteroaryl, wherein each of the heterocycloalkyl and heteroaryl is optionally substituted with one more substituents, wherein each substituent is, independently, amino, halo, cyano, nitro, hydroxy, —NH(CH₂)ₚNH₂ wherein p is 1 to 5, —N(CH₂CH₂NH₂)₂, amidino, guanidino, aminosulfonyl, aminoalkoxy, aminoalkylthio, lower acylamino, or benzyloxycarbonyl; and wherein each of the substituted aryl group and the substituted cycloalkyl group is substituted with one more substituents, wherein each substituent is, independently, amino, cyano, nitro, hydroxy, —NH(CH₂)ₚNH₂ wherein p is 1 to 5, —N(CH₂CH₂NH₂)₂, amidino, guanidino, aminosulfonyl, aminoalkoxy, aminoalkylthio, lower acylamino, or benzyloxycarbonyl.

In some embodiments, each A₂ is phenyl optionally substituted with one or more substituents, wherein each substituent is, independently, O-alkyl, halo, or O—(CH₂)_{pPL}—V, wherein pPL is an integer from 1 to 5; each A₁ is a —(CH₂)— group optionally substituted with one or more substituents, wherein each substituent is, independently, CH₃ or —(CH₂)_{pPL}—V, wherein pPL is an integer from 1 to 5; and each V is, independently, hydroxy, amino, alkylamino, dialkylamino, —NH(CH₂)ₚNH₂ wherein p is 1 to 5, —N(CH₂CH₂NH₂)₂, guanidino, amidino, ureido, carbamoyl, —C(═O)OH, —C(═O)OR^{c}, —C(═O)NH—OH, —O—NH—C(═NH)NH₂, —NH—S(═O)₂OH, S(═O)₂OH, NR^{d}R^{e}, a substituted aryl group, heterocycloalkyl, or heteroaryl, wherein each of the heterocycloalkyl and heteroaryl is optionally substituted with one more substituents, wherein each substituent is, independently, amino, halo, cyano, nitro, hydroxy, —NH(CH₂)ₚNH₂ wherein p is 1 to 5, —N(CH₂CH₂NH₂)₂, amidino, guanidino, aminosulfonyl, aminoalkoxy, aminoalkylthio, lower acylamino, or benzyloxycarbonyl; and wherein the substituted aryl group is substituted with one more substituents, wherein each substituent is, independently, amino, cyano, nitro, hydroxy, —NH(CH₂)ₚNH₂ wherein p is 1 to 5, —N(CH₂CH₂NH₂)₂, amidino, guanidino, aminosulfonyl, aminoalkoxy, aminoalkylthio, lower acylamino, or benzyloxycarbonyl.

In some embodiments, each A₂ is phenyl optionally substituted with one or more substituents, wherein each substituent is, independently, O—(CH₃); halo, or O—(CH₂)₂—V; each A₁ is a —(CH₂)— group optionally substituted with one substituent, wherein each substituent is, independently, CH₃, (CH₂)—V, (CH₂)₂—V, (CH₂)₃—V, —(CH₂)₄—V, or —(CH₂)₅—V; and each V is, independently, hydroxy, amino, alkylamino, arylamino, heteroarylamino, ureido, guanidino, carbamoyl, —C(═O)OH, —C(═O)OR^{c}, —C(═O)NH—OH, —O—NH—C(═NH)NH₂, —NH—S(═O)₂OH, S(═O)₂OH, a 3-8 membered heterocycloalkyl, a 5- to 10-membered heteroaryl, or a 6- to 10-membered substituted aryl, wherein the substituted aryl is substituted with one or more substituents, wherein each substituent is, independently, OH, amino, hydroxylalkyl, or aminoalkyl.

In some embodiments, each A₂ is phenyl optionally substituted with one or more substituents, wherein each substituent is, independently, O—(CH₃), halo, or O—(CH₂)₂—V; each A₁ is a —(CH₂)— group optionally substituted with one substituent, wherein each substituent is, independently, CH₃, (CH₂)—V, (CH₂)₃—V, —(CH₂)₄—V, and —(CH₂)₅—V; and each V is, independently, hydroxyl, amino, heteroarylamino, ureido, guanidino, carbamoyl, C(═O)OH, —C(═O)OR^{c}, —C(═O)NH—OH, —O—NH—C(═NH)NH₂, —NH—S(═O)₂OH, S(═O)₂OH, aziridinyl, azetidinyl, pyrrolidinyl, piperidinyl, piperazinyl, morpholino, azepanyl, azocanyl, tetrazolyl, 1,2,4-oxadiazolyl, 1,3,4-oxadiazolyl, imidazolyl, pyridinyl, indolyl, or a substituted phenyl, wherein the substituted phenyl is substituted with one or more substituents, wherein each substituent is, independently, OH or amino.

In some embodiments, each A₂ is phenyl optionally substituted with one or more substituents, wherein each substituent is, independently, O—(CH₃), halo, or O—(CH₂)₂—V; each A₁ is a —(CH₂)— group optionally substituted with one substituent, wherein each substituent is, independently, (CH₂)—V, (CH₂)₃—V, —(CH₂)₄—V, and —(CH₂)₅—V; and each V, is independently, hydroxyl, amino, ureido, guanidino, carbamoyl, or indolyl.

In some embodiments, each A₂ is phenyl optionally substituted with one or more substituents, wherein each substituent is, independently, O—(CH₃), halo, or O—(CH₂)₂—V; each A₁ is a —(CH₂)— group optionally substituted with one substituent, wherein each substituent is, independently, (CH₂)—V, (CH₂)₃—V, —(CH₂)₄—V, and —(CH₂)₅—V; and each V, is independently, amino, ureido, guanidino, carbamoyl, or indolyl.

In some embodiments, each A₂ is phenyl optionally substituted with one or more substituents, wherein each substituent is, independently, O—(CH₃), halo, or O—(CH₂)₂—V; each A₁ is a —(CH₂)— group optionally substituted with one substituent, wherein each substituent is, independently, CH₃, —(CH₂)—V, —(CH₂)₂—V, —(CH₂)₃—V, —(CH₂)₄—V, or —(CH₂)₅—V; each V is, independently, hydroxyl, amino, heteroarylamino, ureido, guanidino, carbamoyl, C(═O)OH, —C(═O)OR^{c}, —C(═O)NH—OH, —O—NH—C(═NH)NH₂, —NH—S(═O)₂OH, S(═O)₂OH, aziridinyl, azetidinyl, pyrrolidinyl, piperidinyl, piperazinyl, morpholino, azepanyl, azocanyl, tetrazolyl, 1,2,4-oxadiazolyl, 1,3,4-oxadiazolyl, imidazolyl, pyridinyl, indolyl, or a substituted phenyl, wherein the substituted phenyl is substituted with one or more substituents, wherein each substituent is, independently, OH or amino; and at least one of A₁ is a —(CH₂)— group substituted with one substituent, wherein each substituent is, independently, (CH₁)—V¹, (CH₂)₂—V′, —(CH₂)₃—V′, —(CH₂)₄—V¹, or —(CH₂)₅—V′, wherein V¹ is indolyl.

In some embodiments, R¹ is hydrogen, —C(═NR³)—NR³″R⁴′, —C(═O)—(CH₂)_{pNPL}—R⁴′, —C(═O)—(CH₂)_{pPL}—V, —C(═O)-A₂-NH—C(═O)—(CH₂)_{pPL}—V; or —C(═O)-A₂-NH—C(═O)—(CH₂)_{pNPL}—R⁴′; and R² is NH₂, —NH—(CH₂)_{pPL}—V, or —NH-A₁-C(═O)—NH₂.

In some embodiments, R¹ is hydrogen, —C(═NR³)—NR³″R⁴, —C(═O)—(CH₂)_{pNPL}—R⁴, —C(═O)—(CH₂)_{pPL}—V, —C(═O)-A₂-NH—C(═O)—(CH₂)_{pPL}—V, or —C(═O)-A₂-NH—C(═O)—(CH₂)_{pNPL}—R⁴′, wherein each V is, independently, hydroxy, amino, alkylamino, dialkylamino, —NH(CH₂)ₚNH₂ wherein p is 1 to 5, —N(CH₂CH₂NH₂)₂, guanidino, amidino, ureido, carbamoyl, heterocycloalkyl, or heteroaryl, and where R³, R³″, and R⁴′ are each, independently, H or alkyl; and R² is NH₂, —NH(CH₂)ₚNH₂ wherein p is 1 to 5, —NH—(CH₂)_{pPL}—V, or NH-A₁-C(═O)—NH₂, wherein V is hydroxy, amino, alkylamino, dialkylamino, —NH(CH₂)ₚNH₂ wherein p is 1 to 5, —N(CH₂CH₂NH₂)₂, guanidino, amidino, ureido, carbamoyl, heterocycloalkyl, or heteroaryl.

In some embodiments, R¹ is hydrogen, —C(═NH)—NH₂, —C(═O)—R⁴′, —C(═O)—(CH₂)_{pPL}—V, —C(═O)-A₂-NH—C(═O)—(CH₂)_{pPL}—V, or —C(═O)-A₂-NH—C(═O)—R⁴′, wherein each V is, independently, amino, alkylamino, dialkylamino, —NH(CH₂)ₚNH₂ wherein p is 1 to 5, —N(CH₂CH₂NH₂)₂, guanidino, amidino, ureido, carbamoyl, heterocycloalkyl, or heteroaryl, and where R⁴′ is alkyl; and R² is NH₂, —NH(CH₂)ₚNH₂ wherein p is 1 to 5, —NH—(CH₂)_{pPL}—V, or NH-A₁-C(═O)—NH₂, wherein V is amino, alkylamino, dialkylamino, —NH(CH₂)ₚNH₂ wherein p is 1 to 5, —N(CH₂CH₂NH₂)₂, guanidino, amidino, ureido, or carbamoyl.

In some embodiments, m is 3 or 4. In some embodiments, m is 4.

In some embodiments, at least one of A₂ group is different from other A₂ groups. In some embodiments, all A₂ groups are the same.

In some embodiments, at least one of A₁ group is different from other A, groups. In some embodiments, all A₁ groups are the same.

In some embodiments, the compound is a compound of Formula XVIIIa:

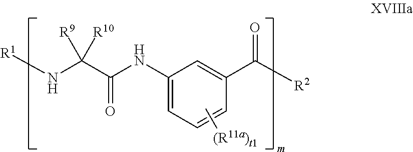

or pharmaceutically acceptable salt thereof, wherein:

each R⁹ is, independently, H, a PL group, or an NPL group;

each R¹⁰ is, independently, H, a PL group, or an NPL group;

each R^{11a} is, independently, a PL group or an NPL group; and

each R^{11a} is, independently, 0, 1, or 2.

In some embodiments, each R⁹ is, independently, a PL group or an NPL group. In some embodiments, each R⁹ is, independently, alkyl or (CH₂)_{pPL}—V wherein pPL is an integer from 1 to 5. In some embodiments, each R⁹ is, independently, (CH₂)_{pPL}—V wherein pPL is an integer from 1 to 5.

In some embodiments, each R¹⁰ is H.

In some embodiments, each R^{11a} is, independently, halo, alkyl, alkoxy, haloalkyl, haloalkoxy, —(CH₂)_{pPL}—V, —O(CH₂)_{pPL}—V, or —S(CH₂)_{pPL}—V, wherein pPL is an integer from 1 to 5. In some embodiments, each R^{11a} is, independently, halo, alkyl, alkoxy, haloalkyl, or haloalkoxy. In some embodiments, each R^{11a} is, independently, alkoxy. In some embodiments, each R¹ is methoxy.

In some embodiments, the compound is a compound of Formula XVIIIa-1, XVIIIa-2, or XVIIIa-3:

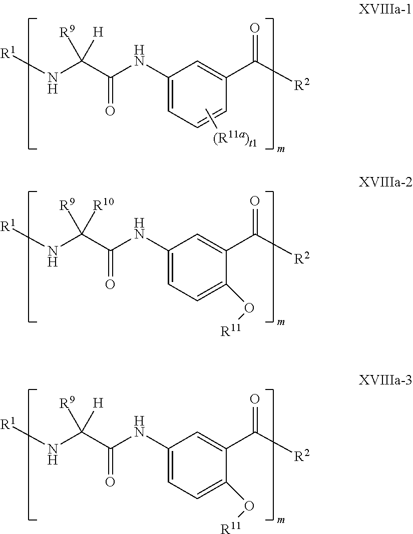

or pharmaceutically acceptable salt thereof, wherein each R¹¹ is, independently, H, alkyl, haloalkyl, or —(CH₂)_{pPL}—V, wherein pPL is an integer from 1 to 5.

In some embodiments, in Formula XVIIIa-2 or XVIIIa-3, or pharmaceutically acceptable salt thereof, each R¹¹ is, independently, alkyl.

In some embodiments, each R¹¹ is methyl.

The compounds of Formula XVIII, XVIIIa, XVIIIa-1, XVIIIa-2, or XVIIIa-3 (such as the polymers and oligomers), or salts thereof, useful in the present invention can be made, for example, by methods described in U.S. Patent Application Publication No. 2006-0041023, U.S. Pat. No. 7,173,102, and International Application No. WO 2005/123660. In some embodiments, the compounds of Formula XVIII, XVIIIa, XVIIIa-1, XVIIIa-2, or XVIIIa-3 (such as the polymers and oligomers), or salts thereof, useful in the present invention can be selected from those described in U.S. Patent Application Publication No. 2006-0041023, U.S. Pat. No. 7,173,102, and International Application No. WO 2005/123660. In some embodiments, the compound of Formula XVIII, XVIIIa, XVIIIa-1, XVIIIa-2, or XVIIIa-3 (such as the polymers and oligomers), or salts thereof, useful in the present invention is a compound or salt thereof selected from those described in U.S. Patent Application Publication No. 2006-0041023, U.S. Pat. No. 7,173,102, and International Application No. WO 2005/123660.

In some embodiments, the immune response is against an oral pathogen. In some embodiments, the oral pathogen is chosen from Aggregatibacter spp. such as, for example, Aggregatibacter actinomycetemcomitans; Porphyromonas spp. such as, for example, Porphyromonas gingivalis; Streptococcus spp. such as, for example, Streptococcus sanguis and Streptococcus mutans, Candida spp. such as, for example, Candida albicans, Candida glabrata, Candida krusei, Candida dubliniensis, Candida parapsilosis, and Candida tropicalis; Actinomyces spp. such as, for example, Actinomyces viscosus; and Lactobacillus spp. such as, for example, Lactobacillus casei. In some embodiments, the immune response is against a bacterial pathogen. In some embodiments, the bacterial pathogen is chosen from Staphylococcus spp., such as, for example, Staphylococcus aureus, methicillin-resistant Staphylococcus aureus, and Staphylococcus epidermidis; Streptococcus spp. such as, for example, Streptococcus pneumoniae, Streptococcus pyogenes, and Streptococcus viridans; Escherichia spp. such as, for example, E. coli; Enterococcus spp. such as, for example, Enterococcus faecalis and Enterococcus faecium; Psuedomonas spp. such as, for example, Pseudomonas aeruginosa; Acinetobacter spp. such as, for example, A. baumannii;

Haemophilus spp. such as, for example, Haemophilus influenzae; Serratia spp. such as, for example, Serratia marcescens; Moraxella spp. such as, for example, Moraxella catarrhalis; Klebsiella spp. such as, for example, Klebsiella pneumoniae; Proteus spp. such as, for example, Proteus vulgaris and Proteus mirabilis; Bacteroides spp. such as, for example, Bacteroides fragalis; Clostridium spp. such as, for example, Clostridium difficile and Clostridium perfringens; and Propionibacterium spp. such as, for example, Propionibacterium acnes.

The present invention also provides methods of modulating an immune response in a mammal comprising administering to the mammal in need thereof a therapeutically effective amount of a compound of Formula XIX:

R¹—[—X-A₁-X—Y-A₂-Y—]ₘ—R² XIX

or a pharmaceutically acceptable salt thereof,
wherein:

each X is, independently, NR⁸, O, S, —N(R⁸)N(R⁸)—, —N(R⁸)—(N═N)—, —(N═N)—N(R⁸)—, —C(R⁷R⁷′)NR⁸—, —C(R⁷R⁷)O—, or —C(R⁷R⁷)S—;

each Y is, independently, C═O, C═S, O═S═O, —C(═O)C(═O)—, C(R⁶R⁶′)C═O, or C(R⁶R⁶′)C═S;

each R⁸ is, independently, hydrogen or alkyl;

each R⁷ and each R⁷′ are, independently, hydrogen or alkyl; or R⁷ and R⁷′ together form —(CH₂)ₚ—, wherein p is 4 to 8;

each R⁶ and each R⁶′ are, independently, hydrogen or alkyl; or R⁶ and R⁶′ together form —(CH₂)₂NR¹²(CH₂)₂—, wherein R¹² is hydrogen, —C(═N)CH₃, or —C(═NH)—NH₂;

A₁ and A₂ are each, independently, optionally substituted arylene or optionally substituted heteroarylene, wherein A₁ and A₂ are each, independently, optionally substituted with one or more PL group(s), one or more NPL group(s), or a combination of one or more PL group(s) and one or more NPL group(s);

or each A₂ is, independently, optionally substituted arylene or optionally substituted heteroarylene, and each A₁ is, independently, optionally substituted C₃ to C₈ cycloalkyl, wherein A₁ and A₂ are each, independently, optionally substituted with one or more PL group(s), one or more NPL group(s), or a combination of one or more PL group(s) and one or more NPL group(s);

R¹ is hydrogen, a PL group, or an NPL group, and R² is —X-A₁-X—R¹, wherein A₁ is as defined above and is optionally substituted with one or more PL group(s), one or more NPL group(s), or a combination of one or more PL group(s) and one or more NPL group(s); or

R¹ is hydrogen, a PL group, or an NPL group, and R² is —X-A′-X—R¹, wherein A′ is C₃ to C₈ cycloalkyl, aryl, or heteroaryl and is optionally substituted with one or more PL group(s), one or more NPL group(s), or a combination of one or more PL group(s) and one or more NPL group(s); or

R¹ is —Y-A₂-Y—R², and each R² is, independently, hydrogen, a PL group, or an NPL group; or

R¹ is —Y-A¹ and R² is —X-A′, wherein each A′ is, independently, C₃ to C₈ cycloalkyl, aryl, or heteroaryl and is optionally substituted with one or more PL group(s), one or more NPL group(s), or a combination of one or more PL group(s) and one or more NPL group(s); or

R¹ and R² are, independently, a PL group or an NPL group; or

R¹ and R² together form a single bond;

each NPL is, independently, —B(OR⁴)₂ or —(NR^{3′})_{q1NPL}—U^{NPL}-LK^{NPL}(NR³″)_{q2NPL}—R⁴′, wherein:

R³, R³′, and R³″ are each, independently, hydrogen, alkyl, or alkoxy;

R⁴ and R⁴′ are each, independently, hydrogen, alkyl, alkenyl, alkynyl, cycloalkyl, aryl, and heteroaryl, wherein each of the alkyl, alkenyl, alkynyl, cycloalkyl, aryl, and heteroaryl is optionally substituted with one or more alkyl or halo groups;

each U^{NPL} is, independently, absent or O, S, S(═O), S(═O)₂, NR³, —C(═O)—, —C(═O)—NR³—, —C(═O)—N═N—NR³—, —C(═O)—NR³—N═N—, —N═N—NR³—, —C(═N—N(R³)₂)—, —C(═NR³)—, —C(═O)O—, —C(═O)S—, —C(═S)—, —O—P(═O)₂O—, —S—C═N—, or —C(═O)—NR³—O—, wherein groups with two chemically nonequivalent termini can adopt both possible orientations;

each LK^{NPL} is, independently, —(CH₂)_{pNPL}— or C₂₋₈ alkenylenyl, wherein each of the —(CH₂)_{pNPL}— and C₂₋₈ alkenylenyl is optionally substituted with one or more substituents, wherein each substituent is, independently, amino, hydroxyl, aminoalkyl, hydroxylalkyl, or alkyl;

each pNPL is, independently, an integer from 0 to 8;

q1NPL and q2NPL are each, independently, 0, 1, or 2;

each PL is, independently, halo, hydroxyethoxymethyl, methoxyethoxymethyl, polyoxyethylene, or —(NR⁵)_{q1PL}—U^{PL}-LK^{PL}—(NR⁵)_{q2PL}—V, wherein:

R⁵, R⁵′, and R⁵″ are each, independently, hydrogen, alkyl, and alkoxy;

each U^{PL} is, independently, absent or O, S, S(═O), S(═O)₂, NR⁵, —C(═O)—, —C(═O)—NR⁵—, —C(═O)—N═N—NR⁵—, —C(═O)—NR⁵—N═N—, —N═N—NR⁵—, —C(═N—N(R⁵)₂)—, —C(═NR⁵)—, —C(═O)O—, —C(═O)S—, —C(═S)—, —O—P(═O)₂O—, —S—C═N—, or —C(═O)—NR⁵—O—, wherein groups with two chemically nonequivalent termini can adopt either of the two possible orientations;

each V is, independently, nitro, cyano, amino, halo, hydroxy, alkoxy, alkylthio, alkylamino, dialkylamino, —NH(CH₂)ₚNH₂ wherein p is 1 to 5, —C(═O)NH(CH₂)ₚNH₂ wherein p is 1 to 5, —C(═O)NH(CH₂)ₚNHC(═NH)NH₂ wherein p is 1 to 5, —C(═O)NH(CH₂)ₚNHC(═O)NH₂ wherein p is 1 to 5, —NHC(═O)-alkyl, —N(CH₂CH₂NH₂)₂, diazamino, amidino, guanidino, ureido, carbamoyl, —C(═O)OH, —C(═O)OR^{c}, —C(═O)NH—OH, —O—NH—C(═NH)NH₂, —NH—S(═O)₂OH, S(═O)₂OH, NR^{d}R^{e}, semicarbazone, aryl, cycloalkyl, heterocycloalkyl, or heteroaryl, wherein each of the aryl and cycloalkyl is substituted with one or more substitutents, wherein each of the heterocycloalkyl, and heteroaryl is optionally substituted with one or more substituents, and wherein each of the substituents for the aryl, cycloalkyl, heterocycloalkyl, and heteroaryl is, independently, nitro, cyano, amino, halo, hydroxy, alkoxy, alkylthio, alkylamino, dialkylamino, —NH(CH₂)ₚNH₂ wherein p is 1 to 5, —N(CH₂CH₂NH₂)₂, diazamino, amidino, guanidino, ureido, carbamoyl, —C(═O)OH, —C(═O)OR^{c}, —C(═O)NH—OH, —O—NH—C(═NH)NH₂, —NH—S(═O)₂OH, S(═O)₂OH, NR^{d}R^{e}, semicarbazone, aminosulfonyl, aminoalkoxy, aminoalkylthio, lower acylamino, or benzyloxycarbonyl;

each LK^{PL} is, independently, —(CH₂)_{pPL}— or C₂₋₈ alkenylenyl, wherein each of the —(CH₂)_{pNPL}— and C₂₋₈ alkenylenyl is optionally substituted with one or more substituents, wherein each substituent is, independently, amino, hydroxyl, aminoalkyl, hydroxylalkyl, or alkyl;

each pPL is, independently, an integer from 0 to 8;

q1 PL and q2PL are each, independently, 0, 1, or 2; and

m is an integer from 1 to about 20.

In some embodiments, each of the moiety of —Y-A₂-Y— is, independently, a moiety of Formula XIX-1, XIX-2, or XIX-3.

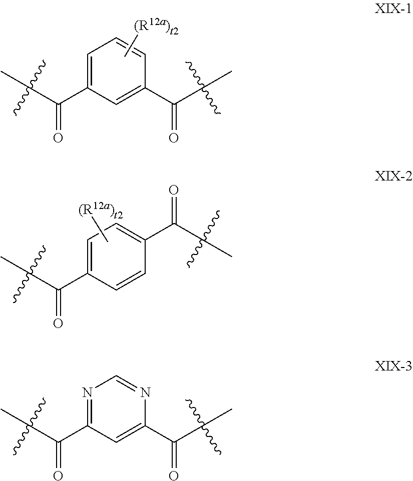

wherein each R^{12a} is, independently, a PL group or an NPL group; and t2 is 0, 1, or 2.

In some embodiments, each of the moiety of is, independently, a moiety of Formula XIX-1 or XIX-2; and each R^{12a} is, independently, halo, alkyl, alkoxy, haloalkyl, haloalkoxy, —(CH₂)_{pPL}—V, —O(CH₂)_{pPL}—V, or —S(CH₂)_{pPL}—V, wherein pPL is an integer from 1 to 5.

In some embodiments, each R^{12a} is, independently, halo, alkyl, alkoxy, haloalkyl, or haloalkoxy. In some embodiments, each R^{12a} is, independently, alkoxy. In some embodiments, each R^{12a} is methoxy.

In some embodiments, each of the moiety of —Y-A₂-Y— is, independently, a moiety of Formula XIX-1 or XIX-2; and t2 is 2.

In some embodiments, each R^{12a} is, independently, alkoxy. In some embodiments, each R^{12a} is methoxy.

In some embodiments, each of the moiety of —Y-A₂-Y— is, independently, a moiety of Formula XIX-1, and the moiety of Formula XIX-1 is a moiety of Formula XIX-1a:

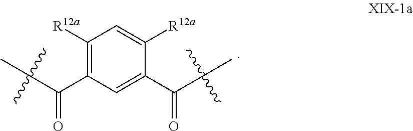

In some embodiments, each of the moiety of —X-A₁-X— is, independently, a moiety of Formula XIX-B:

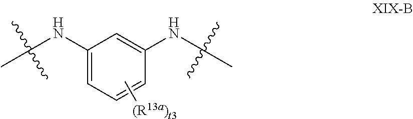

wherein each R^{13a} is, independently, a PL group or an NPL group; and t3 is 0, 1, or 2.

In some embodiments, each of the moiety of —X-A₁-X— is, independently, a moiety of Formula XIX-C:

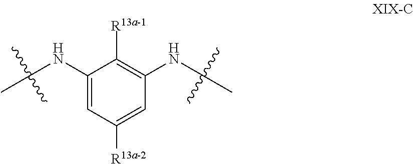

wherein each of R^{13a-1} and R^{13a-2} is, independently, H, a PL group, or an NPL group.

In some embodiments, each of R^{13a-1} and R^{13a-2} is, independently, a PL group or an NPL group. In some embodiments, each of R^{13a-1} and R^{13a-2} is, independently, halo, alkyl, haloalkyl, —O(CH₂)_{pPL}—V, or —S(CH₂)_{pPL}—V, wherein pPL is an integer from 1 to 5. In some embodiments, each of R^{13a-1} and R^{13a-2} is, independently, haloalkyl or —S(CH₂)_{pPL}—V, wherein pPL is an integer from 1 to 5.

In some embodiments, each of the moiety of —X-A₁-X— is, independently, a moiety of Formula XIX-D:

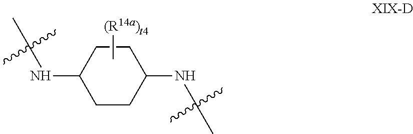

wherein each R^{14a} is, independently, a PL group or an NPL group; and t4 is 0, 1, or 2.

In some embodiments, t4 is 0.

In some embodiments, each moiety of —Y-A₂-Y— is, independently, a moiety of Formula XIX-1, XIX-1a, XIX-2, or XIX-3; and each of the moiety of —X-A₁-X— is, independently, a moiety of Formula XIX-B, XIX-C, or XIX-D. In some embodiments, each moiety of —Y-A₂-Y— is, independently, a moiety of Formula XIX-1 or XIX-1a; and each of the moiety of —X-A₁-X— is, independently, a moiety of Formula XIX-B or XIX-C. In some embodiments, each moiety of —Y-A₂-Y— is, independently, a moiety of Formula XIX-1a; and each of the moiety of —X-A₁-X— is, independently, a moiety of Formula XIX-C. In some embodiments, each moiety of —Y-A₂-Y— is, independently, a moiety of Formula XIX-1, XIX-1a, XIX-2, or XIX-3; and each of the moiety of —X-A₁-X— is, independently, a moiety of Formula XIX-D. In some embodiments, each moiety of —Y-A₂-Y— is, independently, a moiety of Formula XIX-1a.

In some embodiments, the compound is of Formula XIXa:

R¹—X-A₁-X—Y-A₂-Y—X-A₁-X—R² XIXa

or pharmaceutically acceptable salt thereof, wherein:

each X is, independently, NR⁸, O, S, or —N(R⁸)N(R⁸)—;

each Y is, independently, C═O, C═S, or O═S═O;

each R⁸ is, independently, hydrogen or alkyl;

A₁ and A₂ are each, independently, optionally substituted arylene or optionally substituted heteroarylene, wherein A₁ and A₂ are each, independently, optionally substituted with one or more PL group(s), one or more NPL group(s), or a combination of one or more PL group(s) and one or more NPL group(s);

R¹ is a PL group or an NPL group;

R² is R¹;

each NPL is, independently, —(NR³)_{q1NPL}—U^{NPL}-LK^{NPL}—(NR³″)_{q2NPL}—R⁴′, wherein:

R³, R³′, and R³″ are each, independently, hydrogen, alkyl, or alkoxy;

R⁴ and R⁴′ are each, independently, hydrogen, alkyl, alkenyl, alkynyl, cycloalkyl, aryl, or heteroaryl, wherein each of the alkyl, alkenyl, alkynyl, cycloalkyl, aryl, and heteroaryl is optionally substituted with one or more alkyl or halo groups;

U^{NPL} is, independently, absent or O, S, S(═O), S(═O)₂, NR³, —C(═O)—, —C(═O)—N═N—NR³—, —C(═O)—NR³—N═N—, —N═N—NR³—, —C(═N—N(R³)₂)—, —C(═NR³)—, —C(═O)O—, —C(═O)S—, —C(═S)—, —O—P(═O)₂O—, —S—C═N—, or —C(═O)—NR³—O—, wherein groups with two chemically nonequivalent termini can adopt either of the two possible orientations;

each LK^{NPL} is, independently, —(CH₂)_{pNPL}— or C₂₋₈ alkenylenyl, wherein the —(CH₂)_{pNPL}— is optionally substituted with one or more substituents, wherein each substituent is, independently, amino, hydroxyl, or alkyl;

each pNPL is, independently, an integer from 0 to 8;

q1NPL and q2NPL are each, independently, 0, 1, or 2;

each PL is, independently, halo, hydroxyethoxymethyl, methoxyethoxymethyl, polyoxyethylene, or —(NR⁵)_{pPL}—U^{PL}-LK^{PL}—(NR⁵)_{q2PL}—V, wherein:

R⁵, R⁵′, and R⁵″ are each, independently, hydrogen, alkyl, or alkoxy;

each U^{PL} is, independently, absent or O, S, S(═O), S(═O)₂, NR⁵, —C(═O)—, —C(═O)—N═N—NR⁵—, —C(═O)—NR⁵—N═N—, —N═N—NR⁵—, —C(═N—N(R⁵)₂)—, —C(═NR⁵)—, —C(═O)O—, —C(═O)S—, —C(═S)—, —O—P(═O)₂O—, —R⁵O—, —R⁵S—, —S—C═N—, or —C(═O)—NR⁵—O—, wherein groups with two chemically nonequivalent termini can adopt both possible orientations;

each V is, independently, nitro, cyano, amino, halo, hydroxy, alkoxy, alkylthio, alkylamino, dialkylamino, —NH(CH₂)ₚNH₂ wherein p is 1 to 5, —N(CH₂CH₂NH₂)₂, diazamino, amidino, guanidino, ureido, carbamoyl, —C(═O)OH, —C(═O)OR^{c}, —C(═O)NH—OH, —O—NH—C(═NH)NH₂, —NH—S(═O)₂OH, S(═O)₂OH, NR^{d}R^{e}, semicarbazone, aryl, heterocycloalkyl, or heteroaryl, wherein the aryl is substituted with one or more substitutents, wherein each of the heterocycloalkyl and heteroaryl is optionally substituted with one or more substituents, and wherein each of each of the substituents for the aryl, heterocycloalkyl, and heteroaryl is, independently, nitro, cyano, amino, halo, hydroxy, alkoxy, alkylthio, alkylamino, dialkylamino, —NH(CH₂)ₚNH₂ wherein p is 1 to 5, —N(CH₂CH₂NH₂)₂, diazamino, amidino, guanidino, ureido, carbamoyl, —C(═O)OH, —C(═O)OR^{c}, —C(═O)NH—OH, —O—NH—C(═NH)NH₂, —NH—S(═O)₂OH, S(═O)₂OH, NR^{d}R^{e}, semicarbazone, aminosulfonyl, aminoalkoxy, aminoalkylthio, lower acylamino, or benzyloxycarbonyl;

each LK^{PL} is, independently, —(CH₂)_{pPL}— or C₂₋₈ alkenylenyl, wherein the —(CH₂)_{pPL}— is optionally substituted with one or more substituents, wherein each substituent is, independently, amino, hydroxyl, or alkyl;

each pPL is, independently, an integer from 0 to 8; and

q1 PL and q2PL are each, independently, 0, 1, or 2.

In some embodiments, each NPL group is, independently, —B(OR⁴)₂, R⁴′, or OR⁴′, and R⁴ and R⁴′ are each, independently, alkyl, alkenyl, alkynyl, cycloalkyl, or aryl, each is optionally substituted with one or more substitutents, wherein each substituent is, independently, alkyl, halo, or haloalkyl.

In some embodiments, each NPL group is, independently, R⁴′ or OR⁴′, and each R⁴′ is, independently, alkyl, alkenyl, alkynyl, cycloalkyl, or aryl, each is optionally substituted with one or more substitutents, wherein each substituent is, independently, alkyl, halo, or haloalkyl.

In some embodiments, each NPL group is, independently, alkyl, alkenyl, alkynyl, cycloalkyl, aryl, or alkoxy, each is optionally substituted with one or more substitutents, wherein each substituent is, independently, alkyl, halo, or haloalkyl. In some embodiments, each NPL group is, independently, alkyl, haloalkyl, alkoxy, or haloalkoxy.

In some embodiments, each V is, independently, nitro, cyano, amino, hydroxy, alkoxy, alkylthio, alkylamino, dialkylamino, —NH(CH₂)ₚNH₂ wherein p is 1 to 5, —N(CH₂CH₂NH₂)₂, diazamino, amidino, guanidino, ureido, carbamoyl, —C(═O)OH, —C(═O)OR^{c}, —C(═O)NH—OH, —O—NH—C(═NH)NH₂, —NH—S(═O)₂OH, S(═O)₂OH, NR^{d}R^{e}, semicarbazone, aryl, heterocycloalkyl, or heteroaryl, wherein the aryl is substituted with one or more substitutents, wherein each of the heterocycloalkyl and heteroaryl is optionally substituted with one or more substituents, and wherein each of each of the substituents for the aryl, heterocycloalkyl, and heteroaryl is, independently, nitro, cyano, amino, hydroxy, alkoxy, alkylthio, alkylamino, dialkylamino, —NH(CH₂)ₚNH₂ wherein p is 1 to 5, —N(CH₂CH₂NH₂)₂, diazamino, amidino, guanidino, ureido, carbamoyl, —C(═O)OH, —C(═O)OR^{c}, —C(═O)NH—OH, —O—NH—C(═NH)NH₂, —NH—S(═O)₂OH, S(═O)₂OH, NR^{d}R^{e}, semicarbazone, aminosulfonyl, aminoalkoxy, aminoalkylthio, lower acylamino, or benzyloxycarbonyl.

In some embodiments, each V is, independently, hydroxy, amino, alkylamino, dialkylamino, —NH(CH₂)ₚNH₂ wherein p is 1 to 5, —N(CH₂CH₂NH₂)₂, guanidino, amidino, ureido, carbamoyl, —C(═O)OH, —C(═O)OR^{c}, —C(═O)NH—OH, —O—NH—C(═NH)NH₂, —NH—S(═O)₂OH, S(═O)₂OH, NR^{d}R^{e}, a substituted aryl group, heterocycloalkyl, or heteroaryl, wherein each of the heterocycloalkyl and heteroaryl is optionally substituted with one more substituents, wherein each substituent is, independently, alkyl, haloalkyl, alkoxy, haloalkoxy, amino, cyano, nitro, hydroxy, —NH(CH₂)ₚNH₂ wherein p is 1 to 5, —N(CH₂CH₂NH₂)₂, amidino, guanidino, aminosulfonyl, aminoalkoxy, aminoalkylthio, lower acylamino, or benzyloxycarbonyl; and wherein the substituted aryl group is substituted with one more substituents, wherein each substituent is, independently, amino, cyano, nitro, hydroxy, —NH(CH₂)ₚNH₂ wherein p is 1 to 5, —N(CH₂CH₂NH₂)₂, amidino, guanidino, aminosulfonyl, aminoalkoxy, aminoalkylthio, lower acylamino, or benzyloxycarbonyl.

In some embodiments, each V is, independently, hydroxy, amino, alkylamino, dialkylamino, —NH(CH₂)ₚNH₂ wherein p is 1 to 5, —N(CH₂CH₂NH₂)₂, guanidino, amidino, ureido, carbamoyl, —C(═O)OH, —C(═O)OR^{c}, —C(═O)NH—OH, —O—NH—C(═NH)NH₂, —NH—S(═O)₂OH, S(═O)₂OH, NR^{d}R^{e}, a substituted aryl group, heterocycloalkyl, or heteroaryl, wherein each of the heterocycloalkyl and heteroaryl is optionally substituted with one more substituents, wherein each substituent is, independently, amino, cyano, nitro, hydroxy, —NH(CH₂)ₚNH₂ wherein p is 1 to 5, —N(CH₂CH₂NH₂)₂, amidino, guanidino, aminosulfonyl, aminoalkoxy, aminoalkylthio, lower acylamino, or benzyloxycarbonyl; and wherein the substituted aryl group is substituted with one more substituents, wherein each substituent is, independently, amino, cyano, nitro, hydroxy, —NH(CH₂)ₚNH₂ wherein p is 1 to 5, —N(CH₂CH₂NH₂)₂, amidino, guanidino, aminosulfonyl, aminoalkoxy, aminoalkylthio, lower acylamino, or benzyloxycarbonyl.

In some embodiments, each V is, independently, hydroxy, amino, alkylamino, dialkylamino, —NH(CH₂)ₚNH₂ wherein p is 1 to 5, —N(CH₂CH₂NH₂)₂, guanidino, amidino, ureido, carbamoyl, —C(═O)NH—OH, —O—NH—C(═NH)NH₂, —NH—S(═O)₂OH, NR^{d}R^{e}, heterocycloalkyl, or heteroaryl, wherein each of the heterocycloalkyl and heteroaryl is optionally substituted with one more substituents, wherein each substituent is, independently, alkyl, haloalkyl, alkoxy, haloalkoxy, amino, cyano, nitro, hydroxy, —NH(CH₂)ₚNH₂ wherein p is 1 to 5, —N(CH₂CH₂NH₂)₂, amidino, guanidino, aminosulfonyl, aminoalkoxy, aminoalkylthio, lower acylamino, or benzyloxycarbonyl.

In some embodiments, each V is, independently, hydroxy, amino, alkylamino, dialkylamino, —NH(CH₂)ₚNH₂ wherein p is 1 to 5, —N(CH₂CH₂NH₂)₂, guanidino, amidino, ureido, carbamoyl, —C(═O)NH—OH, —O—NH—C(═NH)NH₂, —NH—S(═O)₂OH, NR^{d}R^{e}, heterocycloalkyl, or heteroaryl, wherein each of the heterocycloalkyl and heteroaryl is optionally substituted with one more substituents, wherein each substituent is, independently, amino, cyano, nitro, hydroxy, —NH(CH₂)ₚNH₂ wherein p is 1 to 5, —N(CH₂CH₂NH₂)₂, amidino, guanidino, aminosulfonyl, aminoalkoxy, aminoalkylthio, lower acylamino, or benzyloxycarbonyl.

In some embodiments, each V is, independently, hydroxy, amino, alkylamino, arylamino, heteroarylamino, ureido, carbamoyl, —C(═O)OH, —C(═O)OR^{c}, —C(═O)NH—OH, —O—NH—C(═NH)NH₂, —NH—S(═O)₂OH, S(═O)₂OH, a 3-8 membered heterocycloalkyl, a 5- to 10-membered heteroaryl, or a 6- to 10-membered substituted aryl, wherein the substituted aryl is substituted with one or more substituents, wherein each substituent is, independently, OH, amino, hydroxylalkyl, or aminoalkyl, and wherein each of the 3-8 membered heterocycloalkyl and the 5- to 10-membered heteroaryl is optionally substituted with one or more substituents, wherein each substituent is, independently, alkyl, haloalkyl, alkoxy, haloalkoxy, amino, cyano, nitro, hydroxy, —NH(CH₂)ₚNH₂ wherein p is 1 to 5, —N(CH₂CH₂NH₂)₂, amidino, guanidino, aminosulfonyl, aminoalkoxy, aminoalkylthio, lower acylamino, or benzyloxycarbonyl.

In some embodiments, each V is, independently, hydroxy, amino, alkylamino, arylamino, heteroarylamino, ureido, carbamoyl, —C(═O)OH, —C(═O)OR^{c}, —C(═O)NH—OH, —O—NH—C(═NH)NH₂, —NH—S(═O)₂OH, S(═O)₂OH, a 3-8 membered heterocycloalkyl, a 5- to 10-membered heteroaryl, or a 6- to 10-membered substituted aryl, wherein the substituted aryl is substituted with one or more substituents, wherein each substituent is, independently, OH, amino, hydroxylalkyl, or aminoalkyl.

In some embodiments, each V is, independently, amino, heteroarylamino, ureido, carbamoyl, C(═O)OH, —C(═O)OR^{c}, —C(═O)NH—OH, —O—NH—C(═NH)NH₂, —NH—S(═O)₂OH, S(═O)₂OH, aziridinyl, azetidinyl, pyrrolidinyl, piperidinyl, piperazinyl, morpholino, azepanyl, azocanyl, tetrazolyl, 1,2,4-oxadiazolyl, 1,3,4-oxadiazolyl, imidazolyl, pyridinyl, indolyl, or a substituted phenyl, wherein the substituted phenyl is substituted with one or more substituents, wherein each substituent is, independently, OH or amino.

In some embodiments, each V is, independently, amino, alkylamino, dialkylamino, —NH(CH₂)ₚNH₂ wherein p is 1 to 5, —N(CH₂CH₂NH₂)₂, guanidino, amidino, ureido, heterocycloalkyl, or heteroaryl, wherein each of the heterocycloalkyl and heteroaryl is optionally substituted with one more substituents, wherein each substituent is, independently, amino, cyano, nitro, hydroxy, —NH(CH₂)ₚNH₂ wherein p is 1 to 5, —N(CH₂CH₂NH₂)₂, amidino, guanidino, aminosulfonyl, aminoalkoxy, aminoalkylthio, lower acylamino, or benzyloxycarbonyl.

In some embodiments, each V is, independently, amino, alkylamino, dialkylamino, —NH(CH₂)ₚNH₂ wherein p is 1 to 5, —N(CH₂CH₂NH₂)₂, guanidino, amidino, ureido, pyrrodinyl, piperidinyl, piperazinyl, 4-methylpiperazinyl, pyridinyl, pyrimidinyl, pyrazinyl, or indolyl. In some embodiments, each V is, independently, amino, alkylamino, dialkylamino, —NH(CH₂)ₚNH₂ wherein p is 1 to 5, —N(CH₂CH₂NH₂)₂, guanidino, amidino, ureido, or indolyl.

In some embodiments, each PL is, independently, halo, hydroxyethoxymethyl, methoxyethoxymethyl, polyoxyethylene, and —(NR⁵′)_{q1PL}—U^{PL}—(CH₂)_{pPL}—(NR⁵)_{q2PL}—V.

In some embodiments, each PL group is, independently, halo, —(CH₂)_{pPL}—V, O—(CH₂)_{pPL}—V, or S—(CH₂)_{pPL}—V; each pPL is an integer from 0 to 5; and each V is, independently, hydroxy, amino, halo, alkylamino, dialkylamino, —NH(CH₂)ₚNH₂ wherein p is 1 to 5, —N(CH₂CH₂NH₂)₂, guanidino, amidino, ureido, carbamoyl, —C(═O)OH, —C(═O)OR^{c}, —C(═O)NH—OH, —O—NH—C(═NH)NH₂, —NH—S(═O)₂OH, S(═O)₂OH, NR^{d}R^{e}, a substituted aryl group, heterocycloalkyl, or heteroaryl, wherein each of the heterocycloalkyl and heteroaryl is optionally substituted with one more substituents, wherein each substituent is, independently, amino, halo, cyano, nitro, hydroxy, —NH(CH₂)ₚNH₂ wherein p is 1 to 5, —N(CH₂CH₂NH₂)₂, amidino, guanidino, aminosulfonyl, aminoalkoxy, aminoalkylthio, lower acylamino, or benzyloxycarbonyl; and wherein the substituted aryl group is substituted with one more substituents, wherein each substituent is, independently, amino, halo, cyano, nitro, hydroxy, —NH(CH₂)ₚNH₂ wherein p is 1 to 5, —N(CH₂CH₂NH₂)₂, amidino, guanidino, aminosulfonyl, aminoalkoxy, aminoalkylthio, lower acylamino, or benzyloxycarbonyl.

In some embodiments, each PL group is, independently, halo, —(CH₂)_{pPL}—V, O—(CH₂)_{pPL}—V, or S—(CH₂)_{pPL}—V; each pPL is an integer from 0 to 5; and each V is, independently, hydroxy, amino, alkylamino, dialkylamino, —NH(CH₂)ₚNH₂ wherein p is 1 to 5, —N(CH₂CH₂NH₂)₂, guanidino, amidino, ureido, carbamoyl, —C(═O)NH—OH, —O—NH—C(═NH)NH₂, —NH—S(═O)₂OH, NR^{d}R^{e}, heterocycloalkyl, or heteroaryl, wherein each of the heterocycloalkyl and heteroaryl is optionally substituted with one more substituents, wherein each substituent is, independently, amino, halo, cyano, nitro, hydroxy, —NH(CH₂)ₚNH₂ wherein p is 1 to 5, —N(CH₂CH₂NH₂)₂, amidino, guanidino, aminosulfonyl, aminoalkoxy, aminoalkylthio, lower acylamino, or benzyloxycarbonyl.

In some embodiments, each NPL group is, independently, —B(OR⁴)₂, R⁴′, or OR⁴′, R⁴ and R⁴′ are each, independently, alkyl, alkenyl, alkynyl, cycloalkyl, or aryl, each is optionally substituted with one or more substitutents, wherein each substituent is, independently, alkyl, halo, or haloalkyl; each PL group is, independently, halo, —(CH₂)_{pPL}—V, O—(CH₂)_{pPL}—V, or S—(CH₂)_{pPL}—V; each pPL is an integer from 0 to 5; and each V is, independently, hydroxy, amino, alkylamino, dialkylamino, —NH(CH₂)ₚNH₂ wherein p is 1 to 5, —N(CH₂CH₂NH₂)₂, guanidino, amidino, ureido, carbamoyl, —C(═O)OH, —C(═O)OR^{c}, —C(═O)NH—OH, —O—NH—C(═NH)NH₂, —NH—S(═O)₂OH, S(═O)₂OH, NR^{d}R^{e}, a substituted aryl group, heterocycloalkyl, or heteroaryl, wherein each of the heterocycloalkyl and heteroaryl is optionally substituted with one more substituents, wherein each substituent is, independently, amino, halo, cyano, nitro, hydroxy, —NH(CH₂)ₚNH₂ wherein p is 1 to 5, —N(CH₂CH₂NH₂)₂, amidino, guanidino, aminosulfonyl, aminoalkoxy, aminoalkylthio, lower acylamino, or benzyloxycarbonyl; and wherein the substituted aryl group is substituted with one more substituents, wherein each substituent is, independently, amino, halo, cyano, nitro, hydroxy, —NH(CH₂)ₚNH₂ wherein p is 1 to 5, —N(CH₂CH₂NH₂)₂, amidino, guanidino, aminosulfonyl, aminoalkoxy, aminoalkylthio, lower acylamino, or benzyloxycarbonyl.

In some embodiments, each X is, independently, NR⁸; each Y is C═O; A₁ and A₂ are each, independently, phenyl or a 6-membered heteroaryl, each optionally substituted with one or more substituents, wherein each substituent is, independently, alkyl, haloalkyl, halo, —O-alkyl, O—(CH₂)_{pPL}—V, or S—(CH₂)_{pPL}—V; R¹ is —C(═O)—(CH₂)_{pPL}—V or —C(═O)—(CH₂)_{pPL}—R⁴′; R² is R¹; R⁴′ is H or alkyl; and each V is, independently, hydroxy, amino, alkylamino, dialkylamino, —NH(CH₂)ₚNH₂ wherein p is 1 to 4, —N(CH₂CH₂NH₂)₂, guanidino, amidino, ureido, carbamoyl, heterocycloalkyl, or heteroaryl.

In some embodiments, each X is NH; each Y is C═O; each A₁ is, independently, phenyl optionally substituted with one or two substituents, wherein each substituent is, independently, haloalkyl, halo, —O-alkyl, O—(CH₂)_{pPL}—V, or S—(CH₂)_{pPL}—V; A₂ is phenyl or a 6-membered heteroaryl, each optionally substituted with one or two substituents, wherein each substituent is, independently, —O-alkyl; R¹ is —C(═O)—(CH₂)_{pPL}—V; R² is R¹; and each V is, independently, hydroxy, amino, alkylamino, dialkylamino, —NH(CH₂)ₚNH₂ wherein p is 1 to 5, —N(CH₂CH₂NH₂)₂, guanidino, amidino, ureido, carbamoyl, heterocycloalkyl, or heteroaryl.

In some embodiments, each X is NH; each Y is C═O each A₁ is, independently, phenyl optionally substituted with one or two substituents, wherein each substituent is, independently, haloalkyl, O—(CH₂)_{pPL}—V, or S—(CH₂)_{pPL}—V; A₂ is phenyl or pyrimidinyl, each optionally substituted with one or two substituents, wherein each substituent is, independently, —O-alkyl; R¹ is —C(═O)—(CH₂)_{pPL}—V; R² is R¹; and each V is, independently, amino, alkylamino, dialkylamino, —NH(CH₂)ₚNH₂ wherein p is 1 to 4, —N(CH₂CH₂NH₂)₂, guanidino, amidino, ureido, carbamoyl, or indolyl.

In some embodiments, the moiety of —Y-A₂-Y— is a moiety of Formula XIX-1, XIX-2, or XIX-3:

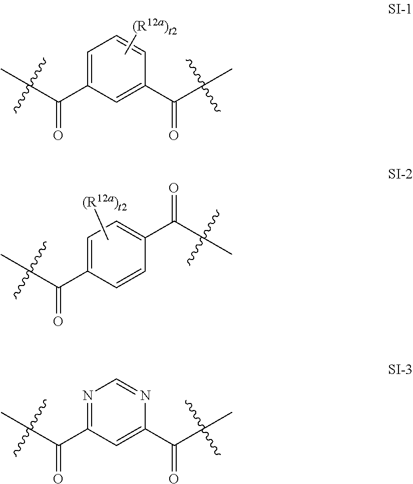

wherein each R^{12a} is, independently, a PL group or an NPL group; and t2 is 0, 1, or 2.

In some embodiments, the moiety of —Y-A₂-Y— is a moiety of Formula XIX-1 or XIX-2; and each R^{12a} is, independently, halo, alkyl, alkoxy, haloalkyl, haloalkoxy, —(CH₂)_{pPL}—V, —O(CH₂)_{pPL}—V, or —S(CH₂)_{pPL}—V, wherein pPL is an integer from 1 to 5.

In some embodiments, each R^{12a} is, independently, halo, alkyl, alkoxy, haloalkyl, or haloalkoxy. In some embodiments, each R^{12a} is, independently, alkoxy. In some embodiments, each R^{12a} is methoxy.

In some embodiments, the moiety of —Y-A₂-Y— is a moiety of Formula XIX-1 or XIX-2; and t2 is 2.

In some embodiments, each R^{12a} is, independently, alkoxy. In some embodiments, each R^{12a} is methoxy.

In some embodiments, the moiety of is a moiety of Formula XIX-1, and the moiety of Formula XIX-1 is a moiety of Formula XIX-1a:

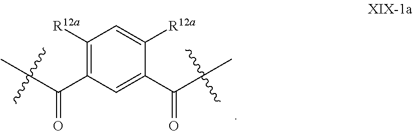

In some embodiments, each of the moiety of —X-A₁-X— is, independently, a moiety of Formula XIX-B:

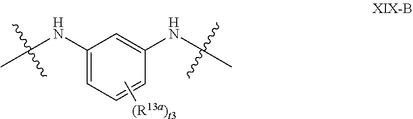

wherein each R^{13a} is, independently, a PL group or an NPL group; and t3 is 0, 1, or 2.

In some embodiments, wherein each of the moiety of —X-A₁-X— is, independently, a moiety of Formula XIX-C:

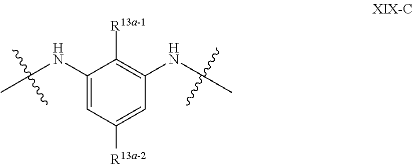

wherein each of R^{13a-1} and R^{13a-2} is, independently, H, a PL group, or an NPL group.

In some embodiments, each of R^{13a-1} and R^{13a-2} are, independently, a PL group or an NPL group. In some embodiments, each of R^{13a-1} and R^{13a-2} are, independently, halo, alkyl, haloalkyl, —O(CH₂)_{pPL}—V, or —S(CH₂)_{pPL}—V, wherein pPL is an integer from 1 to 5. In some embodiments, each of R¹³″¹ and R¹³″² are, independently, haloalkyl or —S(CH₂)_{pPL}—V, wherein pPL is an integer from 1 to 5.

In some embodiments, each A₂ is, independently, optionally substituted arylene or optionally substituted heteroarylene, and each A₁ is, independently, optionally substituted C₃ to C₈ cycloalkyl, wherein A₁ and A₂ are each, independently, optionally substituted with one or more PL group(s), one or more NPL group(s), or a combination of one or more PL group(s) and one or more NPL group(s); R¹ is —Y-A₂-Y—R²; and each R² is, independently, hydrogen, a PL group, or an NPL group. In some embodiments, each X is NH; and each Y is C═O. In some embodiments, m is 1 or 2.

In some embodiments, each A₂ is, independently, optionally substituted phenyl, and each A₁ is, independently, optionally substituted C₃-C₈ cycloalkyl, wherein A₁ and A₂ are each, independently, optionally substituted with one or more PL group(s), one or more NPL group(s), or a combination of one or more PL group(s) and one or more NPL group(s); R¹ is —Y-A₂-Y—R²; and each R² is, independently, hydrogen, a PL group, or an NPL group. In some embodiments, each X is NH; and each Y is C═O. In some embodiments, m is 1 or 2. In some embodiments, each A₁ is, independently, C₅-C₆ cycloalkyl; each A₂ is, independently, phenyl optionally substituted with one or more PL group(s), one or more NPL group(s), or a combination of one or more PL group(s) and one or more NPL group(s); R¹ is —Y-A₂-Y—R²; and each R² is, independently, hydrogen, a PL group, or an NPL group. In some embodiments, each X is NH; and each Y is C═O. In some embodiments, m is 1 or 2.

In some embodiments, each NPL group is, independently, —B(OR⁴)₂, R⁴′, or OR⁴′; R⁴ and R⁴′ are each, independently, alkyl, alkenyl, alkynyl, cycloalkyl, or aryl, each is optionally substituted with one or more substitutents, wherein each substituent is, independently, alkyl, halo, or haloalkyl; each PL group is, independently, halo, —(CH₂)_{pPL}—V, O—(CH₂)_{pPL}—V, or S—(CH₂)_{pPL}—V; each pPL is an integer from 0 to 5; and each V is, independently, hydroxy, amino, alkylamino, dialkylamino, —NH(CH₂)ₚNH₂ wherein p is 1 to 5, —N(CH₂CH₂NH₂)₂, guanidino, amidino, ureido, carbamoyl, —C(═O)OH, —C(═O)OR^{c}, —C(═O)NH—OH, —O—NH—C(═NH)NH₂, —NH—S(═O)₂OH, S(═O)₂OH, NR^{d}R^{e}, a substituted aryl group, heterocycloalkyl, and heteroaryl, wherein each of the heterocycloalkyl and heteroaryl is optionally substituted with one more substituents, wherein each substituent is, independently, amino, halo, cyano, nitro, hydroxy, —NH(CH₂)ₚNH₂ wherein p is 1 to 5, —N(CH₂CH₂NH₂)₂, amidino, guanidino, aminosulfonyl, aminoalkoxy, aminoalkylthio, lower acylamino, or benzyloxycarbonyl; and wherein the substituted aryl group is substituted with one more substituents, wherein each substituent is, independently, amino, halo, cyano, nitro, hydroxy, —NH(CH₂)ₚNH₂ wherein p is 1 to 5, —N(CH₂CH₂NH₂)₂, amidino, guanidino, aminosulfonyl, aminoalkoxy, aminoalkylthio, lower acylamino, or benzyloxycarbonyl. In some embodiments, each X is NH; and each Y is C═O. In some embodiments, m is 1 or 2.

In some embodiments, each A₁ is C₆ cycloalkyl; each A₂ is, independently, phenyl optionally substituted with one or more substituents, wherein each substituent is, independently, haloalkyl, halo, —O-alkyl, O—(CH₂)_{pPL}—V, or S—(CH₂)_{pPL}—V; R¹ is —Y-A₂-Y—R²; each R² is, independently, NH—(CH₂)_{pPL}—V; and each V is, independently, hydroxy, amino, alkylamino, dialkylamino, —NH(CH₂)ₚNH₂ wherein p is 1 to 5, —N(CH₂CH₂NH₂)₂, guanidino, amidino, ureido, carbamoyl, heterocycloalkyl, or heteroaryl. In some embodiments, each X is NH; and each Y is C═O. In some embodiments, m is 1 or 2.

In some embodiments, each A₁ is C₆ cycloalkyl; each A₂ is, independently, phenyl optionally substituted with one or more substituents, wherein each substituent is, independently, haloalkyl, —O-alkyl, O—(CH₂)_{pPL}—V, or S—(CH₂)_{pPL}—V; R¹ is —Y-A₂-Y—R²; each R² is, independently, NH—(CH₂)_{pPL}—V; and each V is, independently, amino, alkylamino, dialkylamino, —NH(CH₂)ₚNH₂ wherein p is 1 to 5, —N(CH₂CH₂NH₂)₂, guanidino, amidino, ureido, carbamoyl, or indolyl. In some embodiments, each X is NH; and each Y is C═O. In some embodiments, m is 1 or 2.

In some embodiments, each of the moiety of —Y-A₂-Y— is a moiety of Formula XIX-1 or XIX-1a:

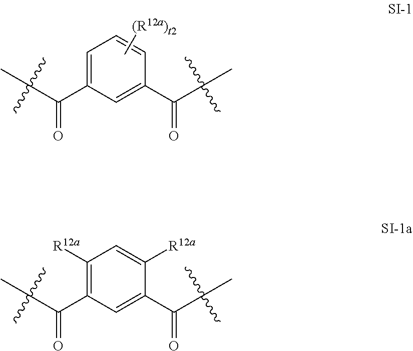

wherein each R^{12a} is, independently, a PL group or an NPL group; and t2 is 0, 1, or 2; and each of the moiety of —X-A₁-X— is, independently, a moiety of Formula XIX-D:

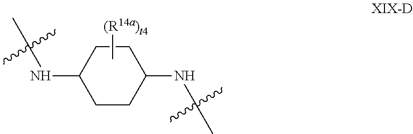

wherein each R¹⁴′ is, independently, a PL group or an NPL group. In some embodiments, each of the moiety of —Y-A₂-Y— is a moiety of Formula XIX-1a, and each of the moiety of —X-A₁-X— is a moiety of Formula XIX-D wherein t4 is 0. In some embodiments, each R^{12a} is, independently, halo, alkyl, alkoxy, haloalkyl, haloalkoxy, —(CH₂)_{pPL}—V, —O(CH₂)_{pPL}—V, or —S(CH₂)_{pPL}—V, wherein pPL is an integer from 1 to 5. In some embodiments, each R^{12a} is, independently, alkoxy or —O(CH₂)_{pPL}—V, wherein pPL is an integer from 1 to 5. In some embodiments, R¹ is —Y-A₂-Y—R²; and each R² is, independently, hydrogen, a PL group, or an NPL group. In some embodiments, m is 1, 2, or 3. In some embodiments, m is 1 or 2.

The present invention also provides methods of modulating an immune response in a mammal comprising administering to the mammal in need thereof a therapeutically effective amount of a compound chosen from:

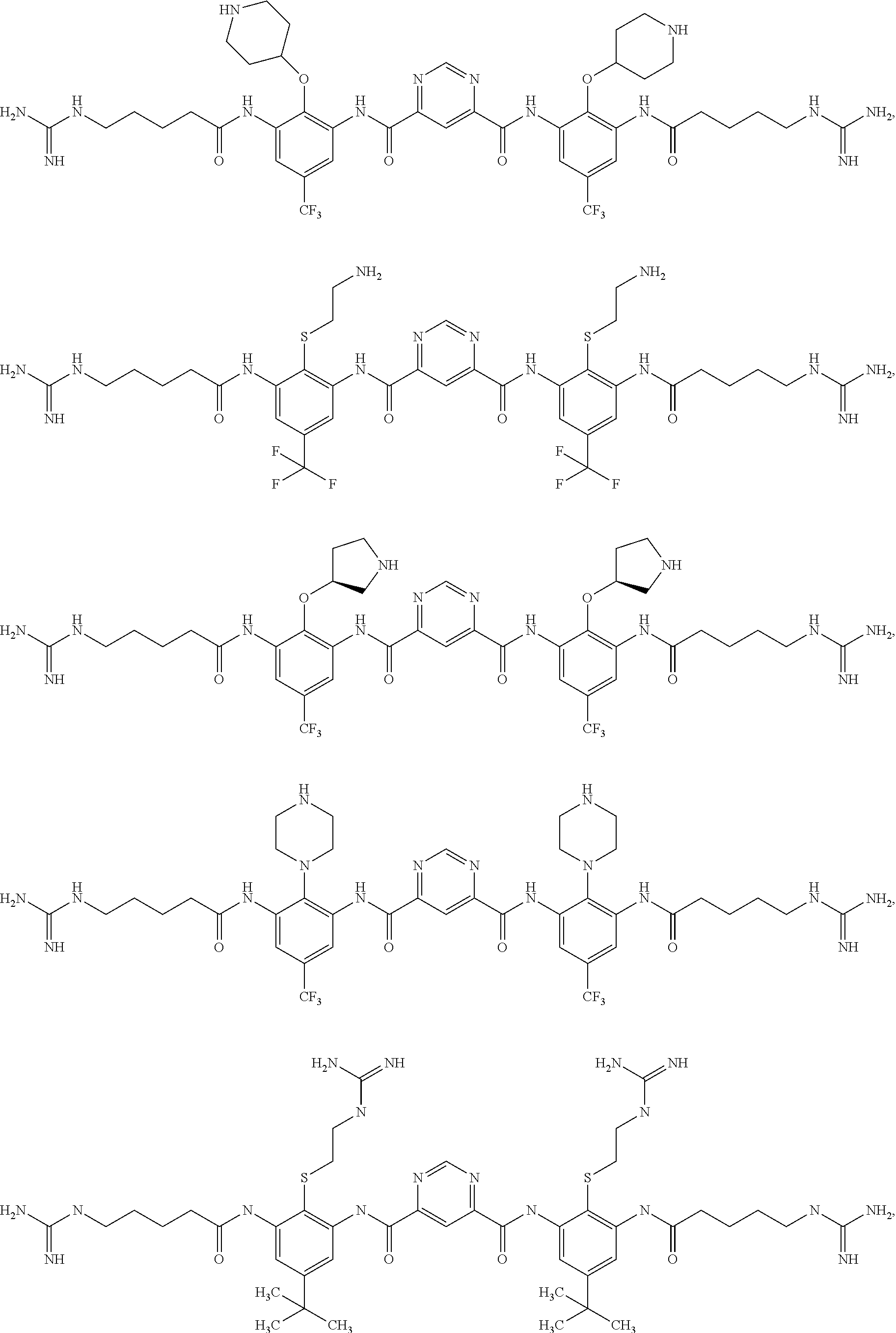

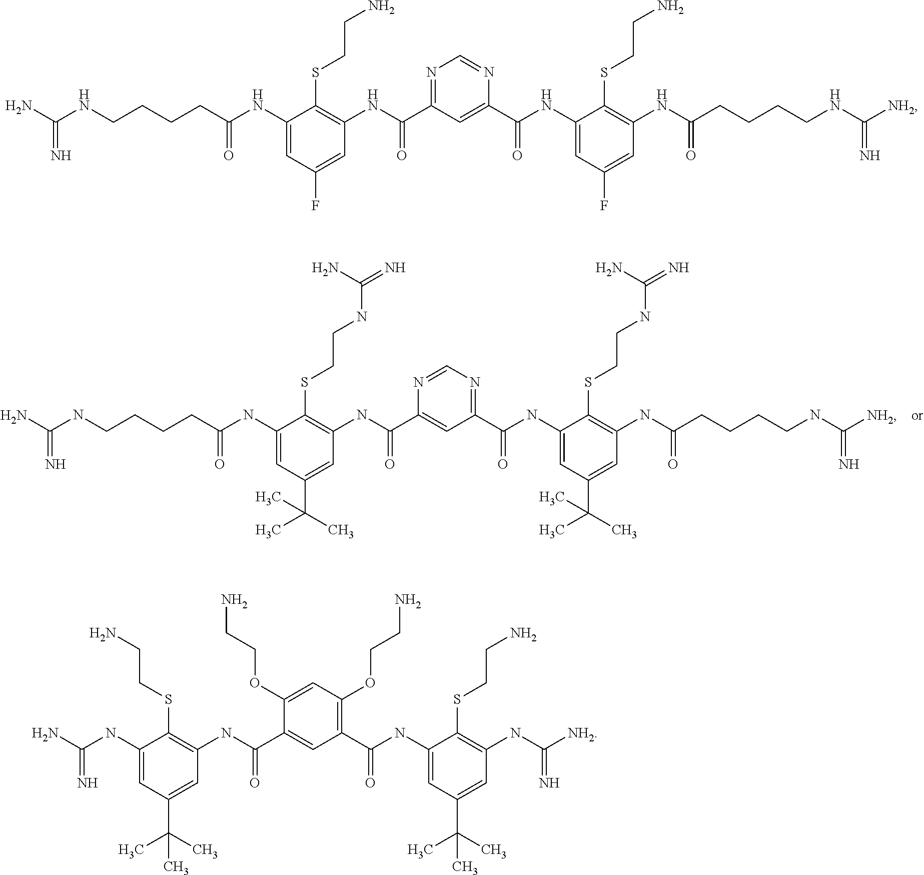

or a pharmaceutically acceptable salt thereof.

In some embodiments, the immune response is against an oral pathogen. In some embodiments, the oral pathogen is chosen from Aggregatibacter spp. such as, for example, Aggregatibacter actinomycetemcomitans; Porphyromonas spp. such as, for example, Porphyromonas gingivalis; Streptococcus spp. such as, for example, Streptococcus sanguis and Streptococcus mutans, Candida spp. such as, for example, Candida albicans, Candida glabrata, Candida krusei, Candida dubliniensis, Candida parapsilosis, and Candida tropicalis; Actinomyces spp. such as, for example, Actinomyces viscosus; and Lactobacillus spp. such as, for example, Lactobacillus casei. In some embodiments, the immune response is against a bacterial pathogen. In some embodiments, the bacterial pathogen is chosen from Staphylococcus spp., such as, for example, Staphylococcus aureus, methicillin-resistant Staphylococcus aureus, and Staphylococcus epidermidis; Streptococcus spp. such as, for example, Streptococcus pneumoniae, Streptococcus pyogenes, and Streptococcus viridans; Escherichia spp. such as, for example, E. coli; Enterococcus spp. such as, for example, Enterococcus faecalis and Enterococcus faecium; Psuedomonas spp. such as, for example, Pseudomonas aeruginosa; Acinetobacter spp. such as, for example, A. baumannii; Haemophilus spp. such as, for example, Haemophilus influenzae; Serratia spp. such as, for example, Serratia marcescens; Moraxella spp. such as, for example, Moraxella catarrhalis; Klebsiella spp. such as, for example, Klebsiella pneumoniae; Proteus spp. such as, for example, Proteus vulgaris and Proteus mirabilis; Bacteroides spp. such as, for example, Bacteroides fragalis; Clostridium spp. such as, for example, Clostridium difficile and Clostridium perfringens; and Propionibacterium spp. such as, for example, Propionibacterium acnes.

The compounds of Formula XIX or XIXa (such as the polymers and oligomers) or pharmaceutically acceptable salts thereof useful in the present invention can be made, for example, by methods described in U.S. Patent Application Publication No. 2006-0041023, U.S. Pat. No. 7,173,102, International Publication No. WO 2004/082643, International Publication No. WO2006093813, and U.S. Patent Application Publication 2010-0081665. In some embodiments, the compounds of Formula XIX or XIXa (such as the polymers and oligomers) or pharmaceutically acceptable salts thereof useful in the present invention can be selected from those described in U.S. Patent Application Publication No. 2006-0041023, U.S. Pat. No. 7,173,102, International Publication No. WO 2004/082643, International Publication No. WO2006093813, and U.S. Patent Application Publication 2010-0081665.

In some embodiments, the compound(s) useful in the method of present invention can be chosen from one or more of the compounds (i.e., genuses, sub-genuses, and species) disclosed in U.S. Patent Application Publication No. 2006-0041023, U.S. Pat. No. 7,173,102, International Publication No. WO 2005/123660, International Publication No. WO 2004/082643, International Publication No. WO 2006/093813, and U.S. Patent Application Publication 2010-0081665, each of which is hereby incorporated by reference in its entirety.

The present invention also provides methods of modulating an immune response in a mammal comprising administering to the mammal in need thereof a therapeutically effective amount of a compound of Formula XX:

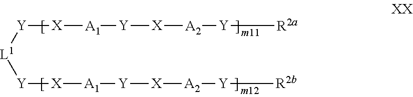

or a pharmaceutically acceptable salt thereof,
wherein:

each X is, independently, NR⁸;

each Y is C═O;

each R⁸ is, independently, hydrogen or alkyl;

each A₂ is optionally substituted arylene or optionally substituted heteroarylene, and each A₁ is —(CH₂)_{q}—, wherein q is 1 to 7, wherein A₁ and A₂ are each, independently, optionally substituted with one or more PL group(s), one or more NPL group(s), or a combination of one or more PL group(s) and one or more NPL group(s);

R² and R^{2a} are each, independently, hydrogen, a PL group, an NPL group or —X-A₁-Y—R¹¹, wherein R¹¹ is hydrogen, a PL group, or an NPL group;

L¹ is C₁₋₁₀ alkylene optionally substituted with one or more substitutents, wherein each substituent is, independently, alkyl, halo, haloalkyl, aminoalkyl, hydroxylalkyl, V, or (CH₂)_{pPL}—V, wherein pPL is an integer from 1 to 5;

each NPL group is, independently, —B(OR⁴)₂ or

—(NR³)_{q1NPL}—U^{NPL}-LK^{NPL}—(NR³″)_{q2NPL}—R⁴′, wherein:

R³, R³′, and R³″ are each, independently, hydrogen, alkyl, or alkoxy;

R⁴ and R⁴′ are each, independently, hydrogen, alkyl, alkenyl, alkynyl, cycloalkyl, aryl, or heteroaryl, wherein each of the alkyl, alkenyl, alkynyl, cycloalkyl, aryl, and heteroaryl is optionally substituted with one or more substitutents, wherein each substituent is, independently, alkyl, halo, or haloalkyl;

each U^{NPL} is, independently, absent or O, S, S(═O), S(═O)₂, NR³, —C(═O)—, —C(═O)—NR³—, —C(═O)—N═N—NR³—, —C(═O)—NR³—N═N—, —N═N—NR³—, —C(═N—N(R³)₂)—, —C(═NR³)—, —C(═O)O—, —C(═O)S—, —C(═S)—, —O—P(═O)₂O—, —S—C═N—, or —C(═O)—NR³—O—, wherein groups with two chemically nonequivalent termini can adopt both possible orientations;

each LK^{NPL} is, independently, —(CH₂)_{pNPL}— and C₂₋₈ alkenylenyl, wherein each of the —(CH₂)_{pNPL} and C₂₋₈ alkenylenyl is optionally substituted with one or more substituents, wherein each substituent is, independently, amino, hydroxyl, aminoalkyl, hydroxylalkyl, or alkyl;

each pNPL is, independently, an integer from 0 to 8;

q1NPL and q2NPL are each, independently, 0, 1, or 2; each PL group is, independently, halo, hydroxyethoxymethyl, methoxyethoxymethyl, polyoxyethylene, or —(NR⁵)_{q1PL}—U^{PL}-LK^{PL}—(NR⁵″)_{q2PL}—V, wherein:

R⁵, R⁵′, and R⁵″ are each, independently, hydrogen, alkyl, or alkoxy;

each U^{PL} is, independently, absent or O, S, S(═O), S(═O)₂, NR⁵, —C(═O)—, —C(═O)—NR⁵—, —C(═O)—N═N—NR⁵—, —C(═O)—NR⁵—N═N—, —N═N—NR⁵—, —C(═N—N(R⁵)₂)—, —C(═NR⁵)—, —C(═O)O—, —C(═O)S—, —C(═S)—, —O—P(═O)₂O—, —S—C═N—, or —C(═O)—NR⁵—O—, wherein groups with two chemically nonequivalent termini can adopt either of the two possible orientations;

each V is, independently, nitro, cyano, amino, halo, hydroxy, alkoxy, alkylthio, alkylamino, dialkylamino, —NH(CH₂)ₚNH₂ wherein p is 1 to 5, —C(═O)NH(CH₂)ₚNH₂ wherein p is 1 to 5, —C(═O)NH(CH₂)ₚNHC(═NH)NH₂ wherein p is 1 to 5, —C(═O)NH(CH₂)ₚNHC(═O)NH₂ wherein p is 1 to 5, —NHC(═O)-alkyl, —N(CH₂CH₂NH₂)₂, diazamino, amidino, guanidino, ureido, carbamoyl, —C(═O)OH, —C(═O)OR^{c}, —C(═O)NH—OH, O—NH—C(═NH)NH₂, —NH—S(═O)₂OH, S(═O)₂OH, NR^{d}R^{e}, semicarbazone, aryl, cycloalkyl, heterocycloalkyl, or heteroaryl, wherein each of the aryl and cycloalkyl is substituted with one or more substitutents, wherein each of the heterocycloalkyl and heteroaryl is optionally substituted with one or more substituents, and wherein each of the substituents for the aryl, cycloalkyl, heterocycloalkyl, and heteroaryl is, independently, nitro, cyano, amino, halo, hydroxy, alkoxy, alkylthio, alkylamino, dialkylamino, —NH(CH₂)ₚNH₂ wherein p is 1 to 5, —N(CH₂CH₂NH₂)₂, diazamino, amidino, guanidino, ureido, carbamoyl, —C(═O)OH, —C(═O)OR^{c}, —C(═O)NH—OH, —O—NH—C(═NH)NH₂, —NH—S(═O)₂OH, S(═O)₂OH, NR^{d}R^{C}, semicarbazone, aminosulfonyl, aminoalkoxy, aminoalkylthio, lower acylamino, or benzyloxycarbonyl;

each R^{c} is, independently, C₁₋₆ alkyl, C₁₋₆ haloalkyl, C₂₋₆ alkenyl, C₂₋₆ alkynyl, aryl, cycloalkyl, heteroaryl, heterocycloalkyl, arylalkyl, heteroarylalkyl, cycloalkylalkyl, or heterocycloalkylalkyl, each optionally substituted by one or more substitutents, wherein each substituent is, independently, OH, amino, halo, C₁₋₆ alkyl, C₁₋₆ haloalkyl, aryl, arylalkyl, heteroaryl, heteroarylalkyl, cycloalkyl, or heterocycloalkyl;

R^{d} and R^{e} are, independently, H, C₁₋₆ alkyl, C₁₋₆ haloalkyl, C₂₋₆ alkenyl, C₂₋₆ alkynyl, aryl, heteroaryl, cycloalkyl, heterocycloalkyl, arylalkyl, heteroarylalkyl, cycloalkylalkyl, or heterocycloalkylalkyl, wherein each of the C₁₋₆ alkyl, C₁₋₆ haloalkyl, alkenyl, C₂₋₆ alkynyl, aryl, heteroaryl, cycloalkyl, heterocycloalkyl, arylalkyl, heteroarylalkyl, cycloalkylalkyl, or heterocycloalkylalkyl is optionally substituted by OH, amino, halo, C₁₋₆ alkyl, C₁₋₆ haloalkyl, C₁₋₆ haloalkyl, aryl, arylalkyl, heteroaryl, heteroarylalkyl, cycloalkyl, or heterocycloalkyl;

or R^{d} and R^{e} together with the N atom to which they are attached form a 4-, 5-, 6-, 7-, or 8-membered heterocycloalkyl;

each LK^{PL} is, independently, —(CH₂)_{pPL}— or C₂₋₈ alkenylenyl, wherein each of the —(CH₂)_{pNPL}— and C₂₋₈ alkenylenyl is optionally substituted with one or more substituents, wherein each substituent is, independently, amino, hydroxyl, aminoalkyl, hydroxylalkyl, or alkyl;

each pPL is, independently, an integer from 0 to 8;

q1PL and q2PL are each, independently, 0, 1, or 2;

m11 is an integer from 1 to about 20; and

m12 is an integer from 1 to about 20.

In some embodiments, each moiety of X-A₁-Y—X-A₂-Y is, independently, a moiety of:

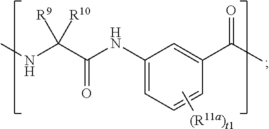

each R⁹ is, independently, H, a PL group, or an NPL group; each R¹⁰ is, independently, H, a PL group, or an NPL group; each R^{11a} is, independently, a PL group or an NPL group; and each t1 is independently 0, 1, or 2.

In some embodiments, each R⁹ is, independently, a PL group or an NPL group; and each R¹⁰ is H. In some embodiments, each R⁹ is, independently, alkyl or (CH₂)_{pPL}—V where pPL is an integer from 1 to 5; each R¹⁰ is H; and each R^{11a} is, independently, halo, alkyl, alkoxy, haloalkyl, haloalkoxy, —(CH₂)_{pPL}—V, —O(CH₂)_{pPL}—V, or —S(CH₂)_{pPL}—V, wherein pPL is an integer from 1 to 5.

In some embodiments, each R⁹ is, independently, alkyl, —(CH₂)—V, —(CH₂)₂—V, —(CH₂)₃—V, —(CH₂)₄—V, or —(CH₂)₅—V; each R¹⁰ is H; each V is, independently, hydroxyl, amino, heteroarylamino, ureido, guanidino, carbamoyl, C(═O)OH, —C(═O)OR^{c}, —C(═O)NH—OH, —O—NH—C(═NH)NH₂, —NH—S(═O)₂OH, S(═O)₂OH, aziridinyl, azetidinyl, pyrrolidinyl, piperidinyl, piperazinyl, morpholino, azepanyl, azocanyl, tetrazolyl, 1,2,4-oxadiazolyl, 1,3,4-oxadiazolyl, imidazolyl, pyridinyl, indolyl, or a substituted phenyl, wherein the substituted phenyl is substituted with one or more substituents, wherein each substituent is, independently, OH or amino; and each R^{11a} is, independently, alkoxy.

In some embodiments, each R⁹ is, independently, CH₃, —(CH₂)—V, —(CH₂)₂—V, —(CH₂)₃—V, —(CH₂)₄—V, and —(CH₂)₅—V; each R¹⁰ is H; each V is, independently, amino, alkylamino, dialkylamino, —NH(CH₂)ₚNH₂ wherein p is 1 to 5, —N(CH₂CH₂NH₂)₂, guanidino, amidino, ureido, or indolyl; and each R^{11a} is, independently, alkoxy.

In some embodiments, each R⁹ is, independently, CH₃, —(CH₂)—V, —(CH₂)₂—V, —(CH₂)₃—V, —(CH₂)₄—V, and —(CH₂)₅—V; each R¹⁰ is H; each V is, independently, amino, alkylamino, dialkylamino, —NH(CH₂)ₚNH₂ wherein p is 1 to 5, —N(CH₂CH₂NH₂)₂, guanidino, amidino, ureido, or indolyl; and each R^{11a} is methoxy.

In some embodiments, each moiety of X-A₁-Y—X-A₂-Y is, independently, a moiety of:

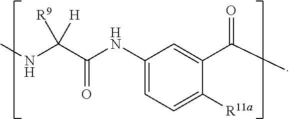

In some embodiments, R² and R^{2a} are each, independently, NH₂, amidino, alkylamino, dialkylamino, —NH(CH₂)ₚNH₂ wherein p is 1 to 5, or —NH—(CH₂)_{pPL}—V¹⁰, wherein V is amino, alkylamino, dialkylamino, —NH(CH₂)NH₂ wherein p is 1 to 5, —N(CH₂CH₂NH₂)₂, guanidino, amidino, ureido, or carbamoyl; and L¹ is C₅₋₁₀alkylene optionally substituted with one or more substitutents, wherein each substituent is, independently, alkyl, halo, haloalkyl, aminoalkyl, or hydroxylalkyl.

In some embodiments, each of R² and R^{2a} is NH₂; and L¹ is C₅₋₁₀ alkylene, such as, for example C₇₋₁₀alkylene or C₇₋₉alkylene.

In some embodiments, m11 is an integer from 1 to about 10; and m12 is an integer from 1 to about 10. In some embodiments, m11 is an integer from 3 to 7; and m12 is an integer from 3 to 7. In some embodiments, m11 is an integer from 3 to 5; and m12 is an integer from 3 to 5.

In some embodiments, the immune response is against an oral pathogen. In some embodiments, the oral pathogen is chosen from Aggregatibacter spp. such as, for example; Aggregatibacter actinomycetemcomitans; Porphyromonas spp. such as, for example, Porphyromonas gingivalis; Streptococcus spp. such as, for example, Streptococcus sanguis and Streptococcus mutans, Candida spp. such as, for example, Candida albicans, Candida glabrata, Candida krusei, Candida dubliniensis, Candida parapsilosis, and Candida tropicalis; Actinomyces spp. such as, for example, Actinomyces viscosus; and Lactobacillus spp. such as, for example, Lactobacillus casei. In some embodiments, the immune response is against a bacterial pathogen. In some embodiments, the bacterial pathogen is chosen from Staphylococcus spp., such as, for example, Staphylococcus aureus, methicillin-resistant Staphylococcus aureus, and Staphylococcus epidermidis; Streptococcus spp. such as, for example, Streptococcus pneumoniae, Streptococcus pyogenes, and Streptococcus viridans; Escherichia spp. such as, for example, E. coli; Enterococcus spp. such as, for example, Enterococcus faecalis and Enterococcus faecium; Psuedomonas spp. such as, for example, Pseudomonas aeruginosa; Acinetobacter spp. such as, for example, A. baumannii; Haemophilus spp. such as, for example, Haemophilus influenzae; Serratia spp. such as, for example, Serratia marcescens; Moraxella spp. such as, for example, Moraxella catarrhalis; Klebsiella spp. such as, for example, Klebsiella pneumoniae; Proteus spp. such as, for example, Proteus vulgaris and Proteus mirabilis; Bacteroides spp. such as, for example, Bacteroides fragalis; Clostridium spp. such as, for example, Clostridium difficile and Clostridium perfringens; and Propionibacterium spp. such as, for example, Propionibacterium acnes.

The present invention also provides methods of modulating an immune response in a mammal comprising administering to the mammal in need thereof a therapeutically effective amount of a compound of Formula XXI:

R¹—[—X-A₁-Y—X-A₂-Y—]ₘ₁₃—X-L¹-Y—[—X-A₁-Y—X-A₂-Y—]ₘ₁₄—R² XXI

or a pharmaceutically acceptable salt thereof,
wherein:

each X is, independently, NR⁸;

each Y is C═O;

each R⁸ is, independently, hydrogen or alkyl;

each A₂ is optionally substituted arylene or optionally substituted heteroarylene, and

each A₁ is —(CH₂)_{q}—, wherein q is 1 to 7, wherein A₁ and A₂ are each, independently, optionally substituted with one or more PL group(s), one or more NPL group(s), or a combination of one or more PL group(s) and one or more NPL group(s);

R¹ is hydrogen, a PL group, or an NPL group, and R² is —X-A₁-Y—R¹¹, wherein R^{H} is hydrogen, a PL group, or an NPL group; or

R¹ and R² are each, independently, hydrogen, a PL group, or an NPL group; or

R¹ and R² together are a single bond; or

R¹ is —Y-A₂-X—R¹², wherein R¹² is hydrogen, a PL group, or an NPL group, and R² is hydrogen, a PL group, or an NPL group;

L¹ is C₁₋₁₀alkylene optionally substituted with one or more substitutents, wherein each substituent is, independently, alkyl, halo, haloalkyl, aminoalkyl, hydroxylalkyl, V, or —(CH₂)_{pPL}—V wherein pPL is an integer from 1 to 5;

each V is, independently, hydroxy, amino, alkylamino, dialkylamino, —NH(CH₂)ₚNH₂ wherein p is 1 to 5, —C(═O)NH(CH₂)ₚNH₂ wherein p is 1 to 5, —C(═O)NH(CH₂)ₚNHC(═NH)NH₂ wherein p is 1 to 5, —C(═O)NH(CH₂)ₚNHC(═O)NH₂ wherein p is 1 to 5, —NHC(═O)-alkyl, —N(CH₂CH₂NH₂)₂, guanidino, amidino, ureido, carbamoyl, —C(═O)OH, —C(═O)OR^{c}, —C(═O)NH—OH, —O—NH—C(═NH)NH₂, —NH—S(═O)₂OH, S(═O)₂OH, NR^{d}R^{e}, a substituted aryl group, heterocycloalkyl, or heteroaryl, wherein each of the heterocycloalkyl and heteroaryl is optionally substituted with one more substituents, wherein each substituent is, independently, amino, halo, cyano, nitro, hydroxy, —NH(CH₂)ₚNH₂ wherein p is 1 to 5, —N(CH₂CH₂NH₂)₂, amidino, guanidino, aminosulfonyl, aminoalkoxy, aminoalkylthio, lower acylamino, or benzyloxycarbonyl; and wherein the substituted aryl group is substituted with one more substituents, wherein each substituent is, independently, amino, halo, cyano, nitro, hydroxy, —NH(CH₂)ₚNH₂ wherein p is 1 to 5, —N(CH₂CH₂NH₂)₂, amidino, guanidino, aminosulfonyl, aminoalkoxy, aminoalkylthio, lower acylamino, or benzyloxycarbonyl;

each NPL group is, independently, —B(OR⁴)₂ or

—(NR³′)_{q1NPL}—U^{NPL}-LK^{NPL}—(NR³″)_{q2NPL}—R⁴′, wherein:

R³, R³′, and R³″ are each, independently, hydrogen, alkyl, or alkoxy;

R⁴ and R⁴′ are each, independently, hydrogen, alkyl, alkenyl, alkynyl, cycloalkyl, aryl, or heteroaryl, wherein each of the alkyl, alkenyl, alkynyl, cycloalkyl, aryl, and heteroaryl is optionally substituted with one or more substitutents, wherein each substituent is, independently, alkyl, halo, or haloalkyl;

each U^{NPL} is, independently, absent or O, S, S(═O), S(═O)₂, NR³, —C(═O)—, —C(═O)—NR³—, —C(═O)—N═N—NR³—, —C(═O)—NR³—N═N—, —N═N—NR³—, —C(═N—N(R³)₂)—, —C(═NR³)—, —C(═O)O—, —C(═O)S—, —C(═S)—, —O—P(═O)₂O—, —S—C═N—, or —C(═O)—NR³—O—, wherein groups with two chemically nonequivalent termini can adopt both possible orientations;

each LK^{NPL} is, independently, —(CH₂)_{pNPL}— or C₂₋₈ alkenylenyl, wherein each of the —(CH₂)_{pNPL} and C₂₋₈ alkenylenyl is optionally substituted with one or more substituents, wherein each substituent is, independently, amino, hydroxyl, aminoalkyl, hydroxylalkyl, or alkyl;

each pNPL is, independently, an integer from 0 to 8;

q1NPL and q2NPL are each, independently, 0, 1, or 2;

each PL group is, independently, halo, hydroxyethoxymethyl, methoxyethoxymethyl, polyoxyethylene, or —(NR⁵′)_{q1PL}—U^{PL}-LK^{PL}—(NR⁵″)_{q2PL}—V, wherein:

R⁵, R⁵′, and R⁵″ are each, independently, hydrogen, alkyl, or alkoxy;

each U^{PL} is, independently, absent or O, S, S(═O), S(═O)₂, NR⁵, —C(═O)—, C(═O)—NR⁵—, —C(═O)—N═N—NR⁵—, —C(═O)—NR⁵—N═N—, —N═N—NR⁵—, —C(═N—N(R⁵)₂)—, —C(═NR⁵)—, —C(═O)O—, —C(═O)S—, —C(═S)—, —O—P(═O)₂O—, —S—C═N—, or —C(═O)—NR⁵—O—, wherein groups with two chemically nonequivalent termini can adopt either of the two possible orientations;

each R^{c} is, independently, C₁₋₆ alkyl, C₁₋₆ haloalkyl, C₂₋₆ alkenyl, C₂₋₆ alkynyl, aryl, cycloalkyl, heteroaryl, heterocycloalkyl, arylalkyl, heteroarylalkyl, cycloalkylalkyl, or heterocycloalkylalkyl, each optionally substituted by one or more substitutents, wherein each substituent is, independently, OH, amino, halo, C₁₋₆ alkyl, haloalkyl, aryl, arylalkyl, heteroaryl, heteroarylalkyl, cycloalkyl, or heterocycloalkyl;

R^{d} and R^{e} are, independently, H, C₁₋₆ alkyl, C₁₋₆ haloalkyl, C₂₋₆ alkenyl, C₂₋₆ alkynyl, aryl, heteroaryl, cycloalkyl, heterocycloalkyl, arylalkyl, heteroarylalkyl, cycloalkylalkyl, or heterocycloalkylalkyl, wherein each of the C₁₋₆ alkyl, C₁₋₆ haloalkyl, C₂₋₆ alkenyl, C₂₋₆ alkynyl, aryl, heteroaryl, cycloalkyl, heterocycloalkyl, arylalkyl, heteroarylalkyl, cycloalkylalkyl, and heterocycloalkylalkyl is optionally substituted by OH, amino, halo, C₁₋₆ alkyl, C₁₋₆ haloalkyl, C₁₋₆ haloalkyl, aryl, arylalkyl, heteroaryl, heteroarylalkyl, cycloalkyl or heterocycloalkyl;

or R^{d} and R^{e} together with the N atom to which they are attached form a 4-, 5-, 6-, 7-, or 8-membered heterocycloalkyl;

each LK^{PL} is, independently, —(CH₂)_{pPL}— or C₂₋₈ alkenylenyl, wherein each of the —(CH₂)_{pNPL}— and C₂₋₈ alkenylenyl is optionally substituted with one or more substituents, wherein each substituent is, independently, amino, hydroxyl, aminoalkyl, hydroxylalkyl, or alkyl;

each pPL is, independently, an integer from 0 to 8;

q1PL and q2PL are each, independently, 0, 1, or 2;

m13 is an integer from 1 to about 10; and

m14 is an integer from 1 to about 10.

In some embodiments, each moiety of X-A₁-Y—X-A₂-Y—X-A₂-Y is, independently, a moiety of:

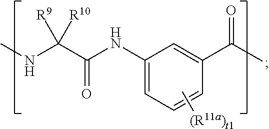

each R⁹ is, independently, H, a PL group, or an NPL group; each R¹⁰ is, independently, H, a PL group, or an NPL group; each R^{11a} is, independently, a PL group or an NPL group; and each t1 is independently 0, 1, or 2.

In some embodiments, each R⁹ is, independently, a PL group or an NPL group; and each R¹⁰ is H. In some embodiments, each R⁹ is, independently, alkyl or (CH₂)_{pPL}—V wherein pPL is an integer from 1 to 5; each R¹⁰ is H; and each R^{11a} is, independently, halo, alkyl, alkoxy, haloalkyl, haloalkoxy, —(CH₂)_{pPL}—V, —O(CH₂)_{pPL}—V, or —S(CH₂)_{pPL}—V, wherein pPL is an integer from 1 to 5.

In some embodiments, each R⁹ is, independently, alkyl, —(CH₂)—V, —(CH₂)₂—V, —(CH₂)₃—V, —(CH₂)₄—V, or —(CH₂)₅—V; each R¹° is H; each V is, independently, hydroxyl, amino, heteroarylamino, ureido, guanidino, carbamoyl, C(═O)OH, —C(═O)OR^{c}, —C(═O)NH—OH, —O—NH—C(═NH)NH₂, —NH—S(═O)₂OH, S(═O)₂OH, aziridinyl, azetidinyl, pyrrolidinyl, piperidinyl, piperazinyl, morpholino, azepanyl, azocanyl, tetrazolyl, 1,2,4-oxadiazolyl, 1,3,4-oxadiazolyl, imidazolyl, pyridinyl, indolyl, or a substituted phenyl, wherein the substituted phenyl is substituted with one or more substituents, wherein each substituent is, independently, OH or amino; and each R^{11a} is, independently, alkoxy.

In some embodiments, each R⁹ is, independently, CH₃, —(CH₂)—V, —(CH₂)₂—V, —(CH₂)₃—V, —(CH₂)₄—V, or —(CH₂)₅—V; each R¹⁰ is H; each V is, independently, amino, alkylamino, dialkylamino, —NH(CH₂)ₚNH₂ wherein p is 1 to 5, —N(CH₂CH₂NH₂)₂, guanidino, amidino, ureido, or indolyl; and each R^{11a} is, independently, alkoxy.

In some embodiments, each R⁹ is, independently, CH₃, —(CH₂)—V, —(CH₂)₂—V, —(CH₂)₃—V, —(CH₂)₄—V, or —(CH₂)₅—V; each R¹⁰ is H; each V is, independently, amino, alkylamino, dialkylamino, —NH(CH₂)ₚNH₂ wherein p is 1 to 5, —N(CH₂CH₂NH₂)₂, guanidino, amidino, ureido, or indolyl; and each R^{11a} is methoxy.

In some embodiments, each moiety of X-A₁-Y—X-A₂-Y is, independently, a moiety of:

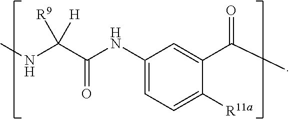

In some embodiments, the moiety of —X-L¹-Y— is a moiety of —NH-L¹-C(═O)—; R¹ is H or alkyl; R² is NH₂, amidino, alkylamino, dialkylamino, —NH(CH₂)ₚNH₂ wherein p is 1 to 5, or —NH—(CH₂)_{pPL}—V¹⁰, wherein V¹⁰ is amino, alkylamino, dialkylamino, —NH(CH₂)ₚNH₂ wherein p is 1 to 5, —N(CH₂CH₂NH₂)₂, guanidino, amidino, ureido, or carbamoyl; and L′ is C₁₋₃alkylene optionally substituted with one or more substitutents, wherein each substituent is, independently, alkyl, halo, haloalkyl, aminoalkyl, hydroxylalkyl, V¹¹, or —(CH₂)_{pPL}—V¹¹ wherein pPL is an integer from 1 to 5, wherein each V¹¹ is, independently, amino, alkylamino, dialkylamino, —NH(CH₂)ₚNH₂ wherein p is 1 to 5, —N(CH₂CH₂NH₂)₂, guanidino, amidino, ureido, or carbamoyl.

In some embodiments, the moiety of —X-L¹-Y— is a moiety of —NH-L¹-C(═O)—; R¹ is H; R² is NH₂; and L¹ is C₁alkylene optionally substituted with one or more substitutents, wherein each substituent is, independently, alkyl, halo, haloalkyl, aminoalkyl, hydroxylalkyl, V¹¹, or —(CH₂)_{pPL}—V¹¹ wherein pPL is an integer from 1 to 5, wherein V¹¹ is amino, alkylamino, dialkylamino, —NH(CH₂)ₚNH₂ wherein p is 1 to 5, —N(CH₂CH₂NH₂)₂, guanidino, amidino, ureido, or carbamoyl.

In some embodiments, m13 is an integer from 1 to about 5; and m14 is an integer from 1 to about 5. In some embodiments, m13 is an integer from 1 to 3; and m12 is an integer from 1 to 3. In some embodiments, the sum of m13 and m14 is an integer from 3 to 5. In some embodiments, the sum of m13 and m14 is 4.

In some embodiments, the immune response is against an oral pathogen. In some embodiments, the oral pathogen is chosen from Aggregatibacter spp. such as, for example, Aggregatibacter actinomycetemcomitans; Porphyromonas spp. such as, for example, Porphyromonas gingivalis; Streptococcus spp. such as, for example, Streptococcus sanguis and Streptococcus mutans, Candida spp. such as, for example, Candida albicans, Candida glabrata, Candida krusei, Candida dubliniensis, Candida parapsilosis, and Candida tropicalis; Actinomyces spp. such as, for example, Actinomyces viscosus; and Lactobacillus spp. such as, for example, Lactobacillus casei. In some embodiments, the immune response is against a bacterial pathogen. In some embodiments, the bacterial pathogen is chosen from Staphylococcus spp., such as, for example, Staphylococcus aureus, methicillin-resistant Staphylococcus aureus, and Staphylococcus epidermidis; Streptococcus spp. such as, for example, Streptococcus pneumoniae, Streptococcus pyogenes, and Streptococcus viridans; Escherichia spp. such as, for example, E. coli; Enterococcus spp. such as, for example, Enterococcus faecalis and Enterococcus faecium; Psuedomonas spp. such as, for example, Pseudomonas aeruginosa; Acinetobacter spp. such as, for example, A. baumannii; Haemophilus spp. such as, for example, Haemophilus influenzae; Serratia spp. such as, for example, Serratia marcescens; Moraxella spp. such as, for example, Moraxella catarrhalis; Klebsiella spp. such as, for example, Klebsiella pneumoniae; Proteus spp. such as, for example, Proteus vulgaris and Proteus mirabilis; Bacteroides spp. such as, for example, Bacteroides fragalis; Clostridium spp. such as, for example, Clostridium difficile and Clostridium perfringens; and Propionibacterium spp. such as, for example, Propionibacterium acnes.

The present invention also provides methods of modulating an immune response in a mammal comprising administering to the mammal in need thereof a therapeutically effective amount of a compound of Formula XXII:

R¹—[—X-A₁X—Z—Y-A₂-Y—Z]ₘ—R² XXII

or a pharmaceutically acceptable salt thereof,
wherein:

X is NR⁸, —NR⁸NR⁸—, C═O, or O;

Y is NR⁸, —NR⁸NR⁸—, C═O, S, or O;

R⁸ is hydrogen or alkyl;

Z is C═O, C═S, O═S═O, —NR⁸NR⁸—, or —C(═O)C(═O)—;

A₁ and A₂ are, independently, optionally substituted arylene or optionally substituted heteroarylene, wherein A₁ and A₂ are, independently, optionally substituted with one or more polar (PL) group(s), one or more non-polar (NPL) group(s), or a combination of one or more polar (PL) group(s) and one or more non-polar (NPL) group(s);

R¹ is
(i) hydrogen, a polar group (PL), or a non-polar group (NPL), and R² is —X-A₁-X—R¹, wherein A, is as defined above and is optionally substituted with one or more polar (PL) group(s), one or more non-polar (NPL) group(s), or a combination of one or more polar (PL) group(s) and one or more non-polar (NPL) group(s); or
(ii) hydrogen, a polar group (PL), or a non-polar group (NPL), and R² is —X-A₁-X—Z—Y-A₂-Y—R¹, wherein A₁ and A₂ are as defined above, and each of which is optionally substituted with one or more polar (PL) group(s), one or more non-polar (NPL) group(s), or a combination of one or more polar (PL) group(s) and one or more non-polar (NPL) group(s); or
(iii) hydrogen, a polar group (PL), or a non-polar group (NPL), and R² is —X-A′-X—R¹, wherein A′ is aryl or heteroaryl and is optionally substituted with one or more polar (PL) group(s), one or more non-polar (NPL) group(s), or a combination of one or more polar (PL) group(s) and one or more non-polar (NPL) group(s); or
(iv) hydrogen, a polar group (PL), or a non-polar group (NPL), and R² is —X-A₁-X—Z—Y-A′-Y—R¹, wherein A₁ is as defined above, A′ is aryl or heteroaryl, and each of A₁ and A′ is optionally substituted with one or more polar (PL) group(s), one or more non-polar (NPL) group(s), or a combination of one or more polar (PL) group(s) and one or more non-polar (NPL) group(s); or
(v) —Z—Y-A¹ and R² is hydrogen, a polar group (PL), or a non-polar group (NPL), wherein A′ is aryl or heteroaryl and is optionally substituted with one or more polar (PL) group(s), one or more non-polar (NPL) group(s), or a combination of one or more polar (PL) group(s) and one or more non-polar (NPL) group(s); or
(vi) —Z—Y-A′, and R² is —X-A″, wherein A′ and A″ are, independently, aryl or heteroaryl, and each of A′ and A″ is optionally substituted with one or more polar (PL) group(s), one or more non-polar (NPL) group(s), or a combination of one or more polar (PL) group(s) and one or more non-polar (NPL) group(s); or
(vii) R¹ and R² are, independently, a polar group (PL) or a non-polar group (NPL); or
(viii) R¹ and R² together form a single bond; NPL is a nonpolar group independently selected from —B(OR⁴)₂ and —(NR³′)_{q1NPL}—U^{NPL}—(CH₂)_{pNPL}—(NR³″)_{q2NPL}—R⁴′, wherein:

R³, R³′, and R³″ are, independently, selected from hydrogen, alkyl, and alkoxy;

R⁴ and R⁴′ are, independently, selected from hydrogen, alkyl, alkenyl, alkynyl, cycloalkyl, aryl, and heteroaryl, any of which is optionally substituted with one or more alkyl or halo groups;

U^{NPL} is absent or selected from O, S, S(═O), S(═O)₂, NR³, —C(═O)—, —C(═O)—N═N—NR³—, —C(═O)—NR³—N═N—, —N═N—NR³—, —C(═N—N(R³)₂)—, —C(═NR³)—, —C(═O)O—, —C(═O)S—, —C(═S)—, —O—P(═O)₂O—, —R³⁰—, —R³S—, —S—C═N—, and —C(═O)—NR³—O—, wherein groups with two chemically nonequivalent termini can adopt both possible orientations;

the —(CH₂)_{pNPL}— alkylene chain is optionally substituted with one or more amino or hydroxy groups, or is unsaturated;

pNPL is 0 to 8;

q1NPL and q2NPL are, independently, 0, 1, or 2;

PL is a polar group selected from halo, hydroxyethoxymethyl, methoxyethoxymethyl, polyoxyethylene, and —(NR⁵)_{q1PL}—U^{PL}—(CH₂)_{pPL}—(NR⁵′)_{q2PL}—V, wherein:

R⁵, R⁵′, and R⁵″ are, independently, selected from hydrogen, alkyl, and alkoxy;

U^{PL} is absent or selected from O, S, S(═O), S(═O)₂, NR⁵, —C(═O)—, —C(═O)—N═N—NR⁵—, —C(═O)—NR⁵—N═N—, —N═N—NR⁵—, —C(═N—N(R⁵)₂)—, —C(═NR⁵)—, —C(═O)O—, —C(═O)S—, —C(═S)—, —O—P(═O)₂O—, —R⁵O—, —S—C═N—, and —C(═O)—NR⁵—O—, wherein groups with two chemically nonequivalent termini can adopt both possible orientations;

V is selected from nitro, cyano, amino, hydroxy, alkoxy, alkylthio, alkylamino, dialkylamino, —NH(CH₂)ₚNH₂ wherein p is 1 to 4, —N(CH₂CH₂NH₂)₂, diazamino, amidino, guanidino, guanyl, semicarbazone, aryl, heterocycle, and heteroaryl, any of which is optionally substituted with one or more of amino, halo, cyano, nitro, hydroxy, —NH(CH₂)ₚNH₂ wherein p is 1 to 4, —N(CH₂CH₂NH₂)₂, amidino, guanidino, guanyl, aminosulfonyl, aminoalkoxy, aminoalkylthio, lower acylamino, or benzyloxycarbonyl;

the —(CH₂)_{pPL}— alkylene chain is optionally substituted with one or more amino or hydroxy groups, or is unsaturated;

pPL is 0 to 8;

q1PL and q2PL are, independently, 0, 1, or 2; and

m is 1 to about 20.

In some embodiments, the compound is a compound of Formula XXIIa, Formula XXIIb, or Formula XXIIc:

R¹—X-A₁-X—Z—Y-A₂-Y—R² XXIIa

R¹—X-A₁-X—Z—Y-A₂-Y—Z—X-A₁-X—R² XXIIb

R¹—X-A₁-X—Z—Y-A₂-Y—Z—X-A₁-X—Z—Y-A₂-Y—R² XXIIc

wherein: X is NR⁸, —NR⁸NR⁸—, C═O, or O; Y is NR⁸, —NR⁸NR⁸—, C═O, S, or O; R⁸ is hydrogen or alkyl; Z is C═O, C═S, O═S═O, —NR⁸NR⁸—, or —C(═O)C(═O)—; A₁ and A₂ are, independently, optionally substituted arylene or optionally substituted heteroarylene, wherein A₁ and A₂ are, independently, optionally substituted with one or more polar (PL) group(s), one or more non-polar (NPL) group(s), or a combination of one or more polar (PL) group(s) and one or more non-polar (NPL) group(s); R¹ is hydrogen, a polar group (PL), or a non-polar group (NPL); R² is R¹; NPL is a nonpolar group —(NR³′)_{q1NPL}—U^{NPL}—(CH₂)_{pNPL}—(NR³″)_{q2NPL}—R⁴′, wherein: R³, R³′, and R³″ are, independently, selected from hydrogen, alkyl, and alkoxy; R⁴ and R⁴′ are, independently, selected from hydrogen, alkyl, alkenyl, alkynyl, cycloalkyl, aryl, and heteroaryl, any of which is optionally substituted with one or more alkyl or halo groups; U^{NPL} is absent or selected from O, S, S(═O), S(═O)₂, NR³, —C(═O)—, —C(═O)—N═N—NR³—, —C(═O)—NR³—N═N—, —N═N—NR³—, —C(═N—N(R³)₂)—, —C(═NR³)—, —C(═O)O—, —C(═O)S—, —C(═S)—, —O—P(═O)₂O—, —R³⁰—, —R³S—, —S—C═N—, and —C(═O)—NR³—O—, wherein groups with two chemically nonequivalent termini can adopt both possible orientations; the —(CH₂)_{pNPL}— alkylene chain is optionally substituted with one or more amino or hydroxy groups, or is unsaturated; pNPL is 0 to 8; q1NPL and q2NPL are, independently, 0, 1, or 2; PL is a polar group selected from halo, hydroxyethoxymethyl, methoxyethoxymethyl, polyoxyethylene, and —(NR⁵′)_{q1PL}—U^{PL}—(CH₂)_{pPL}—(NR⁵′)_{q2PL}—V, wherein: R⁵, R⁵′, and R⁵″ are, independently, selected from hydrogen, alkyl, and alkoxy; U^{PL} is absent or selected from O, S, S(═O), S(═O)₂, NR⁵, —C(═O)—, —C(═O)—N═N—NR⁵—, —C(═O)—NR⁵—N═N—, —N═N—NR⁵—, —C(═N—N(R⁵)₂)—, —C(═NR⁵)—, —C(═O)O—, —C(═O)S—, —C(═S)—, —O—P(═O)₂O—, —R⁵O—, —R⁵S—, —S—C═N—, and —C(═O)—NR⁵—O—, wherein groups with two chemically nonequivalent termini can adopt both possible orientations; V is selected from nitro, cyano, amino, hydroxy, alkoxy, alkylthio, alkylamino, dialkylamino, —NH(CH₂)ₚNH₂ wherein p is 1 to 4, —N(CH₂CH₂NH₂)₂, diazamino, amidino, guanidino, guanyl, semicarbazone, aryl, heterocycle, and heteroaryl, any of which is optionally substituted with one or more of amino, halo, cyano, nitro, hydroxy, —NH(CH₂)ₚNH₂ wherein p is 1 to 4, —N(CH₂CH₂NH₂)₂, amidino, guanidino, guanyl, aminosulfonyl, aminoalkoxy, aminoalkylthio, lower acylamino, or benzyloxycarbonyl; the —(CH₂)_{pPL}— alkylene chain is optionally substituted with one or more amino or hydroxy groups, or is unsaturated; pPL is 0 to 8; and q 1 PL and q2PL are, independently, 0, 1, or 2.

In some embodiments, the immune response is against an oral pathogen. In some embodiments, the oral pathogen is chosen from Aggregatibacter spp. such as, for example, Aggregatibacter actinomycetemcomitans; Porphyromonas spp. such as, for example, Porphyromonas gingivalis; Streptococcus spp. such as, for example, Streptococcus sanguis and Streptococcus mutans, Candida spp. such as, for example, Candida albicans, Candida glabrata, Candida krusei, Candida dubliniensis, Candida parapsilosis, and Candida tropicalis; Actinomyces spp. such as, for example, Actinomyces viscosus; and Lactobacillus spp. such as, for example, Lactobacillus casei. In some embodiments, the immune response is against a bacterial pathogen. In some embodiments, the bacterial pathogen is chosen from Staphylococcus spp., such as, for example, Staphylococcus aureus, methicillin-resistant Staphylococcus aureus, and Staphylococcus epidermidis; Streptococcus spp. such as, for example, Streptococcus pneumoniae, Streptococcus pyogenes, and Streptococcus viridans; Escherichia spp. such as, for example, E. coli; Enterococcus spp. such as, for example, Enterococcus faecalis and Enterococcus faecium; Psuedomonas spp. such as, for example, Pseudomonas aeruginosa; Acinetobacter spp. such as, for example, A. baumannii; Haemophilus spp. such as, for example, Haemophilus influenzae; Serratia spp. such as, for example, Serratia marcescens; Moraxella spp. such as, for example, Moraxella catarrhalis; Klebsiella spp. such as, for example, Klebsiella pneumoniae; Proteus spp. such as, for example, Proteus vulgaris and Proteus mirabilis; Bacteroides spp. such as, for example, Bacteroides fragalis; Clostridium spp. such as, for example, Clostridium difficile and Clostridium perfringens; and Propionibacterium spp. such as, for example, Propionibacterium acnes.

The present invention also provides methods of modulating an immune response in a mammal comprising administering to the mammal in need thereof a therapeutically effective amount of a compound of Formula XXIII:

R¹-[-A₁-W-A₂-W-]ₘ—R² XXIII

or a pharmaceutically acceptable salt thereof,
wherein:

A₁ and A₂ are, independently, optionally substituted arylene or optionally substituted heteroarylene, wherein:

(i) A₁ and A₂ are, independently, optionally substituted with one or more polar (PL) group(s), one or more non-polar (NPL) group(s), or a combination of one or more polar (PL) group(s) and one or more non-polar (NPL) group(s); or

(ii) one of A₁ or A₂ is as defined above and is optionally substituted with one or more polar (PL) group(s), one or more non-polar (NPL) group(s), or a combination of one or more polar (PL) group(s) and one or more non-polar (NPL) group(s); and the other of A₁ or A₂ is the group —C≡C(CH₂)ₚC≡C—, wherein p is 0 to 8, and the —(CH₂)ₚ— alkylene chain is optionally substituted with one or more amino or hydroxyl groups;

W is absent, or represents —CH₂—, —CH₂—CH₂—, —CH═CH—, or —C≡C—;

R¹ is
(i) hydrogen, a polar group (PL), or a non-polar group (NPL), and R² is -A₁-R¹, wherein A₁ is as defined above and is optionally substituted with one or more polar (PL) group(s), one or more non-polar (NPL) group(s), or a combination of one or more polar (PL) group(s) and one or more non-polar (NPL) group(s); or
(ii) hydrogen, a polar group (PL), or a non-polar group (NPL), and R² is -A₁-W-A₂-R¹, wherein each of A₁ and A₂ is as defined above and is optionally substituted with one or more polar (PL) group(s), one or more non-polar (NPL) group(s), or a combination of one or more polar (PL) group(s) and one or more non-polar (NPL) group(s); or
(iii) A′-W— and R² is -A₁-W-A′, wherein A′ is aryl or heteroaryl, either of which is optionally substituted with one or more polar (PL) group(s), one or more non-polar (NPL) group(s), or a combination of one or more polar (PL) group(s) and one or more non-polar (NPL) group(s); or
(iv) A′-W— and R² is -A′, wherein A′ is aryl or heteroaryl, either of which is optionally substituted with one or more polar (PL) group(s), one or more non-polar (NPL) groups(s), or a combination of one or more polar (PL) group(s) and one or more non-polar (NPL) group(s); or
(iv) R¹ and R² together form a single bond;

NPL is a nonpolar group independently selected from —B(OR⁴)₂ or

—(NR³′)_{q1NPL}—U^{NPL}—(CH₂)_{pNPL}—(NR³″)_{q2NPL}—R⁴, wherein:

R³, R³′, and R³″ are, independently, selected from hydrogen, alkyl, and alkoxy;

R⁴ is selected from hydrogen, alkyl, alkenyl, alkynyl, cycloalkyl, aryl, and heteroaryl, any of which is optionally substituted with one or more alkyl or halo groups;

U^{NPL} is absent or selected from O, S, S(═O), S(═O)₂, NR³, —(C═O)—, —(C═O)—N═N—NR³—, —(C═O)—NR³—N═N—, —N═N—NR³—, —C(═N—N(R³)₂)—, —C(═NR³)—, —C(═O)O—, —C(═O)S—, —C(═S)—, —O—P(═O)₂O—, —R³⁰—, —R³S—, —S—C═N— and —(C═O)—NR³—O—, wherein groups with two chemically nonequivalent termini can adopt both possible orientations;

the —(CH₂)_{pNPL}— alkylene chain is optionally substituted with one or more alkyl, amino or hydroxyl groups, or the alkylene chain is unsaturated;

pNPL is 0 to 8;

q1NPL and q2NPL are, independently, 0 to 2;

PL is a polar group selected from halo, hydroxyethoxymethyl, methoxyethoxymethyl, polyoxyethylene, and —(NR⁵′)_{q1PL}—U^{PL}—(CH₂)_{pPL}—(NR⁵′)_{q2PL}—V, wherein:

R⁵, R⁵′, and R⁵″ are, independently, selected from hydrogen, alkyl, and alkoxy;

U^{PL} is absent or selected from O, S, S(═O), S(═O)₂, NR⁵, —(C═O)—, —(C═O)—N═N—NR⁵—, —(C═O)—NR⁵—N═N—, —N═N—NR⁵—, —C(═N—N(R⁵)₂)—, —C(═NR⁵)—, —C(═O)O—, —C(═O)S—, —C(═S)—, —O—P(═O)₂O—, —R⁵O—, —R⁵S—, —S—C═N—, and —(C═O)—NR⁵—O—, wherein groups with two chemically nonequivalent termini can adopt both possible orientations;

V is selected from nitro, cyano, amino, hydroxyl, alkoxy, alkylthio, alkylamino, dialkylamino, —NH(CH₂)ₚNH₂, —N(CH₂CH₂NH₂)₂, diazamino, amidino, guanidino, guanyl, semicarbazone, aryl, heterocycle, and heteroaryl, any of which is optionally substituted with one or more of amino, halo, cyano, nitro, hydroxyl, —NH(CH₂)ₚNH₂, —N(CH₂CH₂NH₂)₂, amidino, guanidino, guanyl, aminosulfonyl, aminoalkoxy, aminoalkylthio, lower acylamino, or benzyloxycarbonyl; the —(CH₂)_{pPL}— alkylene chain is optionally substituted with one or more amino or hydroxyl groups, or the alkylene chain is unsaturated;

pPL is 0 to 8;

q1 PL and q2PL are, independently, 0 to 2; and

m is 1 to about 25.

In some embodiments, the compound of Formula XXIII is of Formula XXIIIa:

R¹-A₁-W-A₂-W-A₁-R² XXIIIa

wherein:

A₁ and A₂ are, independently, optionally substituted arylene or optionally substituted heteroarylene, wherein:
(i) A₁ and A₂ are, independently, optionally substituted with one or more polar (PL) group(s), one or more non-polar (NPL) group(s), or a combination of one or more polar (PL) group(s) and one or more non-polar (NPL) group(s); or
(ii) one of A₁ or A₂ is as defined above and is optionally substituted with one or more polar (PL) group(s), one or more non-polar (NPL) group(s), or a combination of one or more polar (PL) group(s) and one or more non-polar (NPL) group(s); and the other of A₁ or A₂ is the group —C≡C(CH₂)ₚC≡C—, wherein p is 0 to 8, and the —(CH₂)ₚ— alkylene chain is optionally substituted with one or more amino or hydroxyl groups;

W is —C≡C—;

R¹ is hydrogen, a polar group (PL), a non-polar group (NPL), or —W-A′, wherein A′ is aryl or heteroaryl, either of which is optionally substituted with one or more polar (PL) group(s), one or more non-polar (NPL) group(s), or a combination of one or more polar (PL) group(s) and one or more non-polar (NPL) group(s);

R² is R¹;

NPL is a nonpolar group —(NR³)_{q1NPL}—U^{NPL}—(CH₂)_{pNPL}—(NR³″)_{q2NPL}—R⁴;

R³, R³′, and R³″ are, independently, selected from hydrogen, alkyl, and alkoxy;

R⁴ is selected from hydrogen, alkyl, alkenyl, alkynyl, cycloalkyl, aryl, and heteroaryl, any of which is optionally substituted with one or more alkyl or halo groups;

U^{NPL} is absent or selected from O, S, S(═O), S(═O)₂, NR³, —(C═O)—, —(C═O)—N═N—NR³—, —(C═O)—NR³—N═N—, —N═N—NR³—, —C(═N—N(R³)₂)—, —C(═NR³)—, —C(═O)O—, —C(═O)S—, —C(═S)—, —O—P(═O)₂O—, —R³—O—, —R³—S—, —S—C═N—, and —(C═O)—NR³—O—, wherein groups with two chemically nonequivalent termini can adopt both possible orientations;

the alkylene chain —(CH₂)_{pNPL}— is optionally substituted with one or more alkyl, amino or hydroxyl groups, or the alkylene chain is unsaturated;

pNPL is 0 to 8;

q1NPL and q2NPL are, independently, 0 to 2;

PL is a polar group selected from halo, hydroxyethoxymethyl, methoxyethoxymethyl, polyoxyethylene, and —(NR⁵)_{q1PL}—U^{PL}—(CH₂)_{pPL}—(NR⁵′)_{q2PL}—V,

wherein:

R⁵, R⁵′, and R⁵″ are, independently, selected from hydrogen, alkyl, and alkoxy;

U^{PL} is absent or selected from O, S, S(═O), S(═O)₂, NR⁵, —(C═O)O—, —(C═O)—N═N—NR⁵—, —(C═O)—NR⁵—N═N—, —N═N—NR⁵—, —C(═N—N(R⁵)₂)—, —C(═NR⁵)—, —C(═O)O—, —C(═O)S—, —C(═S)—, —O—P(═O)₂O—, —R⁵O—, —R⁵S—, —S—C═N—, and —(C═O)—NR⁵—O—, wherein groups with two chemically nonequivalent termini can adopt both possible orientations;

V is selected from nitro, cyano, amino, hydroxyl, alkoxy, alkylthio, alkylamino, dialkylamino, —NH(CH₂)ₚNH₂, —N(CH₂CH₂NH₂)₂, diazamino, amidino, guanidino, guanyl, semicarbazone, aryl, heterocycle, and heteroaryl, any of which is optionally substituted with one or more of amino, halo, cyano, nitro, hydroxyl, —NH(CH₂)ₚNH₂, —N(CH₂CH₂NH₂)₂, amidino, guanidino, guanyl, aminosulfonyl, aminoalkoxy, aminoalkylthio, lower acylamino, or benzyloxycarbonyl;

the alkylene chain —(CH₂)_{pPL}— is optionally substituted with one or more amino or hydroxyl groups, or the alkylene chain is unsaturated;

pPL is 0 to 8; and

q1PL and q2PL are, independently, 0 to 2.

In some embodiments, A₁ and A₂ are, independently, optionally substituted m-phenylene, wherein A₁ is optionally substituted with two polar (PL) groups, and A₂ is unsubstituted; R¹ is a polar group; PL is independently halo or —(NR⁵′)_{q1PL}—U^{PL}—(CH₂)_{pPL}—(NR⁵′)_{q2PL}—V, wherein: U^{PL} is absent or selected from O, S, NR⁵, and —C(═O)—; V is selected from amino, amidino, and guanidino, any of which is optionally substituted with one or more of amino, halo, —NH(CH₂)ₚNH₂ wherein p is 1 to 4, —N(CH₂CH₂NH₂)₂, am idino, guanidino, guanyl, aminosulfonyl, aminoalkoxy, aminoalkylthio, and lower acylamino; pPL is 0 to 8; and q1PL and q2PL are 0.

In some embodiments, R¹ is halo; PL is or —U^{PL}—(CH₂)_{pPL}—V, wherein: U^{PL} is absent; V is selected from amino, amidino, and guanidino, any of which is optionally substituted with one or more of amino and halo; and pPL is 0 to 6.

The present invention also provides methods of modulating an immune response in a mammal comprising administering to the mammal in need thereof a therapeutically effective amount of a compound chosen from:

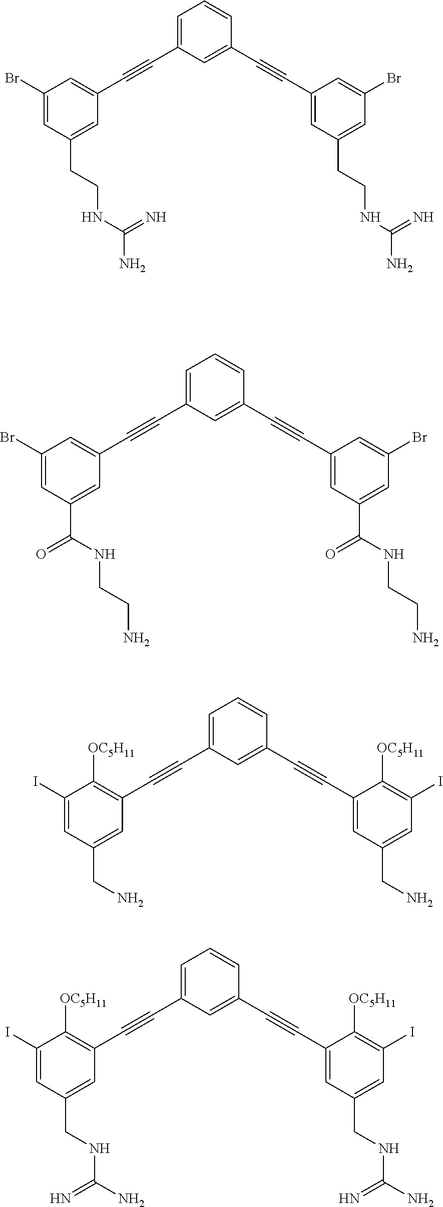

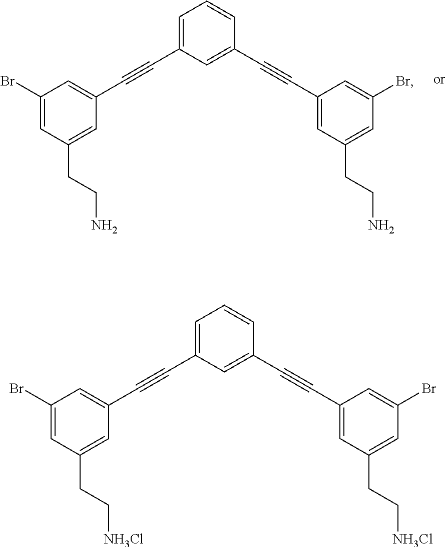

or a pharmaceutically acceptable salt thereof.

In some embodiments, the immune response is against an oral pathogen. In some embodiments, the oral pathogen is chosen from Aggregatibacter spp. such as, for example, Aggregatibacter actinomycetemcomitans; Porphyromonas spp. such as, for example, Porphyromonas gingivalis; Streptococcus spp. such as, for example, Streptococcus sanguis and Streptococcus mutans, Candida spp. such as, for example, Candida albicans, Candida glabrata, Candida krusei, Candida dubliniensis, Candida parapsilosis, and Candida tropicalis; Actinomyces spp. such as, for example, Actinomyces viscosus; and Lactobacillus spp. such as, for example, Lactobacillus casei. In some embodiments, the immune response is against a bacterial pathogen. In some embodiments, the bacterial pathogen is chosen from Staphylococcus spp., such as, for example, Staphylococcus aureus, methicillin-resistant Staphylococcus aureus, and Staphylococcus epidermidis; Streptococcus spp. such as, for example, Streptococcus pneumoniae, Streptococcus pyogenes, and Streptococcus viridans; Escherichia spp. such as, for example, E. coli; Enterococcus spp. such as, for example, Enterococcus faecalis and Enterococcus faecium; Psuedomonas spp. such as, for example, Pseudomonas aeruginosa; Acinetobacter spp. such as, for example, A. baumannii; Haemophilus spp. such as, for example, Haemophilus influenzae; Serratia spp. such as, for example, Serratia marcescens; Moraxella spp. such as, for example, Moraxella catarrhalis; Klebsiella spp. such as, for example, Klebsiella pneumoniae; Proteus spp. such as, for example, Proteus vulgaris and Proteus mirabilis; Bacteroides spp. such as, for example, Bacteroides fragalis; Clostridium spp. such as, for example, Clostridium difficile and Clostridium perfringens; and Propionibacterium spp. such as, for example, Propionibacterium acnes.

The present invention also provides methods of modulating an immune response in a mammal comprising administering to the mammal in need thereof a therapeutically effective amount of a compound of Formula XXIV:

R¹—X-A₁-X—Y-A₂-Y—X-A₁-X—R² XXIV

or a pharmaceutically acceptable salt thereof,
wherein:

X is NR⁸, O, S, or —N(R⁸)N(R⁸)—;

Y is C═O, C═S, or O═S═O;

R⁸ is hydrogen or alkyl;

A₁ and A₂ are, independently, optionally substituted arylene or optionally substituted heteroarylene, wherein A₁ and A₂ are, independently, optionally substituted with one or more polar (PL) group(s), one or more non-polar (NPL) group(s), or a combination of one or more polar (PL) group(s) and one or more non-polar (NPL) group(s);

R¹ is a polar group (PL) or a non-polar group (NPL);

R² is R¹;

NPL is a nonpolar group independently selected from —B(OR⁴)₂ and —(NR³′)_{q1NPL}—U^{NPL}—(CH₂)_{pNPL}—(NR³″)_{q2NPL}—R^{4′}, wherein:

R³, R³′, and R³″ are, independently, selected from hydrogen, alkyl, and alkoxy;

R⁴ and R⁴′ are, independently, selected from the group consisting of hydrogen, alkyl, alkenyl, alkynyl, cycloalkyl, aryl, and heteroaryl, any of which is optionally substituted with one or more alkyl or halo groups;

U^{NPL} is absent or selected from O, S, S(═O), S(═O)₂, NR³, —C(═O)—, C(═O)—N═N—NR³—, —C(═O)—NR³—N═N—, —N═N—NR³—, —C(═N—N(R³)₂)—, —C(═NR³)—, —C(═O)O—, —C(═O)S—, —C(═S)—, —O—P(═O)₂O—, —R³⁰—, —R³S—, —S—C═N—, and —C(═O)—NR³—O—, wherein groups with two chemically nonequivalent termini can adopt both possible orientations;

the —(CH₂)_{pNPL}— alkylene chain is optionally substituted with one or more amino or hydroxy groups, or is unsaturated;

pNPL is 0 to 8;

q1NPL and q2NPL are, independently, 0, 1, or 2;

PL is a polar group selected from halo, hydroxyethoxymethyl, methoxyethoxymethyl, polyoxyethylene, and —(NR⁵)_{q1PL}—U^{PL}—(CH₂)_{pPL}—(NR⁵)_{q2PL}—V,

wherein:

R⁵, R⁵′, and R⁵″ are, independently, selected from hydrogen, alkyl, and alkoxy;

U^{PL} is absent or selected from O, S, S(═O), S(═O)₂, NR⁵, —C(═O)—, —C(═O)—N═N—NR⁵—, —C(═O)—NR⁵—N═N—, —N═N—NR⁵—, —C(═N—N(R⁵)₂)—, —C(═NR⁵)—, —C(═O)O—, —C(═O)S—, —C(═S)—, —O—P(═O)₂O—, —R⁵O—, —R⁵S—, —S—C═N—, and —C(═O)—NR⁵—O—, wherein groups with two chemically nonequivalent termini can adopt both possible orientations;

V is selected from nitro, cyano, amino, hydroxy, alkoxy, alkylthio, alkylamino, dialkylamino, —NH(CH₂)ₚNH₂ wherein p is 1 to 4, —N(CH₂CH₂NH₂)₂, diazamino, amidino, guanidino, guanyl, semicarbazone, aryl, heterocycle and heteroaryl, any of which is optionally substituted with one or more of amino, halo, cyano, nitro, hydroxy, —NH(CH₂)ₚNH₂ wherein p is 1 to 4, —N(CH₂CH₂NH₂)₂, amidino, guanidino, guanyl, aminosulfonyl, aminoalkoxy, aminoalkylthio, lower acylamino, or benzyloxycarbonyl;

the —(CH₂)_{pPL}— alkylene chain is optionally substituted with one or more amino or hydroxy groups, or is unsaturated;

pPL is 0 to 8; and

q1PL and q2PL are, independently, 0, 1, or 2.

In some embodiments, A₁ is m-phenylene substituted with one (PL) group and one non-polar (NPL) group; A₂ is unsubstituted m-pyrimidinylene or m-pyrimidinylene substituted with one or two polar (PL) group(s); NPL is R⁴′, wherein R⁴′ is (C₁-C₆)alkyl optionally substituted with one or more halo groups; PL is —U^{PL}—(CH₂)_{pPL}—V, wherein: U^{PL} is O or S; V is selected from amino, amidino, and guanidino; and pPL is 0 to 6.

In some embodiments, A, is m-phenylene substituted with one (PL) group and one non-polar (NPL) group; A₂ is unsubstituted m-phenylene or m-phenylene substituted with one or two polar (PL) group(s); NPL is R⁴′, wherein R⁴′ is (C₁-C₆)alkyl optionally substituted with one or more halo groups; PL is —U^{PL}—(CH₂)_{pPL}—V, wherein: U^{PL} is O or S; V is selected from amino, amidino, and guanidino; and pPL is 0 to 6.

The present invention also provides methods of modulating an immune response in a mammal comprising administering to the mammal in need thereof a therapeutically effective amount of a compound chosen from:

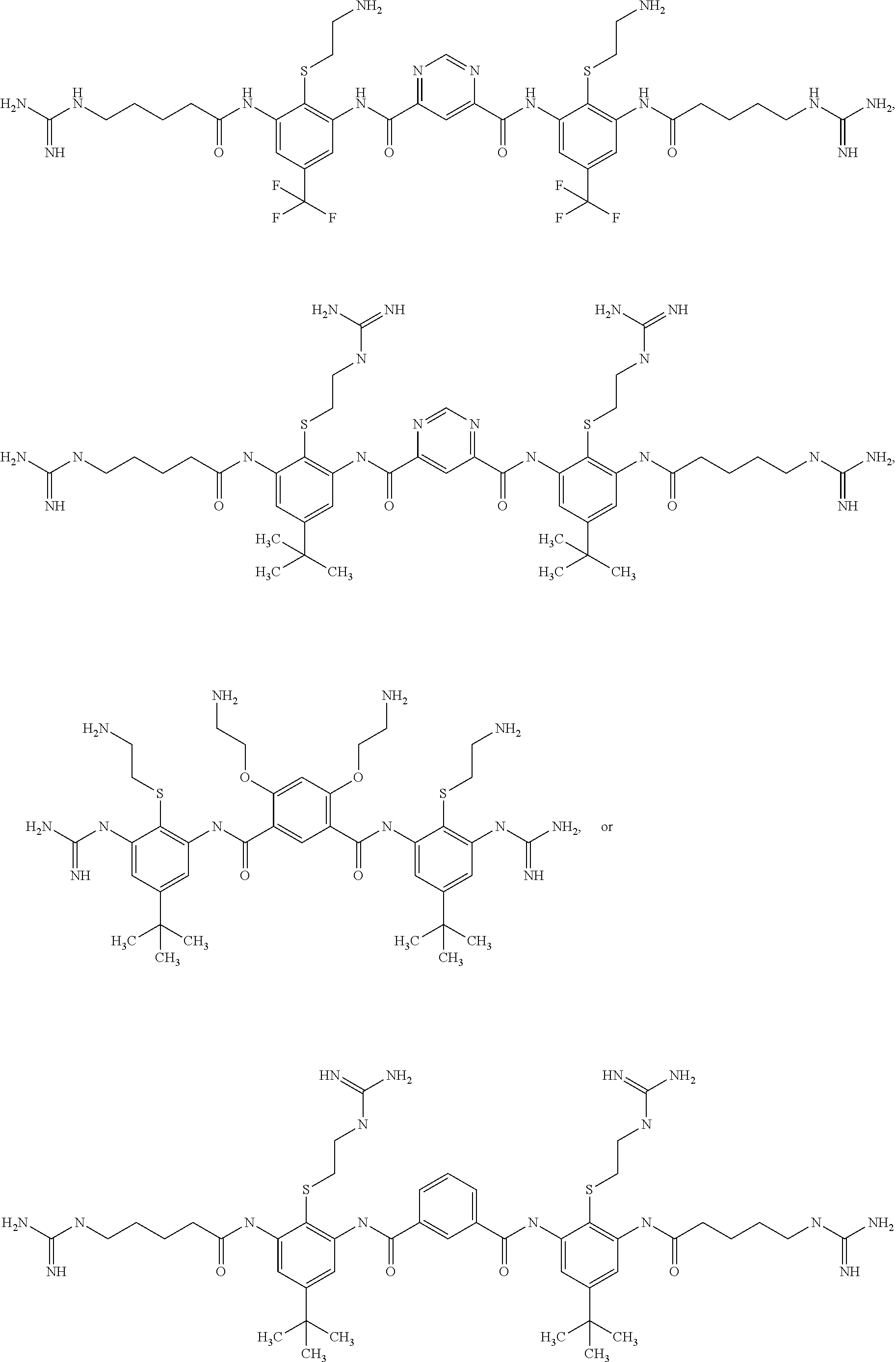

or a pharmaceutically acceptable salt thereof.

In some embodiments, the immune response is against an oral pathogen. In some embodiments, the oral pathogen is chosen from Aggregatibacter spp. such as, for example, Aggregatibacter actinomycetemcomitans; Porphyromonas spp. such as, for example, Porphyromonas gingivalis; Streptococcus spp. such as, for example, Streptococcus sanguis and Streptococcus mutans, Candida spp. such as, for example, Candida albicans, Candida glabrata, Candida krusei, Candida dubliniensis, Candida parapsilosis, and Candida tropicalis; Actinomyces spp. such as, for example, Actinomyces viscosus; and Lactobacillus spp. such as, for example, Lactobacillus casei. In some embodiments, the immune response is against a bacterial pathogen. In some embodiments, the bacterial pathogen is chosen from Staphylococcus spp., such as, for example, Staphylococcus aureus, methicillin-resistant Staphylococcus aureus, and Staphylococcus epidermidis; Streptococcus spp. such as, for example, Streptococcus pneumoniae, Streptococcus pyogenes, and Streptococcus viridans; Escherichia spp. such as, for example, E. coli; Enterococcus spp. such as, for example, Enterococcus faecalis and Enterococcus faecium; Psuedomonas spp. such as, for example, Pseudomonas aeruginosa; Acinetobacter spp. such as, for example, A. baumannii; Haemophilus spp. such as, for example, Haemophilus influenzae; Serratia spp. such as, for example, Serratia marcescens; Moraxella spp. such as, for example, Moraxella catarrhalis; Klebsiella spp. such as, for example, Klebsiella pneumoniae; Proteus spp. such as, for example, Proteus vulgaris and Proteus mirabilis; Bacteroides spp. such as, for example, Bacteroides fragalis; Clostridium spp. such as, for example, Clostridium difficile and Clostridium perfringens; and Propionibacterium spp. such as, for example, Propionibacterium acnes.

The present invention discloses compositions comprising any of the compounds described herein or any combination thereof. Polymers are generally defined as synthetic compounds assembled from monomer subunits that are polydisperse in molecular weight, and are most commonly prepared by one-pot synthetic procedures. The term “polymer” as used herein refers to a macromolecule comprising a plurality of repeating units or monomers. The term includes homopolymers, which are formed from a single type of monomer, and copolymers, which are formed from two or more different monomers. In copolymers, the monomers may be distributed randomly (random copolymer), in alternating fashion (alternating copolymers), or in blocks (block copolymer). The polymers of the present invention are either homopolymers or alternating copolymers having about 2 monomer units to about 500 monomer units, with average molecular weights that range from about 300 Daltons to about 1,000,000 Daltons, or from about 400 Daltons to about 120,000 Daltons. Preferred polymers are those having about 5 to about 100 monomer units, with average molecular weights that range from about 1,000 Daltons to about 25,000 Daltons.

The term “oligomer” as used herein refers to a homogenous polymer with a defined sequence and molecular weight. Modern methods of solid phase organic chemistry have allowed the synthesis of homodisperse, sequence-specific oligomers with molecular weights approaching 5,000 Daltons. An oligomer, in contrast to a polymer, has a defined sequence and molecular weight and is usually synthesized either by solid phase techniques or by step-wise solution chemistry and purified to homogeneity. Oligomers of the present invention are those having about 2 monomer units to about 25 monomer units, with molecular weights that range from about 300 Daltons to about 6,000 Daltons. Suitable oligomers are those having about 2 monomer units to about 10 monomer units, with molecular weights that range from about 300 Daltons to about 2,500 Daltons.

The present invention also provides methods of modulating an immune response in a mammal comprising administering to the mammal in need thereof a therapeutically effective amount of a compound of Formula XXV:

A-(B)ₙ₁-(D)ₘ₁-H XXV

or a pharmaceutically acceptable salt thereof,
wherein:

A is the residue of a chain transfer agent;

B is —[CH₂—C(R¹¹)(B₁₁)]—, wherein B₁₁ is —X₁₁—Y₁₁—Z₁₁, wherein

X₁₁ is carbonyl (—C(═O)—) or optionally substituted C₁₋₆ alkylene; or X₁₁ is absent;

Y₁₁ is O, NH, or optionally substituted C₁₋₆ alkylene; or Y₁₁ is absent;

Z₁₁ is —Z_{11A}—Z_{11B}, wherein Z_{11A} is alkylene, arylene, or heteroarylene, any of which is optionally substituted; or Z_{11A} is absent; and Z_{11B} is -guanidine, -amidino, —N(R³)(R⁴), or —N⁺(R³)(R⁴)(R⁵), wherein R³, R⁴, and R⁵ are, independently, hydrogen, alkyl, aminoalkyl, aryl, heteroaryl, heterocyclic, or aralkyl; or

Z₁₁ is pyridinium

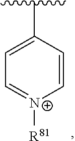

or phosphonium

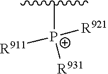

wherein R⁸¹, R⁹¹¹, R⁹²¹, and R⁹³¹ are, independently, hydrogen or alkyl;

R¹¹ is hydrogen or C₁₋₄ alkyl;

D is —[CH₂—C(R²¹)(D₂₁)]-, wherein D₂₁ is —X₂₁—Y₂₁—Z₂₁, wherein

X₂₁ is carbonyl (—C(═O)—) or optionally substituted C₁₋₆ alkylene; or X₂₁ is absent;

Y₂₁ is O, NH, or optionally substituted C₁₋₆ alkylene, or Y₂₁ is absent;

Z₂₁ is alkyl, cycloalkyl, alkoxy, aryl, or aralkyl, any of which is optionally substituted;

R²¹ is hydrogen or C₁₋₄ alkyl;

m₁, the mole fraction of D, is about 0.1 to about 0.9; and

n₁, the mole fraction of B, is 1−m₁;

wherein the compound is a random copolymer of B and D, and

wherein the copolymer has a degree of polymerization of about 5 to about 50.

In some embodiments, A is C₁₋₄ alkoxycarbonyl(C₁₋₄)alkylthio; X₁₁ and X₂₁ are carbonyl; Y₁₁ and Y₂₁ are 0; Z₁₁ is —Z_{11A}—Z_{11B}, wherein Z_{11A} is C₁₋₆ alkylene optionally substituted with C₁₋₄ alkyl or aryl; and Z_{11B} is —N(R³¹)(R⁴¹) or —N⁺(R³¹)(R⁴¹)(R⁵¹), wherein R³¹, R⁴¹, and R⁵¹ are independently hydrogen C₁₋₄ alkyl; Z₂₁ is C₁₋₆ alkyl, C₁₋₆ aryl, or C₁₋₆ ar(C₁₋₄)alkyl; and R¹¹ and R²¹ are, independently, hydrogen or methyl; m₁ is about 0.35 to about 0.60; and wherein the copolymer has a degree of polymerization of about 5 to about 10.

In some embodiments, the copolymer has a molecular weight from about 2,000 Daltons to about 15,000 Daltons. In some embodiments, the copolymer has a molecular weight from about 2,000 Daltons to about 3,000 Daltons. In some embodiments, the copolymer has a molecular weight from about 3,000 Daltons to about 4,000 Daltons. In some embodiments, the copolymer has a molecular weight from about 4000 Daltons to about 5,000 Daltons. In some embodiments, the copolymer has a molecular weight from about 5000 Daltons to about 6,000 Daltons. In some embodiments, the copolymer has a molecular weight from about 6,000 Daltons to about 7,000 Daltons. In some embodiments, the copolymer has a molecular weight from about 7,000 Daltons to about 8,000 Daltons. In some embodiments, the copolymer has a molecular weight from about 8,000 Daltons to about 9,000 Daltons. In some embodiments, the copolymer has a molecular weight from about 9,000 Daltons to about 10,000 Daltons. In some embodiments, the copolymer has a molecular weight from about 10,000 Daltons to about 11,000 Daltons. In some embodiments, the copolymer has a molecular weight from about 11,000 Daltons to about 12,000 Daltons.

In some embodiments, the copolymer is a polymethcrylate. In some embodiments, one of B and D is amino-ethyl methacrylate the other of B and D is butyl-methacrylate, ethyl-methacrylate, or methyl-methacrylate.

The present invention also provides methods of modulating an immune response in a mammal comprising administering to the mammal in need thereof a therapeutically effective amount of a compound chosen from:

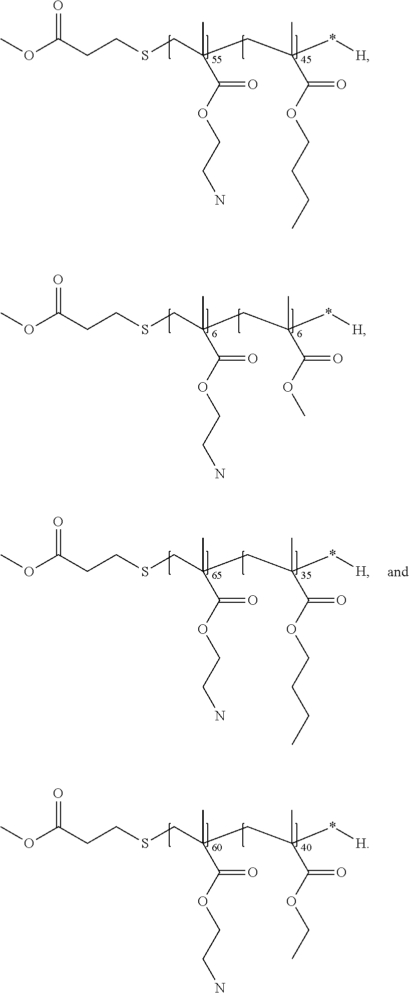

In some embodiments, the immune response is against an oral pathogen. In some embodiments, the oral pathogen is chosen from Aggregatibacter spp. such as, for example, Aggregatibacter actinomycetemcomitans; Porphyromonas spp. such as, for example, Porphyromonas gingivalis; Streptococcus spp. such as, for example, Streptococcus sanguis and Streptococcus mutans, Candida spp. such as, for example, Candida albicans, Candida glabrata, Candida krusei, Candida dubliniensis, Candida parapsilosis, and Candida tropicalis; Actinomyces spp. such as, for example, Actinomyces viscosus; and Lactobacillus spp. such as, for example, Lactobacillus casei. In some embodiments, the immune response is against a bacterial pathogen. In some embodiments, the bacterial pathogen is chosen from Staphylococcus spp., such as, for example, Staphylococcus aureus, methicillin-resistant Staphylococcus aureus, and Staphylococcus epidermidis; Streptococcus spp. such as, for example, Streptococcus pneumoniae, Streptococcus pyogenes, and Streptococcus viridans; Escherichia spp. such as, for example, E. coli; Enterococcus spp. such as, for example, Enterococcus faecalis and Enterococcus faecium; Psuedomonas spp. such as, for example, Pseudomonas aeruginosa; Acinetobacter spp. such as, for example, A. baumannii; Haemophilus spp. such as, for example, Haemophilus influenzae; Serratia spp. such as, for example, Serratia marcescens; Moraxella spp. such as, for example, Moraxella catarrhalis; Klebsiella spp. such as, for example, Klebsiella pneumoniae; Proteus spp. such as, for example, Proteus vulgaris and Proteus mirabilis; Bacteroides spp. such as, for example, Bacteroides fragalis; Clostridium spp. such as, for example, Clostridium difficile and Clostridium perfringens; and Propionibacterium spp. such as, for example, Propionibacterium acnes.

The present invention also provides methods of modulating an immune response in a mammal comprising administering to the mammal in need thereof a therapeutically effective amount of a compound chosen from:

| Compd. | |
|---|---|
| No. | Structure |
| 1 | 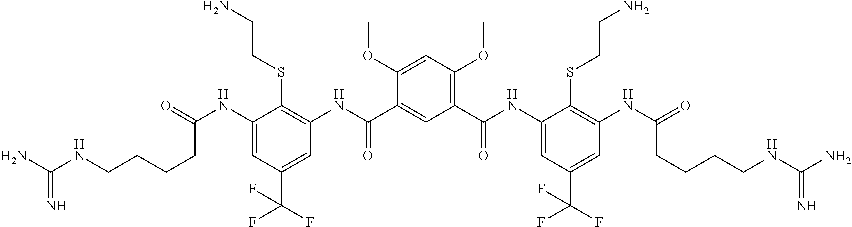 |
| 2 | 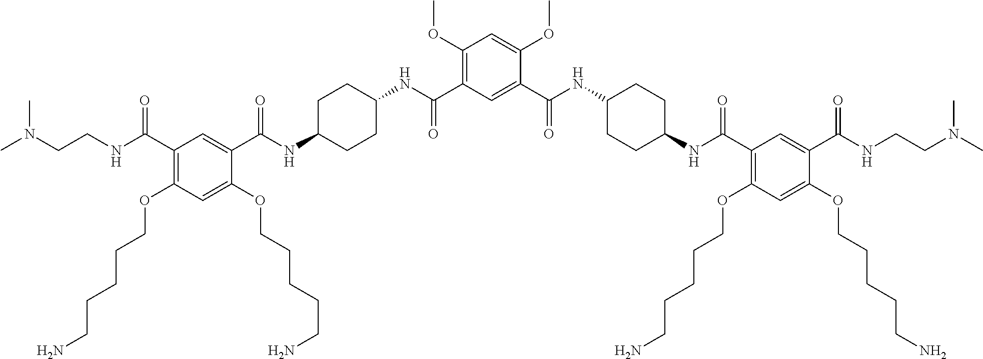 |
| 3 | 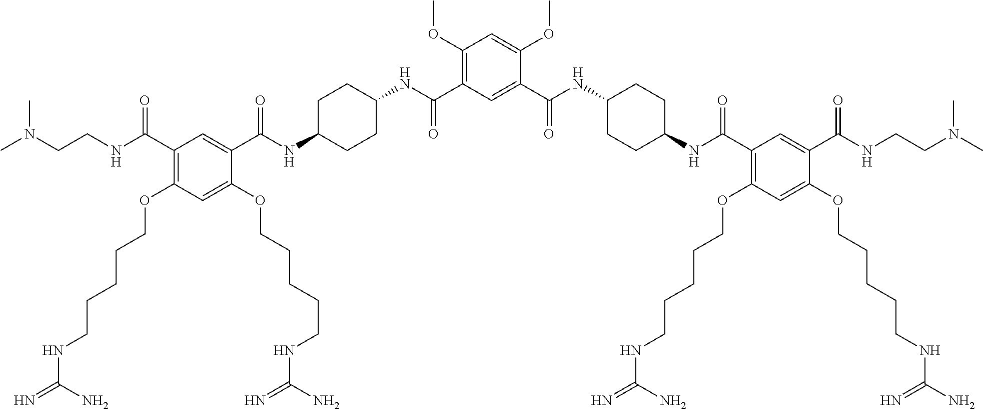 |
| 4 | 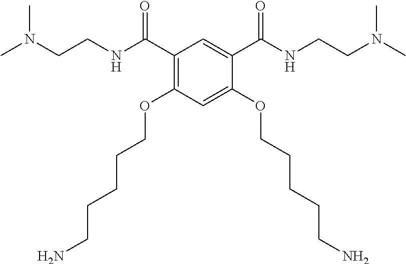 |
| 5 | 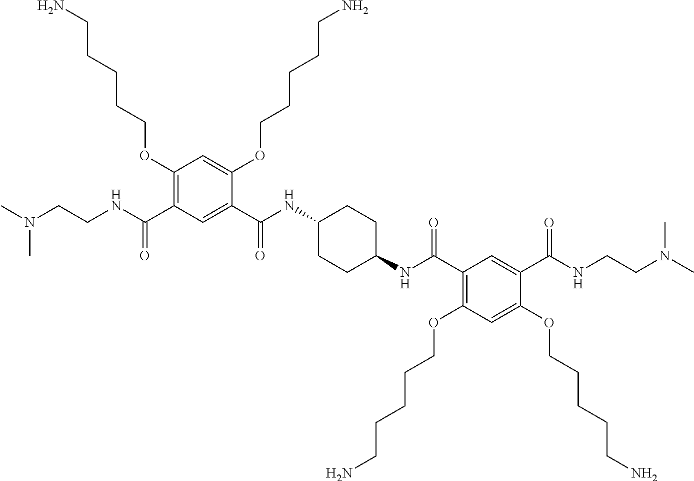 |
| 6 | 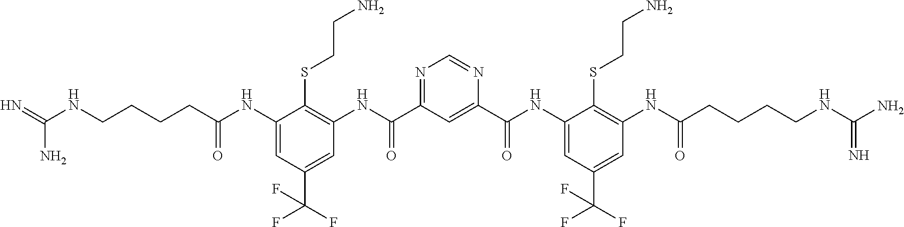 |
| 7 | 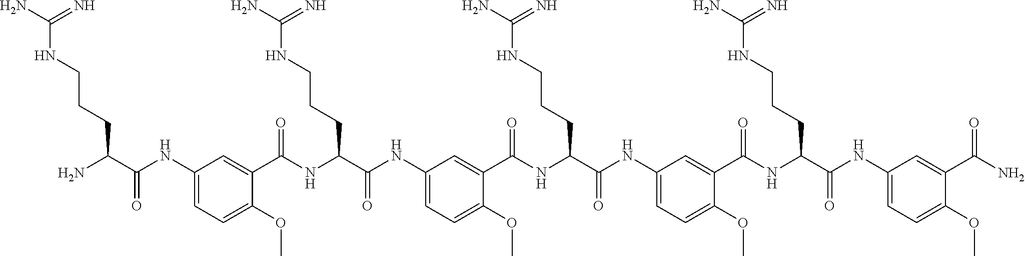 |
| 8 | 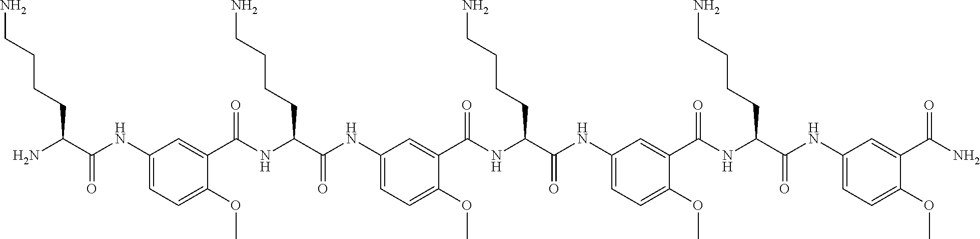 |
| 9 | 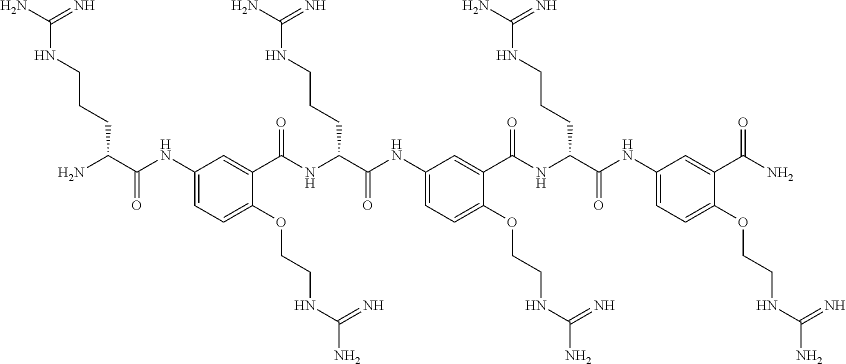 |
| 10 | 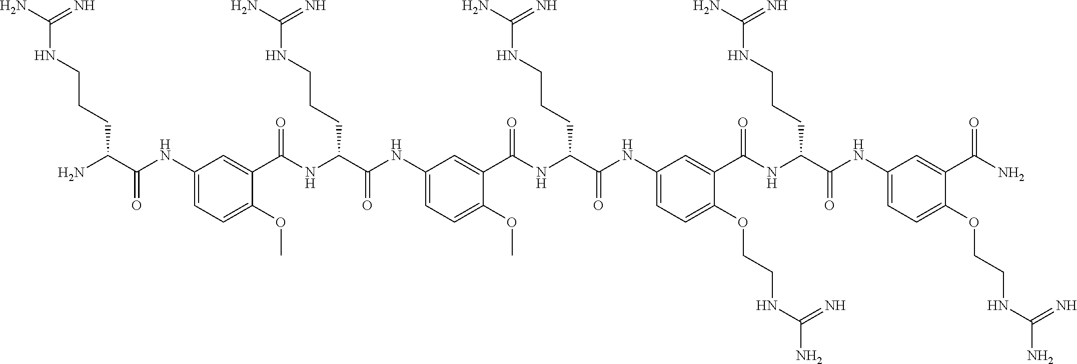 |
| 11 | 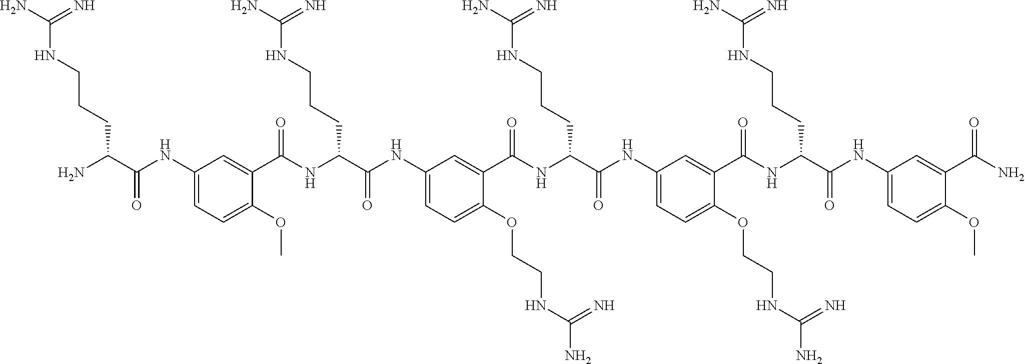 |
| 12 | 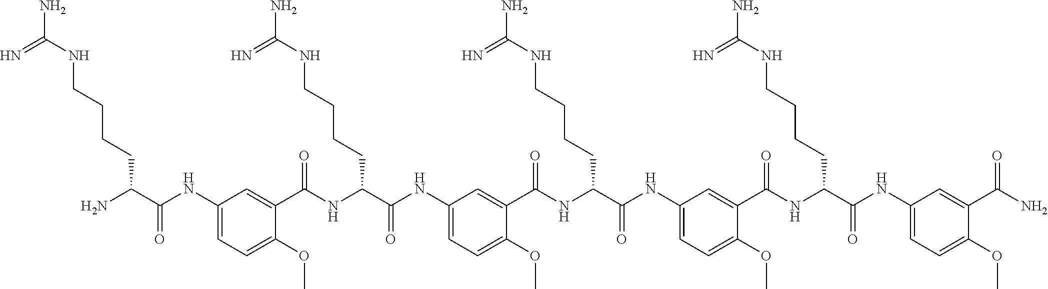 |
| 13 | 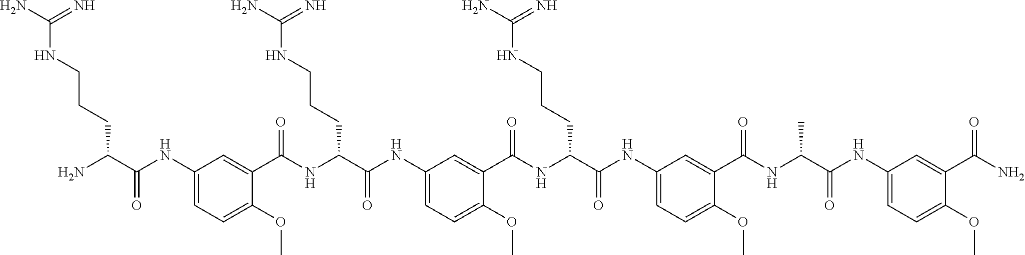 |
| 14 | 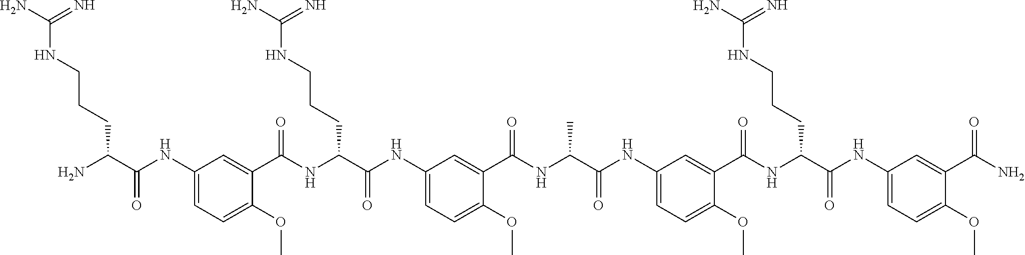 |
| 15 | 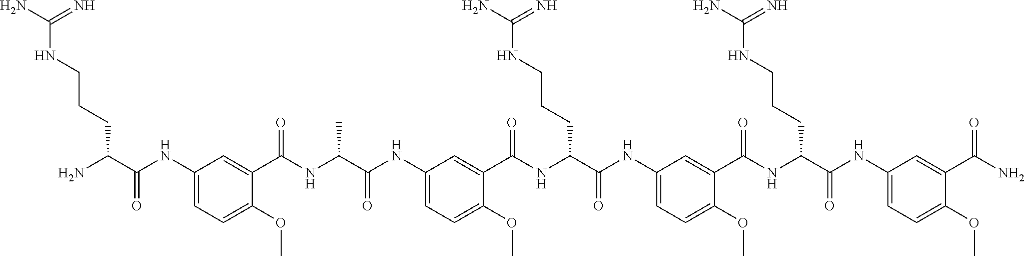 |
| 16 | 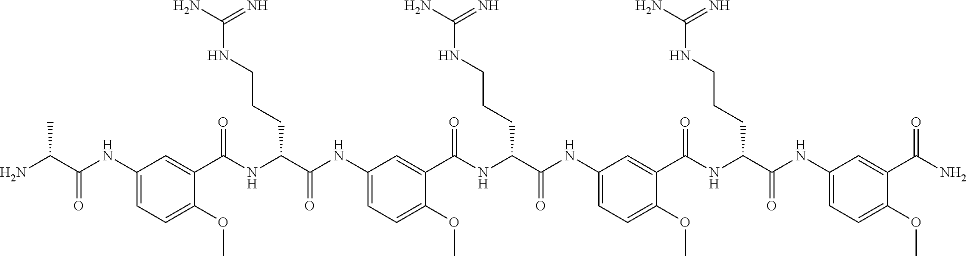 |
| 17 | 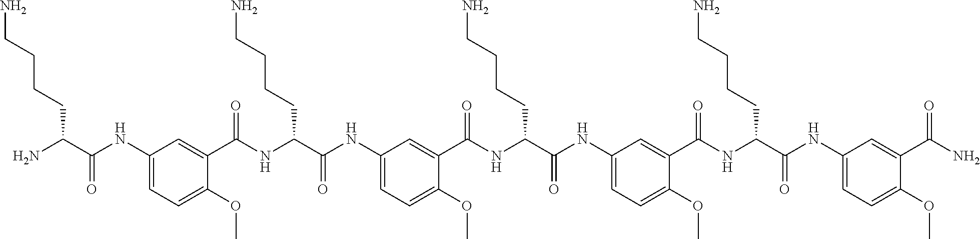 |
| 18 | 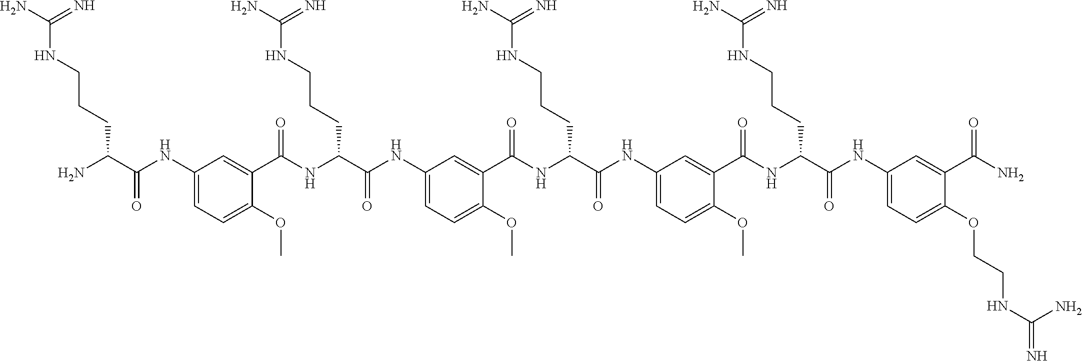 |
| 19 | 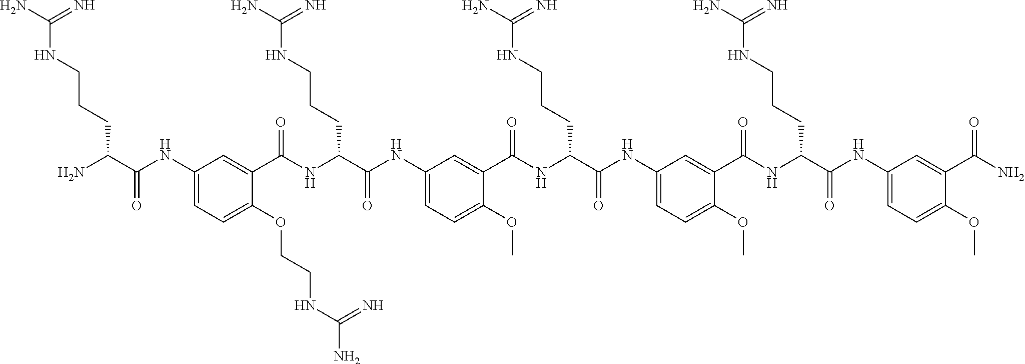 |
| 20 | 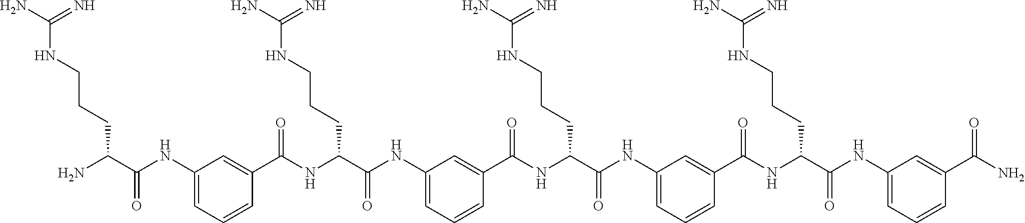 |
| 21 | 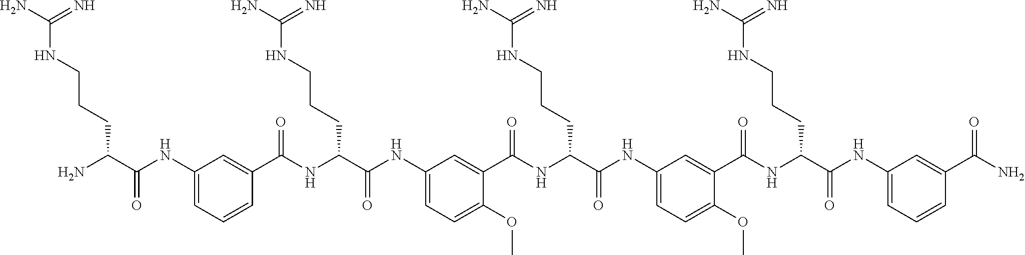 |
| 22 | 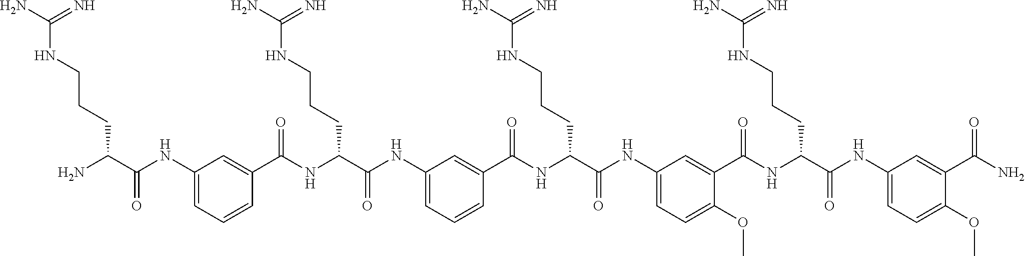 |
| 23 | 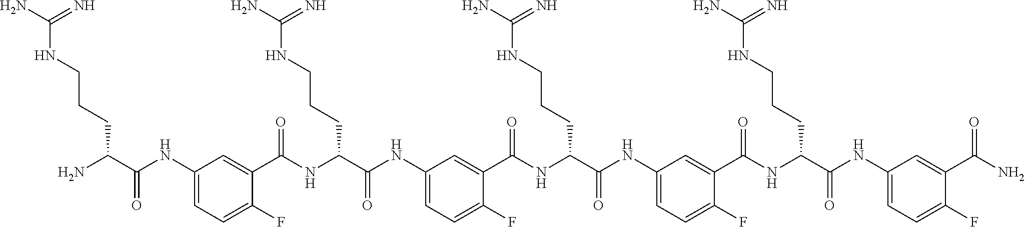 |
| 24 | 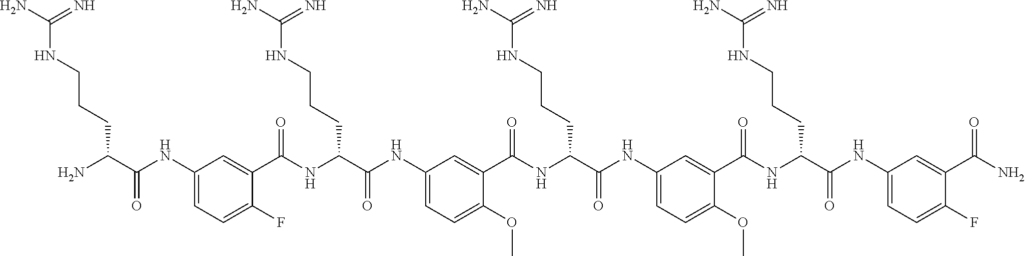 |
| 25 | 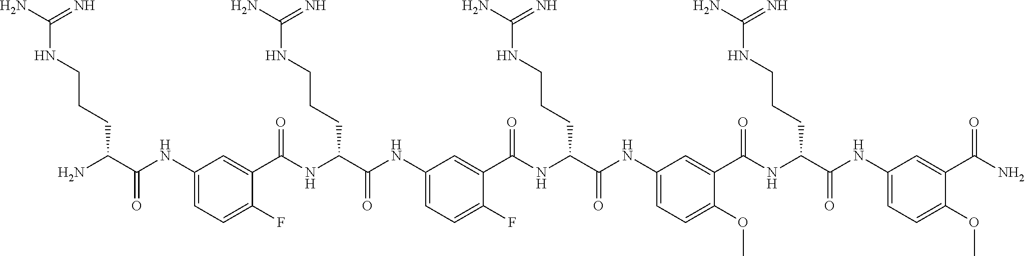 |
| 26 | 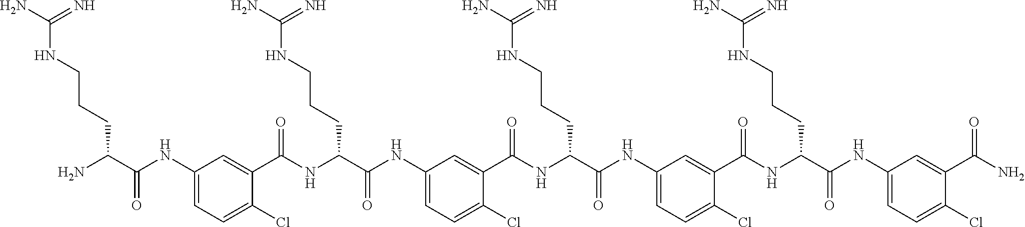 |
| 27 | 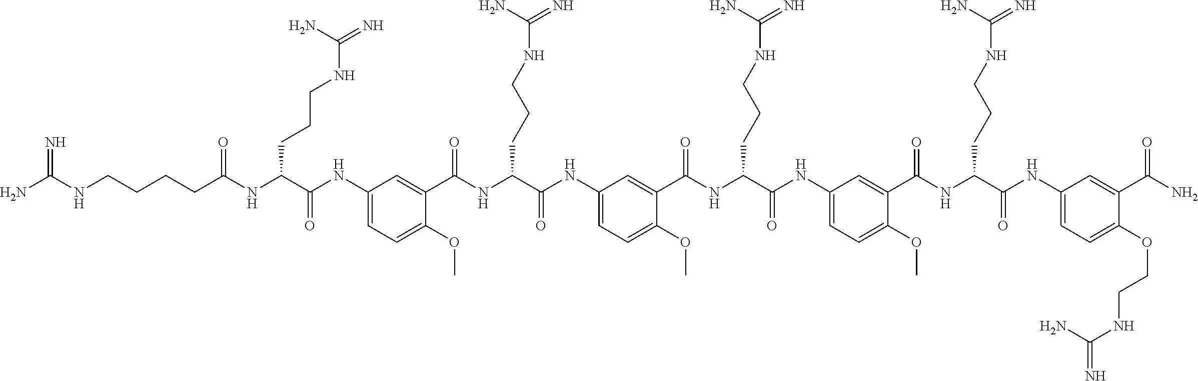 |
| 28 | 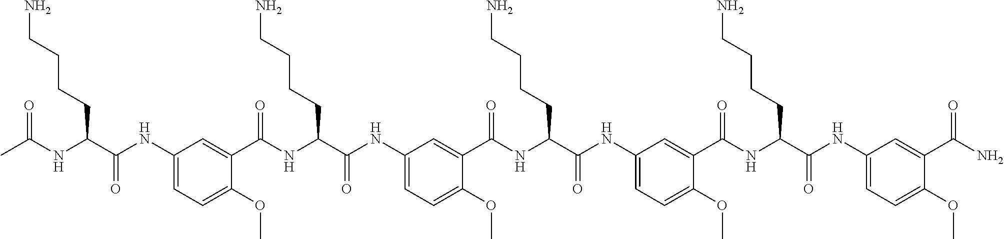 |
| 29 | 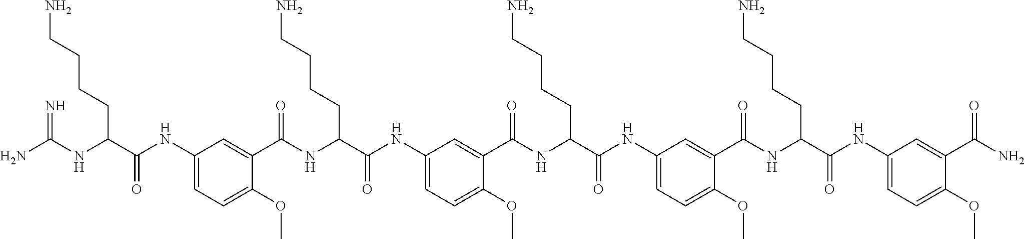 |
| 30 | 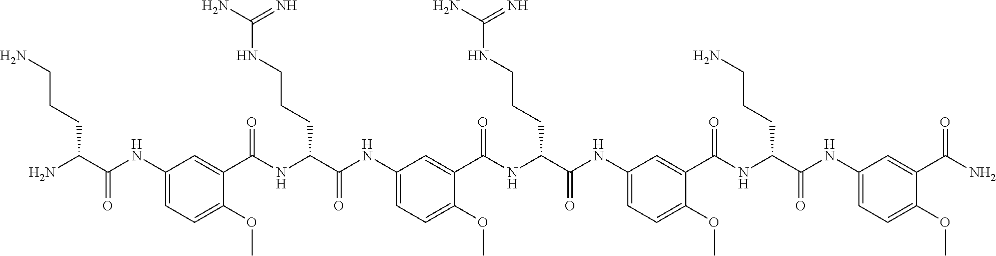 |
| 31 | 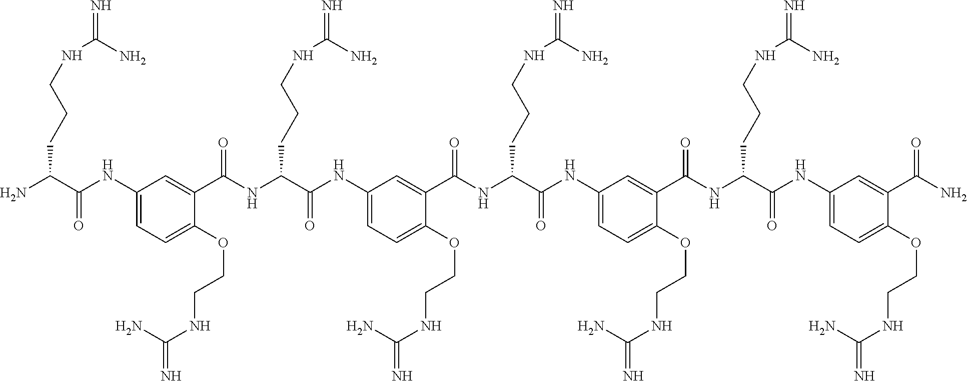 |
| 32 | 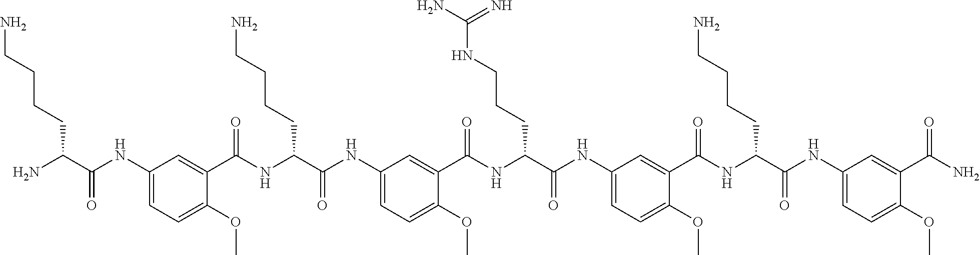 |
| 33 | 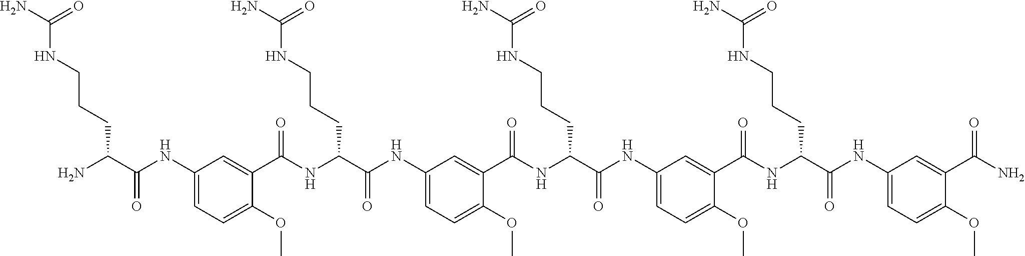 |
| 34 | 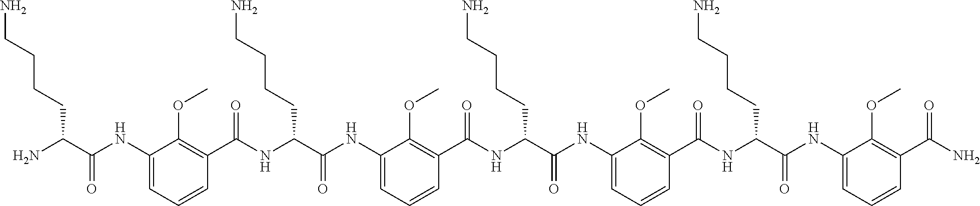 |
| 35 | 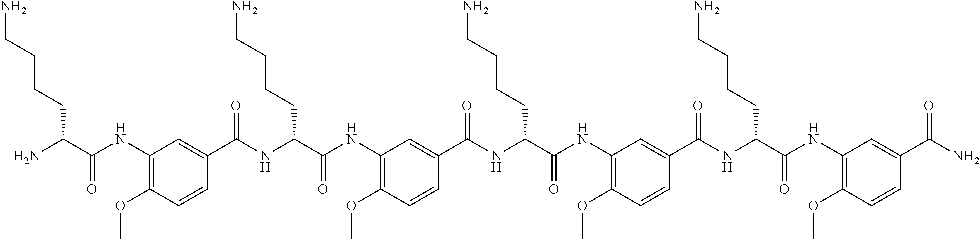 |
| 36 | 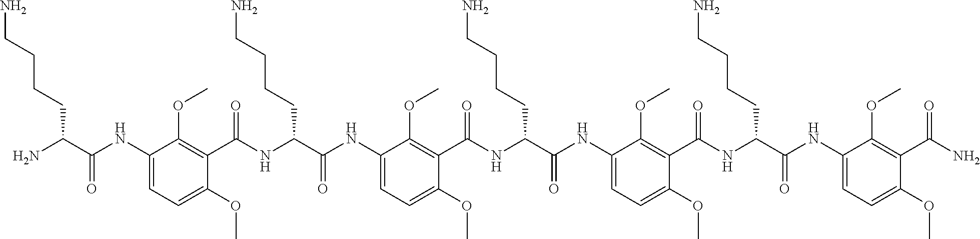 |
| 37 | 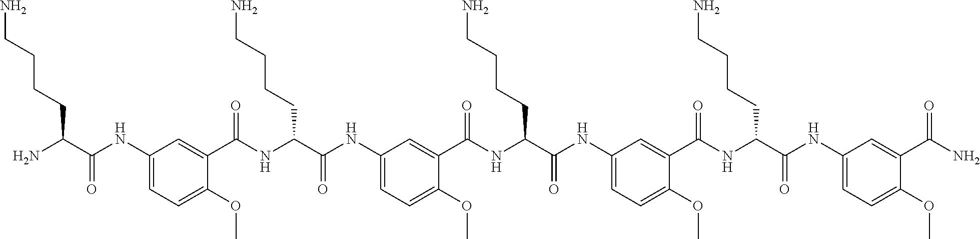 |
| 38 | 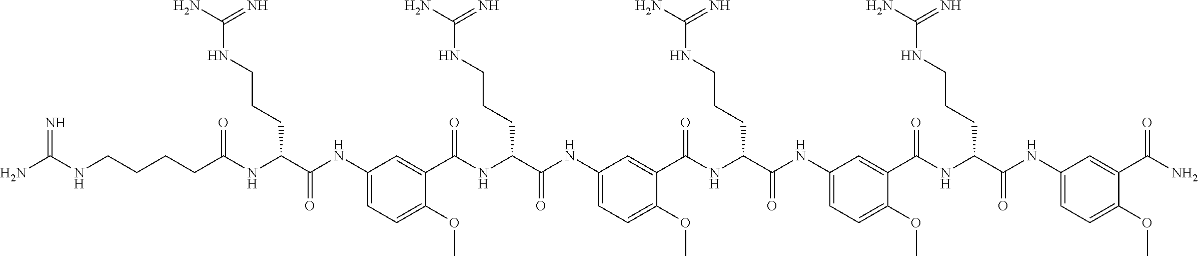 |
| 39 | 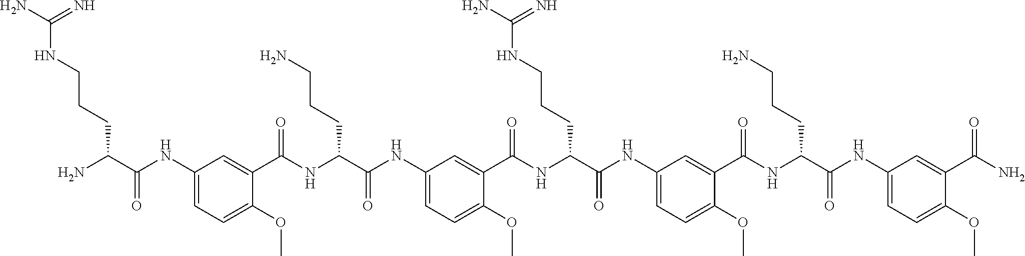 |
| 40 | 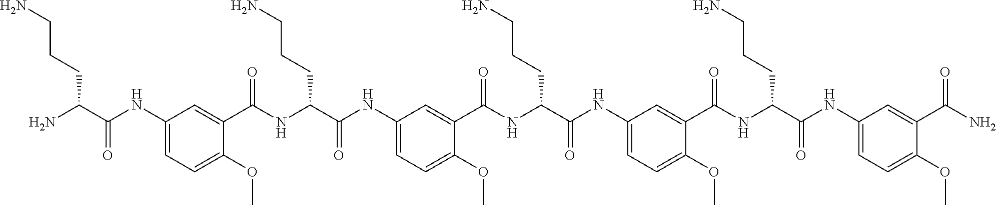 |
| 41 | 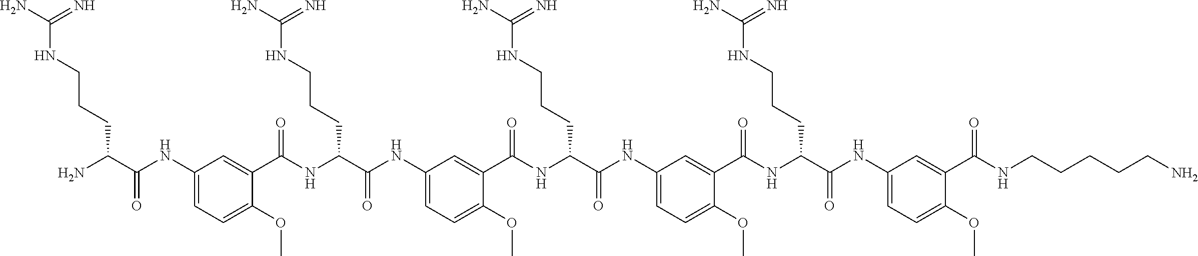 |
| 42 | 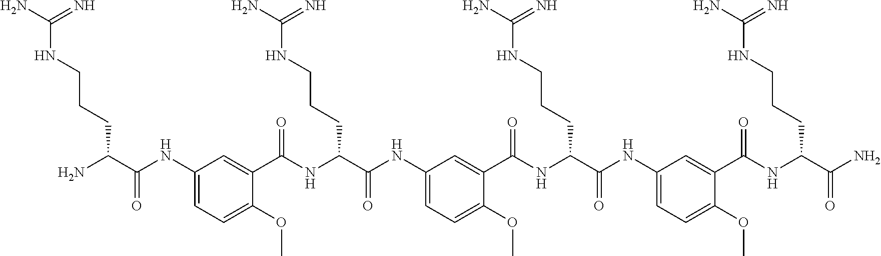 |
| 43 | 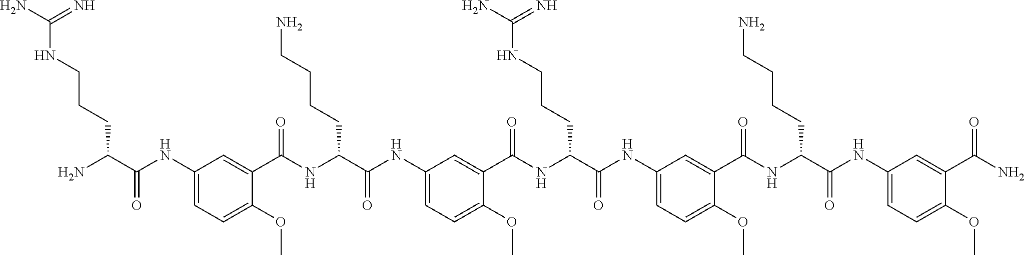 |
| 44 | 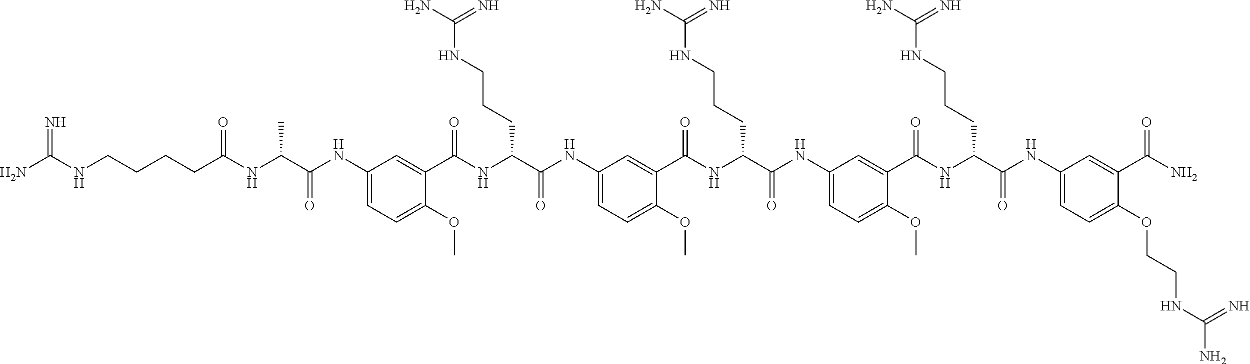 |
| 45 | 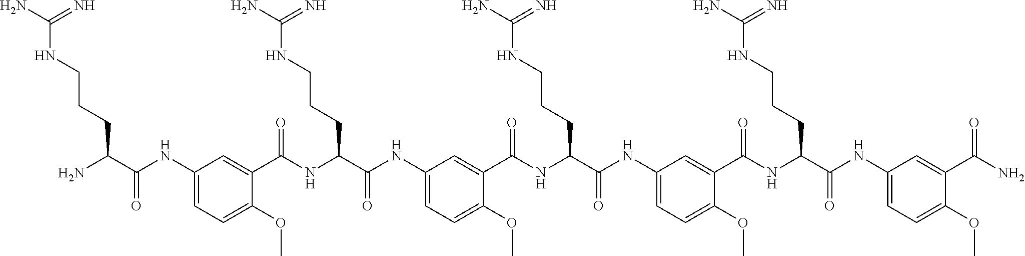 |
| 46 | 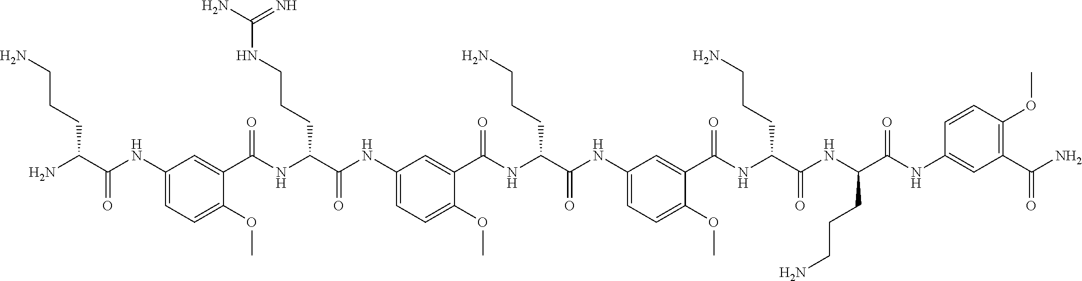 |
| 47 | 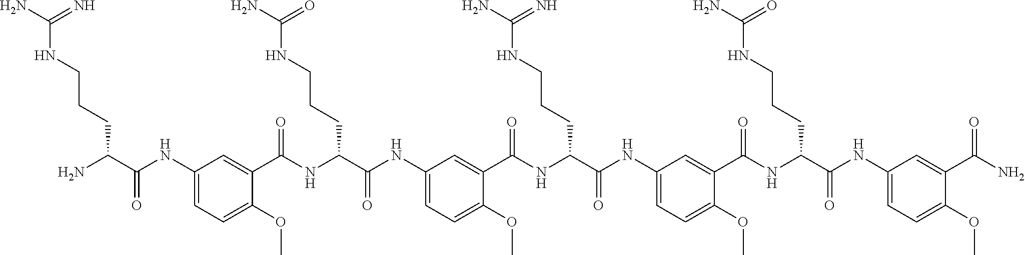 |
| 48 | 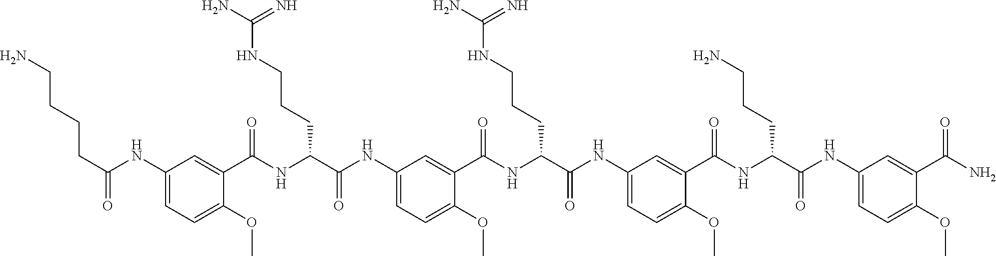 |
| 49 | 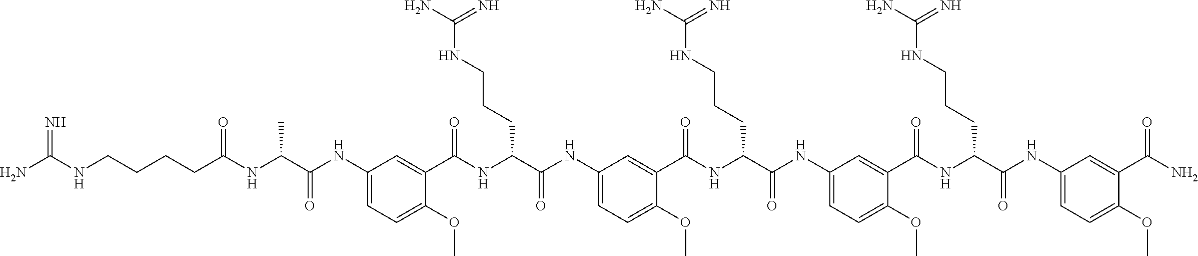 |
| 50 | 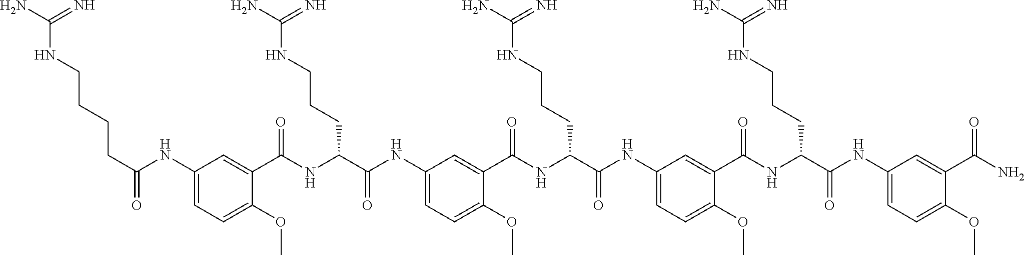 |
| 51 | 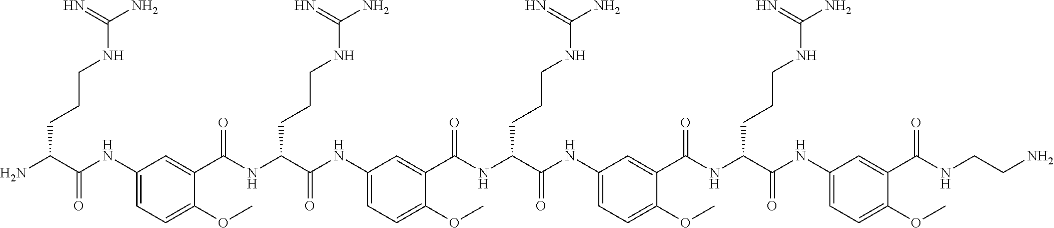 |
| 52 | 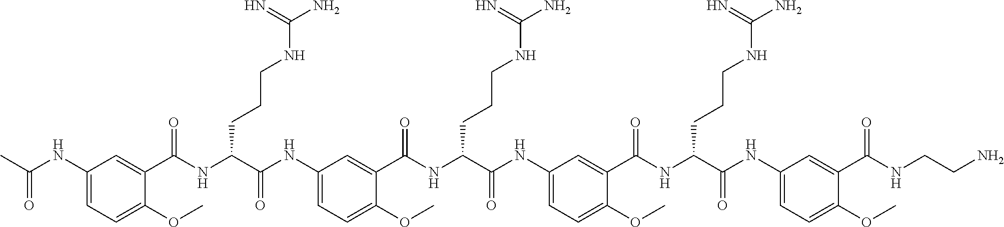 |
| 53 | 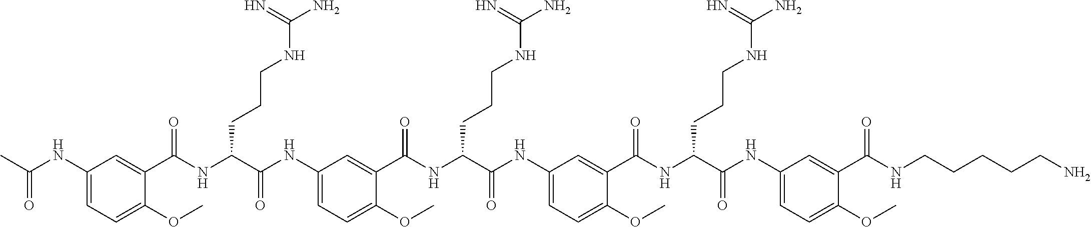 |
| 54 | 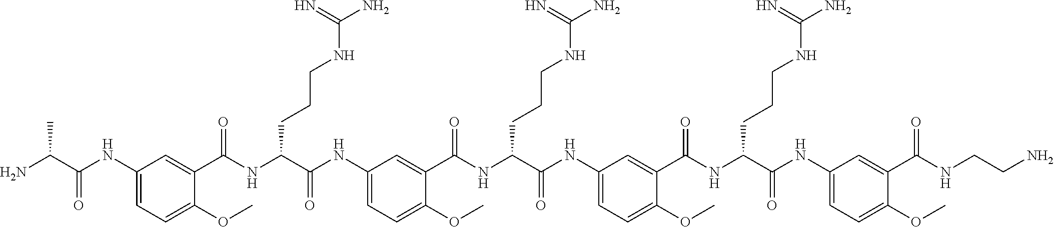 |
| 55 | 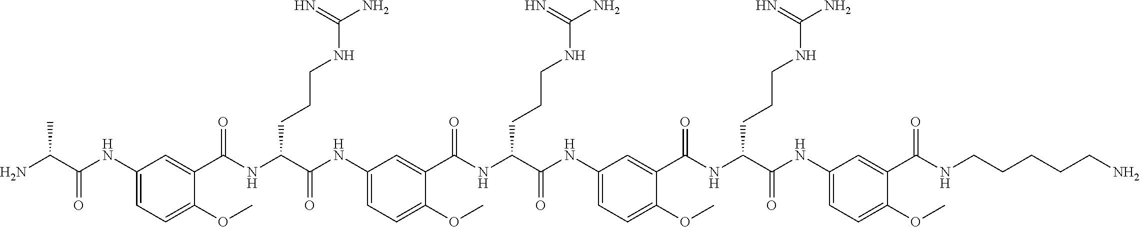 |
| 56 | 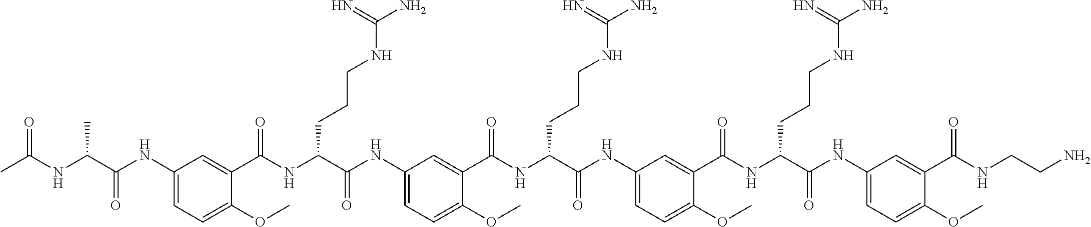 |
| 57 | 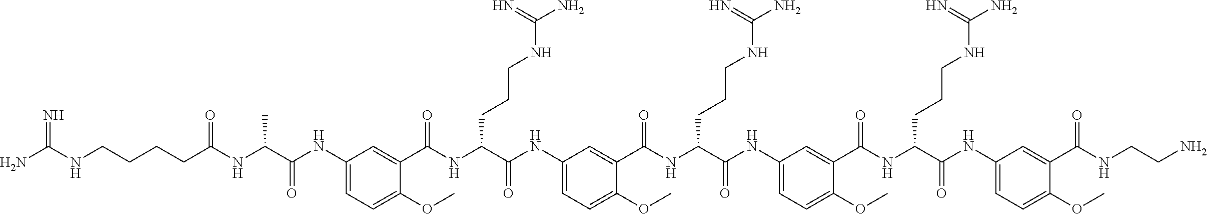 |
| 58 | 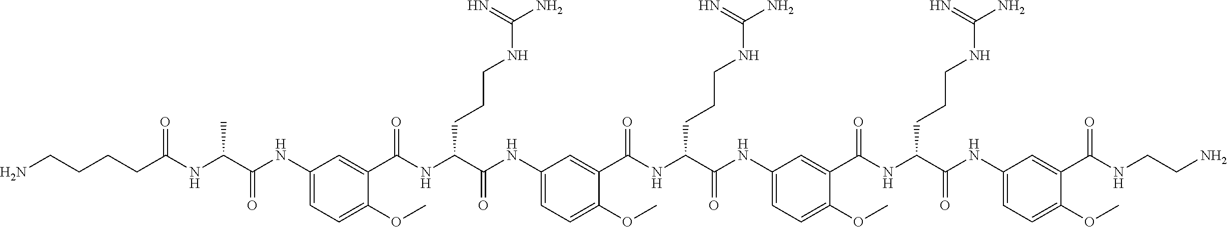 |
| 59 | 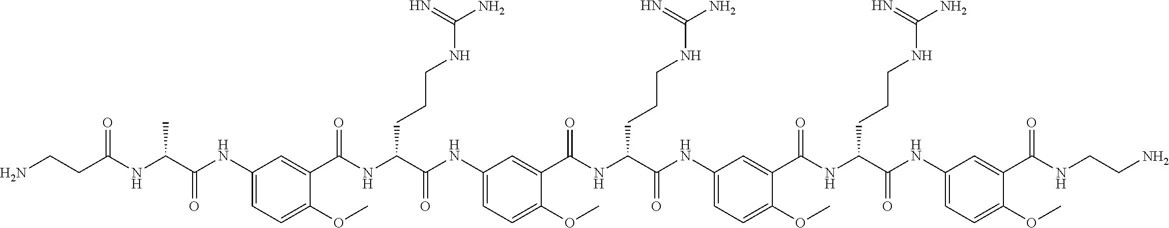 |
| 60 | 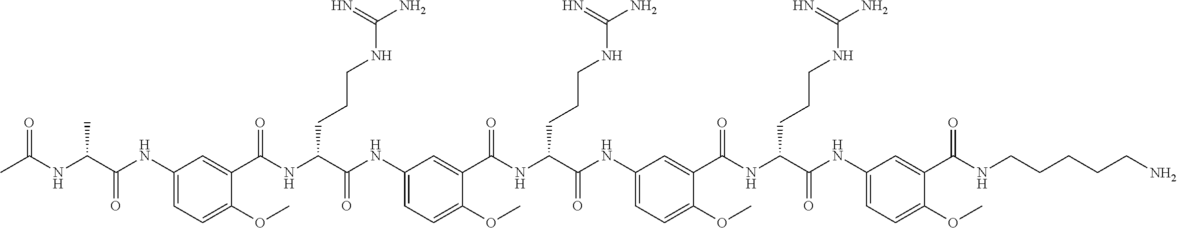 |
| 61 | 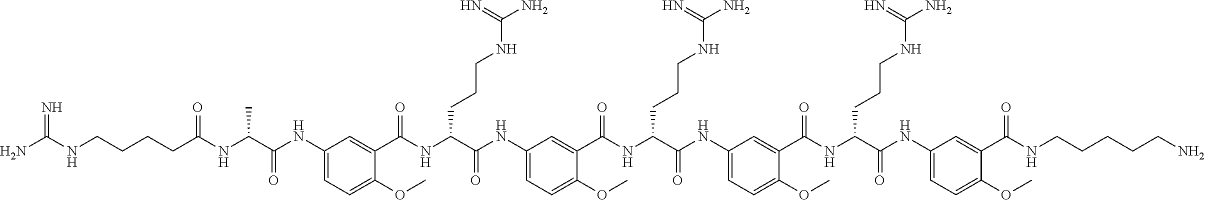 |
| 62 | 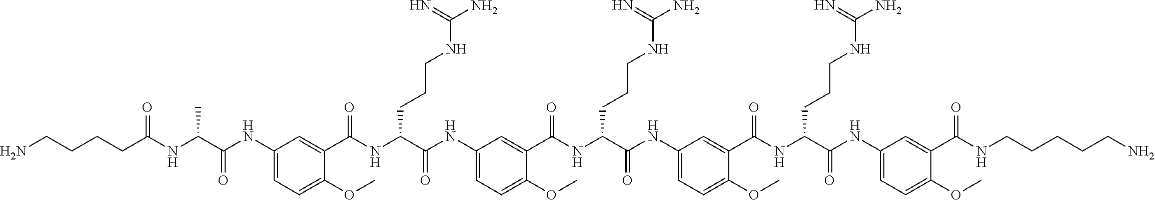 |
| 63 | 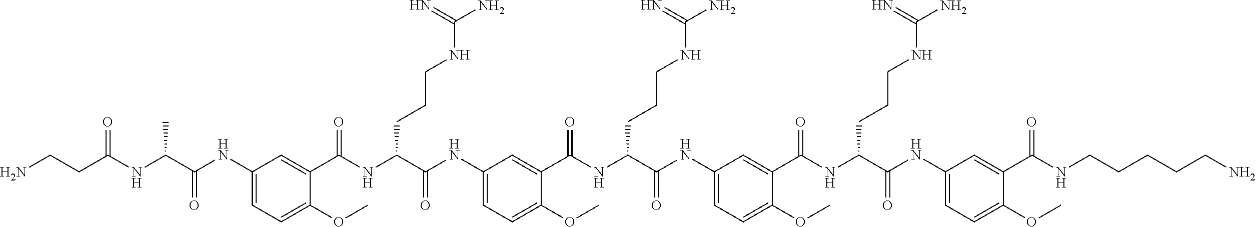 |
| 64 | 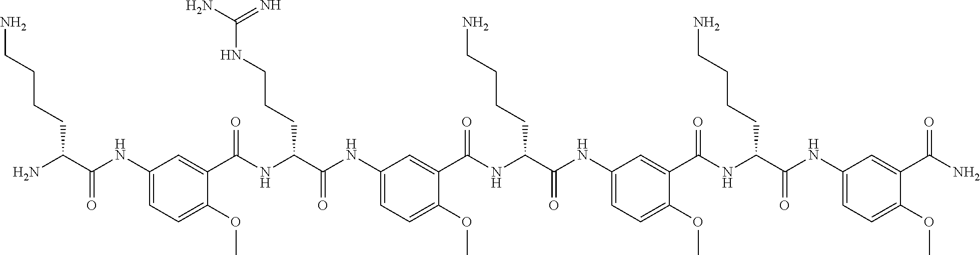 |
| 65 | 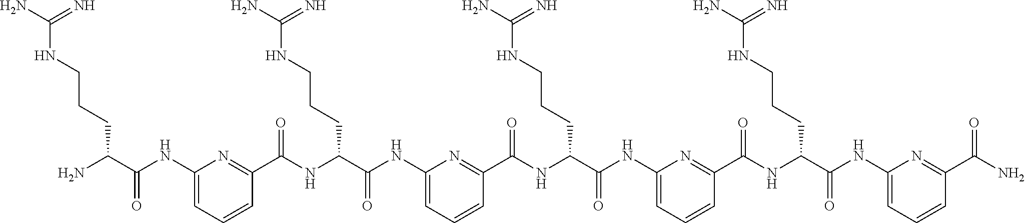 |
| 66 | 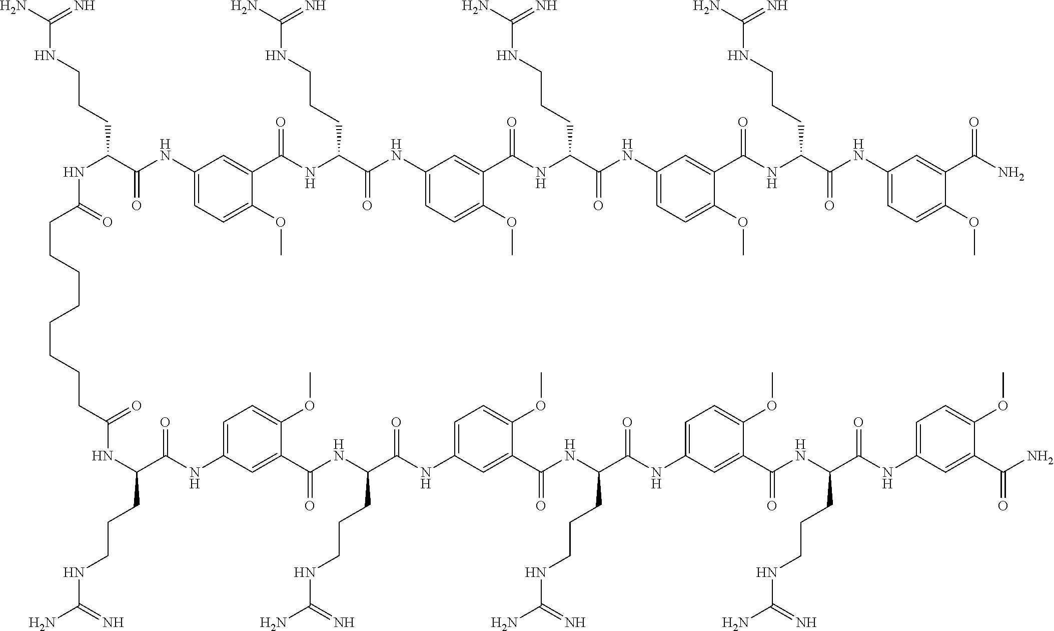 |
| 67 | 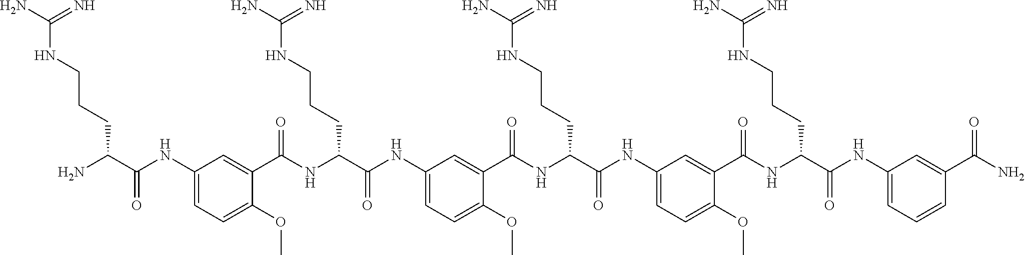 |
| 68 | 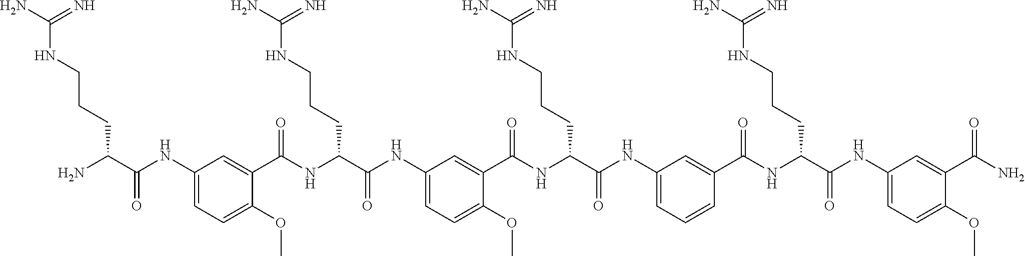 |
| 69 | 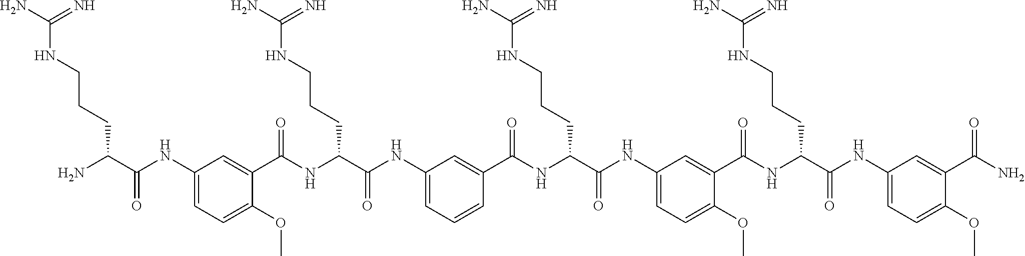 |
| 70 | 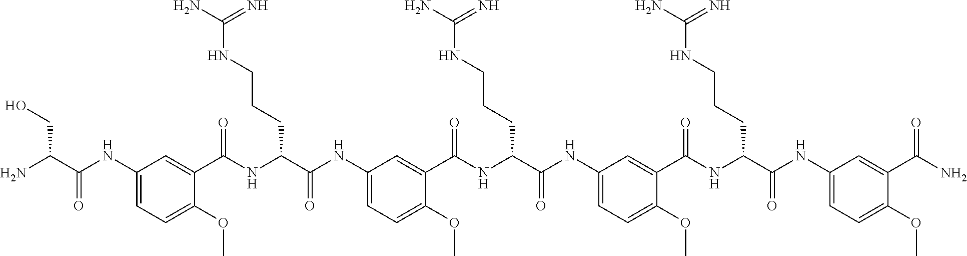 |
| 71 | 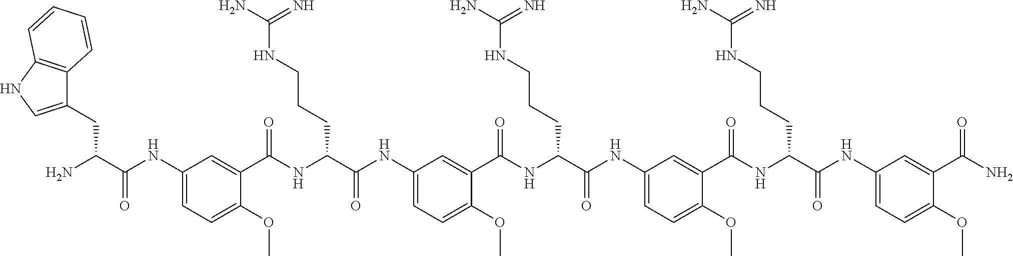 |
| 72 | 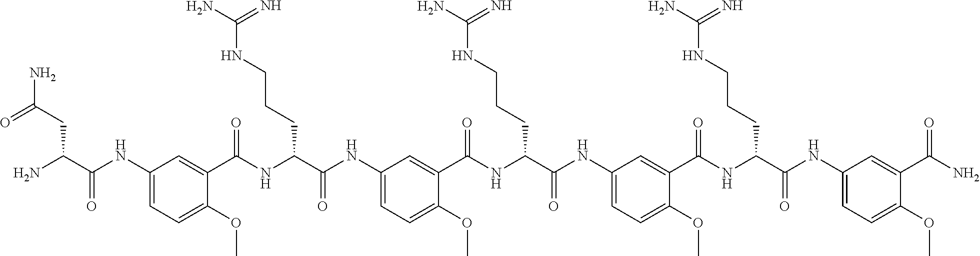 |
| 73 | 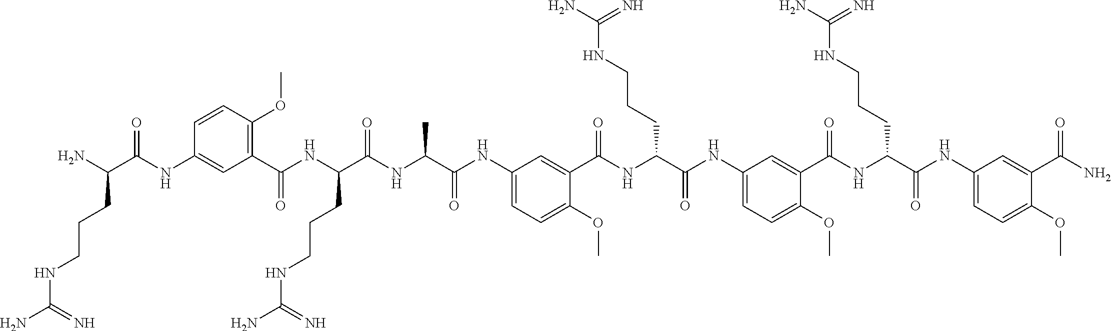 |
| 74 | 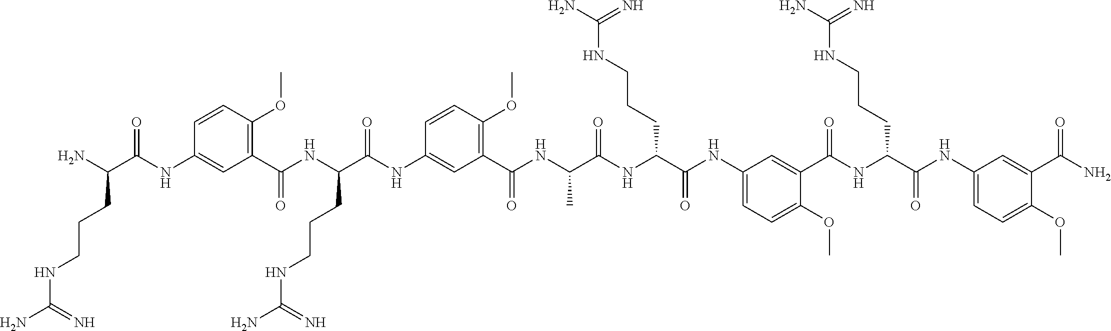 |
| 75 | 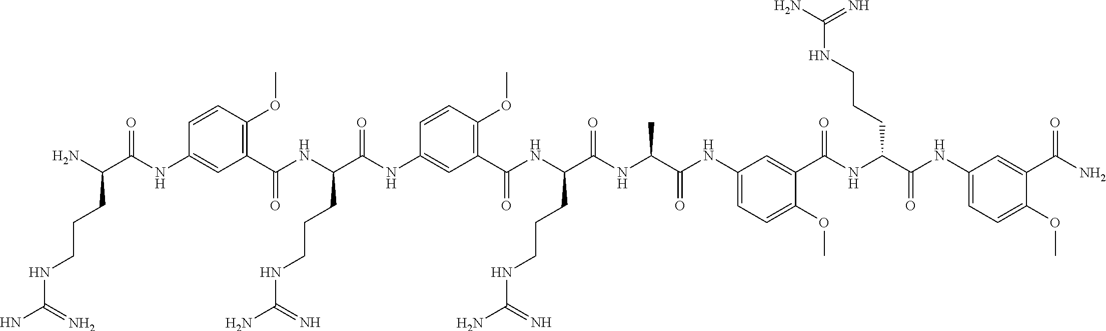 |
| 76 | 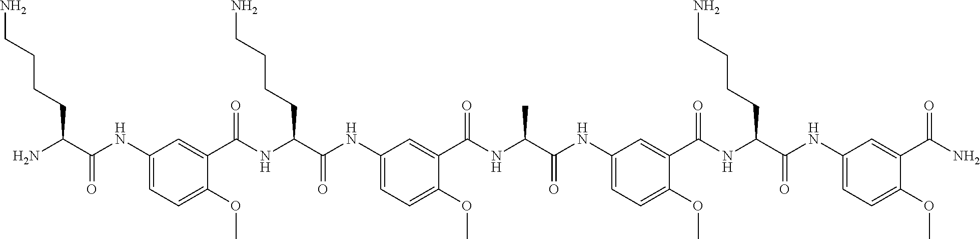 |
| 77 | 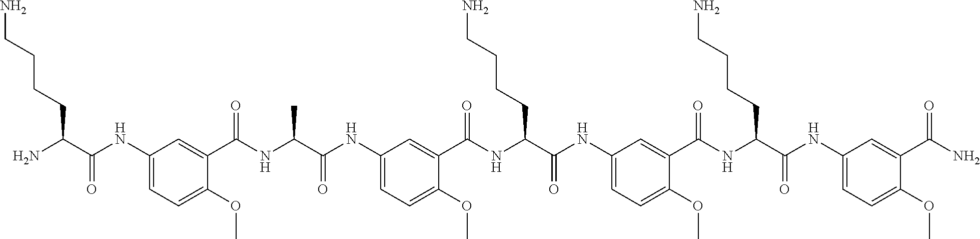 |
| 78 | 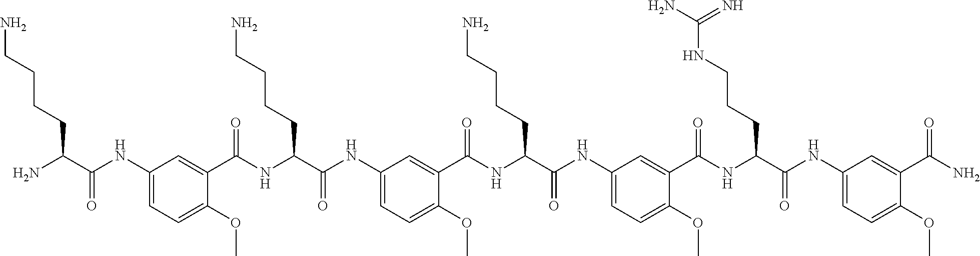 |
| 79 | 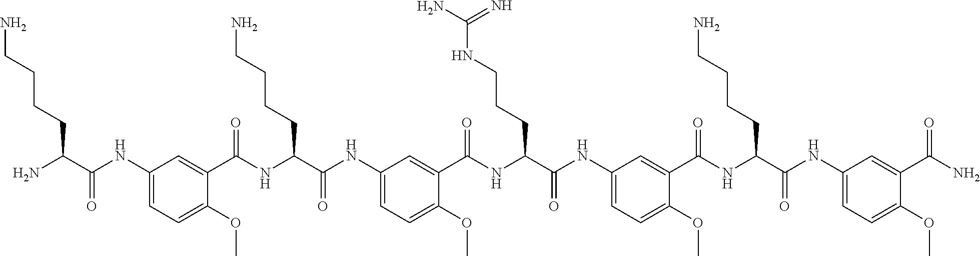 |
| 80 | 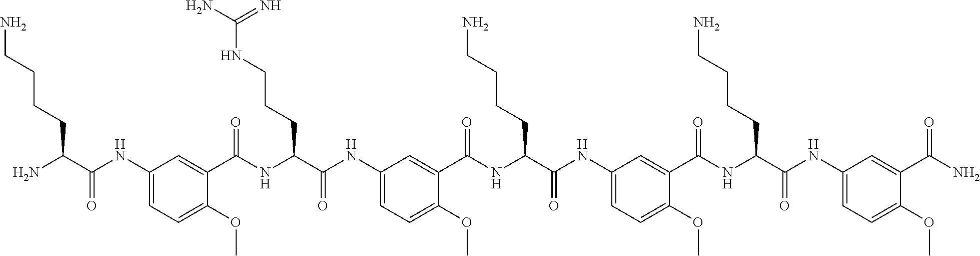 |
| 81 | 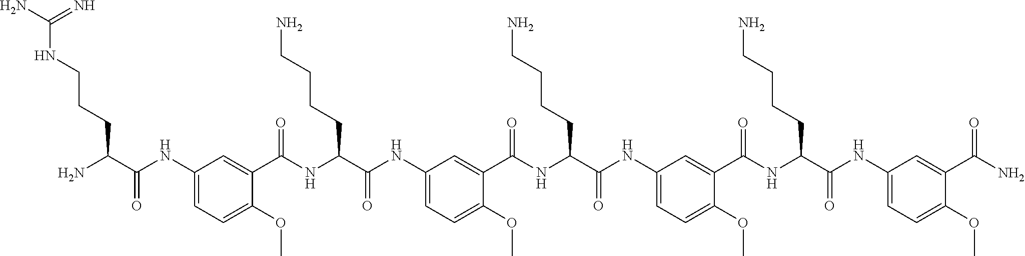 |
| 82 | 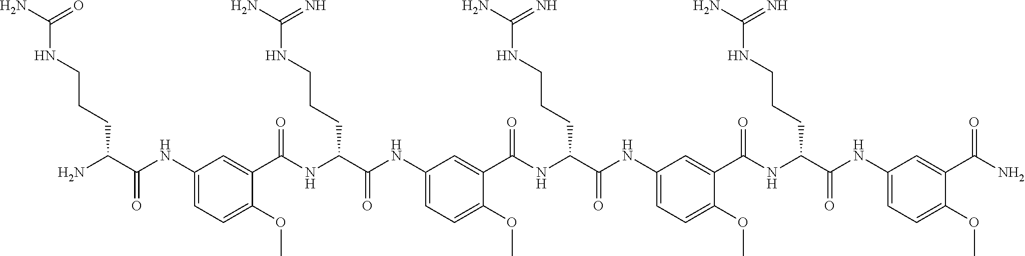 |
| 83 | 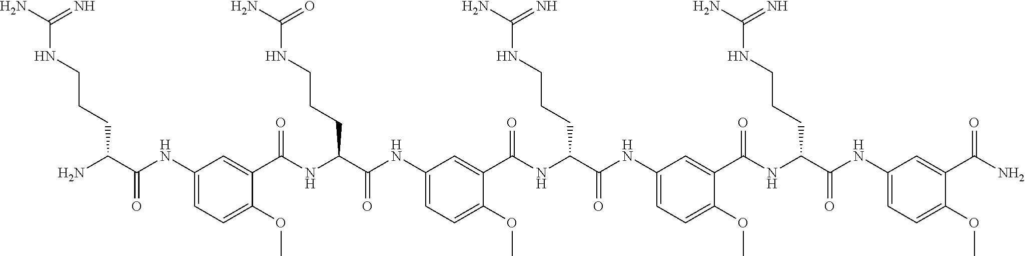 |
| 84 | 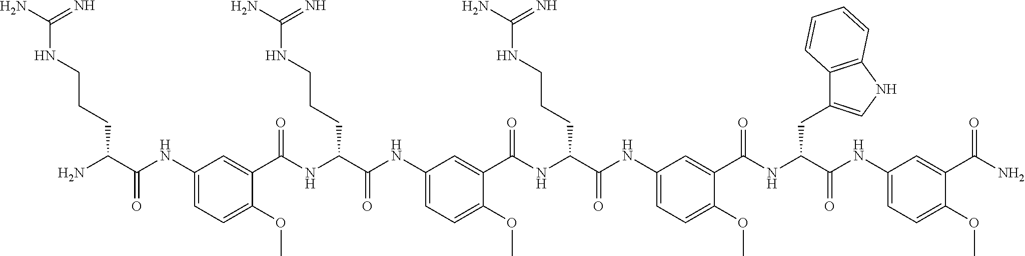 |
| 85 | 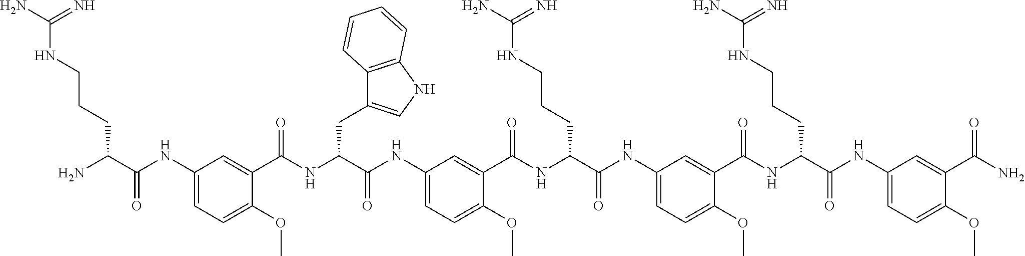 |
| 86 | 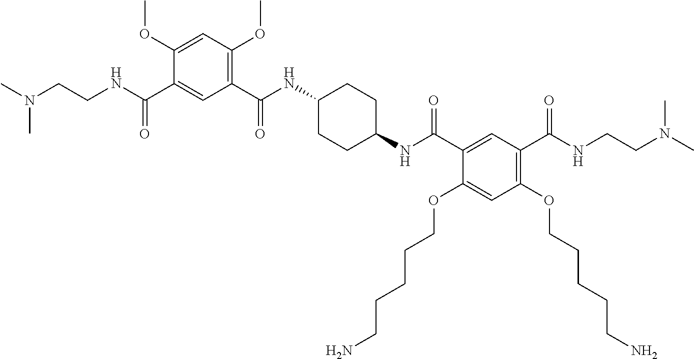 |
| 87 | 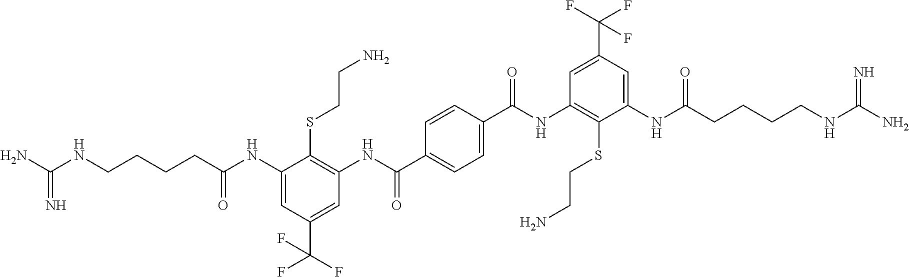 |
| 88 | 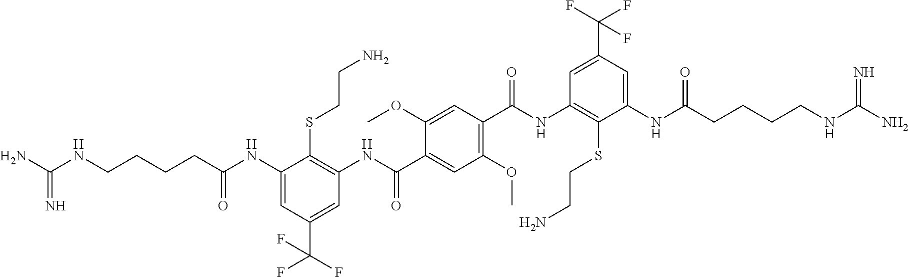 |
| 89 | 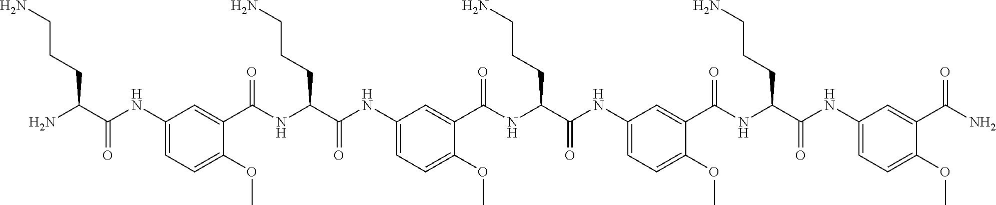 |
| 90 | 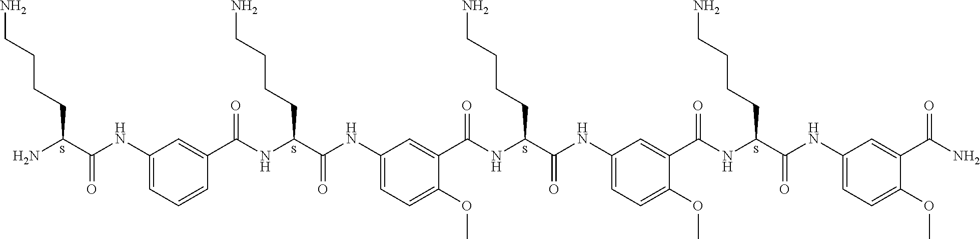 |
| 91 | 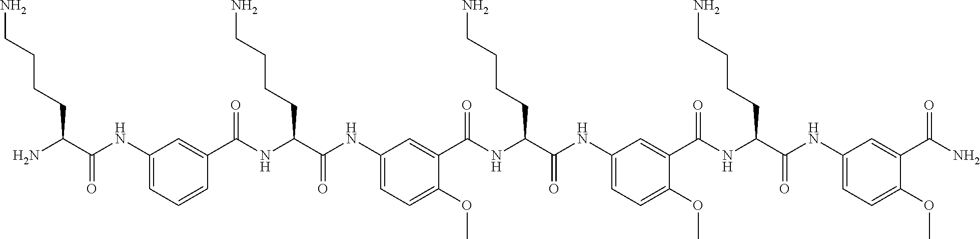 |
| 92 | 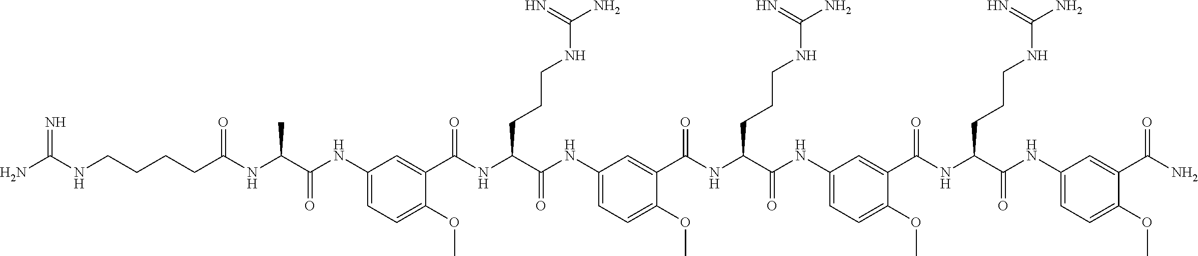 |
| 93 | 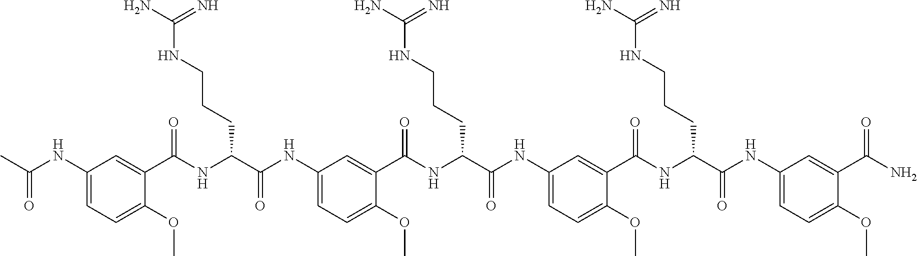 |
| 94 | 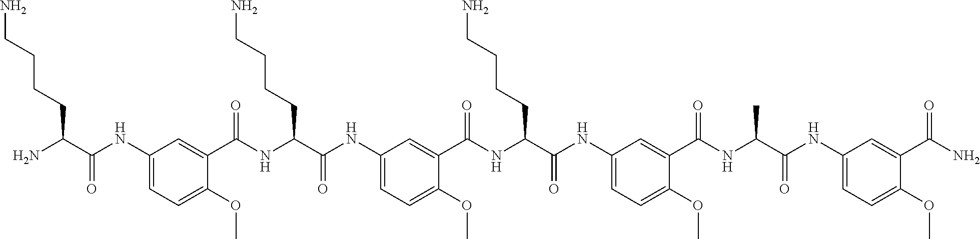 |
| 95 | 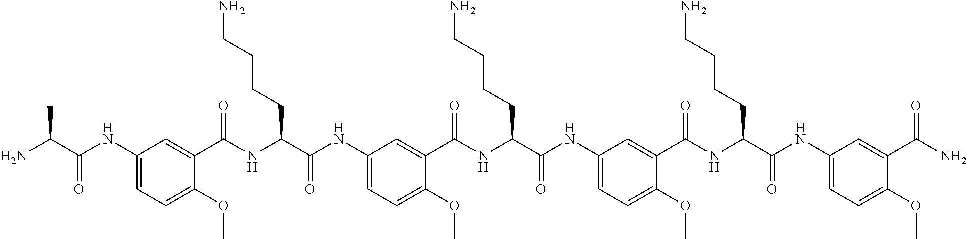 |
| 96 | 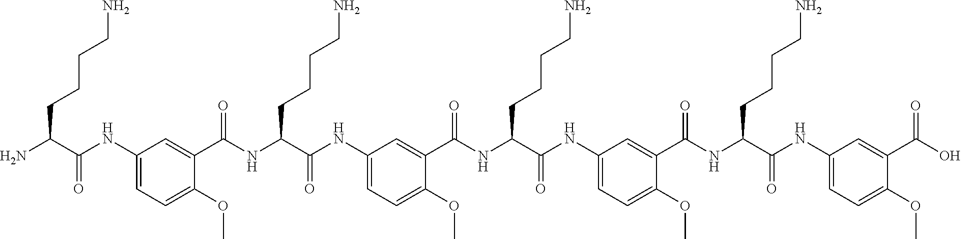 |
| 97 | 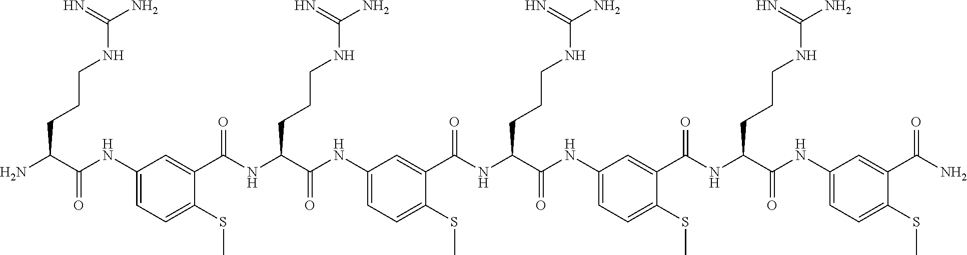 |
| 98 | 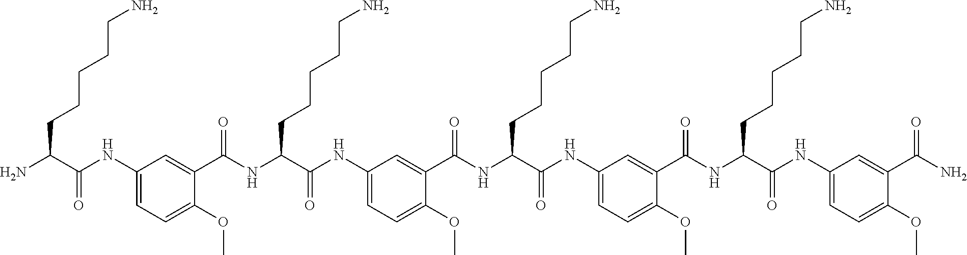 |
| 99 | 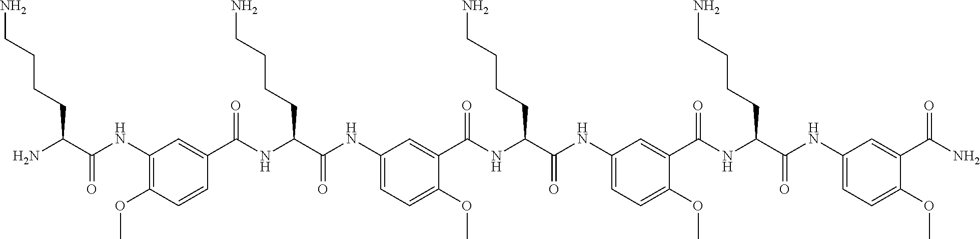 |
| 100 | 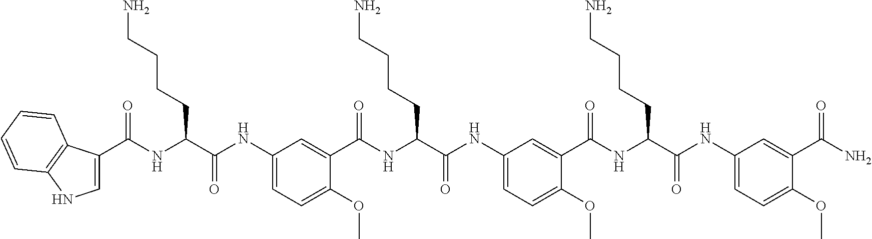 |
| 101 | 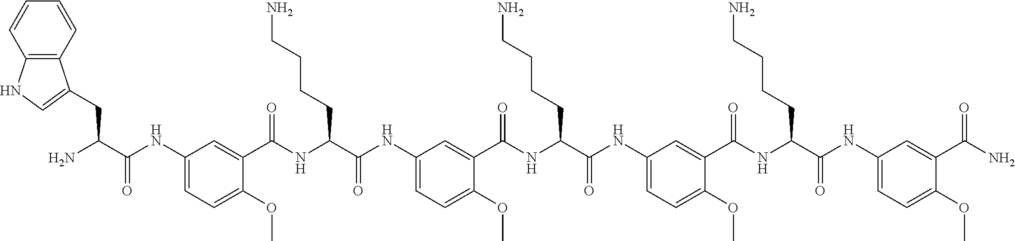 |
| 102 | 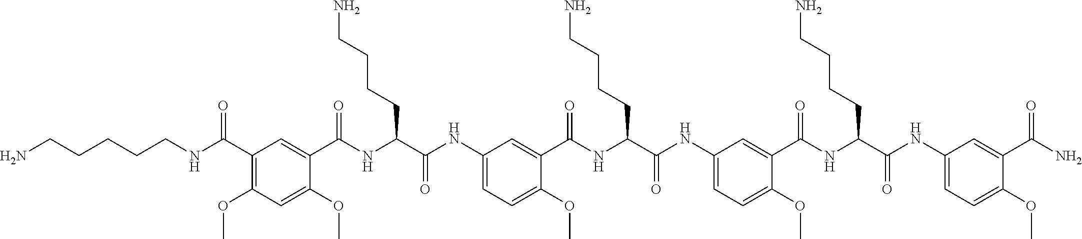 |
| 103 | 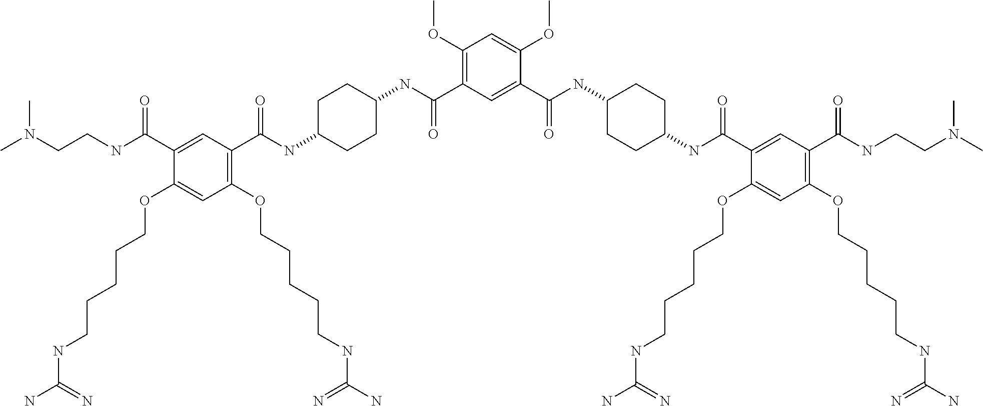 |
| 104 | 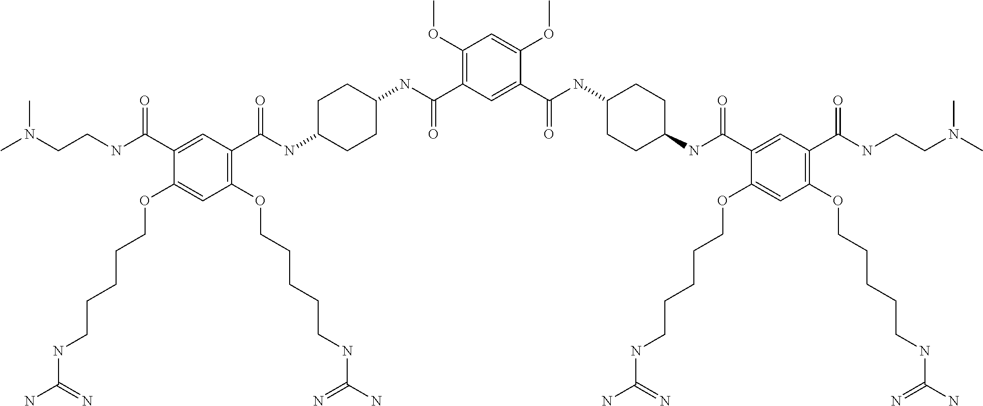 |
| 105 | 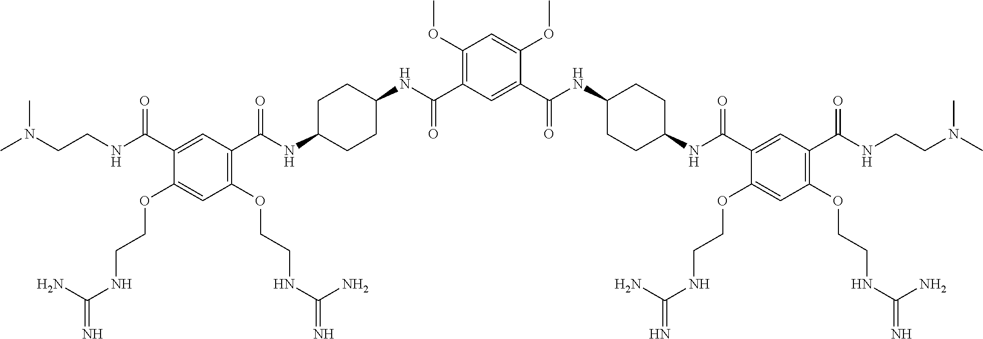 |
| 106 | 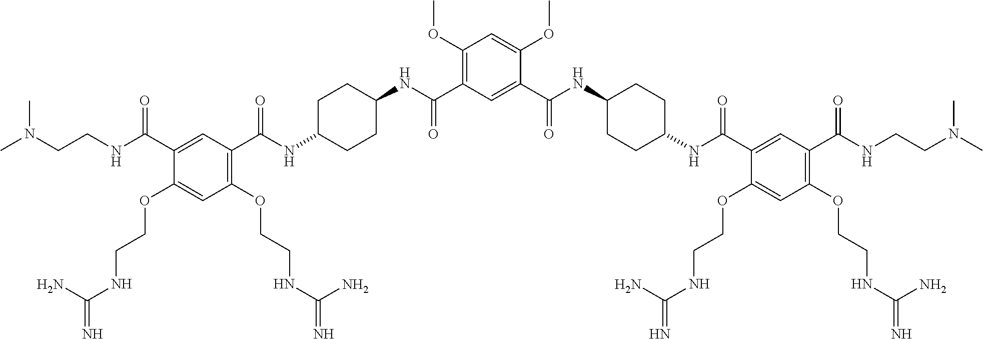 |
| 107 | 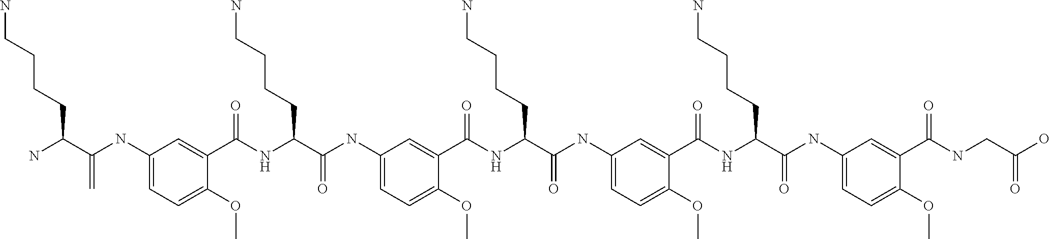 |
| 108 | 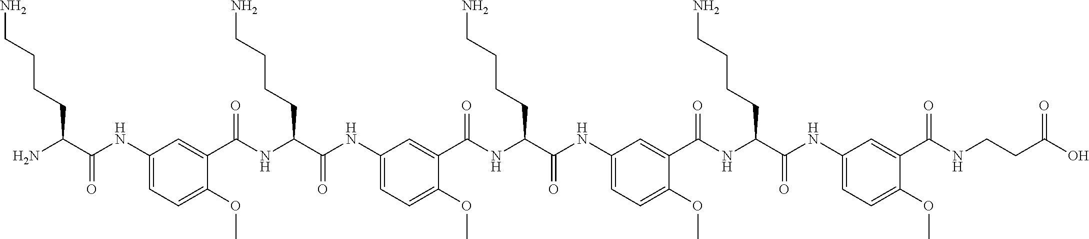 |
| 109 | 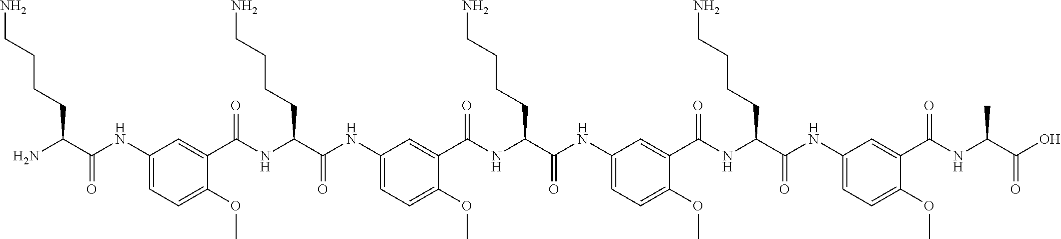 |
| 110 | 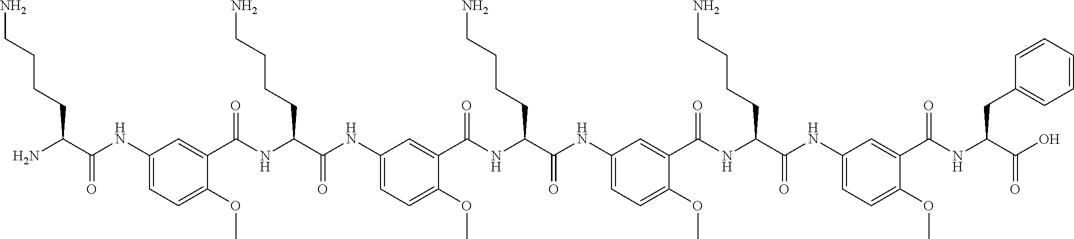 |
| 111 | 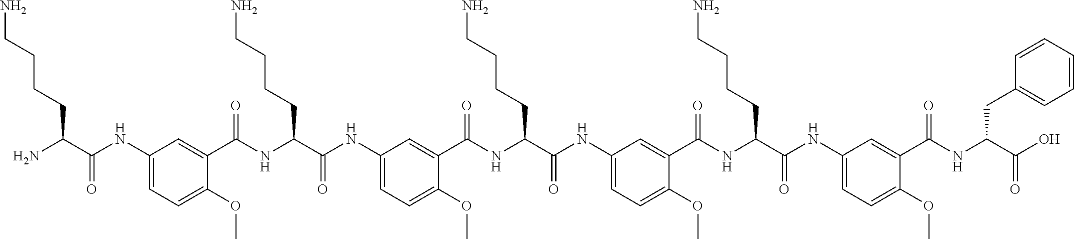 |
| 112 | 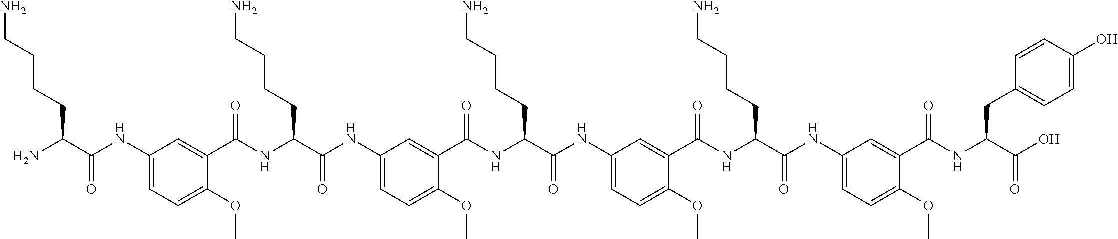 |
| 113 | 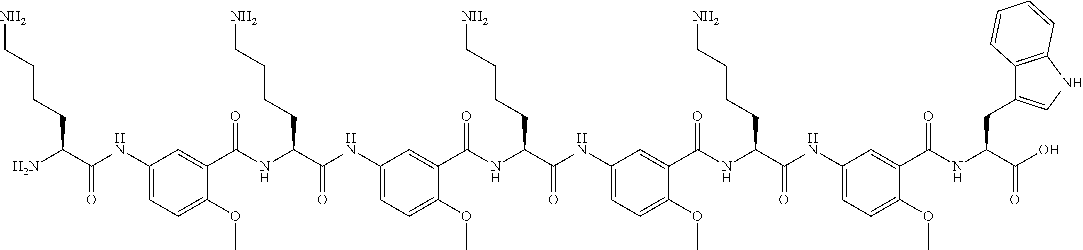 |
| 114 | 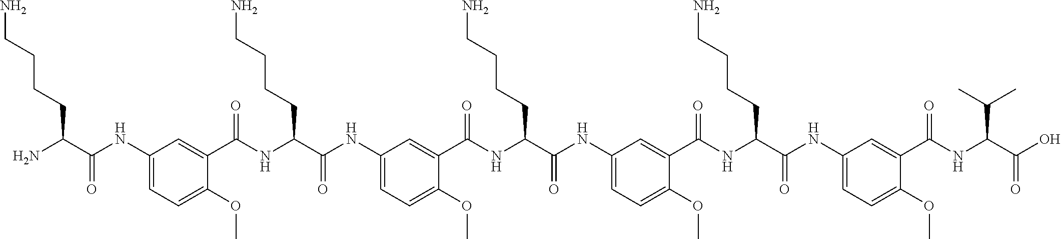 |
| 115 | 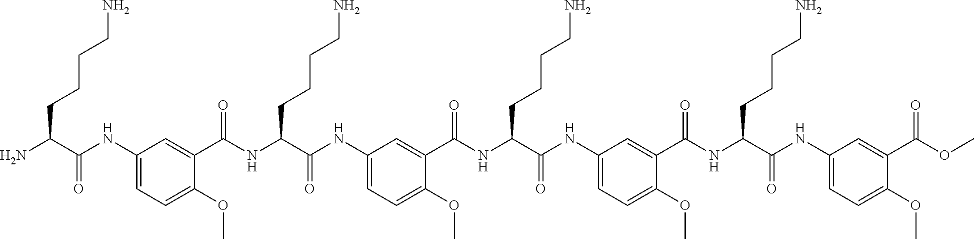 |
| 116 | 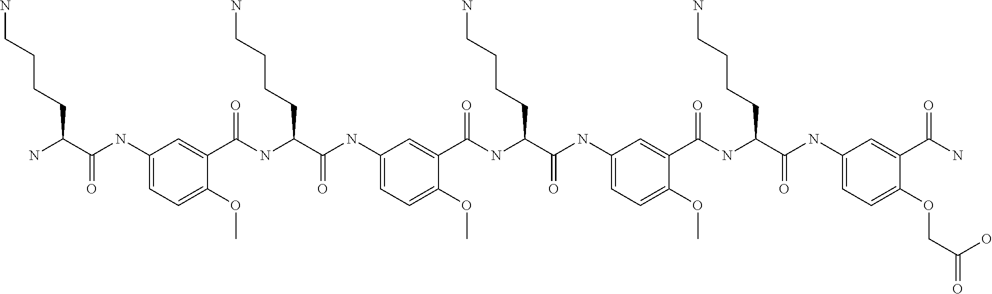 |
| 117 | 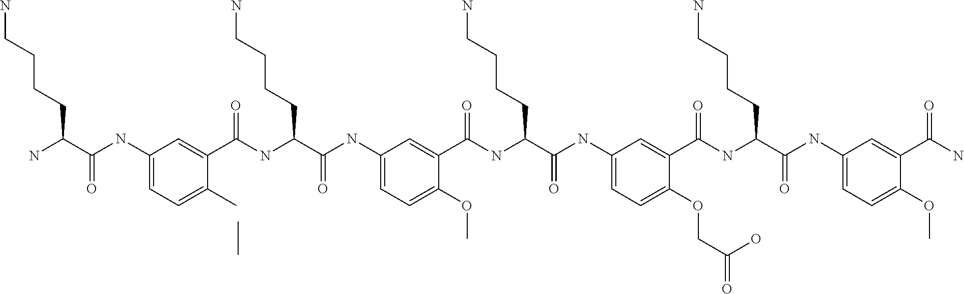 |
| 118 | 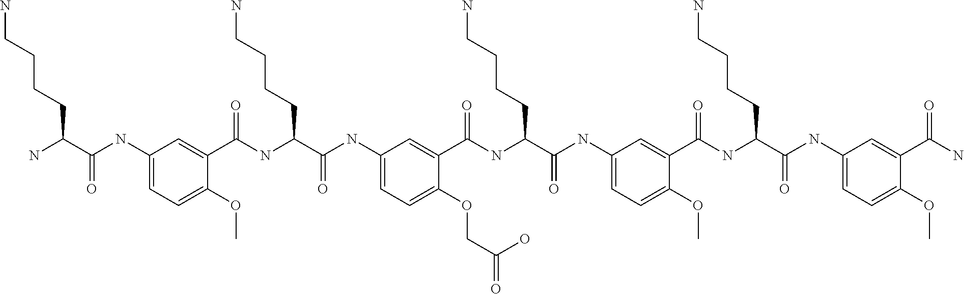 |
| 119 | 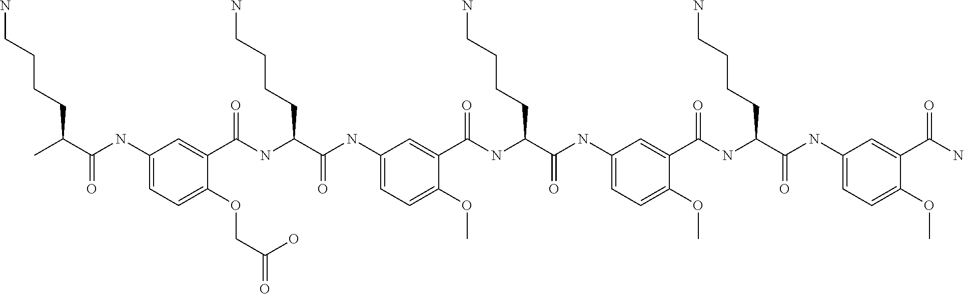 |
| 120 | 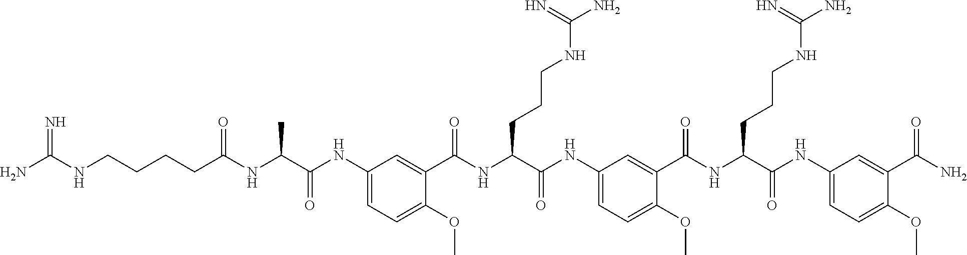 |
| 121 | 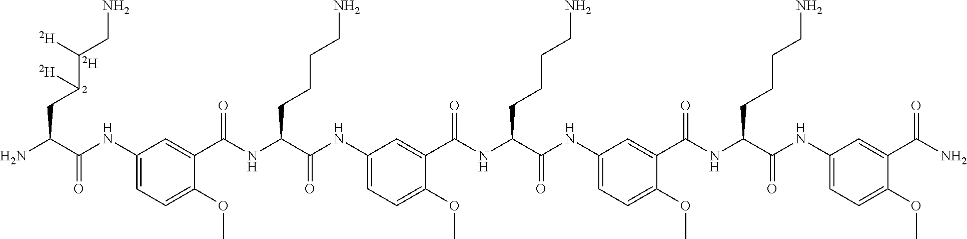 |
| 122 | 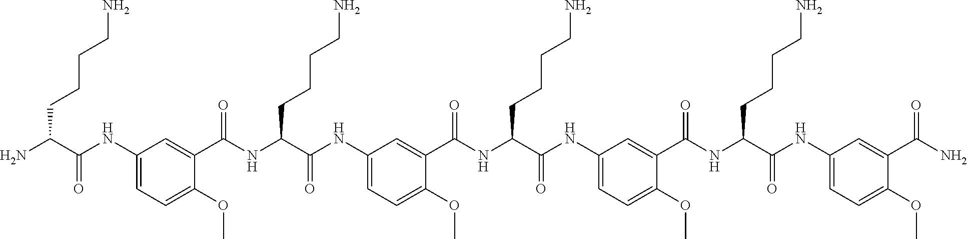 |
| 123 | 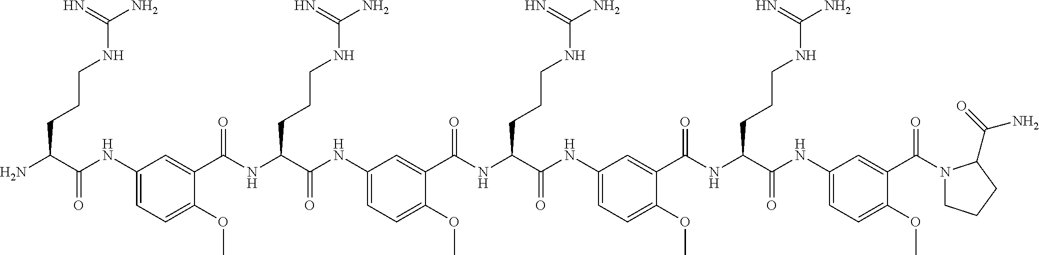 |
| 124 | 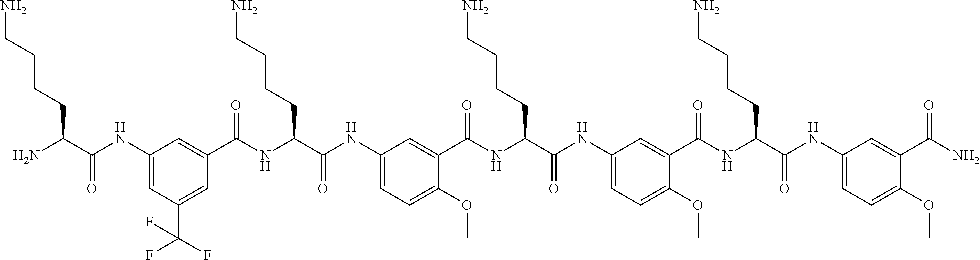 |
| 125 | 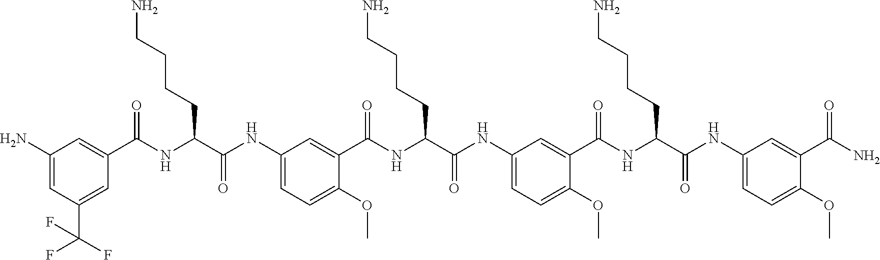 |
| 126 | 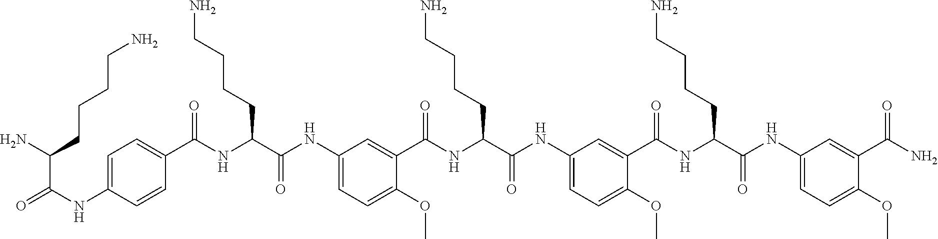 |
| 127 | 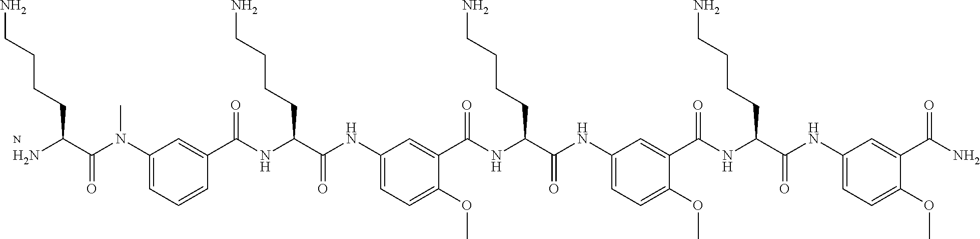 |
| 128 | 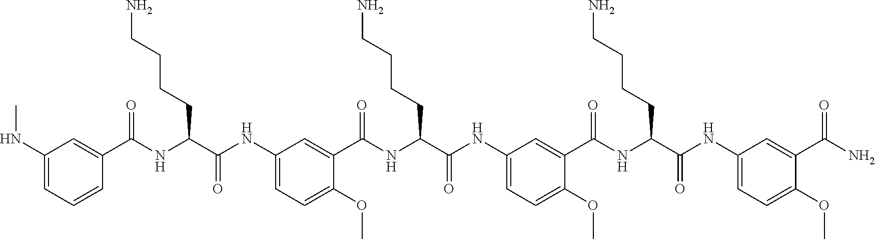 |
| 129 | 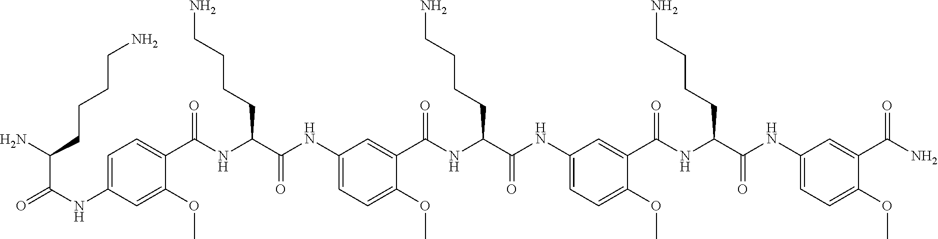 |
| 130 | 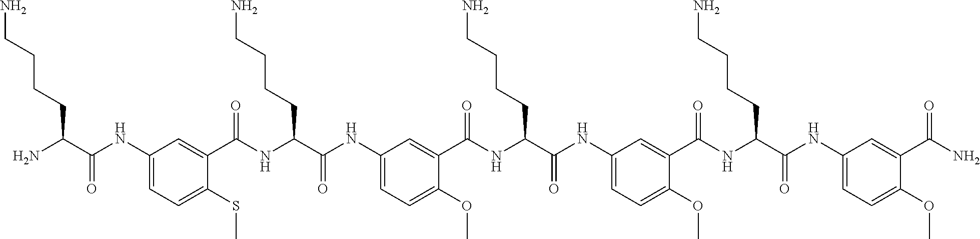 |
| 131 | 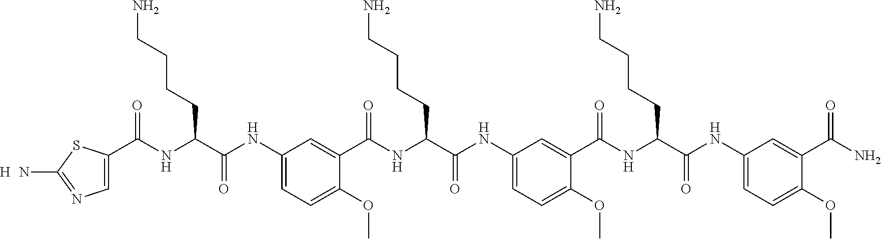 |
| 132 | 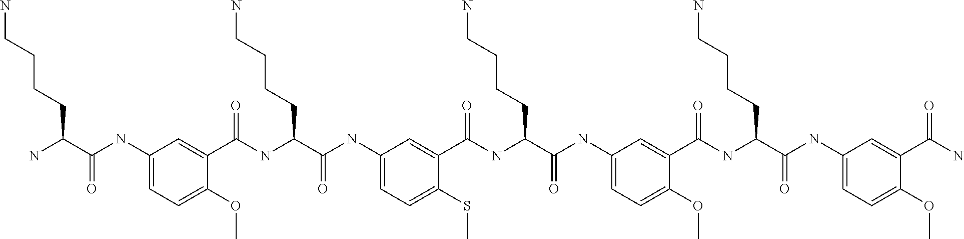 |
| 133 | 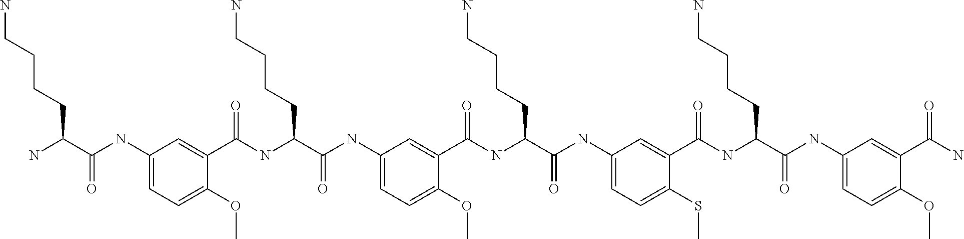 |
| 134 | 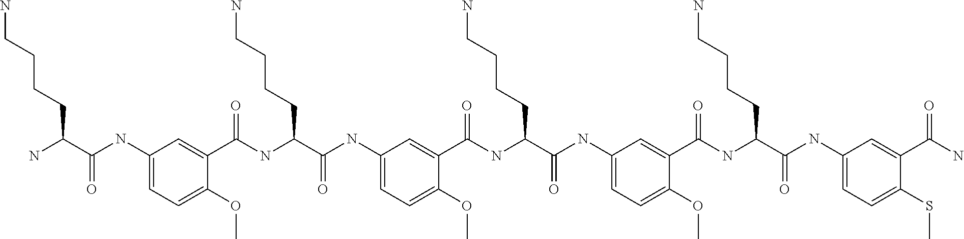 |
| 135 | 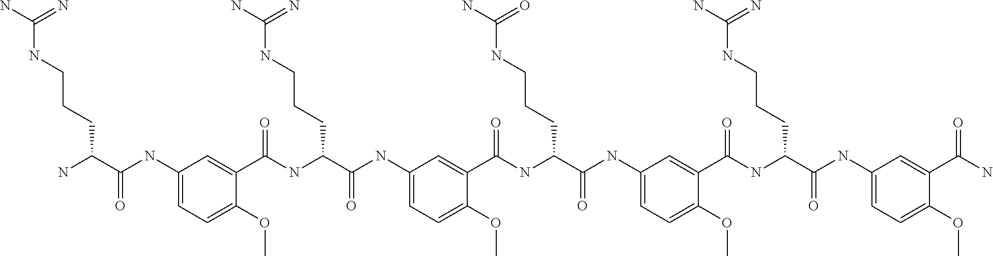 |
| 136 | 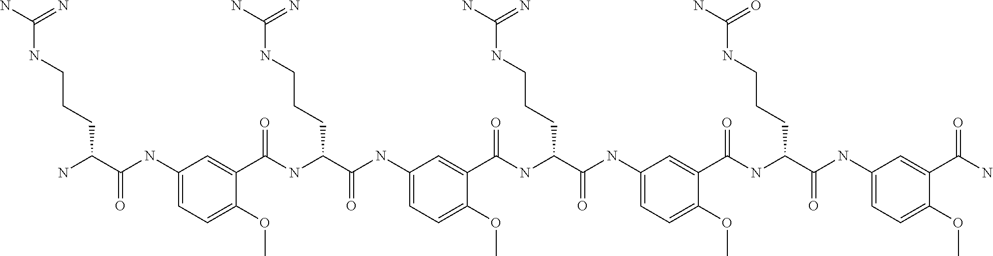 |
| 137 | 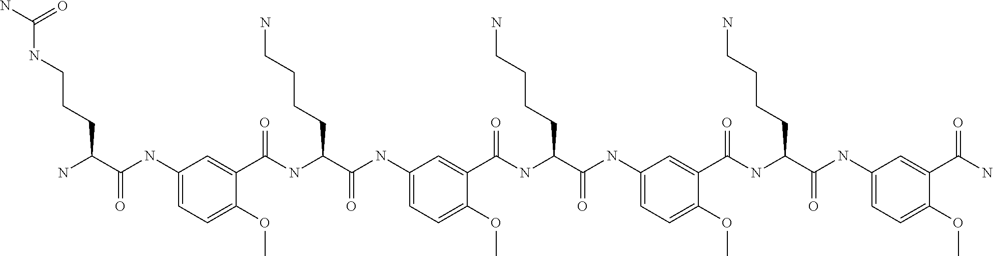 |
| 138 | 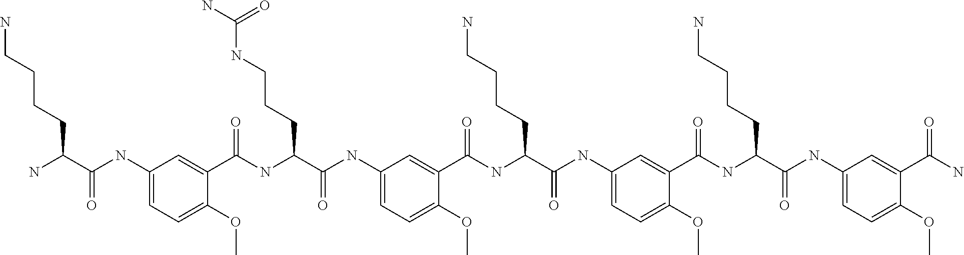 |
| 139 | 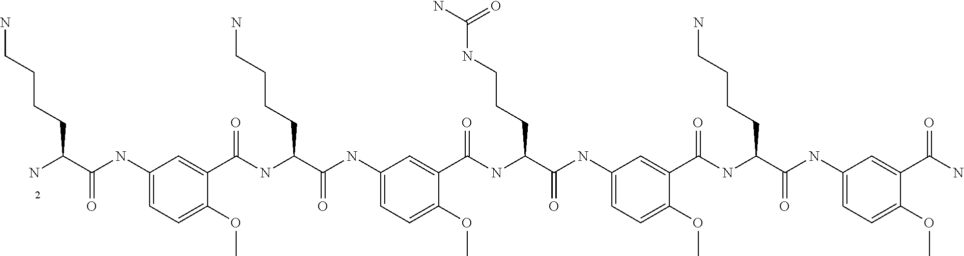 |
| 140 | 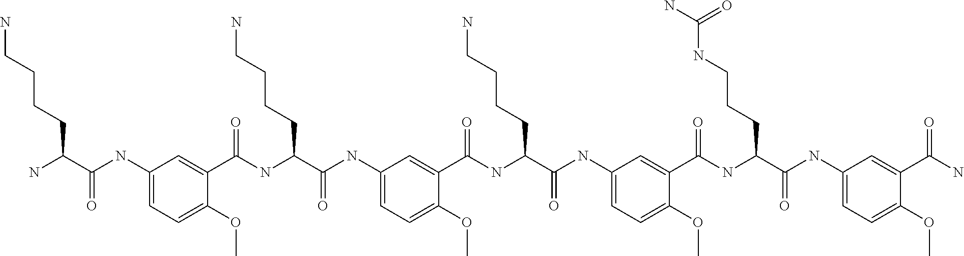 |
| 141 | 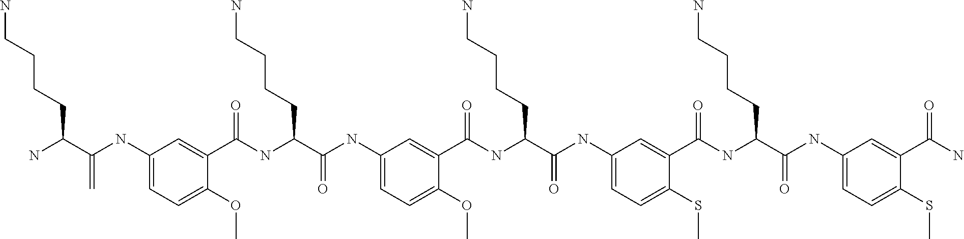 |
| 142 | 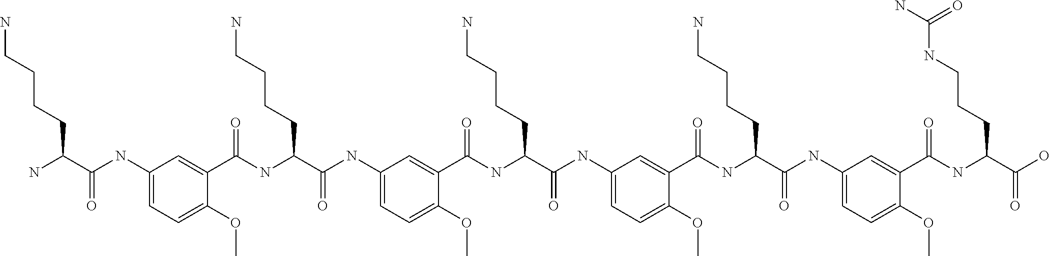 |
| 143 | 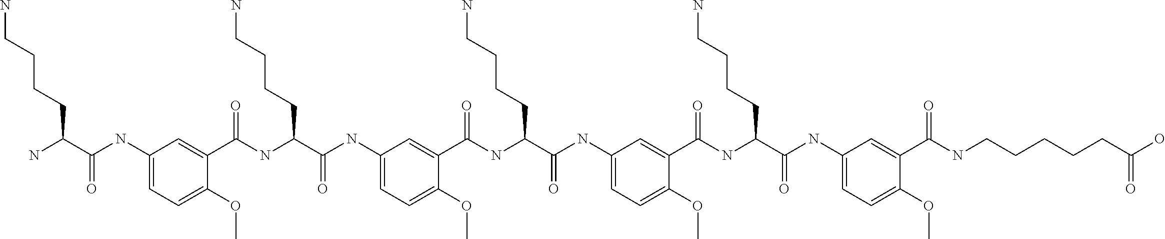 |
| 144 | 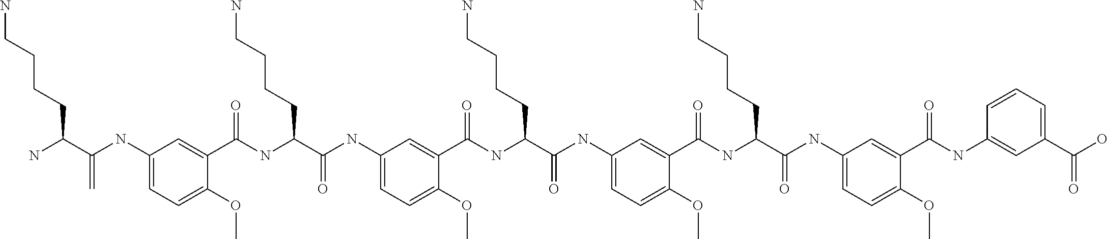 |
| 145 | 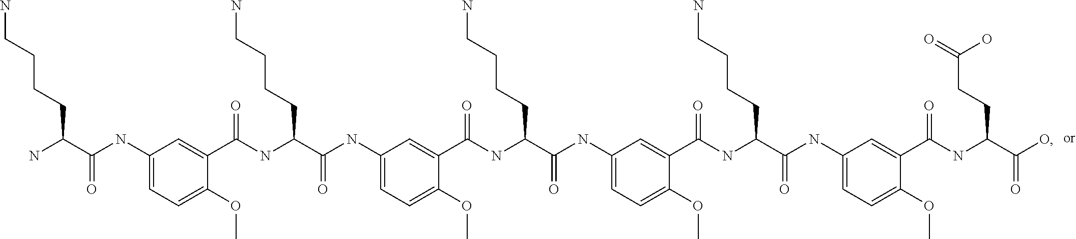 |
| 146 | 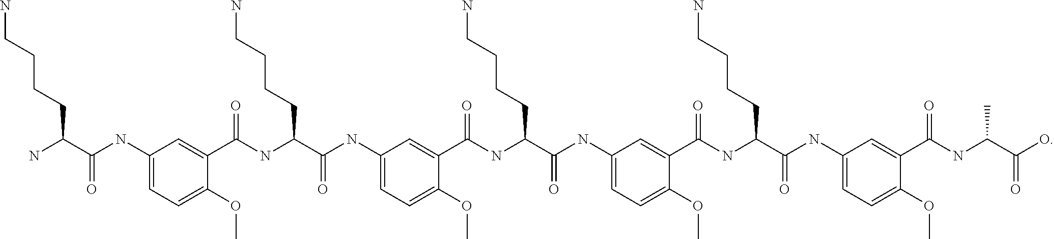 |

In some embodiments, the immune response is against an oral pathogen. In some embodiments, the oral pathogen is chosen from Aggregatibacter spp. such as, for example, Aggregatibacter actinomycetemcomitans; Porphyromonas spp. such as, for example, Porphyromonas gingivalis; Streptococcus spp. such as, for example, Streptococcus sanguis and Streptococcus mutans, Candida spp. such as, for example, Candida albicans, Candida glabrata, Candida krusei, Candida dubliniensis, Candida parapsilosis, and Candida tropicalis; Actinomyces spp. such as, for example, Actinomyces viscosus; and Lactobacillus spp. such as, for example, Lactobacillus casei. In some embodiments, the immune response is against a bacterial pathogen. In some embodiments, the bacterial pathogen is chosen from Staphylococcus spp., such as, for example, Staphylococcus aureus, methicillin-resistant Staphylococcus aureus, and Staphylococcus epidermidis; Streptococcus spp. such as, for example, Streptococcus pneumoniae, Streptococcus pyogenes, and Streptococcus viridans; Escherichia spp. such as, for example, E. coli; Enterococcus spp. such as, for example, Enterococcus faecalis and Enterococcus faecium; Psuedomonas spp. such as, for example, Pseudomonas aeruginosa; Acinetobacter spp. such as, for example, A. baumannii; Haemophilus spp. such as, for example, Haemophilus influenzae; Serratia spp. such as, for example, Serratia marcescens; Moraxella spp. such as, for example, Moraxella catarrhalis; Klebsiella spp. such as, for example, Klebsiella pneumoniae; Proteus spp. such as, for example, Proteus vulgaris and Proteus mirabilis; Bacteroides spp. such as, for example, Bacteroides fragalis; Clostridium spp. such as, for example, Clostridium difficile and Clostridium perfringens; and Propionibacterium spp. such as, for example, Propionibacterium acnes.

Although the disclosed compounds are suitable, other functional groups can be incorporated into the compound with an expectation of similar results. In particular, thioamides and thioesters are anticipated to have very similar properties. The distance between aromatic rings can impact the geometrical pattern of the compound and this distance can be altered by incorporating aliphatic chains of varying length, which can be optionally substituted or can comprise an amino acid, a dicarboxylic acid or a diamine. The distance between and the relative orientation of monomers within the compounds can also be altered by replacing the amide bond with a surrogate having additional atoms. Thus, replacing a carbonyl group with a dicarbonyl alters the distance between the monomers and the propensity of dicarbonyl unit to adopt an anti arrangement of the two carbonyl moiety and alter the periodicity of the compound. Pyromellitic anhydride represents still another alternative to simple amide linkages which can alter the conformation and physical properties of the compound. Modern methods of solid phase organic chemistry (E. Atherton and R. C. Sheppard, Solid Phase Peptide Synthesis A Practical Approach IRL Press Oxford 1989) now allow the synthesis of homodisperse compounds with molecular weights approaching 5,000 Daltons. Other substitution patterns are equally effective.

The compounds of the invention also include derivatives referred to as prodrugs. As used herein, the term “prodrug” refers to a derivative of a known direct acting drug, which derivative has enhanced delivery characteristics and therapeutic value as compared to the drug, and is transformed into the active drug by an enzymatic or chemical process.

It is understood that the present invention encompasses the use, where applicable, of stereoisomers, diastereomers and optical stereoisomers of the compounds of the invention, as well as mixtures thereof, for modulating an immune response. Additionally, it is understood that stereoisomers, diastereomers, and optical stereoisomers of the compounds of the invention, and mixtures thereof, are within the scope of the invention. By way of non-limiting example, the mixture may be a racemate or the mixture may comprise unequal proportions of one particular stereoisomer over the other. Additionally, the compounds of the invention can be provided as a substantially pure stereoisomers, diastereomers and optical stereoisomers (such as epimers).

The compounds of the invention can be provided in the form of an acceptable salt (i.e., a pharmaceutically acceptable salt) for modulating an immune response. Salts can be provided for pharmaceutical use, or as an intermediate in preparing the pharmaceutically desired form of the compounds of the invention. One example of a salt that can be considered to be acceptable is the hydrochloride acid addition salt. Hydrochloride acid addition salts are often acceptable salts when the pharmaceutically active agent has an amine group that can be protonated. Since the compounds of the invention may be polyionic, such as a polyamine, the acceptable salt can be provided in the form of a poly(amine hydrochloride).

The compounds of the invention may be useful as modulators of an immune response. For example, compounds of the invention may be used therapeutically to modulate an immune response in patients such as animals, including humans and non-human vertebrates such as wild, domestic and farm animals. In some embodiments, the modulation of an immune response decreases or eliminates an immune response. In some embodiments, the methods of the present invention can decrease an immune response by greater than about 50%, 60%, 70%, 80%, 85%, 88%, 90%, 92%, 95%, 98%, 99%, 99.2%, 99.5%, 99.8%, or 99.9%. The % decrease in an immune response can be measured by routine immune assays such as, for example, measuring the amount of a particular cytokine produced (at the protein level, nucleic acid level, or protein activity level).

In some embodiments, the modulation or decrease of the immune response takes place in an epithelial cell and/or a myeloid-derived cell. In some embodiments, the cell is a T cell, B cell, or monocyte such as a macrophage. In some embodiments, the cell is a neutrophil.

In some embodiments, the methods of modulating an immune response comprises decreasing the production of a cytokine. In some embodiments, the cytokine is chosen from TNFalpha, IL-1Beta, IL-1alpha, IL-8, IL-6, IL-10, IL-11, IL-12, TGF-Beta, and IFNgamma. In some embodiments, more than one cytokine is decreased. A decrease in a cytokine can be either at the nucleic acid level, the protein level, or the activity of the protein.

In some embodiments, the immune response is against an oral pathogen. In some embodiments, the oral pathogen is chosen from 1n some embodiments, the immune response is against an oral pathogen. In some embodiments, the oral pathogen is chosen from Aggregatibacter spp. such as, for example, Aggregatibacter actinomycetemcomitans; Porphyromonas spp. such as, for example, Porphyromonas gingivalis; Streptococcus spp. such as, for example, Streptococcus sanguis and Streptococcus mutans, Candida spp. such as, for example, Candida albicans, Candida glabrata, Candida krusei, Candida dubliniensis, Candida parapsilosis, and Candida tropicalis; Actinomyces spp. such as, for example, Actinomyces viscosus; and Lactobacillus spp. such as, for example, Lactobacillus casei, or any combination thereof.

In some embodiments, the immune response is against a bacterial pathogen. In some embodiments, the bacterial pathogen is chosen from Staphylococcus spp., such as, for example, Staphylococcus aureus, methicillin-resistant Staphylococcus aureus, and Staphylococcus epidermidis; Streptococcus spp. such as, for example, Streptococcus pneumoniae, Streptococcus pyogenes, and Streptococcus viridans; Escherichia spp. such as, for example, E. coli; Enterococcus spp. such as, for example, Enterococcus faecalis and Enterococcus faecium; Psuedomonas spp. such as, for example, Pseudomonas aeruginosa; Acinetobacter spp. such as, for example, A. baumannii; Haemophilus spp. such as, for example, Haemophilus influenzae; Serratia spp. such as, for example, Serratia marcescens; Moraxella spp. such as, for example, Moraxella catarrhalis; Klebsiella spp. such as, for example, Klebsiella pneumoniae; Proteus spp. such as, for example, Proteus vulgaris and Proteus mirabilis; Bacteroides spp. such as, for example, Bacteroides fragalis; Clostridium spp. such as, for example, Clostridium difficile and Clostridium perfringens; and Propionibacterium spp. such as, for example, Propionibacterium acnes, or any combination thereof.

In some embodiments, suitable dosage ranges for intravenous (i.v.) administration are 0.01 mg to 500 mg per kg body weight, 0.1 mg to 100 mg per kg body weight, 1 mg to 50 mg per kg body weight, or 10 mg to 35 mg per kg body weight. Suitable dosage ranges for other modes of administration can be calculated based on the forgoing dosages as known by those skilled in the art. For example, recommended dosages for intradermal, intramuscular, intraperitoneal, subcutaneous, epidural, sublingual, intracerebral, intravaginal, transdermal administration or administration by inhalation are in the range of 0.001 mg to 200 mg per kg of body weight, 0.01 mg to 100 mg per kg of body weight, 0.1 mg to 50 mg per kg of body weight, or 1 mg to 20 mg per kg of body weight. Effective doses may be extrapolated from dose-response curves derived from in vitro or animal model test systems. Such animal models and systems are well known in the art.

Polyamides and polyesters that are useful for the present invention can be prepared by typical condensation polymerization and addition polymerization processes (see, for example, G. Odian, Principles of Polymerization, John Wiley & Sons, Third Edition (1991), and M. Steven, Polymer Chemistry, Oxford University Press (1999)). Most commonly, the polyamides are prepared by a) thermal dehydration of amine salts of carboxylic acids, b) reaction of acid chlorides with amines, and c) aminolysis of esters. Methods a) and c) are of limited use in polymerizations of aniline derivatives which are generally prepared utilizing acid chlorides. The skilled chemist, however, will recognize that there are many alternative active acylating agents, for example phosphoryl anhydrides, active esters or azides, which may replace an acid chloride and which, depending of the particular polymer being prepared, may be superior to an acid chloride. The acid chloride route is probably the most versatile and has been used extensively for the synthesis of aromatic polyamides.

Homopolymers derived from substituted aminobenzoic acid derivatives can also prepared in a stepwise fashion. A stepwise process comprises coupling an N-protected amino acid to an amine (or hydroxy group) and subsequently removing the amine-protecting group and repeating the process. These techniques have been highly refined for synthesis of specific peptides, allow for the synthesis of specific sequences, and both solid-phase and solution techniques for peptide synthesis are directly applicable to the present invention. An alternative embodiment of the present invention is the corresponding polysulfonamides that can be prepared in analogous fashion by substituting sulfonyl chlorides for carboxylic acid chlorides.

The most common method for the preparation of polyureas is the reaction of diamines with diisocyanates (see, Yamaguchi et al., Polym. Bull., 2000, 44, 247). This exothermic reaction can be carried out by solution techniques or by interfacial techniques. One skilled in organic and polymer chemistry will appreciate that the diisocyanate can be replaced with a variety of other bis-acylating agents, such as phosgene or N,N′-(diimidazolyl)carbonyl, with similar results. Polyurethanes are prepared by comparable techniques using a diisocyanate and a dialcohol or by reaction of a diamine with a bis-chloroformate.

The syntheses of compounds of the invention can be carried out by routine and/or known methods such as those disclosed in, for example, U.S. Patent Application Publication Nos. 2005-0287108, 2006-0041023, U.S. Pat. No. 7,173,102, International Publication Nos. WO 2005/123660, WO 2004/082643, and WO 2006/093813, and U.S. Application Publication No. 2010-0081665, each of which is incorporated herein by reference in its entirety. Numerous pathways are available to incorporate polar and nonpolar side chains. Phenolic groups on the monomer can be alkylated. Alkylation of the commercially available phenol will be accomplished with standard Williamson ether synthesis for the non-polar side chain with ethyl bromide as the alkylating agent. Polar sidechains can be introduced with bifunctional alkylating agents such as BOC—NH(CH₂)₂Br. Alternately, the phenol group can be alkylated to install the desired polar side chain function by employing the Mitsonobu reaction with BOC—NH(CH₂)₂—OH, triphenyl phosphine, and diethyl acetylenedicarboxylate. Standard conditions for reduction of the nitro groups and hydrolysis of the ester afford the amino acid. With the aniline and benzoic acid in hand, coupling can be effected under a variety of conditions. Alternatively, the hydroxy group of the (di)nitrophenol can be converted to a leaving group and a functionality introduced under nucleophilic aromatic substitution conditions. Other potential scaffolds that can be prepared with similar sequences are methyl 2-nitro-4-hydroxybenzoate and methyl 2-hydroxy-4-nitrobenzoate.

The compounds of the invention can also be designed using computer-aided computational techniques, such as de novo design techniques, to embody the amphiphilic properties. In general, de novo design of compounds is performed by defining a three-dimensional framework of the backbone assembled from a repeating sequence of monomers using molecular dynamics and quantum force field calculations. Next, side groups are computationally grafted onto the backbone to maximize diversity and maintain drug-like properties. The best combinations of functional groups are then computationally selected to produce a cationic, amphiphilic structures. Representative compounds can be synthesized from this selected library to verify structures and test their biological activity. Novel molecular dynamic and coarse grain modeling programs have also been developed for this approach because existing force fields developed for biological molecules, such as peptides, were unreliable in these oligomer applications (see, Car et al., Phys. Rev. Lett., 1985, 55, 2471-2474; Siepmann et al., Mol. Phys., 1992, 75, 59-70; Martin et al., J. Phys. Chem., 1999, 103, 4508-4517; and Brooks et al., J. Comp. Chem., 1983, 4, 187-217). Several chemical structural series of compounds have been prepared. See, for example, International Publication No. WO 2002/100295, which is incorporated herein by reference in its entirety. The compounds of the invention can be prepared in a similar manner. Molecular dynamic and coarse grain modeling programs can be used for a design approach. See, for example, U.S. Application Publication No. 2004-0107056, and U.S. Application Publication No. 2004-0102941, each of which is incorporated herein by reference in its entirety.

An example of the design, synthesis, and testing of arylamide polymers and oligomers, a related group of compounds of the invention, is presented in Tew et al., Proc. Natl. Acad. Sci. USA, 2002, 99, 5110-5114, which is incorporated herein by reference in its entirety.

Compounds of the invention can be synthesized by solid-phase synthetic procedures well know to those of skill in the art (see, Tew et al., Proc. Natl. Acad. Sci. USA, 2002, 99, 5110-5114; Barany et al., Int. J. Pept. Prot. Res., 1987, 30, 705-739; Solid-phase Synthesis: A Practical Guide, Kates, S. A., and Albericio, F., eds., Marcel Dekker, New York (2000); and Dörwald, F. Z., Organic Synthesis on Solid Phase: Supports, Linkers, Reactions, 2nd Ed., Wiley-VCH, Weinheim (2002)).

The compounds of the invention can be administered in any conventional manner by any route where they are active. Administration can be systemic, topical, or oral. For example, administration can be, but is not limited to, parenteral, subcutaneous, intravenous, intramuscular, intraperitoneal, transdermal, oral, buccal, or ocular routes, or intravaginally, by inhalation, by depot injections, or by implants. Thus, modes of administration for the compounds of the invention (either alone or in combination with other pharmaceuticals) can be, but are not limited to, sublingual, injectable (including short-acting, depot, implant and pellet forms injected subcutaneously or intramuscularly), or by use of vaginal creams, suppositories, pessaries, vaginal rings, rectal suppositories, intrauterine devices, and transdermal forms such as patches and creams. The selection of the specific route of administration and the dose regimen is to be adjusted or titrated by the clinician according to methods known to the clinician to obtain the desired clinical response. The amount of compounds of the invention to be administered is that amount which is therapeutically effective. The dosage to be administered will depend on the characteristics of the subject being treated, e.g., the particular animal treated, age, weight, health, types of concurrent treatment, if any, and frequency of treatments, and can be easily determined by one of skill in the art (e.g., by the clinician).

The pharmaceutical compositions and/or formulations containing the compounds of the invention and a suitable carrier can be solid dosage forms which include, but are not limited to, tablets, capsules, cachets, pellets, pills, powders and granules; topical dosage forms which include, but are not limited to, solutions, powders, fluid emulsions, fluid suspensions, semi-solids, ointments, pastes, creams, gels and jellies, and foams; and parenteral dosage forms which include, but are not limited to, solutions, suspensions, emulsions, and dry powder; comprising an effective amount of a compound of the invention. It is also known in the art that the active ingredients can be contained in such formulations with pharmaceutically acceptable diluents, fillers, disintegrants, binders, lubricants, surfactants, hydrophobic vehicles, water soluble vehicles, emulsifiers, buffers, humectants, moisturizers, solubilizers, preservatives and the like. The means and methods for administration are known in the art and an artisan can refer to various pharmacologic references for guidance (see, for example, Modern Pharmaceutics, Banker & Rhodes, Marcel Dekker, Inc. (1979); and Goodman & Gilman's The Pharmaceutical Basis of Therapeutics, 6th Edition, MacMillan Publishing Co., New York (1980)).

The compounds of the invention can be formulated for parenteral administration by injection, such as by bolus injection or continuous infusion. The compounds of the invention can be administered by continuous infusion subcutaneously over a period of about 15 minutes to about 24 hours. Formulations for injection can be presented in unit dosage form, such as in ampoules or in multi-dose containers, with an added preservative. The compositions can take such forms as suspensions, solutions or emulsions in oily or aqueous vehicles, and can contain formulatory agents such as suspending, stabilizing and/or dispersing agents.

For oral administration, the compounds of the invention can be formulated readily by combining these compounds with pharmaceutically acceptable carriers well known in the art. Such carriers enable the compounds of the invention to be formulated as tablets, pills, dragees, capsules, liquids, gels, syrups, slurries, suspensions and the like, for oral ingestion by a patient to be treated. Pharmaceutical preparations for oral use can be obtained by, for example, adding a solid excipient, optionally grinding the resulting mixture, and processing the mixture of granules, after adding suitable auxiliaries, if desired, to obtain tablets or dragee cores. Suitable excipients include, but are not limited to, fillers such as sugars, including, but not limited to, lactose, sucrose, mannitol, and sorbitol; cellulose preparations such as, but not limited to, maize starch, wheat starch, rice starch, potato starch, gelatin, gum tragacanth, methyl cellulose, hydroxypropylmethyl-cellulose, sodium carboxymethylcellulose, and polyvinylpyrrolidone (PVP). If desired, disintegrating agents can be added, such as, but not limited to, the cross-linked polyvinyl pyrrolidone, agar, or alginic acid or a salt thereof such as sodium alginate.

Dragee cores can be provided with suitable coatings. For this purpose, concentrated sugar solutions can be used, which can optionally contain gum arabic, talc, polyvinyl pyrrolidone, carbopol gel, polyethylene glycol, and/or titanium dioxide, lacquer solutions, and suitable organic solvents or solvent mixtures. Dyestuffs or pigments can be added to the tablets or dragee coatings for identification or to characterize different combinations of active compound doses.

Pharmaceutical preparations which can be used orally include, but are not limited to, push-fit capsules made of gelatin, as well as soft, sealed capsules made of gelatin and a plasticizer, such as glycerol or sorbitol. The push-fit capsules can contain the active ingredients in admixture with filler such as lactose, binders such as starches, and/or lubricants such as talc or magnesium stearate and, optionally, stabilizers. In soft capsules, the active compounds can be dissolved or suspended in suitable liquids, such as fatty oils, liquid paraffin, or liquid polyethylene glycols. In addition, stabilizers can be added. All formulations for oral administration should be in dosages suitable for such administration.

For buccal administration, the compositions can take the form of, such as, tablets or lozenges formulated in a conventional manner.

For administration by inhalation, the compounds of the invention for use according to the present invention are conveniently delivered in the form of an aerosol spray presentation from pressurized packs or a nebulizer, with the use of a suitable propellant, such as dichlorodifluoromethane, trichlorofluoromethane, dichlorotetrafluoroethane, carbon dioxide or other suitable gas. In the case of a pressurized aerosol the dosage unit can be determined by providing a valve to deliver a metered amount. Capsules and cartridges of, such as gelatin for use in an inhaler or insufflator can be formulated containing a powder mix of the compound and a suitable powder base such as lactose or starch.

The compounds of the invention can also be formulated in rectal compositions such as suppositories or retention enemas, such as containing conventional suppository bases such as cocoa butter or other glycerides.

In addition to the formulations described previously, the compounds of the invention can also be formulated as a depot preparation. Such long acting formulations can be administered by implantation (for example subcutaneously or intramuscularly) or by intramuscular injection. Depot injections can be administered at about 1 to about 6 months or longer intervals. Thus, for example, the compounds can be formulated with suitable polymeric or hydrophobic materials (for example as an emulsion in an acceptable oil) or ion exchange resins, or as sparingly soluble derivatives, for example, as a sparingly soluble salt.

In transdermal administration, the compounds of the invention, for example, can be applied to a plaster, or can be applied by transdermal, therapeutic systems that are consequently supplied to the organism.

The pharmaceutical compositions of the compounds of the invention also can comprise suitable solid or gel phase carriers or excipients. Examples of such carriers or excipients include, but are not limited to, calcium carbonate, calcium phosphate, various sugars, starches, cellulose derivatives, gelatin, and polymers such as polyethylene glycols.

The compounds of the invention can also be administered in combination with other active ingredients such as, for example, anti-inflammatory agents known to those skilled in the art.

The present invention also provides compounds of the invention, or compositions comprising the same, for use in modulating an immune response in a patient. The present invention also provides compounds of the invention, or compositions comprising the same, for use in modulating an immune response. The present invention also provides compounds of the invention, or compositions comprising the same, for use in preparation of a medicament for modulating an immune response in a patient.

In order that the invention disclosed herein may be more efficiently understood, examples are provided below. It should be understood that these examples are for illustrative purposes only and are not to be construed as limiting the invention in any manner. Throughout these examples, molecular cloning reactions, and other standard recombinant DNA techniques, were carried out according to methods described in Maniatis et al., Molecular Cloning-A Laboratory Manual, 2nd ed., Cold Spring Harbor Press (1989), using commercially available reagents, except where otherwise noted.

### EXAMPLES

### Example 1

### Bacterial Strains and Culture

Aggregatibacter actinomycetemcomitans 1005 (Aa) (obtained from Dr. Helen Schreiner, New Jersey Dental School) were cultured on TSB agar (4% trypticase soy broth, 0.6% yeast extract, 0.8% dextrose, 0.4% NaHCO₃, 75 μg/mL bactracin, 5 μg/mL vancomycin) at 37° C., 10% CO₂. Single colonies were inoculated to TSB broth in 75-cm² tissue culture flasks. Biofilm was harvest upon the 90% confluence and resuspended into 1 mL PBS. Resuspension was vortexed vigorously for 1 minute and allowed to settle for 10 minutes. The supernatant was then diluted to 2.5×10⁷ before seeded to 96-well plates to obtain even biofilms. Porphyromonas gingivalis W381 (obtained from Dr. Christopher Cutler, Stony Brook University Dental School) were cultured on TSB-blood agar (3% trypticase soy broth, 5% defibrinated sheep blood, 5 hemin, 0.5 μg/mL menadione, and 0.2 mg/mL KNO₃) in an anaerobic chamber (80% N₂, 10% H₂, and 10% CO₂) at 37° C. For biofilm formation, the same protocol as Aa under anaerobic condition was used.

### Example 2

### Antimicrobial Assays

Aa biofilms were cultured into 96-well plates (tissue culture treated, Falcon) for 18 hours. Serial dilutions of the mimetic compounds were made in 100 μL RPMI-1640 without Phenol red and added directly to the wells. Plates were cultured at 37° C., 10% CO₂ for 24 hours. Medium was removed, and cell viability was evaluated by XTT assay using the In Vitro Toxicology Assay Kit (Sigma) according to the manufacturer's protocol. Metabolic activity was measured by reading in a plate-reader at 450 nm. To determine cell viability by plating, the wells were scraped and resuspended in growth medium, and plated onto TSB agar. Colonies were counted after 72 hours. All assays were performed in duplicate.

### Example 3

### Cell Culture and Stimulation

The oral keratinocyte cell line OKF6/TERT (obtained from Dr. James Rhinewald, Harvard University) was cultured in Keratinocyte growth medium (Lonza) with hEGF, BPE (Bovine Pituitary Extract). Cells were subcultured in 6-well dishes 18 hours before stimulation. Cells were treated with 2 μg/mL, 5 μg/mL mPE with and without IL-1β stimulation (100 ng/mL, 24 hours) for 2 hours, 4 hours and 18 hours. THP-1 cells were grown in suspension at RPMI 1640 with 10% FBS, and stimulated similarly.

### Example 4

### Cytokine and Inflammation Assays

Growth medium from stimulated cultures was collected either by aspiration (from keratinocytes) or after centrifugation at 1000 rpm for 15 minutes (for THP-1 cells). Cell debris was removed by centrifugation at 8,000 g (12,000 rpm) for 10 minutes at 4° C. To quantify IL-8 levels, the Human IL-8 Single Analyte ELISArray Kit (SA bioscience, MD) was used according to the manufacturer's protocol. The Cellular Activation of Signaling ELISA kit IKBα (SA bioscience, MD) was used to quantify both phosphorylated and whole IkBα levels in OKF6/TERT cells grown in a 96-well plate. All assays were performed in duplicate.

### Example 5

### PCR

Total cellular RNA was isolated from cultures using QIAshredder and RNeasy Mini Kit (Qiagen Valencia, Calif.). Total RNA was reversed transcribed using Superscript II reverse transcriptase kit as described by the manufacturer (Invitrogen, CA). Quantitative PCR (qPCR) was carried out using SYBR Green in a MyiQ iCycler (Bio-Rad). A total of 1 μl of cDNA (described above) was analyzed using final concentration of 100 nM of primers, 2×SYBR Green PCR Master Mix (Applied Biosystems, Foster City, Calif., USA) in volume of 20 μl. Prmer sequences were:

| hBD-2: | |
|---|---|
| (SEQ ID NO: 1) | |
| Forward | 5′-GATGCCTCTTCCAGGTGTTTTTGG-3′ |
| (SEQ ID NO: 2) | |
| Reverse | 5′-TTG TTCCAGGACCACAGGTG-3′ |
| IL-8: | |
| (SEQ ID NO: 3) | |
| Forward | 5′-GCAGCTCTGTGTGAAGGTGCAGTTTTGC-3′ |
| (SEQ ID NO: 4) | |
| Reverse | 5′-TTTCTGTGTTGGCGCAGTGTGGTCC-3′ |
| b-2-microgloublin (control): | |
| (SEQ ID NO: 5) | |
| Forward | 5′-CTCCGTGGCCTTAGCTGTG-3′ |
| (SEQ ID NO: 6) | |
| Reverse | 5′-TTGGAGTACGCTGGATAGCCT-3′ |

Amplification was carried out for 50 cycles (95° C., 15 seconds; 60° C., 60 seconds). The relative for mRNA expression in each sample was calculated based on its Ct value comparison to Ct of a housekeeping gene. The data were presented as 2^{-DDCt}, an arbitrary unit. All amplified products showed single peak in the dissociation curve test. RTQ-PCR was performed in triplicates for each sample. This procedure was conducted in at least three independent experiments.

### Example 6

### Activity Against A. actinomycetemcomitans and P. gingivalis

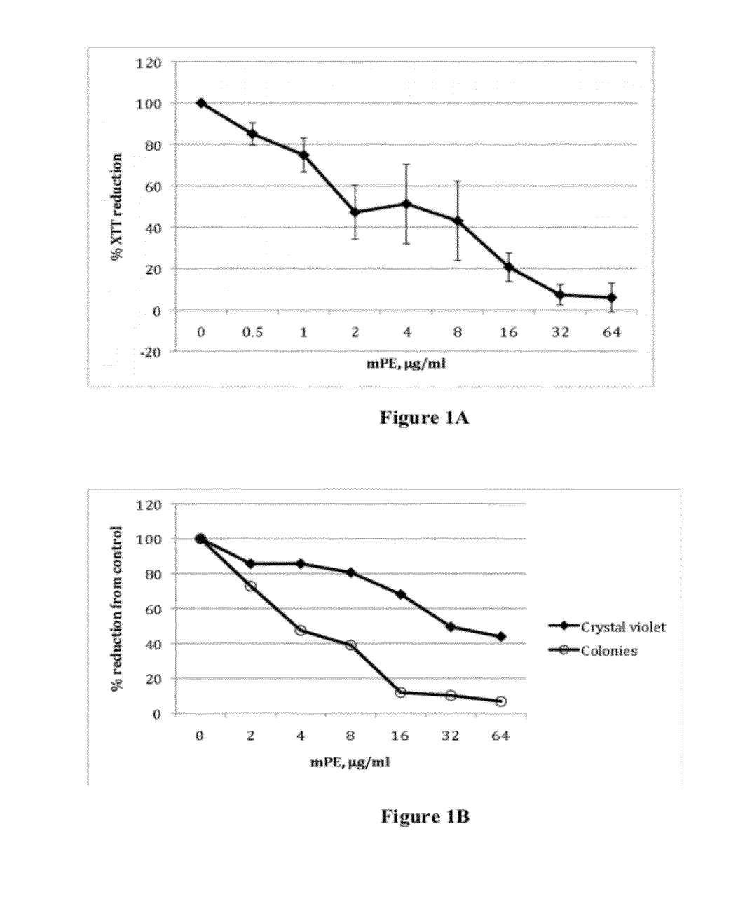

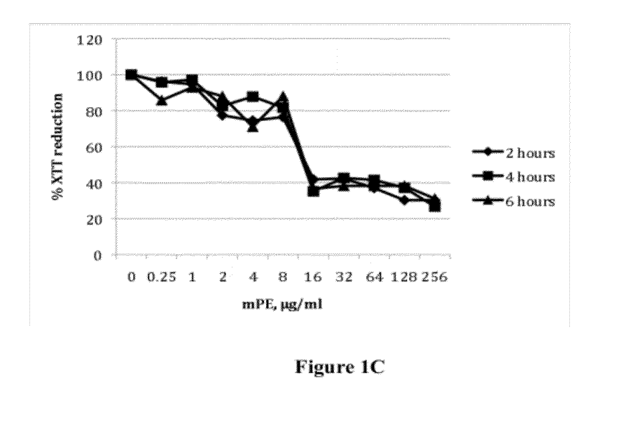

To quantify the activity of AMP mimetics on biofilms, the activity against two bacterial species associated with periodontitis, A. actinomycetemcomitans and P. gingivalis was measured under conditions that lead to biofilm formation (Kaplan et al., J. Bacteriol. 2003, 185, 1399-1404; Davey, Periodontol 2000, 2006, 42, 27-35). The MIC of mPE against these species in planktonic form is 0.4 μg/ml for A. actinomycetemcomitans and 2.5 μg/ml for P. gingivalis (Beckloff et al., Antimicrob. Agents Chemother., 2007, 51, 4125-4132). Aa strain IDH781 was grown in AAGM in 96-well plates until complete confluence. To assess the activity against A. actinomycetemcomitans biofilms, mPE was added at decreasing concentrations in two-fold dilutions as in a standard MIC assay. After 24 hours, the growth medium was replaced with RPMI (without Phenol Red) and an XTT assay was carried out to quantify the metabolic activity. Metabolic activity was quantified by measuring the OD at 450 nm and 600 nm. Results are shown as % reduction in the A450-A600 from untreated cultures. Experiment in (A) was carried out in triplicate, and error bars=±SD. The results in FIG. 1A demonstrate that mPE exhibits potent antimicrobial activity against A. actinomycetemcomitans grown in biofilms. To confirm that a reduction in metabolic activity led to a reduction in biomass and viable bacteria, the wells from a separate experiment were stained with Crystal violet and plated the bacteria from a parallel experiment to that shown in FIG. 1A, and show that the reduction in viable colonies paralleled the results from the XTT assay (FIG. 1B). In particular, in panel B, parallel wells were stained with Crystal violet, destained and quantified by reading A600, and bacteria were removed and plated on AAGM agar plates to quantify viable colonies. Results are shown as % reduction from untreated control. Killing of A. actinomycetemcomitans occurs rapidly as demonstrated in FIG. 1C, where even a 2-hour exposure was sufficient to reduce metabolic activity by 60% at 16 ng/ml.

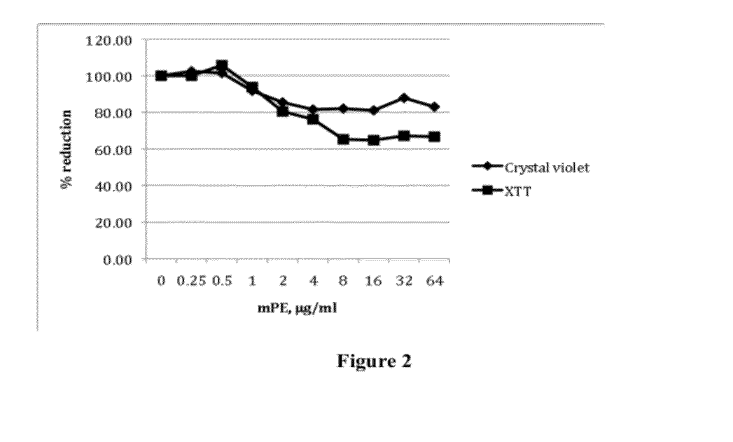

To test the activity against P. gingivalis biofilms, strain 381 was grown in 96-well plates under conditions (i.e., grown in a 96-well plate for 21 days in an anaerobic chamber in Brain Heart Infusion (BHI) medium) that favor biofilm formation (Davey, Periodontol 2000, 2006, 42, 27-35). mPE was added in serial dilutions, incubated anaerobically for 24 hours, and the medium was replaced with XTT in RPMI. Metabolic activity was quantified as above. To confirm the ability of XTT to measure activity in the biofilm, the growth medium was removed, and biomass was quantified by crystal violet staining, followed by destaining and quantification of the optical density. Staining was quantified by reading A600. The results shown in FIG. 2 show that there is a decrease in both biofilm and metabolic activity to a baseline at 4-8 μg/ml mPE. Values represent mean of duplicate experiments.

### Example 7

### The Effect of mPE on Inflammatory Response

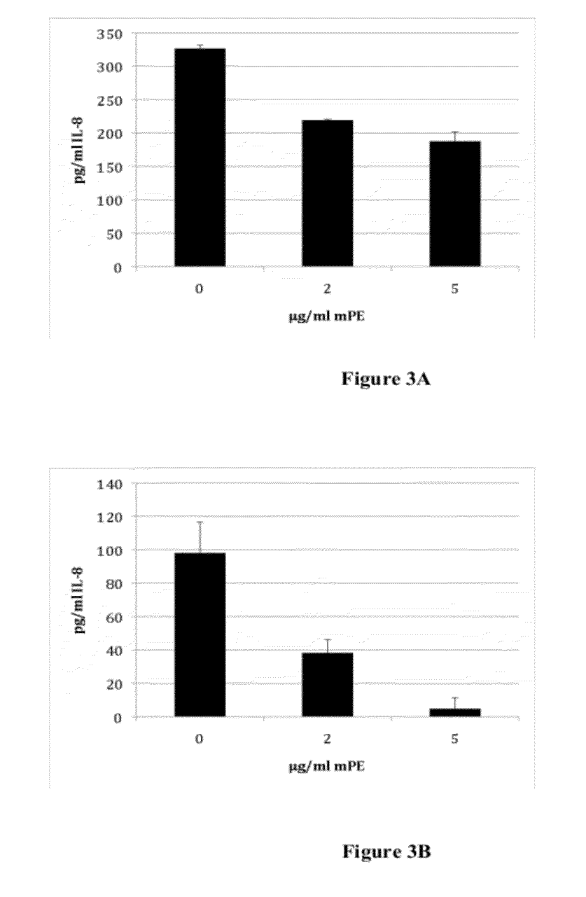

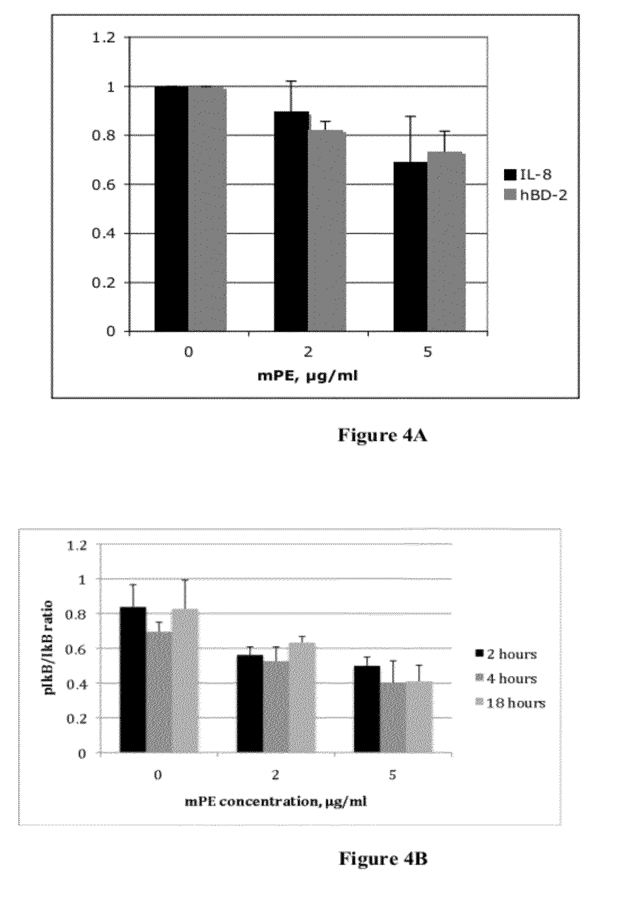

To examine the effect of mPE on the inflammatory response, gingival epithelial cells (the OKF6/TERT cell line) and the monocytic cell line, THP-1, were treated with rhIL-1β (100 ng/ml) in the presence of increasing concentrations (0, 2, or 5 ng/ml) of mPE. Secreted levels of IL-8 were measured by ELISA. No IL-8 was observed in either case in the absence of IL-1β (not shown). The experiment was carried out in quadruplicate; error bars represent±SD. The inhibition by mPE was significant at both concentrations with p<0.01. The results shown in FIG. 3 demonstrate a dose-dependent inhibition of IL-8 secretion by mPE. This was not a result of cytotoxicity, as cell viability (as measured by XTT assay and trypan blue exclusion) was no lower than 93-96% at the highest concentration of mPE. To determine whether this was due to an effect on IL-8 secretion or on gene regulation, mRNA was isolated from treated cells IL-8 mRNA levels were quantified by QPCR. The results in FIG. 4A, which mirror the reduction of IL-8 protein, show that the inhibitory effect is at the level of gene expression.

OKF6/TERT cells were treated with mPE as above in the presence or absence of IL-1β. Total mRNA was isolated and IL-8 and hBD-2 mRNA levels were quantified by QPCR normalized to β2-Microglobulin. Levels are shown relative to the no-mPE sample for each group. The experiment was carried out in triplicate; error bars represent±SD. There was a similar inhibitory response in steady-state mRNA levels of another IL-1β-stimulated host defense gene, hBD-2 (FIG. 4A). To determine whether this was due to an inhibition of NF-κB activation, gingival epithelial cells were treated with mPE in the presence or absence of 100 ng/ml IL-1β, and IκB phosphorylation levels were quantified using the CASE assay (SA Biosciences, MD), and quantified relative to total IκB levels). In particular, OKF6/TERT cells were grown in 96-well plates, treated with 100 ng/ml IL-1β for 2 or 4 hours in the presence of 0, 2 or 5 μg/ml mPE. Shown are phosphorylated IκB levels/total IκB of IL-1β-treated cultures compared to untreated cultures. The experiment was carried out twice in quadruplicate. Reductions in pIκB/total IκB are significant at p<0.002. The results shown in FIG. 4B demonstrate a rapid, dose-dependent reduction in IL-1β-stimulated phosphorylated IκB levels by mPE.

A computational model of mPE demonstrates predicted binding to LPS, and incubation of mPE with macrophage cells inhibited LPS-mediated TNF-α production, similar to that seen with the LPS-binding antibiotic Polymyxin B (Beckloff et al., Antimicrob. Agents Chemother., 2007, 51, 4125-4132). Thus, it was surprising that mPE demonstrated similar anti-inflammatory properties inhibiting the IL-1β-induced inflammatory response in both epithelial and myeloid cells. The data that mPE inhibited both IL-8 protein secretion and mRNA levels, as well as hBD-2 expression and IκB-phosphorylation suggests that it acts on the NF-κB signal transduction pathway induced by IL-1β.

Various modifications of the invention, in addition to those described herein, will be apparent to those skilled in the art from the foregoing description. Such modifications are also intended to fall within the scope of the appended claims. Each reference (including, but not limited to, journal articles, U.S. and non-U.S. patents, patent application publications, international patent application publications, gene bank accession numbers, and the like) cited in the present application is incorporated herein by reference in its entirety.

What is claimed is:

1. A method of modulating an immune response in a mammal comprising administering to the mammal in need thereof a therapeutically effective amount of a compound of:
a) Formula I: 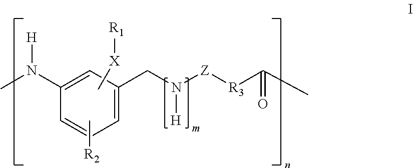
or a pharmaceutically acceptable salt thereof,
wherein:
X is O or S;
R₁ is C₁-C₉ straight or branched chain alkyl, optionally substituted with one or more —NH₂ or —NH—C(═NH)NH₂;
Y is a bond or a carbonyl;
Z is a bond or a carbonyl;
R₂ is hydrogen or C₁-C₉ straight or branched chain alkyl optionally substituted with one or more —NH₂ or —NH—C(═NH)NH₂;
or R₂ is —X—R₁;
R₃ is methylene or 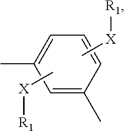
wherein the methylene is substituted with C₁-C₉ straight or branched chain alkyl, wherein the C₁-C₉ straight or branched chain alkyl is optionally substituted with one or more —NH₂ or —NH—C(═NH)NH₂;
n is 2-10; and
m is 1 or 2;
b) Formula II: 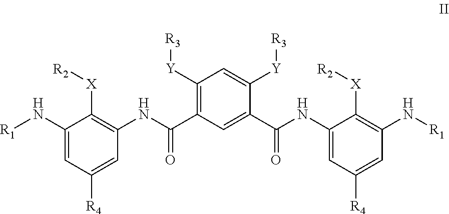
or a pharmaceutically acceptable salt thereof,
wherein:
X is O or S;
Y is O or S;
R₁ is H or —C(═O)-A, where A is C₁-C₉ straight or branched alkyl optionally substituted with one or more —NH₂, —N(CH₃)₂ or —NH—C(═NH)NH₂;
R₂ is C₁-C₉ straight or branched alkyl optionally substituted with one or more —NH₂, —N(CH₃)₂ or —NH—C(═NH)NH₂;
R₃ is C₁-C₉ straight or branched alkyl optionally substituted with one or more —NH₂, —N(CH₃)₂ or —NH—C(═NH)NH₂; and
R₄ is H, —B, or —C(═O)—O—B, where B is C₁-C₉ straight or branched alkyl;
c) Formula III: 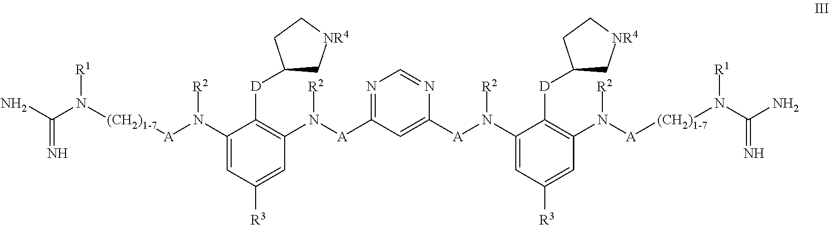
or a pharmaceutically acceptable salt thereof,
wherein:
each A is, independently, —C═O, —C═S, or CH₂;
each D is, independently, O or S;
each R¹ is, independently, hydrogen, C₁₋₃alkyl, C₁₋₃alkoxy, halo, or haloC₁₋₃alkyl;
each R² is, independently, hydrogen, C₁₋₃alkyl, C₁₋₃alkoxy, halo, or halo C₁₋₃alkyl;
each R³ is, independently, hydrogen, C₁₋₄alkyl, C₁₋₄alkoxy, halo, or haloC₁₋₄alkyl; and
each R⁴ is, independently, hydrogen, C₁₋₃alkyl, C₁₋₃alkoxy, halo, or haloC₁₋₃alkyl;
d) Formula IV: 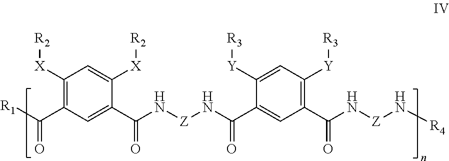
or a pharmaceutically acceptable salt thereof,
wherein:
n=1 to 10;
X is O or S;
Y is O or S;
Z is a bond, C₁-C₉ straight or branched alkyl, or a 1,4-cyclohexyl;
R₁ is NH₂ or NH-A, where A is C₁-C₉ straight or branched alkyl, where A is optionally substituted with —NH₂, —N(CH₃)₂ or —NH—C(═NH)NH₂;
R₂ is C₁-C₉ straight or branched alkyl, where R₂ is optionally substituted with one or more —NH₂, —N(CH₃)₂ or —NH—C(═NH)NH₂;
R₃ is C₁-C₉ straight or branched alkyl, where R₃ is optionally substituted with one or more —NH₂, —N(CH₃)₂ or —NH—C(═NH)NH₂;
R₄ is H or 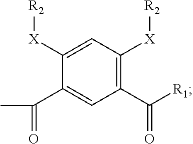
e) Formula V: 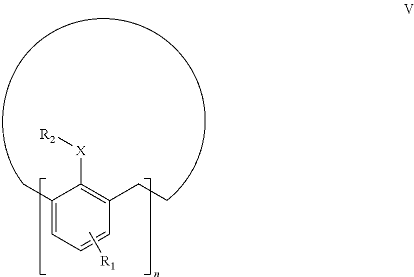
or a pharmaceutically acceptable salt thereof,
wherein:
n is 2-8;
X is a bond, O or —O—CH₂—C(═O)—O—,
R₁ is -A or —O-A, where A is C₁-C₉ straight or branched alkyl; and
R₂ is C₁-C₉ straight or branched alkyl, where R₂ is optionally substituted with one or more —NH₂, —N(CH₃)₂, or —NH—C(═NH)NH₂;
f) Formula VI: 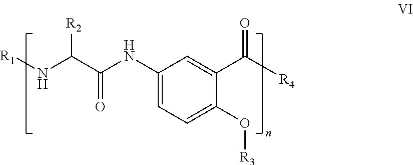
or a pharmaceutically acceptable salt thereof,
wherein:
n is 2 to 10;
R₁ is H or 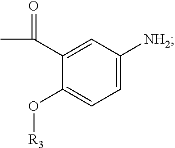
R₂ is C₁-C₉ straight or branched alkyl, where R₂ is optionally substituted with one or more —NH₂, —N(CH₃)₂ or —NH—C(═NH)NH₂;
R₃ is C₁-C₉ straight or branched alkyl, where R₂ is optionally substituted with one or more —NH₂, —N(CH₃)₂ or —NH—C(═NH)NH₂;
R₄ is OH, NH₂ or 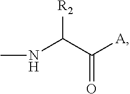
where A is OH or NH₂;
g) Formula VII: 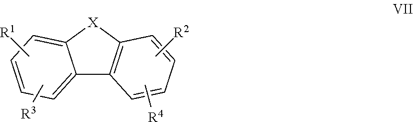
or a pharmaceutically acceptable salt thereof,
wherein:
X is C(R⁷)C(R⁸), C(═O), N(R⁹), O, S, S(═O), or S(═O)₂;
R⁷, R⁸, and R⁹ are, independently, H, C₁-C₈alkyl, C₁-C₈alkoxy, halo, OH, CF₃, or aromatic group;
R¹ and R² are, independently, H, C₁-C₈alkyl, C₁-C₈alkoxy, halo, OH, haloC₁-C₈alkyl, or CN;
R³ and R⁴ are, independently, carbocycle(R⁵)(R⁶);
each R⁵ and each R⁶ are, independently, H, C₁-C₈alkyl, C₁-C₈alkoxy, halo, OH, CF₃, aromatic group, heterocycle, or the free base or salt form of —(CH₂)ₙ—NH₂, or —(CH₂)ₙ—NH—(CH₂)ₙ—NH₂, or —(CH₂)ₙ—NH—C(═NH)NH₂, where each n is, independently, 1 to 8;
or a pharmaceutically acceptable salt thereof;
h) Formula VIII: 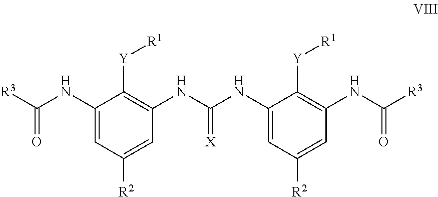
or a pharmaceutically acceptable salt thereof,
wherein:
X is O or S;
each Y is, independently, O, S, or N;
each R¹ is, independently, H, 5- or 6-membered heterocycle, or the free base or salt form of —(CH₂)ₙ—NH₂ or —(CH₂)ₙ—NH—C(═NH)NH₂, where each n is, independently, 1 to 4; or each R¹ is, independently, together with Y a 5- or 6-membered heterocycle;
each R² is, independently, H, CF₃, C(CH₃)₃, halo, or OH; and
each R³ is, independently, —(CH₂)ₙ—NH₂ or —(CH₂)ₙ—NH—C(═NH)NH₂, where each n is, independently, 1 to 4;
or a pharmaceutically acceptable salt thereof;
i) Formula IX:
Q—X—Z—X-Q IX
or a pharmaceutically acceptable salt thereof,
wherein:
Z is 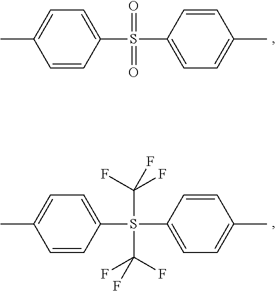
or phenyl;
each Q is, independently, 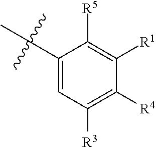
or —C(═O)—(CH₂)_{b}—NH—C(═NH)—NH₂, where each b is, independently, 1 to 4;
each X is, independently, O, S, or N;
each R¹ is, independently, H, CF₃, C(CH₃)₃, halo, or OH;
each R³ is, independently, H, —NH—R², —(CH₂)ₙ—NH₂, —NH₂, —NH—(CH₂)_{w}—NH₂, or 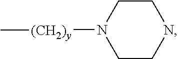
where each r is, independently, 1 or 2, each w is, independently, 1 to 3, and each y is, independently, 1 or 2;
each R² is, independently, H, or the free base or salt form of —(CH₂)ₙ—NH₂ or (CH₂)ₙ—NH—C(═NH)NH₂, where each n is, independently, 1 to 4;
each R⁴ is, independently, H, —NH—C(═O)—(CH₂)ₚ—NH—C(═NH)—NH₂ or 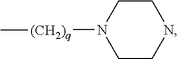
where each p is, independently, 1 to 6, and each q is, independently, 1 or 2; and
each R⁵ is, independently, H or CF₃;
or a pharmaceutically acceptable salt thereof;
j) Formula X: 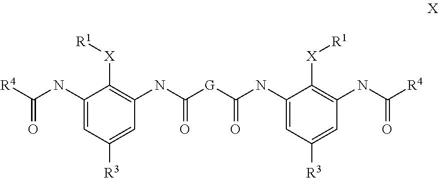
or a pharmaceutically acceptable salt thereof,
wherein:
G is 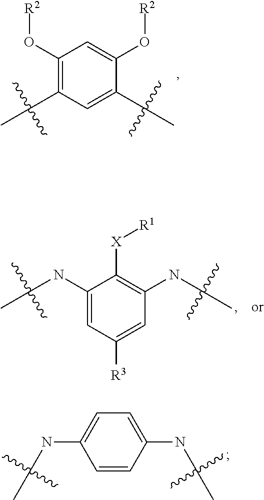
each X is, independently, O or S;
each R¹ is, independently, 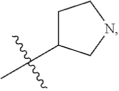
or the free base or salt form of —(CH₂)ₙ—NH₂ or —(CH₂)ₙ—NH—C(═NH)NH₂, where each n is, independently, 1 to 4;
each R² is, independently, H, C₁-C₈alkyl, or the free base or salt form of —(CH₂)ₙ—NH₂ or —(CH₂)ₙ—NH—C(═NH)NH₂, where each n is, independently, 1 to 4;
each R³ is, independently, H, CF₃, C(CH₃)₃, halo, or OH; and
each R⁴ is, independently, —(CH₂)ₙ—NH₂ or —(CH₂)ₙ—NH—C(═NH)NH₂, where each n is, independently, 1 to 4;
k) Formula XI: 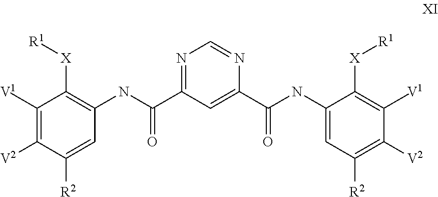
or a pharmaceutically acceptable salt thereof,
wherein:
each X is, independently, O, S, or S(═O)₂;
each R¹ is, independently, —(CH₂)ₙ—NH₂, —(CH₂)ₙ—NH—C(═NH)NH₂, or —(CH₂)ₙ—NH—C(═O)—R⁴, where each n is, independently, 1 to 4, and each R⁴ is, independently, H, C₁-C₃alkyl, or —(CH₂)ₚ—NH₂, where each p is, independently, 1 or 2;
each R² is, independently, H, halo, CF₃, or C(CH₃)₃; and
each V² is H, and each V¹ is, independently, —N—C(═O)—R³, where each R³ is, independently, —(CH₂)ₙ—NH₂ or —(CH₂)ₙ—NH—C(═NH)NH₂, where each n is, independently, 1 to 4; or each V¹ is H and each V² is, independently, —S—R⁵, where each R⁵ is, independently, —(CH₂)ₙ—NH₂ or —(CH₂)ₙ—NH—C(═NH)NH₂, where each n is, independently, 1 to 4;
or a pharmaceutically acceptable salt thereof;
l) Formula XII: 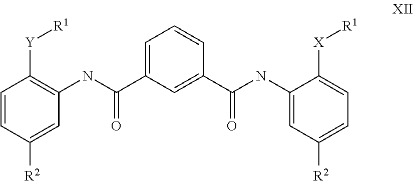
or a pharmaceutically acceptable salt thereof,
wherein:
each Y is, independently, O, S, or NH;
each R¹ is, independently, —(CH₂)ₙ—NH₂ or —(CH₂)ₙ—NH—C(═NH)NH₂, where each n is, independently, 1 to 4; and
each R² is, independently, H, halo, CF₃, or C(CH₃)₃;
or a pharmaceutically acceptable salt thereof;
m) Formula XIII: 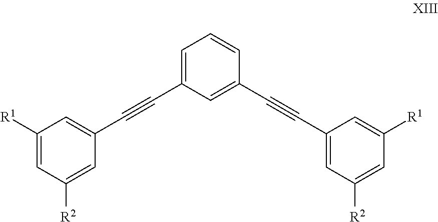
or a pharmaceutically acceptable salt thereof,
wherein:
each R¹ is, independently, H, C₁-C₈alkyl, C₁-C₈alkoxy, halo, OH, CF₃, or CN;
each R² is, independently, —(CH₂)ₙ—NH₂ or —(CH₂)ₙ—NH—C(═NH)NH₂, where each n is, independently, 1 to 4;
or a pharmaceutically acceptable salt thereof;
n) Formula XIV: 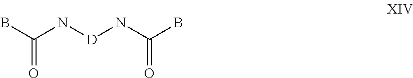
or a pharmaceutically acceptable salt thereof,
wherein:
D is 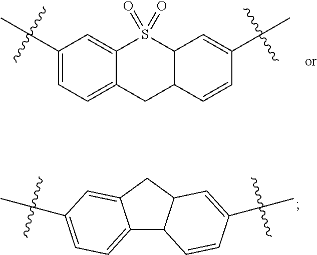
each B is, independently, —(CH₂)ₙ—NH—C(═NH)NH₂, where each n is, independently, 1 to 4 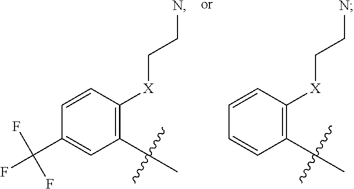
and
each X is, independently, O or S;
or a pharmaceutically acceptable salt thereof;
o) Formula XV: 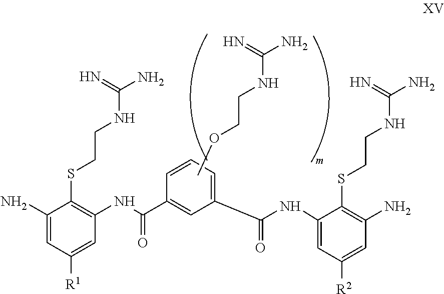
or a pharmaceutically acceptable salt thereof,
wherein:
R¹ is H or C₁₋₁₀ alkyl;
R² is H or C₁₋₁₀ alkyl; and
m is 1 or 2;
p) Formula XVI: 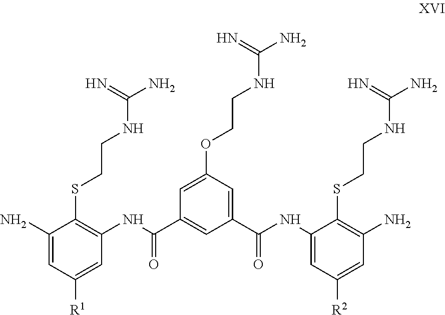
or a pharmaceutically acceptable salt thereof,
wherein:
R¹ is H or C₁₋₈ alkyl; and
R² is H or C₁₋₈ alkyl;
q) Formula XVII: 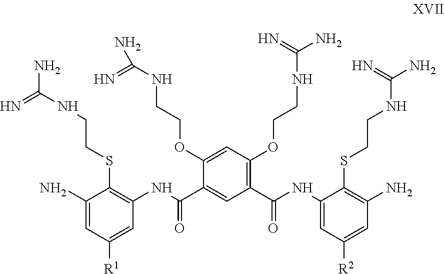
or a pharmaceutically acceptable salt thereof,
wherein:
R¹ is H or C₁₋₈ alkyl; and
R² is H or C₁₋₈ alkyl;
r) Formula XVIII:
R¹—[—X-A₁-Y—X-A₂-Y—]ₘR² XVIII
or a pharmaceutically acceptable salt thereof,
wherein:
each X is, independently, NR⁸, —N(R⁸)N(R⁸)—, O, or S;
each Y is, independently, C═O, C═S, O═S═O, —C(═O)C(═O)—, or —CR^{a}R^{b}—;
R^{a} and R^{b} are each, independently, hydrogen, a PL group, or an NPL group;
each R⁸ is, independently, hydrogen or alkyl;
A₁ and A₂ are each, independently, optionally substituted arylene or optionally substituted heteroarylene, wherein A₁ and A₂ are, independently, optionally substituted with one or more PL group(s), one or more NPL group(s), or a combination of one or more PL group(s) and one or more NPL group(s); or
each A₁ is, independently, optionally substituted arylene or optionally substituted heteroarylene, and each A₂ is a C₃ to C₈ cycloalkyl or —(CH₂)_{q}—, wherein q is 1 to 7, wherein A₁ and A₂ are, independently, optionally substituted with one or more PL group(s), one or more NPL group(s), or a combination of one or more PL group(s) and one or more NPL group(s); or
each A₂ is optionally substituted arylene or optionally substituted heteroarylene, and each A₁ is a C₃ to C₈ cycloalkyl or —(CH₂)_{q}—, wherein q is 1 to 7, wherein A₁ and A₂ are each, independently, optionally substituted with one or more PL group(s), one or more NPL group(s), or a combination of one or more PL group(s) and one or more NPL group(s);
R¹ is hydrogen, a PL group, or an NPL group, and R² is —X-A₁-Y—R¹¹, wherein R¹¹ is hydrogen, a PL group, or an NPL group; or
R¹ and R² are each, independently, hydrogen, a PL group, or an NPL group; or
R¹ and R² together are a single bond; or
R¹ is —Y-A₂-X—R¹², wherein R¹² is hydrogen, a PL group, or an NPL group, and R² is hydrogen, a PL group, or an NPL group;
each NPL group is, independently, —B(OR⁴)₂ or —(NR³′)_{q1NPL}—U^{NPL}-LK^{NPL}—(NR³″)_{q2NPL}—R⁴′, wherein:
R³, R³′, and R³″ are each, independently, hydrogen, alkyl, or alkoxy;
R⁴ and R⁴′ are each, independently, hydrogen, alkyl, alkenyl, alkynyl, cycloalkyl, aryl, or heteroaryl, wherein each of the alkyl, alkenyl, alkynyl, cycloalkyl, aryl, and heteroaryl is optionally substituted with one or more substitutents, wherein each substituent is, independently, alkyl, halo, or haloalkyl;
each U^{NPL} is, independently, absent or O, S, S(═O), S(═O)₂, NR³, —C(═O)—, —C(═O)—NR³—, —C(═O)—N═N—NR³—, —C(═O)—NR³—N═N—, —N═N—NR³—, —C(═N—N(R³)₂)—, —C(═NR³)—, —C(═O)O—, —C(═O)S—, —C(═S)—, —O—P(═O)₂O—, —S—C═N—, or —C(═O)—NR³—O—, wherein groups with two chemically nonequivalent termini can adopt both possible orientations;
each LK^{NPL} is, independently, —(CH₂)_{pNPL}— or C₂₋₈ alkenylenyl, wherein each of the —(CH₂)_{pNPL} and C₂₋₈ alkenylenyl is optionally substituted with one or more substituents, wherein each substituent is, independently, amino, hydroxyl, aminoalkyl, hydroxylalkyl, or alkyl;
each pNPL is, independently, an integer from 0 to 8;
q1NPL and q2NPL are each, independently, 0, 1, or 2;
each PL group is, independently, halo, hydroxyethoxymethyl, methoxyethoxymethyl, polyoxyethylene, or —(NR⁵′)_{q2PL}—U^{PL}-LK^{PL}—(NR⁵″)_{q2PL}—V, wherein:
R⁵, R⁵′, and R⁵″ are each, independently, hydrogen, alkyl, or alkoxy;
each U^{PL} is, independently, absent or O, S, S(═O), S(═O)₂, NR⁵, —C(═O)—, —C(═O)—NR⁵—, —C(═O)—N═N—NR⁵—, —C(═O)—NR⁵—N═N—, —N═N—NR⁵—, —C(═N—N(R⁵)₂)—, —C(═NR⁵)—, —C(═O)O—, —C(═O)S—, —C(═S)—, —O—P(═O)₂O—, —S—C═N—, or —C(═O)—NR⁵—O—, wherein groups with two chemically nonequivalent termini can adopt either of the two possible orientations;
each V is, independently, nitro, cyano, amino, halo, hydroxy, alkoxy, alkylthio, alkylamino, dialkylamino, —NH(CH₂)ₚNH₂ wherein p is 1 to 5, —C(═O)NH(CH₂)ₚNH₂ wherein p is 1 to 5, —C(═O)NH(CH₂)ₚNHC(═NH)NH₂ wherein p is 1 to 5, —C(═O)NH(CH₂)ₚNHC(═O)NH₂ wherein p is 1 to 5, —NHC(═O)-alkyl, —N(CH₂CH₂NH₂)₂, diazamino, amidino, guanidino, ureido, carbamoyl, —C(═O)OH, —C(═O)OR^{c}, —C(═O)NH—OH, —O—NH—C(═NH)NH₂, —NH—S(═O)₂OH, S(═O)₂OH, NR^{d}R^{e}, semicarbazone, aryl, cycloalkyl, heterocycloalkyl, or heteroaryl, wherein each of the aryl and cycloalkyl is substituted with one or more substitutents, wherein each of the heterocycloalkyl and heteroaryl is optionally substituted with one or more substituents, and wherein each of the substituents for the aryl, cycloalkyl, heterocycloalkyl, and heteroaryl is, independently, nitro, cyano, amino, halo, hydroxy, alkoxy, alkylthio, alkylamino, dialkylamino, —NH(CH₂)ₚNH₂ wherein p is 1 to 5, —N(CH₂CH₂NH₂)₂, diazamino, amidino, guanidino, ureido, carbamoyl, —C(═O)OH, —C(═O)OR^{c}, —C(═O)NH—OH, —O—NH—C(═NH)NH₂, —NH—S(═O)₂OH, S(═O)₂OH, NR^{d}R^{e}, semicarbazone, aminosulfonyl, aminoalkoxy, aminoalkylthio, lower acylamino, or benzyloxycarbonyl;
each R^{c} is, independently, C₁₋₆ alkyl, C₁₋₆ haloalkyl, C₂₋₆ alkenyl, C₂₋₆ alkynyl, aryl, cycloalkyl, heteroaryl, heterocycloalkyl, arylalkyl, heteroarylalkyl, cycloalkylalkyl, or heterocycloalkylalkyl, each optionally substituted by one or more substitutents, wherein each substituent is, independently, OH, amino, halo, C₁₋₆ alkyl, C₁₋₆ haloalkyl, aryl, arylalkyl, heteroaryl, heteroarylalkyl, cycloalkyl, or heterocycloalkyl;
R^{d} and R^{e} are, independently, H, C₁₋₆ alkyl, C₁₋₆ haloalkyl, C₂₋₆ alkenyl, C₂₋₆ alkynyl, aryl, heteroaryl, cycloalkyl, heterocycloalkyl, arylalkyl, heteroarylalkyl, cycloalkylalkyl, or heterocycloalkylalkyl, wherein each of the C₁₋₆ alkyl, C₁₋₆ haloalkyl, C₂₋₆ alkenyl, C₂₋₆ alkynyl, aryl, heteroaryl, cycloalkyl, heterocycloalkyl, arylalkyl, heteroarylalkyl, cycloalkylalkyl and heterocycloalkylalkyl is optionally substituted by OH, amino, halo, C₁₋₆ alkyl, C₁₋₆haloalkyl, C₁₋₆haloalkyl, aryl, arylalkyl, heteroaryl, heteroarylalkyl, cycloalkyl, or heterocycloalkyl;
or R^{d} and R^{e} together with the N atom to which they are attached form a 4-, 5-, 6-, 7-, or 8-membered heterocycloalkyl;
each LK^{PL} is, independently, —(CH₂)_{pPL}— or C₂₋₈ alkenylenyl, wherein each of the —(CH₂)_{pNPL}— and C₂₋₈ alkenylenyl is optionally substituted with one or more substituents, wherein each substituent is, independently, amino, hydroxyl, aminoalkyl, hydroxylalkyl, or alkyl;
each pPL is, independently, an integer from 0-8;
q1PL and q2PL are each, independently, 0, 1, or 2; and
m is an integer from 1 to about 20;
s) Formula XIX:
R¹—[—X-A₁-X—Y-A₂-Y-]ₘR² XIX
or a pharmaceutically acceptable salt thereof,
wherein:
each X is, independently, NR⁸, O, S, —N(R⁸)N(R⁸)—, —N(R⁸)—(N═N)—, —(N═N)—N(R⁸)—, —C(R⁷R⁷′)NR⁸—, —C(R⁷R⁷′)O—, or —C(R⁷R⁷′)S—;
each Y is, independently, C═O, C═S, O═S═O, —C(═O)C(═O)—, C(R⁶R⁶′)C═O, or C(R⁶R⁶′)C═S;
each R⁸ is, independently, hydrogen or alkyl;
each R⁷ and each R⁷′ are, independently, hydrogen or alkyl; or R⁷ and R⁷′ together form —(CH₂)ₚ—, wherein p is 4 to 8;
each R⁶ and each R⁶′ are, independently, hydrogen or alkyl; or R⁶ and R⁶′ together form —(CH₂)₂NR¹²(CH₂)₂—, wherein R¹² is hydrogen, —C(═N)CH₃, or —C(═NH)—NH₂;
A₁ and A₂ are each, independently, optionally substituted arylene or optionally substituted heteroarylene, wherein A₁ and A₂ are each, independently, optionally substituted with one or more PL group(s), one or more NPL group(s), or a combination of one or more PL group(s) and one or more NPL group(s);
or each A₂ is, independently, optionally substituted arylene or optionally substituted heteroarylene, and each A₁ is, independently, optionally substituted C₃ to C₈ cycloalkyl, wherein A₁ and A₂ are each, independently, optionally substituted with one or more PL group(s), one or more NPL group(s), or a combination of one or more PL group(s) and one or more NPL group(s);
R¹ is hydrogen, a PL group, or an NPL group, and R² is —X-A₁-X—R¹, wherein A₁ is as defined above and is optionally substituted with one or more PL group(s), one or more NPL group(s), or a combination of one or more PL group(s) and one or more NPL group(s); or
R¹ is hydrogen, a PL group, or an NPL group, and R² is —X-A′-X—R¹, wherein A′ is C₃ to C₈ cycloalkyl, aryl, or heteroaryl and is optionally substituted with one or more PL group(s), one or more NPL group(s), or a combination of one or more PL group(s) and one or more NPL group(s); or
R¹ is —Y-A₂-Y—R², and each R² is, independently, hydrogen, a PL group, or an NPL group; or
R¹ is —Y-A¹ and R² is —X-A′, wherein each A′ is, independently, C₃ to C₈ cycloalkyl, aryl, or heteroaryl and is optionally substituted with one or more PL group(s), one or more NPL group(s), or a combination of one or more PL group(s) and one or more NPL group(s); or
R¹ and R² are, independently, a PL group or an NPL group; or
R¹ and R² together form a single bond;
each NPL is, independently, —B(OR⁴)₂ or —(NR³′)_{q1NPL}—U^{NPL}-LK^{NPL}—(NR³″)_{q2NPL}—R⁴′, wherein:
R³, R³′, and R³″ are each, independently, hydrogen, alkyl, or alkoxy;
R⁴ and R⁴′ are each, independently, hydrogen, alkyl, alkenyl, alkynyl, cycloalkyl, aryl, and heteroaryl, wherein each of the alkyl, alkenyl, alkynyl, cycloalkyl, aryl, and heteroaryl is optionally substituted with one or more alkyl or halo groups;
each U^{NPL} is, independently, absent or O, S, S(═O), S(═O)₂, NR³, —C(═O)—, —C(═O)—NR³—, —C(═O)—N═N—NR³—, —C(═O)—NR³—N═N—, —N═N—NR³—, —C(═N—N(R³)₂)—, —C(═NR³)—, —C(═O)O—, —C(═O)S—, —C(═S)—, —O—P(═O)₂O—, —S—C═N—, or —C(═O)—NR³—O—, wherein groups with two chemically nonequivalent termini can adopt both possible orientations;
each LK^{NPL} is, independently, —(CH₂)_{pNPL}— or C₂₋₈ alkenylenyl, wherein each of the (CH₂)_{pNPL}— and C₂₋₈ alkenylenyl is optionally substituted with one or more substituents, wherein each substituent is, independently, amino, hydroxyl, aminoalkyl, hydroxylalkyl, or alkyl;
each pNPL is, independently, an integer from 0 to 8;
q1NPL and q2NPL are each, independently, 0, 1, or 2;
each PL is, independently, halo, hydroxyethoxymethyl, methoxyethoxymethyl, polyoxyethylene, or —(NR⁵′)_{q1PL}—U^{PL}-LK^{PL}—(NR⁵′)_{q2PL}—V, wherein:
R⁵, R⁵′, and R⁵″ are each, independently, hydrogen, alkyl, and alkoxy;
each U^{PL} is, independently, absent or O, S, S(═O), S(═O)₂, NR⁵, —C(═O)—, —C(═O)—NR⁵—, —C(═O)—N═N—NR⁵—, —C(═O)—NR⁵—N═N—, —N═N—NR⁵—, —C(═N—N(R⁵)₂)—, —C(═NR⁵)—, —C(═O)O—, —C(═O)S—, —C(═S)—, —O—P(═O)₂O—, —S—C═N—, or —C(═O)—NR⁵—O—, wherein groups with two chemically nonequivalent termini can adopt either of the two possible orientations;
each V is, independently, nitro, cyano, amino, halo, hydroxy, alkoxy, alkylthio, alkylamino, dialkylamino, —NH(CH₂)ₚNH₂ wherein p is 1 to 5, —C(═O)NH(CH₂)ₚNH₂ wherein p is 1 to 5, —C(═O)NH(CH₂)ₚNHC(═NH)NH₂ wherein p is 1 to 5, —C(═O)NH(CH₂)ₚNHC(═O)NH₂ wherein p is 1 to 5, —NHC(═O)-alkyl, —N(CH₂CH₂NH₂)₂, diazamino, amidino, guanidino, ureido, carbamoyl, —C(═O)OH, —C(═O)OR^{c}, —C(═O)NH—OH, —O—NH—C(═NH)NH₂, —NH—S(═O)₂OH, S(═O)₂OH, NR^{d}R^{e}, semicarbazone, aryl, cycloalkyl, heterocycloalkyl, or heteroaryl, wherein each of the aryl and cycloalkyl is substituted with one or more substitutents, wherein each of the heterocycloalkyl, and heteroaryl is optionally substituted with one or more substituents, and wherein each of the substituents for the aryl, cycloalkyl, heterocycloalkyl, and heteroaryl is, independently, nitro, cyano, amino, halo, hydroxy, alkoxy, alkylthio, alkylamino, dialkylamino, —NH(CH₂)ₚNH₂ wherein p is 1 to 5, —N(CH₂CH₂NH₂)₂, diazamino, amidino, guanidino, ureido, carbamoyl, —C(═O)OH, —C(═O)OR^{c}, —C(═O)NH—OH, —O—NH—C(═NH)NH₂, —NH—S(═O)₂OH, S(═O)₂OH, NR^{d}R^{e}, semicarbazone, aminosulfonyl, aminoalkoxy, aminoalkylthio, lower acylamino, or benzyloxycarbonyl;
each LK^{PL} is, independently, —(CH₂)_{pPL}— or C₂₋₈ alkenylenyl, wherein each of the —(CH₂)_{pNPL}— and C₂₋₈ alkenylenyl is optionally substituted with one or more substituents, wherein each substituent is, independently, amino, hydroxyl, aminoalkyl, hydroxylalkyl, or alkyl;
each pPL is, independently, an integer from 0 to 8;
q1PL and q2PL are each, independently, 0, 1, or 2; and
m is an integer from 1 to about 20;
t) Formula XIXa:
R¹—X-A₁-X—Y-A₂-Y—X-A₁-X—R² XIXa
or pharmaceutically acceptable salt thereof, wherein:
each X is, independently, NR⁸, O, S, or —N(R⁸)N(R⁸)—;
each Y is, independently, C═O, C═S, or O═S═O;
each R⁸ is, independently, hydrogen or alkyl;
A₁ and A₂ are each, independently, optionally substituted arylene or optionally substituted heteroarylene, wherein A₁ and A₂ are each, independently, optionally substituted with one or more PL group(s), one or more NPL group(s), or a combination of one or more PL group(s) and one or more NPL group(s);
R¹ is a PL group or an NPL group;
R² is R¹;
each NPL is, independently, —(NR³′)_{q1NPL}—U^{NPL}-LK^{NPL}—(NR³″)_{q2NPL}—R⁴′, wherein:
R³, R³′, and R³″ are each, independently, hydrogen, alkyl, or alkoxy;
R⁴ and R⁴′ are each, independently, hydrogen, alkyl, alkenyl, alkynyl, cycloalkyl, aryl, or heteroaryl, wherein each of the alkyl, alkenyl, alkynyl, cycloalkyl, aryl, and heteroaryl is optionally substituted with one or more alkyl or halo groups;
U^{NPL} is, independently, absent or O, S, S(═O), S(═O)₂, NR³, —C(═O)—, —C(═O)—N═N—NR³—, —C(═O)—NR³—N═N—, —N═N—NR³—, —C(═N—N(R³)₂)—, —C(═NR³)—, —C(═O)O—, —C(═O)S—, —C(═S)—, —O—P(═O)₂O—, —S—C═N—, or —C(═O)—NR³—O—, wherein groups with two chemically nonequivalent termini can adopt either of the two possible orientations;
each LK^{NPL} is, independently, —(CH₂)_{pNPL}— or C₂₋₈ alkenylenyl, wherein the —(CH₂)_{pNPL}— is optionally substituted with one or more substituents, wherein each substituent is, independently, amino, hydroxyl, or alkyl;
each pNPL is, independently, an integer from 0 to 8;
q1NPL and q2NPL are each, independently, 0, 1, or 2;
each PL is, independently, halo, hydroxyethoxymethyl, methoxyethoxymethyl, polyoxyethylene, or —(NR⁵′)_{q1PL}—U^{PL}-LK^{PL}—(NR⁵′)_{q2PL}—V, wherein:
R⁵, R⁵′, and R⁵″ are each, independently, hydrogen, alkyl, or alkoxy;
each U^{PL} is, independently, absent or O, S, S(═O), S(═O)₂, NR⁵, —C(═O)—, —C(═O)—N═N—NR⁵—, —C(═O)—NR⁵—N═N—, —N═N—NR⁵—, —C(═N—N(R⁵)₂)—, —C(═NR⁵)—, —C(═O)O—, —C(═O)S—, —C(═S)—, —O—P(═O)₂O—, —R⁵O—, —R⁵S—, —S—C═N—, or —C(═O)—NR⁵—O—, wherein groups with two chemically nonequivalent termini can adopt both possible orientations;
each V is, independently, nitro, cyano, amino, halo, hydroxy, alkoxy, alkylthio, alkylamino, dialkylamino, —NH(CH₂)ₚNH₂ wherein p is 1 to 5, —N(CH₂CH₂NH₂)₂, diazamino, amidino, guanidino, ureido, carbamoyl, —C(═O)OH, —C(═O)OR^{c}, —C(═O)NH—OH, —O—NH—C(═NH)NH₂, —NH—S(═O)₂OH, S(═O)₂OH, NR^{d}R^{e}, semicarbazone, aryl, heterocycloalkyl, or heteroaryl, wherein the aryl is substituted with one or more substitutents, wherein each of the heterocycloalkyl and heteroaryl is optionally substituted with one or more substituents, and wherein each of each of the substituents for the aryl, heterocycloalkyl, and heteroaryl is, independently, nitro, cyano, amino, halo, hydroxy, alkoxy, alkylthio, alkylamino, dialkylamino, —NH(CH₂)ₚNH₂ wherein p is 1 to 5, —N(CH₂CH₂NH₂)₂, diazamino, amidino, guanidino, ureido, carbamoyl, —C(═O)OH, —C(═O)OR^{c}, —C(═O)NH—OH, —O—NH—C(═NH)NH₂, —NH—S(═O)₂OH, S(═O)₂OH, NR^{d}R^{e}, semicarbazone, aminosulfonyl, aminoalkoxy, aminoalkylthio, lower acylamino, or benzyloxycarbonyl;
each LK^{PL} is, independently, —(CH₂)_{pPL}— or C₂₋₈ alkenylenyl, wherein the —(CH₂)_{pNPL}— is optionally substituted with one or more substituents, wherein each substituent is, independently, amino, hydroxyl, or alkyl;
each pPL is, independently, an integer from 0 to 8; and
q1PL and q2PL are each, independently, 0, 1, or 2;
u) Formula XX: 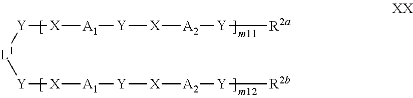
or a pharmaceutically acceptable salt thereof,
wherein:
each X is, independently, NR⁸;
each Y is C═O;
each R⁸ is, independently, hydrogen or alkyl;
each A₂ is optionally substituted arylene or optionally substituted heteroarylene, and each A₁ is —(CH₂)_{q}—, wherein q is 1 to 7, wherein A₁ and A₂ are each, independently, optionally substituted with one or more PL group(s), one or more NPL group(s), or a combination of one or more PL group(s) and one or more NPL group(s);
R² and R^{2a} are each, independently, hydrogen, a PL group, an NPL group or —X-A₁-Y—R¹¹, wherein R¹¹ is hydrogen, a PL group, or an NPL group;
L¹ is C₁₋₁₀alkylene optionally substituted with one or more substitutents, wherein each substituent is, independently, alkyl, halo, haloalkyl, aminoalkyl, hydroxylalkyl, V, or —(CH₂)_{pPL}—V, wherein pPL is an integer from 1 to 5;
each NPL group is, independently, —B(OR⁴)₂ or
—(NR³′)_{q1NPL}—U^{NPL}-LK^{NPL}—(NR³)_{q2NPL}—R⁴′, wherein:
R³, R³′, and R³″ are each, independently, hydrogen, alkyl, or alkoxy;
R⁴ and R⁴′ are each, independently, hydrogen, alkyl, alkenyl, alkynyl, cycloalkyl, aryl, or heteroaryl, wherein each of the alkyl, alkenyl, alkynyl, cycloalkyl, aryl, and heteroaryl is optionally substituted with one or more substitutents, wherein each substituent is, independently, alkyl, halo, or haloalkyl;
each U^{NPL} is, independently, absent or O, S, S(═O), S(═O)₂, NR³, —C(═O)—, —C(═O)—NR³—, —C(═O)—N═N—NR³—, —C(═O)—NR³—N═N—, —N═N—NR³—, —C(═N—N(R³)₂)—, —C(═NR³)—, —C(═O)O—, —C(═O)S—, —C(═S)—, —O—P(═O)₂O—, —S—C═N—, or —C(═O)—NR³—O—, wherein groups with two chemically nonequivalent termini can adopt both possible orientations;
each LK^{NPL} is, independently, —(CH₂)_{pNPL}— and C₂₋₈ alkenylenyl, wherein each of the —(CH₂)_{pNPL} and C₂₋₈ alkenylenyl is optionally substituted with one or more substituents, wherein each substituent is, independently, amino, hydroxyl, aminoalkyl, hydroxylalkyl, or alkyl;
each pNPL is, independently, an integer from 0 to 8;
q1NPL and q2NPL are each, independently, 0, 1, or 2;
each PL group is, independently, halo, hydroxyethoxymethyl, methoxyethoxymethyl, polyoxyethylene, or —(NR⁵′)_{q1PL}—U^{PL}-LK^{PL}—(NR⁵″)_{q2PL}—V, wherein:
R⁵, R⁵′, and R⁵″ are each, independently, hydrogen, alkyl, or alkoxy;
each U^{PL} is, independently, absent or O, S, S(═O), S(═O)₂, NR⁵, —C(═O)—, —C(═O)—NR⁵—, —C(═O)—N═N—NR⁵—, —C(═O)—NR⁵—N═N—, —N═N—NR⁵—, —C(═N—N(R⁵)₂)—, C(═NR⁵)—, —C(═O)O—, —C(═O)S—, —C(═S)—, —O—P(═O)₂O—, —S—C═N—, or —C(═O)—NR⁵—O—, wherein groups with two chemically nonequivalent termini can adopt either of the two possible orientations;
each V is, independently, nitro, cyano, amino, halo, hydroxy, alkoxy, alkylthio, alkylamino, dialkylamino, —NH(CH₂)ₚNH₂ wherein p is 1 to 5, —C(═O)NH(CH₂)ₚNH₂ wherein p is 1 to 5, —C(═O)NH(CH₂)ₚNHC(═NH)NH₂ wherein p is 1 to 5, —C(═O)NH(CH₂)ₚNHC(═O)NH₂ wherein p is 1 to 5, —NHC(═O)-alkyl, —N(CH₂CH₂NH₂)₂, diazamino, amidino, guanidino, ureido, carbamoyl, —C(═O)OH, —C(═O)OR^{c}, —C(═O)NH—OH, —O—NH—C(═NH)NH₂, —NH—S(═O)₂OH, S(═O)₂OH, NR^{d}R^{e}, semicarbazone, aryl, cycloalkyl, heterocycloalkyl, or heteroaryl, wherein each of the aryl and cycloalkyl is substituted with one or more substitutents, wherein each of the heterocycloalkyl and heteroaryl is optionally substituted with one or more substituents, and wherein each of the substituents for the aryl, cycloalkyl, heterocycloalkyl, and heteroaryl is, independently, nitro, cyano, amino, halo, hydroxy, alkoxy, alkylthio, alkylamino, dialkylamino, —NH(CH₂)ₚNH₂ wherein p is 1 to 5, —N(CH₂CH₂NH₂)₂, diazamino, amidino, guanidino, ureido, carbamoyl, —C(═O)OH, —C(═O)OR^{c}, —C(═O)NH—OH, —O—NH—C(═NH)NH₂, —NH—S(═O)₂OH, S(═O)₂OH, NR^{d}R^{e}, semicarbazone, aminosulfonyl, aminoalkoxy, aminoalkylthio, lower acylamino, or benzyloxycarbonyl;
each R^{c} is, independently, C₁₋₆ alkyl, C₁₋₆ haloalkyl, C₂₋₆ alkenyl, C₂₋₆ alkynyl, aryl, cycloalkyl, heteroaryl, heterocycloalkyl, arylalkyl, heteroarylalkyl, cycloalkylalkyl, or heterocycloalkylalkyl, each optionally substituted by one or more substitutents, wherein each substituent is, independently, OH, amino, halo, C₁₋₆ alkyl, C₁₋₆ haloalkyl, aryl, arylalkyl, heteroaryl, heteroarylalkyl, cycloalkyl, or heterocycloalkyl;
R^{d} and R^{e} are, independently, H, C₁₋₆ alkyl, C₁₋₆ haloalkyl, C₂₋₆ alkenyl, C₂₋₆ alkynyl, aryl, heteroaryl, cycloalkyl, heterocycloalkyl, arylalkyl, heteroarylalkyl, cycloalkylalkyl, or heterocycloalkylalkyl, wherein each of the C₁₋₆ alkyl, C₁₋₆ haloalkyl, C₂₋₆ alkenyl, C₂₋₆ alkynyl, aryl, heteroaryl, cycloalkyl, heterocycloalkyl, arylalkyl, heteroarylalkyl, cycloalkylalkyl, or heterocycloalkylalkyl is optionally substituted by OH, amino, halo, C₁₋₆ alkyl, C₁₋₆ haloalkyl, C₁₋₆ haloalkyl, aryl, arylalkyl, heteroaryl, heteroarylalkyl, cycloalkyl, or heterocycloalkyl;
or R^{d} and R^{e} together with the N atom to which they are attached form a 4-, 5-, 6-, 7-, or 8-membered heterocycloalkyl;
each LK^{PL} is, independently, —(CH₂)_{pPL}— or C₂₋₈ alkenylenyl, wherein each of the —(CH₂)_{pNPL}— and C₂₋₈ alkenylenyl is optionally substituted with one or more substituents, wherein each substituent is, independently, amino, hydroxyl, aminoalkyl, hydroxylalkyl, or alkyl;
each pPL is, independently, an integer from 0 to 8;
q1PL and q2PL are each, independently, 0, 1, or 2;
m11 is an integer from 1 to about 20; and
m12 is an integer from 1 to about 20;
v) Formula XXI:
R¹—[—X-A₁-Y—X-A₂-Y—]ₘ₁₃—X-L¹-Y—[—X-A₁-Y—X-A₂-Y—]ₘ₁₄—R² XXI
or a pharmaceutically acceptable salt thereof,
wherein:
each X is, independently, NR⁸;
each Y is C═O;
each R⁸ is, independently, hydrogen or alkyl;
each A₂ is optionally substituted arylene or optionally substituted heteroarylene, and each A₁ is —(CH₂)_{q}—, wherein q is 1 to 7, wherein A₁ and A₂ are each, independently, optionally substituted with one or more PL group(s), one or more NPL group(s), or a combination of one or more PL group(s) and one or more NPL group(s);
R¹ is hydrogen, a PL group, or an NPL group, and R² is —X-A₁-Y—R¹¹, wherein R¹¹ is hydrogen, a PL group, or an NPL group; or
R¹ and R² are each, independently, hydrogen, a PL group, or an NPL group; or
R¹ and R² together are a single bond; or
R¹ is —Y-A₂-X—R¹², wherein R¹² is hydrogen, a PL group, or an NPL group, and R² is hydrogen, a PL group, or an NPL group;
L¹ is C₁₋₁₀alkylene optionally substituted with one or more substitutents, wherein each substituent is, independently, alkyl, halo, haloalkyl, aminoalkyl, hydroxylalkyl, V, or —(CH₂)_{pPL}—V wherein pPL is an integer from 1 to 5;
each V is, independently, hydroxy, amino, alkylamino, dialkylamino, —NH(CH₂)ₚNH₂ wherein p is 1 to 5, —C(═O)NH(CH₂)ₚNH₂ wherein p is 1 to 5, C(═O)NH(CH₂)ₚNHC(═NH)NH₂ wherein p is 1 to 5, —C(═O)NH(CH₂)ₚNHC(═O)NH₂ wherein p is 1 to 5, —NHC(═O)-alkyl, —N(CH₂CH₂NH₂)₂, guanidino, amidino, ureido, carbamoyl, —C(═O)OH, —C(═O)OR^{c}, —C(═O)NH—OH, —O—NH—C(═NH)NH₂, —NH—S(═O)₂OH, S(═O)₂OH, NR^{d}R^{e}, a substituted aryl group, heterocycloalkyl, or heteroaryl, wherein each of the heterocycloalkyl and heteroaryl is optionally substituted with one more substituents, wherein each substituent is, independently, amino, halo, cyano, nitro, hydroxy, —NH(CH₂)ₚNH₂ wherein p is 1 to 5, —N(CH₂CH₂NH₂)₂, amidino, guanidino, aminosulfonyl, aminoalkoxy, aminoalkylthio, lower acylamino, or benzyloxycarbonyl; and wherein the substituted aryl group is substituted with one more substituents, wherein each substituent is, independently, amino, halo, cyano, nitro, hydroxy, —NH(CH₂)ₚNH₂ wherein p is 1 to 5, —N(CH₂CH₂NH₂)₂, amidino, guanidino, aminosulfonyl, aminoalkoxy, aminoalkylthio, lower acylamino, or benzyloxycarbonyl;
each NPL group is, independently, —B(OR⁴)₂ or
—(NR³′)_{q1NPL}—U^{NPL}-LK^{NPL}—(NR³″)_{q2NPL}—R⁴′, wherein:
R³, R³′, and R³″ are each, independently, hydrogen, alkyl, or alkoxy;
R⁴ and R⁴′ are each, independently, hydrogen, alkyl, alkenyl, alkynyl, cycloalkyl, aryl, or heteroaryl, wherein each of the alkyl, alkenyl, alkynyl, cycloalkyl, aryl, and heteroaryl is optionally substituted with one or more substitutents, wherein each substituent is, independently, alkyl, halo, or haloalkyl;
each U^{NPL} is, independently, absent or O, S, S(═O), S(═O)₂, NR³, —C(═O)—, —C(═O)—NR³—, —C(═O)—N═N—NR³—, —C(═O)—NR³—N═N—, —N═N—NR³—, —C(═N—N(R³)₂)—, —C(═NR³)—, —C(═O)O—, —C(═O)S—, —C(═S)—, —O—P(═O)₂O—, —S—C═N—, or —C(═O)—NR³—O—, wherein groups with two chemically nonequivalent termini can adopt both possible orientations;
each LK^{NPL} is, independently, —(CH₂)_{pNPL}— or C₂₋₈ alkenylenyl, wherein each of the —(CH₂)_{pNPL} and C₂₋₈ alkenylenyl is optionally substituted with one or more substituents, wherein each substituent is, independently, amino, hydroxyl, aminoalkyl, hydroxylalkyl, or alkyl;
each pNPL is, independently, an integer from 0 to 8;
q1NPL and q2NPL are each, independently, 0, 1, or 2;
each PL group is, independently, halo, hydroxyethoxymethyl, methoxyethoxymethyl, polyoxyethylene, or —(NR⁵′)_{q1PL}—U^{PL}-LK^{PL}—(NR⁵″)_{q2PL}—V, wherein:
R⁵, R⁵′, and R⁵″ are each, independently, hydrogen, alkyl, or alkoxy;
each U^{PL} is, independently, absent or O, S, S(═O), S(═O)₂, NR⁵, —C(═O)—, —C(═O)—NR⁵—, —C(═O)—N═N—NR⁵—, —C(═O)—NR⁵—N═N—, —N═N—NR⁵—, —C(═N—N(R⁵)₂)—, —C(═NR⁵)—, —C(═O)O—, —C(═O)S—, —C(═S)—, —O—P(═O)₂O—, —S—C═N—, or —C(═O)—NR⁵—O—, wherein groups with two chemically nonequivalent termini can adopt either of the two possible orientations;
each R^{c} is, independently, C₁₋₆ alkyl, C₁₋₆ haloalkyl, C₂₋₆ alkenyl, C₂₋₆ alkynyl, aryl, cycloalkyl, heteroaryl, heterocycloalkyl, arylalkyl, heteroarylalkyl, cycloalkylalkyl, or heterocycloalkylalkyl, each optionally substituted by one or more substitutents, wherein each substituent is, independently, OH, amino, halo, C₁₋₆ alkyl, C₁₋₆ haloalkyl, aryl, arylalkyl, heteroaryl, heteroarylalkyl, cycloalkyl, or heterocycloalkyl;
R^{d} and R^{e} are, independently, H, C₁₋₆ alkyl, C₁₋₆ haloalkyl, C₂₋₆ alkenyl, C₂₋₆ alkynyl, aryl, heteroaryl, cycloalkyl, heterocycloalkyl, arylalkyl, heteroarylalkyl, cycloalkylalkyl, or heterocycloalkylalkyl, wherein each of the C₁₋₆ alkyl, C₁₋₆ haloalkyl, C₂₋₆ alkenyl, C₂₋₆ alkynyl, aryl, heteroaryl, cycloalkyl, heterocycloalkyl, arylalkyl, heteroarylalkyl, cycloalkylalkyl, and heterocycloalkylalkyl is optionally substituted by OH, amino, halo, C₁₋₆ alkyl, C₁₋₆ haloalkyl, C₁₋₆ haloalkyl, aryl, arylalkyl, heteroaryl, heteroarylalkyl, cycloalkyl or heterocycloalkyl;
or R^{d} and R^{e} together with the N atom to which they are attached form a 4-, 5-, 6-, 7-, or 8-membered heterocycloalkyl;
each LK^{PL} is, independently, —(CH₂)_{pPL}— or C₂₋₈ alkenylenyl, wherein each of the —(CH₂)_{pNPL}— and C₂₋₈ alkenylenyl is optionally substituted with one or more substituents, wherein each substituent is, independently, amino, hydroxyl, aminoalkyl, hydroxylalkyl, or alkyl;
each pPL is, independently, an integer from 0 to 8;
q1PL and q2PL are each, independently, 0, 1, or 2;
m13 is an integer from 1 to about 10; and
m14 is an integer from 1 to about 10;
w) Formula XXII:
R¹—[—X-A₁-X—Z—Y-A₂-Y—Z]ₘ—R² XXII
or a pharmaceutically acceptable salt thereof,
wherein:
X is NR⁸, —NR⁸NR⁸—, C═O, or O;
Y is NR⁸, —NR⁸NR⁸—, C═O, S, or O;
R⁸ is hydrogen or alkyl;
Z is C═O, C═S, O═S═O, —NR⁸NR⁸—, or —C(═O)C(═O)—;
A₁ and A₂ are, independently, optionally substituted arylene or optionally substituted heteroarylene, wherein A₁ and A₂ are, independently, optionally substituted with one or more polar (PL) group(s), one or more non-polar (NPL) group(s), or a combination of one or more polar (PL) group(s) and one or more non-polar (NPL) group(s);
R¹ is
(i) hydrogen, a polar group (PL), or a non-polar group (NPL), and R² is —X-A₁-X—R¹, wherein A₁ is as defined above and is optionally substituted with one or more polar (PL) group(s), one or more non-polar (NPL) group(s), or a combination of one or more polar (PL) group(s) and one or more non-polar (NPL) group(s); or
(ii) hydrogen, a polar group (PL), or a non-polar group (NPL), and R² is —X-A₁-X—Z—Y-A₂-Y—R¹, wherein A₁ and A₂ are as defined above, and each of which is optionally substituted with one or more polar (PL) group(s), one or more non-polar (NPL) group(s), or a combination of one or more polar (PL) group(s) and one or more non-polar (NPL) group(s); or
(iii) hydrogen, a polar group (PL), or a non-polar group (NPL), and R² is —X-A′-X—R¹, wherein A′ is aryl or heteroaryl and is optionally substituted with one or more polar (PL) group(s), one or more non-polar (NPL) group(s), or a combination of one or more polar (PL) group(s) and one or more non-polar (NPL) group(s); or
(iv) hydrogen, a polar group (PL), or a non-polar group (NPL), and R² is —X-A₁-X—Z—Y-A′-Y—R¹, wherein A₁ is as defined above, A′ is aryl or heteroaryl, and each of A₁ and A′ is optionally substituted with one or more polar (PL) group(s), one or more non-polar (NPL) group(s), or a combination of one or more polar (PL) group(s) and one or more non-polar (NPL) group(s); or
(v) —Z—Y-A¹ and R² is hydrogen, a polar group (PL), or a non-polar group (NPL), wherein A′ is aryl or heteroaryl and is optionally substituted with one or more polar (PL) group(s), one or more non-polar (NPL) group(s), or a combination of one or more polar (PL) group(s) and one or more non-polar (NPL) group(s); or
(vi) —Z—Y-A′, and R² is —X-A″, wherein A′ and A″ are, independently, aryl or heteroaryl, and each of A and A″ is optionally substituted with one or more polar (PL) group(s), one or more non-polar (NPL) group(s), or a combination of one or more polar (PL) group(s) and one or more non-polar (NPL) group(s); or
(vii) R¹ and R² are, independently, a polar group (PL) or a non-polar group (NPL); or
(viii) R¹ and R² together form a single bond;
NPL is a nonpolar group independently selected from —B(OR⁴)₂ and
—(NR³′)_{q1NPL}—U^{NPL}—(CH₂)_{pNPL}—(NR³″)_{q2NPL}—R⁴′, wherein:
R³, R³′, and R³″ are, independently, selected from hydrogen, alkyl, and alkoxy;
R⁴ and R⁴′ are, independently, selected from hydrogen, alkyl, alkenyl, alkynyl, cycloalkyl, aryl, and heteroaryl, any of which is optionally substituted with one or more alkyl or halo groups;
U^{NPL} is absent or selected from O, S, S(═O), S(═O)₂, NR³, —C(═O)—, —C(═O)—N═N—NR³—, —C(═O)—NR³—N═N—, —N═N—NR³—, —C(═N—N(R³)₂)—, —C(═NR³)—, —C(═O)O—, —C(═O)S—, —C(═S)—, —O—P(═O)₂O—, —R³⁰—, —R³S—, —S—C═N—, and —C(═O)—NR³—O—, wherein groups with two chemically nonequivalent termini can adopt both possible orientations;
the —(CH₂)_{pNPL}— alkylene chain is optionally substituted with one or more amino or hydroxy groups, or is unsaturated;
pNPL is 0 to 8;
q1NPL and q2NPL are, independently, 0, 1, or 2;
PL is a polar group selected from halo, hydroxyethoxymethyl, methoxyethoxymethyl, polyoxyethylene, and —(NR⁵′)_{q1PL}—U^{PL}—(CH₂)_{pPL}—NR⁵′)_{q2PL}—V, wherein:
R⁵, R⁵′, and R⁵″ are, independently, selected from hydrogen, alkyl, and alkoxy;
U^{PL} is absent or selected from O, S, S(═O), S(═O)₂, NR⁵, —C(═O)—, —C(═O)—N═N—NR⁵—, —C(═O)—NR⁵—N═N—, —N═N—NR⁵—, —C(═N—N(R⁵)₂)—, —C(═NR⁵)—, —C(═O)O—, —C(═O)S—, —C(═S)—, —O—P(═O)₂O—, —R⁵O—, —R⁵S—, —S—C═N—, and —C(═O)—NR⁵—O—, wherein groups with two chemically nonequivalent termini can adopt both possible orientations;
V is selected from nitro, cyano, amino, hydroxy, alkoxy, alkylthio, alkylamino, dialkylamino, —NH(CH₂)ₚNH₂ wherein p is 1 to 4, —N(CH₂CH₂NH₂)₂, diazamino, amidino, guanidino, guanyl, semicarbazone, aryl, heterocycle, and heteroaryl, any of which is optionally substituted with one or more of amino, halo, cyano, nitro, hydroxy, —NH(CH₂)ₚNH₂ wherein p is 1 to 4, —N(CH₂CH₂NH₂)₂, amidino, guanidino, guanyl, aminosulfonyl, aminoalkoxy, aminoalkylthio, lower acylamino, or benzyloxycarbonyl;
the —(CH₂)_{pPL}— alkylene chain is optionally substituted with one or more amino or hydroxy groups, or is unsaturated;
pPL is 0 to 8;
q1PL and q2PL are, independently, 0, 1, or 2; and
m is 1 to about 20;
x) Formula XXIIa, Formula XXIIb, or Formula XXIIc:
R¹—X-A₁-X—Z—Y-A₂-Y—R² XXIIa
R¹—X-A₁-X—Z—Y-A₂-Y—Z—X-A₁-X—R² XXIIb
R¹—X-A₁-X—Z—Y-A₂-Y—Z—X-A₁-X—Z—Y-A₂-Y—R² XXIIc
wherein:
X is NR⁸, —NR⁸NR⁸—, C═O, or O;
Y is NR⁸, —NR⁸NR⁸—, C═O, S, or O;
R⁸ is hydrogen or alkyl;
Z is C═O, C═S, O═S═O, —NR⁸NR⁸—, or —C(═O)C(═O)—;
A₁ and A₂ are, independently, optionally substituted arylene or optionally substituted heteroarylene, wherein A₁ and A₂ are, independently, optionally substituted with one or more polar (PL) group(s), one or more non-polar (NPL) group(s), or a combination of one or more polar (PL) group(s) and one or more non-polar (NPL) group(s);
R¹ is hydrogen, a polar group (PL), or a non-polar group (NPL);
R² is R¹;
NPL is a nonpolar group —(NR³′)_{q1NPL}—U^{NPL}—(CH₂)_{pNPL}—(NR³″)_{q2NPL}—R⁴′, wherein:
R³, R³′, and R³″ are, independently, selected from hydrogen, alkyl, and alkoxy;
R⁴ and R⁴′ are, independently, selected from hydrogen, alkyl, alkenyl, alkynyl, cycloalkyl, aryl, and heteroaryl, any of which is optionally substituted with one or more alkyl or halo groups;
U^{NPL} is absent or selected from O, S, S(═O), S(═O)₂, NR³, —C(═O)—, —C(═O)—N═N—NR³—, —C(═O)—NR³—N═N—, —N═N—NR³—, —C(═N—N(R³)₂)—, —C(═NR³)—, —C(═O)O—, —C(═O)S—, —C(═S)—, —O—P(═O)₂O—, —R³⁰—, —R³S—, —S—C═N—, and —C(═O)—NR³—O—, wherein groups with two chemically nonequivalent termini can adopt both possible orientations;
the —(CH₂)_{pNPL}— alkylene chain is optionally substituted with one or more amino or hydroxy groups, or is unsaturated;
pNPL is 0 to 8;
q1NPL and q2NPL are, independently, 0, 1, or 2;
PL is a polar group selected from halo, hydroxyethoxymethyl, methoxyethoxymethyl, polyoxyethylene, and —(NR⁵′)_{q1PL}—U^{PL}—(CH₂)_{pPL}—(NR⁵′)_{q2PL}—V,
wherein:
R⁵, R⁵′, and R⁵″ are, independently, selected from hydrogen, alkyl, and alkoxy;
U^{PL} is absent or selected from O, S, S(═O), S(═O)₂, NR⁵, —C(═O)—, —C(═O)—N═N—NR⁵—, —C(═O)—NR⁵—N═N—, —N═N—NR⁵—, —C(═N—N(R⁵)₂)—, —C(═NR⁵)—, —C(═O)O—, —C(═O)S—, —C(═S)—, —O—P(═O)₂O—, —R⁵O—, —R⁵S—, —S—C═N—, and —C(═O)—NR⁵—O—, wherein groups with two chemically nonequivalent termini can adopt both possible orientations;
V is selected from nitro, cyano, amino, hydroxy, alkoxy, alkylthio, alkylamino, dialkylamino, —NH(CH₂)ₚNH₂ wherein p is 1 to 4, —N(CH₂CH₂NH₂)₂, diazamino, amidino, guanidino, guanyl, semicarbazone, aryl, heterocycle, and heteroaryl, any of which is optionally substituted with one or more of amino, halo, cyano, nitro, hydroxy, —NH(CH₂)ₚNH₂ wherein p is 1 to 4, —N(CH₂CH₂NH₂)₂, amidino, guanidino, guanyl, aminosulfonyl, aminoalkoxy, aminoalkylthio, lower acylamino, or benzyloxycarbonyl;
the —(CH₂)_{pPL}— alkylene chain is optionally substituted with one or more amino or hydroxy groups, or is unsaturated;
pPL is 0 to 8; and
q1PL and q2PL are, independently, 0, 1, or 2;
y) Formula XXIII:
R¹-[-A₁-W-A₂-W—]ₘR² XXIII
or a pharmaceutically acceptable salt thereof,
wherein:
A₁ and A₂ are, independently, optionally substituted arylene or optionally substituted heteroarylene, wherein:
(i) A₁ and A₂ are, independently, optionally substituted with one or more polar (PL) group(s), one or more non-polar (NPL) group(s), or a combination of one or more polar (PL) group(s) and one or more non-polar (NPL) group(s); or
(ii) one of A₁ or A₂ is as defined above and is optionally substituted with one or more polar (PL) group(s), one or more non-polar (NPL) group(s), or a combination of one or more polar (PL) group(s) and one or more non-polar (NPL) group(s); and the other of A₁ or
A₂ is the group —C≡C(CH₂)ₚC≡C—, wherein p is 0 to 8, and the —(CH₂)ₚ— alkylene chain is optionally substituted with one or more amino or hydroxyl groups;
W is absent, or represents —CH₂—, —CH₂—CH₂—, —CH═CH—, or —C≡C—;
R¹ is
(i) hydrogen, a polar group (PL), or a non-polar group (NPL), and R² is -A₁-R¹, wherein A₁ is as defined above and is optionally substituted with one or more polar (PL) group(s), one or more non-polar (NPL) group(s), or a combination of one or more polar (PL) group(s) and one or more non-polar (NPL) group(s); or
(ii) hydrogen, a polar group (PL), or a non-polar group (NPL), and R² is -A₁-W-A₂-R¹, wherein each of A₁ and A₂ is as defined above and is optionally substituted with one or more polar (PL) group(s), one or more non-polar (NPL) group(s), or a combination of one or more polar (PL) group(s) and one or more non-polar (NPL) group(s); or
(iii) A′-W— and R² is -A₁-W-A′, wherein A′ is aryl or heteroaryl, either of which is optionally substituted with one or more polar (PL) group(s), one or more non-polar (NPL) group(s), or a combination of one or more polar (PL) group(s) and one or more non-polar (NPL) group(s); or
(iv) A′-W— and R² is -A′, wherein A′ is aryl or heteroaryl, either of which is optionally substituted with one or more polar (PL) group(s), one or more non-polar (NPL) groups(s), or a combination of one or more polar (PL) group(s) and one or more non-polar (NPL) group(s); or
(iv) R¹ and R² together form a single bond;
NPL is a nonpolar group independently selected from —B(OR⁴)₂ or
—(NR³′)_{q1NPL}—U^{NPL}—(CH₂)_{pNPL}—(NR³″)_{q2NPL}—R⁴, wherein:
R³, R³′, and R³″ are, independently, selected from hydrogen, alkyl, and alkoxy;
R⁴ is selected from hydrogen, alkyl, alkenyl, alkynyl, cycloalkyl, aryl, and heteroaryl, any of which is optionally substituted with one or more alkyl or halo groups;
U^{NPL} is absent or selected from O, S, S(═O), S(═O)₂, NR³, —(C═O)O—, —(C═O)—N═N—NR³—, —(C═O)—NR³—N═N—, —N═N—NR³—, —C(═N—N(R³)₂)—, —C(═NR³)—, —C(═O)O—, —C(═O)S—, —C(═S)—, —O—P(═O)₂O—, —R³⁰—, —R³S—, —S—C═N— and —(C═O)—NR³—O—, wherein groups with two chemically nonequivalent termini can adopt both possible orientations;
the —(CH₂)_{pNPL}— alkylene chain is optionally substituted with one or more alkyl, amino or hydroxyl groups, or the alkylene chain is unsaturated;
pNPL is 0 to 8;
q1NPL and q2NPL are, independently, 0 to 2;
PL is a polar group selected from halo, hydroxyethoxymethyl, methoxyethoxymethyl, polyoxyethylene, and —(NR⁵′)_{q1PL}—U^{PL}—(CH₂)_{pPL}—(NR⁵′)_{q2PL}—V,
wherein:
R⁵, R⁵′, and R⁵″ are, independently, selected from hydrogen, alkyl, and alkoxy;
U^{PL} is absent or selected from O, S, S(═O), S(═O)₂, NR⁵, —(C═O)O—, —(C═O)—N═N—NR⁵—, —(C═O)—NR⁵—N═N—, —N═N—NR⁵—, —C(═N—N(R⁵)₂)—, —C(═NR⁵)—, —C(═O)O—, —C(═O)S—, —C(═S)—, —O—P(═O)₂O—, —R⁵O—, —R⁵S—, —S—C═N—, and —(C═O)—NR⁵—O—, wherein groups with two chemically nonequivalent termini can adopt both possible orientations;
V is selected from nitro, cyano, amino, hydroxyl, alkoxy, alkylthio, alkylamino, dialkylamino, —NH(CH₂)ₚNH₂, —N(CH₂CH₂NH₂)₂, diazamino, amidino, guanidino, guanyl, semicarbazone, aryl, heterocycle, and heteroaryl, any of which is optionally substituted with one or more of amino, halo, cyano, nitro, hydroxyl, —NH(CH₂)ₚNH₂, —N(CH₂CH₂NH₂)₂, amidino, guanidino, guanyl, aminosulfonyl, aminoalkoxy, aminoalkylthio, lower acylamino, or benzyloxycarbonyl;
the —(CH₂)_{pPL}— alkylene chain is optionally substituted with one or more amino or hydroxyl groups, or the alkylene chain is unsaturated;
pPL is 0 to 8;
q1PL and q2PL are, independently, 0 to 2; and
m is 1 to about 25;
z) Formula XXIIIa:
R¹-A₁-W-A₂-W-A₁-R² XXIIIa
wherein:
A₁ and A₂ are, independently, optionally substituted arylene or optionally substituted heteroarylene, wherein:
(i) A₁ and A₂ are, independently, optionally substituted with one or more polar (PL) group(s), one or more non-polar (NPL) group(s), or a combination of one or more polar (PL) group(s) and one or more non-polar (NPL) group(s); or
(ii) one of A₁ or A₂ is as defined above and is optionally substituted with one or more polar (PL) group(s), one or more non-polar (NPL) group(s), or a combination of one or more polar (PL) group(s) and one or more non-polar (NPL) group(s); and the other of A₁ or A₂ is the group —C≡C(CH₂)ₚC≡C—, wherein p is 0 to 8, and the —(CH₂)ₚ— alkylene chain is optionally substituted with one or more amino or hydroxyl groups;
W is —C≡C—;
R¹ is hydrogen, a polar group (PL), a non-polar group (NPL), or —W-A′, wherein A′ is aryl or heteroaryl, either of which is optionally substituted with one or more polar (PL) group(s), one or more non-polar (NPL) group(s), or a combination of one or more polar (PL) group(s) and one or more non-polar (NPL) group(s);
R² is R¹;
NPL is a nonpolar group —(NR₃′)_{q1NPL}—U^{NPL}—(CH₂)_{pNPL}—(NR₃″)_{q2NPL}—R⁴;
R³, R³′, and R³″ are, independently, selected from hydrogen, alkyl, and alkoxy;
R⁴ is selected from hydrogen, alkyl, alkenyl, alkynyl, cycloalkyl, aryl, and heteroaryl, any of which is optionally substituted with one or more alkyl or halo groups;
U^{NPL} is absent or selected from O, S, S(═O), S(═O)₂, NR³, —(C═O)O—, —(C═O)—N═N—NR³—, —(C═O)—NR³—N═N—, —N═N—NR³—, —C(═N—N(R³)₂)—, —C(═NR³)—, —C(═O)O—, —C(═O)S—, —C(═S)—, —O—P(═O)₂O—, —R³—O—, —R³—S—, —S—C═N—, and —(C═O)—NR³—O—, wherein groups with two chemically nonequivalent termini can adopt both possible orientations;
the alkylene chain —(CH₂)_{pNPL}— is optionally substituted with one or more alkyl, amino or hydroxyl groups, or the alkylene chain is unsaturated;
pNPL is 0 to 8;
q1NPL and q2NPL are, independently, 0 to 2;
PL is a polar group selected from halo, hydroxyethoxymethyl, methoxyethoxymethyl, polyoxyethylene, and —(NR⁵′)_{q1PL}—U^{PL}—(CH₂)_{pPL}—(NR⁵′)_{q2PL}—V,
wherein:
R⁵, R⁵′, and R⁵″ are, independently, selected from hydrogen, alkyl, and alkoxy;
U^{PL} is absent or selected from O, S, S(═O), S(═O)₂, NR⁵, —(C═O)O—, —(C═O)—N═N—NR⁵—, —(C═O)—NR⁵—N═N—, —N═N—NR⁵—, —C(═N—N(R⁵)₂)—, —C(═NR⁵)—, —C(═O)O—, —C(═O)S—, —C(═S)—, —O—P(═O)₂O—, —R⁵O—, —R⁵S—, —S—C═N—, and —(C═O)—NR⁵—O—, wherein groups with two chemically nonequivalent termini can adopt both possible orientations;
V is selected from nitro, cyano, amino, hydroxyl, alkoxy, alkylthio, alkylamino, dialkylamino, —NH(CH₂)ₚNH₂, —N(CH₂CH₂NH₂)₂, diazamino, amidino, guanidino, guanyl, semicarbazone, aryl, heterocycle, and heteroaryl, any of which is optionally substituted with one or more of amino, halo, cyano, nitro, hydroxyl, —NH(CH₂)ₚNH₂, —N(CH₂CH₂NH₂)₂, amidino, guanidino, guanyl, aminosulfonyl, aminoalkoxy, aminoalkylthio, lower acylamino, or benzyloxycarbonyl;
the alkylene chain —(CH₂)_{pPL}— is optionally substituted with one or more amino or hydroxyl groups, or the alkylene chain is unsaturated;
pPL is 0 to 8; and
q1PL and q2PL are, independently, 0 to 2;
aa) Formula XXIV:
R¹—X-A₁-X—Y-A₂-Y—X-A₁-X—R² XXIV
or a pharmaceutically acceptable salt thereof,
wherein:
X is NR⁸, O, S, or —N(R⁸)N(R⁸)—;
Y is C═O, C═S, or O═S═O;
R⁸ is hydrogen or alkyl;
A₁ and A₂ are, independently, optionally substituted arylene or optionally substituted heteroarylene, wherein A₁ and A₂ are, independently, optionally substituted with one or more polar (PL) group(s), one or more non-polar (NPL) group(s), or a combination of one or more polar (PL) group(s) and one or more non-polar (NPL) group(s);
R¹ is a polar group (PL) or a non-polar group (NPL);
R² is R¹;
NPL is a nonpolar group independently selected from —B(OR⁴)₂ and —(NR³′)_{q1NPL}—U^{NPL}—(CH₂)_{pNPL}—(NR³″)_{q2NPL}—R⁴′, wherein:
R³, R³′, and R³″ are, independently, selected from hydrogen, alkyl, and alkoxy;
R⁴ and R⁴′ are, independently, selected from the group consisting of hydrogen, alkyl, alkenyl, alkynyl, cycloalkyl, aryl, and heteroaryl, any of which is optionally substituted with one or more alkyl or halo groups;
U^{NPL} is absent or selected from O, S, S(═O), S(═O)₂, NR³, —C(═O)—, —C(═O)—N═N—NR³—, —C(═O)—NR³—N═N—, —N═N—NR³—, —C(═N—N(R³)₂)—, —C(═NR³)—, —C(═O)O—, —C(═O)S—, —C(═S)—, —O—P(═O)₂O—, —R³⁰—, —R³S—, —S—C═N—, and —C(═O)—NR³—O—, wherein groups with two chemically nonequivalent termini can adopt both possible orientations;
the —(CH₂)_{pNPL}— alkylene chain is optionally substituted with one or more amino or hydroxy groups, or is unsaturated;
pNPL is 0 to 8;
q1NPL and q2NPL are, independently, 0, 1, or 2;
PL is a polar group selected from halo, hydroxyethoxymethyl, methoxyethoxymethyl, polyoxyethylene, and —(NR⁵′)_{q1PL}—U^{PL}—(CH₂)_{pPL}—(NR⁵′)_{q2PL}—V,
wherein:
R⁵, R⁵′, and R⁵″ are, independently, selected from hydrogen, alkyl, and alkoxy;
U^{PL} is absent or selected from O, S, S(═O), S(═O)₂, NR⁵, —C(═O)—, —C(═O)—N═N—NR⁵—, —C(═O)—NR⁵—N═N—, —N═N—NR⁵—, —C(═N—N(R⁵)₂)—, —C(═NR⁵)—, —C(═O)O—, —C(═O)S—, —C(═S)—, —O—P(═O)₂O—, —R⁵O—, —R⁵S—, —S—C═N—, and —C(═O)—NR⁵—O—, wherein groups with two chemically nonequivalent termini can adopt both possible orientations;
V is selected from nitro, cyano, amino, hydroxy, alkoxy, alkylthio, alkylamino, dialkylamino, —NH(CH₂)ₚNH₂ wherein p is 1 to 4, —N(CH₂CH₂NH₂)₂, diazamino, amidino, guanidino, guanyl, semicarbazone, aryl, heterocycle and heteroaryl, any of which is optionally substituted with one or more of amino, halo, cyano, nitro, hydroxy, —NH(CH₂)ₚNH₂ wherein p is 1 to 4, —N(CH₂CH₂NH₂)₂, amidino, guanidino, guanyl, aminosulfonyl, aminoalkoxy, aminoalkylthio, lower acylamino, or benzyloxycarbonyl;
the —(CH₂)_{pPL}— alkylene chain is optionally substituted with one or more amino or hydroxy groups, or is unsaturated;
pPL is 0 to 8; and
q1PL and q2PL are, independently, 0, 1, or 2; or
bb) Formula XXV:
A-(B)ₙ₁-(D)ₘ₁-H XXV
or a pharmaceutically acceptable salt thereof,
wherein:
A is the residue of a chain transfer agent;
B is —[CH₂—C(R¹¹)(B₁₁)], wherein B₁₁ is —X₁₁—Y₁₁—Z₁₁, wherein X₁₁ is carbonyl (—C(═O)—) or optionally substituted C₁₋₆ alkylene; or X₁₁ is absent;
Y₁₁ is O, NH, or optionally substituted C₁₋₆ alkylene; or Y₁₁ is absent;
Z₁₁ is —Z_{11A}—Z_{11B}, wherein Z_{11A} is alkylene, arylene, or heteroarylene, any of which is optionally substituted; or Z_{11A} is absent; and Z_{11B} is -guanidino, -amidino, —N(R³)(R⁴), or —N⁺(R³)(R⁴)(R⁵), wherein R³, R⁴, and R⁵ are, independently, hydrogen, alkyl, aminoalkyl, aryl, heteroaryl, heterocyclic, or aralkyl; or
Z₁₁ is pyridinium 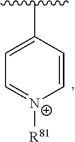
or phosphonium 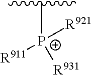
wherein R⁸¹, R⁹¹¹, R⁹²¹, and R⁹³¹ are, independently, hydrogen or alkyl;
R¹¹ is hydrogen or C₁₋₄ alkyl;
D is —[CH₂—C(R²¹)(D₂₁)]-, wherein D₂₁ is —X₂₁—Y₂₁—Z₂₁, wherein
X₂₁ is carbonyl (—C(═O)—) or optionally substituted C₁₋₆ alkylene; or X₂₁ is absent;
Y₂₁ is O, NH, or optionally substituted C₁₋₆ alkylene, or Y₂₁ is absent;
Z₂₁ is alkyl, cycloalkyl, alkoxy, aryl, or aralkyl, any of which is optionally substituted;
R²¹ is hydrogen or C₁₋₄ alkyl;
m₁, the mole fraction of D, is about 0.1 to about 0.9; and
n₁, the mole fraction of B, is 1−m₁;
wherein the compound is a random copolymer of B and D, and
wherein the copolymer has a degree of polymerization of about 5 to about 50.

2. The method of claim 1 wherein the method of modulating an immune response comprises decreasing the production of a cytokine.

3. The method of claim 2 wherein the cytokine is chosen from TNFalpha, IL-1Beta, IL-1alpha, IL-8, IL-6, IL-10, IL-11, IL-12, TGF-Beta, and IFNgamma.

4. The method of claim 1 wherein the immune response is against an oral pathogen.

5. The method of claim 4 wherein the oral pathogen is chosen from Aggregatibacter actinomycetemcomitans, Porphyromonas gingivalis, Streptococcus sanguis, Candida albicans, Actinomyces viscosus, Lactobacillus casei, and Strept. mutans.

6. The method of claim 1 wherein the immune response is against a bacterial pathogen.

7. The method of claim 6 wherein the bacterial pathogen is chosen from S. aureus, methicillin-resistant S. aureus, S. epidermidis, Strept. pneumoniae, Strept. pyogenes, Strept. viridans, E. coli, E. faecalis, E. faecium, P. aeruginosa, A. baumannii, Haemophilus influenzae, Serratia marcescens, Moraxella catarrhalis, Klebsiella pneumoniae, Proteus vulgaris, Proteus mirabilis, Bacteroides fragalis, Clostridium difficile, Clostridium perfringens, and P. acnes.
